SEQUENCE LISTING (27 sequences)

SEQ ID NO: 1 - DNA, 1680 bp, Human betaherpesvirus 5
  source 1..1680: mol_type = genomic DNA; note = UL82 gene, Human herpesvirus 5 strain TR (GenBank Accession No. KF021605.1:118811 120490)
ctagatgcggggtcgactgcgtggggtgctggaagtggaagcggtgctgatgggtgagggtcgtggcgcgggcacggaccgcaacgtgctgctgatgtctgccgcggtacgcacgtcgccgtccatgtcgctgcgcagataagaggtaggtcgtaatgcggcgtgctgcacgctcaccgttaatggtaccaagtcgtcaaggctcgcaaagacgtgccacgaggggatgacgagcgtgagagccccgttgttaccgcttcgacgtctttgtccggtcaggatcagtgcccgggacagtccggcttgggtgtccgagtcctcgtcgccgctggcctcctcgaagccggcaaacatggcttcggacaggggggtcggcgtcggtgtggaggagaggtcatcttcgtcgtcctcttcctcttcttcctcctcttcctcggtgggtggtaatccgggggactgcgggagaaactcggagacggcgccgcgcacgacgtcgctccgtggaaagagaccggcgcgcagctgcacctggggacgcttgattttgtccggtttaccgggtgtgagagtccaaaacccacggcggaaaaagtggatgcggcctagcggctgtcggtgttccaaatgaacggcctggtcgccggtcagcgtgacgcggagggtgattcgcacacgatcgggtagcgggccggcttctatggagacgcccgggatgttttccgggaaaaagatggtgtcgtgagtctgattggtttcgaaagcattctggatctgcacgatgtactcgggatgtatgcgcgtcagcgtaaaacttttgggaatcaacagctggaagccgttgtccggcaagcgtcgtaggtgcgggtacggattgtgtcgcgccaccacctcggcgcgatgcgtgtaaaccgaaaagtgcagaaacacgctggtcggcgggtgcggtgagtcgtgatgcagaaacagcatgatccattggcctcgttcgtccgtctccgttttgtggatgtacgtgttagggtccgaacaggccagctgctccagggcgtctaccagcgtcagcgggatggcgccggcgcgaaaggcgaactggctgacaaagatctgccctgcctccaaactgctgtcggttctgcggcgccagttcggcgtcacggtcagtcgcacggcccagtggtgagccgtgcggcggatgatggcgcgcgcctccattcgcggccgattttcttcgccgccgcgccgctggctctgaaagaggtgcagtccgctaacgggcacgcggtctagcggcagcgcaaaggccagcaccgagaccgtgttgttttctgagcctggcgtcaggcgtcgtgggccaaagttgttgaggtccaccagcagtcggtcccgttcgcccaccacgcagcggcccttgatgtttaggtcggtcaggtctacggtgtcgtgcggagatttgttctcctgaaaacagcagagaaccgagggtcggctcacctctatgttggtacgcaggtccaggagtcgcagacgaccggcttccagcgagccgccttccacgttggtgatgagccgaagcacctggcagtgcaggcgaccaaagctgccgctggcggcttcggcctcgctgatcgcggccgcttccgacgagggtccctcaccgggcgaggacgatgcctgagacat

SEQ ID NO: 2 - protein, 559 aa, Human betaherpesvirus 5
  source 1..559: note = tegument protein pp71, Human betaherpesvirus 5 (GenBank Accession No. AGL96671.1)
MSQASSSPGEGPSSEAAAISEAEAASGSFGRLHCQVLRLITNVEGGSLEAGRLRLLDLRTNIEVSRPSVLCCFQENKSPHDTVDLTDLNIKGRCVVGERDRLLVDLNNFGPRRLTPGSENNTVSVLAFALPLDRVPVSGLHLFQSQRRGGEENRPRMEARAIIRRTAHHWAVRLTVTPNWRRRTDSSLEAGQIFVSQFAFRAGAIPLTLVDALEQLACSDPNTYIHKTETDERGQWIMLFLHHDSPHPPTSVFLHFSVYTHRAEVVARHNPYPHLRRLPDNGFQLLIPKSFTLTRIHPEYIVQIQNAFETNQTHDTIFFPENIPGVSIEAGPLPDRVRITLRVTLTGDQAVHLEHRQPLGRIHFFRRGFWTLTPGKPDKIKRPQVQLRAGLFPRSDVVRGAVSEFLPQSPGLPPTEEEEEEEEEDDEDDLSSTPTPTPLSEAMFAGFEEASGDEDSDTQAGLSRALILTGQRRRSGNNGALTLVIPSWHVFASLDDLVPLTVSVQHAALRPTSYLRSDMDGDVRTAADISSTLRSVPAPRPSPISTASTSSTPRSRPRI

SEQ ID NO: 3 - protein, 559 aa, Human betaherpesvirus 5; identical to SEQ ID NO: 2
  source 1..559: note = Tegument protein pp71, Human betaherpesvirus 5 (UniProtKB R4SH92)

SEQ ID NO: 4 - RNA, 2114 nt, synthetic construct
  source 1..2114: mol_type = other RNA; note = pp71 Construct B, Full length (FT)
agggccacccgccgcgcacgcgcttaagacgactctataaaaacccacgtccactcagacacgcgacttttgggcgccacacctgtcgccgctgctatatttgcgacagttgccggaacccttcccgacctcccacgaagacccgttcacctttgcgcatcccctgaccctccccccatcccgccttcgcaatgtctcaggcatcgtcctcgcccggtgagggaccctcgtcggaagcggccgcgatcagcgaggccgaagccgccagcggcagctttggtcgcctgcactgccaggtgcttcggctcatcaccaacgtggaaggcggctcgctggaagccggtcgtctgcgactcctggacctgcgtaccaacatagaggtgagccgaccctcggttctctgctgttttcaggagaacaaatctccgcacgacaccgtagacctgaccgacctaaacatcaagggccgctgcgtggtgggcgaacgggaccgactgctggtggacctcaacaactttggcccacgacgcctgacgccaggctcagaaaacaacacggtctcggtgctggcctttgcgctgccgctagaccgcgtgcccgttagcggactgcacctctttcagagccagcggcgcggcggcgaagaaaatcggccgcgaatggaggcgcgcgccatcatccgccgcacggctcaccactgggccgtgcgactgaccgtgacgccgaactggcgccgcagaaccgacagcagtttggaggcagggcagatctttgtcagccagttcgcctttcgcgccggcgccatcccgctgacgctggtagacgccctggagcagctggcctgttcggaccctaacacgtacatccacaaaacggagacggacgaacgaggccaatggatcatgctgtttctgcatcacgactcaccgcacccgccgaccagcgtgtttctgcacttttcggtttacacgcatcgcgccgaggtggtggcgcgacacaatccgtacccgcacctacgacgcttgccggacaacggcttccagctgttgattcccaaaagttttacgctgacgcgcatacatcccgagtacatcgtgcagatccagaatgctttcgaaaccaatcagactcacgacaccatctttttcccggaaaacatcccgggcgtctccatagaagccggcccgctacccgatcgtgtgcgaatcaccctccgcgtcacgctgaccggcgaccaggccgttcatttggaacaccgacagccgctaggccgcatccactttttccgccgtgggttttggactctcacacccggtaaaccggacaaaatcaagcgtccccaggtgcagctgcgcgccggtctctttccacggagcgacgtcgtgcgcggcgccgtctccgagtttctcccgcagtcccccggattaccacccaccgaggaagaggaggaagaagaggaagaggacgacgaagatgacctctcctccacaccgacgccgacccccctgtccgaagccatgtttgccggcttcgaggaggccagcggcgacgaggactcggacacccaagccggactgtcccgggcactgatcctgaccggacaaagacgtcgaagcggtaacaacggggctctcacgctcgtcatcccctcgtggcacgtctttgcgagccttgacgacttggtaccattaacggtgagcgtgcagcacgccgcattacgacctacctcttatctgcgcagcgacatggacggcgacgtgcgtaccgcggcagacatcagcagcacgttgcggtccgtgcccgcgccacgaccctcacccatcagcaccgcttccacttccagcaccccacgcagtcgaccccgcatctagagagagacttctttgtttttcccccgcgtgtttttcccattccctgtatttatttctaaataataaaaacacagagacgttgataataaccgcagtgtgctttattagggtatcacggtgtagaaaaaaaaaagagagggaaaccctaaatatagcgtctctcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - RNA, 2045 nt, synthetic construct
  source 1..2045: mol_type = other RNA; note = pp71 Construct B.2, Full length (FT)
gccacccgccgcgcacgcgcttaagacgactctataaaaacccacgtccactcagacacgcgacttttgggcgccacacctgtcgccgctgctatatttgcgacagttgccggaacccttcccgacctcccacgaagacccgttcacctttgcgcatcccctgaccctccccccatcccgccttcgcaatgtctcaggcatcgtcctcgcccggtgagggaccctcgtcggaagcggccgcgatcagcgaggccgaagccgccagcggcagctttggtcgcctgcactgccaggtgcttcggctcatcaccaacgtggaaggcggctcgctggaagccggtcgtctgcgactcctggacctgcgtaccaacatagaggtgagccgaccctcggttctctgctgttttcaggagaacaaatctccgcacgacaccgtagacctgaccgacctaaacatcaagggccgctgcgtggtgggcgaacgggaccgactgctggtggacctcaacaactttggcccacgacgcctgacgccaggctcagaaaacaacacggtctcggtgctggcctttgcgctgccgctagaccgcgtgcccgttagcggactgcacctctttcagagccagcggcgcggcggcgaagaaaatcggccgcgaatggaggcgcgcgccatcatccgccgcacggctcaccactgggccgtgcgactgaccgtgacgccgaactggcgccgcagaaccgacagcagtttggaggcagggcagatctttgtcagccagttcgcctttcgcgccggcgccatcccgctgacgctggtagacgccctggagcagctggcctgttcggaccctaacacgtacatccacaaaacggagacggacgaacgaggccaatggatcatgctgtttctgcatcacgactcaccgcacccgccgaccagcgtgtttctgcacttttcggtttacacgcatcgcgccgaggtggtggcgcgacacaatccgtacccgcacctacgacgcttgccggacaacggcttccagctgttgattcccaaaagttttacgctgacgcgcatacatcccgagtacatcgtgcagatccagaatgctttcgaaaccaatcagactcacgacaccatctttttcccggaaaacatcccgggcgtctccatagaagccggcccgctacccgatcgtgtgcgaatcaccctccgcgtcacgctgaccggcgaccaggccgttcatttggaacaccgacagccgctaggccgcatccactttttccgccgtgggttttggactctcacacccggtaaaccggacaaaatcaagcgtccccaggtgcagctgcgcgccggtctctttccacggagcgacgtcgtgcgcggcgccgtctccgagtttctcccgcagtcccccggattaccacccaccgaggaagaggaggaagaagaggaagaggacgacgaagatgacctctcctccacaccgacgccgacccccctgtccgaagccatgtttgccggcttcgaggaggccagcggcgacgaggactcggacacccaagccggactgtcccgggcactgatcctgaccggacaaagacgtcgaagcggtaacaacggggctctcacgctcgtcatcccctcgtggcacgtctttgcgagccttgacgacttggtaccattaacggtgagcgtgcagcacgccgcattacgacctacctcttatctgcgcagcgacatggacggcgacgtgcgtaccgcggcagacatcagcagcacgttgcggtccgtgcccgcgccacgaccctcacccatcagcaccgcttccacttccagcaccccacgcagtcgaccccgcatctagagagagacttctttgtttttcccccgcgtgtttttcccattccctgtatttatttctaaataataaaaacacagagacgttgataataaccgcagtgtgctttattagggtatcacggtgtagaaaaaaaaagagagggaaaccctaaatatagcgtctctcagcctgagtaggaag

SEQ ID NO: 6 - RNA, 2031 nt, synthetic construct
  source 1..2031: mol_type = other RNA; note = pp71 Construct B.3, Full length (FT)
gccacccgccgcgcacgcgcttaagacgactctataaaaacccacgtccactcagacacgcgacttttgggcgccacacctgtcgccgctgctatatttgcgacagttgccggaacccttcccgacctcccacgaagacccgttcacctttgcgcatcccctgaccctccccccatcccgccttcgcaatgtctcaggcatcgtcctcgcccggtgagggaccctcgtcggaagcggccgcgatcagcgaggccgaagccgccagcggcagctttggtcgcctgcactgccaggtgcttcggctcatcaccaacgtggaaggcggctcgctggaagccggtcgtctgcgactcctggacctgcgtaccaacatagaggtgagccgaccctcggttctctgctgttttcaggagaacaaatctccgcacgacaccgtagacctgaccgacctaaacatcaagggccgctgcgtggtgggcgaacgggaccgactgctggtggacctcaacaactttggcccacgacgcctgacgccaggctcagaaaacaacacggtctcggtgctggcctttgcgctgccgctagaccgcgtgcccgttagcggactgcacctctttcagagccagcggcgcggcggcgaagaaaatcggccgcgaatggaggcgcgcgccatcatccgccgcacggctcaccactgggccgtgcgactgaccgtgacgccgaactggcgccgcagaaccgacagcagtttggaggcagggcagatctttgtcagccagttcgcctttcgcgccggcgccatcccgctgacgctggtagacgccctggagcagctggcctgttcggaccctaacacgtacatccacaaaacggagacggacgaacgaggccaatggatcatgctgtttctgcatcacgactcaccgcacccgccgaccagcgtgtttctgcacttttcggtttacacgcatcgcgccgaggtggtggcgcgacacaatccgtacccgcacctacgacgcttgccggacaacggcttccagctgttgattcccaaaagttttacgctgacgcgcatacatcccgagtacatcgtgcagatccagaatgctttcgaaaccaatcagactcacgacaccatctttttcccggaaaacatcccgggcgtctccatagaagccggcccgctacccgatcgtgtgcgaatcaccctccgcgtcacgctgaccggcgaccaggccgttcatttggaacaccgacagccgctaggccgcatccactttttccgccgtgggttttggactctcacacccggtaaaccggacaaaatcaagcgtccccaggtgcagctgcgcgccggtctctttccacggagcgacgtcgtgcgcggcgccgtctccgagtttctcccgcagtcccccggattaccacccaccgaggaagaggaggaagaagaggaagaggacgacgaagatgacctctcctccacaccgacgccgacccccctgtccgaagccatgtttgccggcttcgaggaggccagcggcgacgaggactcggacacccaagccggactgtcccgggcactgatcctgaccggacaaagacgtcgaagcggtaacaacggggctctcacgctcgtcatcccctcgtggcacgtctttgcgagccttgacgacttggtaccattaacggtgagcgtgcagcacgccgcattacgacctacctcttatctgcgcagcgacatggacggcgacgtgcgtaccgcggcagacatcagcagcacgttgcggtccgtgcccgcgccacgaccctcacccatcagcaccgcttccacttccagcaccccacgcagtcgaccccgcatctagagagagacttctttgtttttcccccgcgtgtttttcccattccctgtatttatttctaaataataaaaacacagagacgttgataataaccgcagtgtgctttattagggtatcacggtgtagaaaaaaaaaagagagggaaaccctaaatatagcgtctctc

SEQ ID NO: 7 - RNA, 1928 nt, synthetic construct
  source 1..1928: mol_type = other RNA; note = pp71 Construct C, Immediate early (IE1)
aggtcgtttagtgaaccgtcagatcgcctggagacgccatccacgctgttttgacctccatagaagacaccgggaccgatccagcctccgcggccgggaacggtgcattggaacgcggattccccgtgccaagagtgacatgtctcaggcatcgtcctcgcccggtgagggaccctcgtcggaagcggccgcgatcagcgaggccgaagccgccagcggcagctttggtcgcctgcactgccaggtgcttcggctcatcaccaacgtggaaggcggctcgctggaagccggtcgtctgcgactcctggacctgcgtaccaacatagaggtgagccgaccctcggttctctgctgttttcaggagaacaaatctccgcacgacaccgtagacctgaccgacctaaacatcaagggccgctgcgtggtgggcgaacgggaccgactgctggtggacctcaacaactttggcccacgacgcctgacgccaggctcagaaaacaacacggtctcggtgctggcctttgcgctgccgctagaccgcgtgcccgttagcggactgcacctctttcagagccagcggcgcggcggcgaagaaaatcggccgcgaatggaggcgcgcgccatcatccgccgcacggctcaccactgggccgtgcgactgaccgtgacgccgaactggcgccgcagaaccgacagcagtttggaggcagggcagatctttgtcagccagttcgcctttcgcgccggcgccatcccgctgacgctggtagacgccctggagcagctggcctgttcggaccctaacacgtacatccacaaaacggagacggacgaacgaggccaatggatcatgctgtttctgcatcacgactcaccgcacccgccgaccagcgtgtttctgcacttttcggtttacacgcatcgcgccgaggtggtggcgcgacacaatccgtacccgcacctacgacgcttgccggacaacggcttccagctgttgattcccaaaagttttacgctgacgcgcatacatcccgagtacatcgtgcagatccagaatgctttcgaaaccaatcagactcacgacaccatctttttcccggaaaacatcccgggcgtctccatagaagccggcccgctacccgatcgtgtgcgaatcaccctccgcgtcacgctgaccggcgaccaggccgttcatttggaacaccgacagccgctaggccgcatccactttttccgccgtgggttttggactctcacacccggtaaaccggacaaaatcaagcgtccccaggtgcagctgcgcgccggtctctttccacggagcgacgtcgtgcgcggcgccgtctccgagtttctcccgcagtcccccggattaccacccaccgaggaagaggaggaagaagaggaagaggacgacgaagatgacctctcctccacaccgacgccgacccccctgtccgaagccatgtttgccggcttcgaggaggccagcggcgacgaggactcggacacccaagccggactgtcccgggcactgatcctgaccggacaaagacgtcgaagcggtaacaacggggctctcacgctcgtcatcccctcgtggcacgtctttgcgagccttgacgacttggtaccattaacggtgagcgtgcagcacgccgcattacgacctacctcttatctgcgcagcgacatggacggcgacgtgcgtaccgcggcagacatcagcagcacgttgcggtccgtgcccgcgccacgaccctcacccatcagcaccgcttccacttccagcaccccacgcagtcgaccccgcatctaggcggccgcttaattaagctgccttctgcggggcttgccttctggccatgcccttcttctctcccttgcacctgtacctcttggtctttgaataaagcctgagtaggaag

SEQ ID NO: 8 - RNA, 1909 nt, synthetic construct
  source 1..1909: mol_type = other RNA; note = pp71 Construct D, short
agggcgccacacctgtcgccgctgctatatttgcgacagttgccggaacccttcccgacctcccacgaagacccgttcacctttgcgcatcccctgaccctccccccatcccgccttcgcaatgtctcaggcatcgtcctcgcccggtgagggaccctcgtcggaagcggccgcgatcagcgaggccgaagccgccagcggcagctttggtcgcctgcactgccaggtgcttcggctcatcaccaacgtggaaggcggctcgctggaagccggtcgtctgcgactcctggacctgcgtaccaacatagaggtgagccgaccctcggttctctgctgttttcaggagaacaaatctccgcacgacaccgtagacctgaccgacctaaacatcaagggccgctgcgtggtgggcgaacgggaccgactgctggtggacctcaacaactttggcccacgacgcctgacgccaggctcagaaaacaacacggtctcggtgctggcctttgcgctgccgctagaccgcgtgcccgttagcggactgcacctctttcagagccagcggcgcggcggcgaagaaaatcggccgcgaatggaggcgcgcgccatcatccgccgcacggctcaccactgggccgtgcgactgaccgtgacgccgaactggcgccgcagaaccgacagcagtttggaggcagggcagatctttgtcagccagttcgcctttcgcgccggcgccatcccgctgacgctggtagacgccctggagcagctggcctgttcggaccctaacacgtacatccacaaaacggagacggacgaacgaggccaatggatcatgctgtttctgcatcacgactcaccgcacccgccgaccagcgtgtttctgcacttttcggtttacacgcatcgcgccgaggtggtggcgcgacacaatccgtacccgcacctacgacgcttgccggacaacggcttccagctgttgattcccaaaagttttacgctgacgcgcatacatcccgagtacatcgtgcagatccagaatgctttcgaaaccaatcagactcacgacaccatctttttcccggaaaacatcccgggcgtctccatagaagccggcccgctacccgatcgtgtgcgaatcaccctccgcgtcacgctgaccggcgaccaggccgttcatttggaacaccgacagccgctaggccgcatccactttttccgccgtgggttttggactctcacacccggtaaaccggacaaaatcaagcgtccccaggtgcagctgcgcgccggtctctttccacggagcgacgtcgtgcgcggcgccgtctccgagtttctcccgcagtcccccggattaccacccaccgaggaagaggaggaagaagaggaagaggacgacgaagatgacctctcctccacaccgacgccgacccccctgtccgaagccatgtttgccggcttcgaggaggccagcggcgacgaggactcggacacccaagccggactgtcccgggcactgatcctgaccggacaaagacgtcgaagcggtaacaacggggctctcacgctcgtcatcccctcgtggcacgtctttgcgagccttgacgacttggtaccattaacggtgagcgtgcagcacgccgcattacgacctacctcttatctgcgcagcgacatggacggcgacgtgcgtaccgcggcagacatcagcagcacgttgcggtccgtgcccgcgccacgaccctcacccatcagcaccgcttccacttccagcaccccacgcagtcgaccccgcatctagagagagacttctttgtttttcccccgcgtgtttttcccattccctgtatttatttctaaataataaaaacacagagacgttgataataaccgcagcctgagtaggaag

SEQ ID NO: 9 - RNA, 4005 nt, synthetic construct
  source 1..4005: mol_type = other RNA; note = pp71 Construct E pp65
aggacagaggaccctggtgttgggaaacggacactaggcgtccgcgcgatacggggttaaaacaaaaaaaatcggtggtggtgtgtgctggggtgtggtgacggtggggctttgcctctttttttttgtaataaaaaaagacactcaataatccgcggttgtctctgtgtagaacgtttttatttcgggttccgcgtttggtcgcctgcctgtgtaaggcggcggccgcaaagggcgcgccgctcagtcgcctacacccgtacgcgcaggcagcatggagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaggagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcagatcttcctggaggtacaagcgatacgcgagaccgtggaactgcgtcagtacgatcccgtggctgcactcttctttttcgatatcgacttgctgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagaagccctgcccgggccatgcatcgcctcgacgcccaaaaagcatcgaggttgagccacccgccgcgcacgcgcttaagacgactctataaaaacccacgtccactcagacacgcgacttttgggcgccacacctgtcgccgctgctatatttgcgacagttgccggaacccttcccgacctcccacgaagacccgttcacctttgcgcatcccctgaccctccccccatcccgccttcgcaatgtctcaggcatcgtcctcgcccggtgagggaccctcgtcggaagcggccgcgatcagcgaggccgaagccgccagcggcagctttggtcgcctgcactgccaggtgcttcggctcatcaccaacgtggaaggcggctcgctggaagccggtcgtctgcgactcctggacctgcgtaccaacatagaggtgagccgaccctcggttctctgctgttttcaggagaacaaatctccgcacgacaccgtagacctgaccgacctaaacatcaagggccgctgcgtggtgggcgaacgggaccgactgctggtggacctcaacaactttggcccacgacgcctgacgccaggctcagaaaacaacacggtctcggtgctggcctttgcgctgccgctagaccgcgtgcccgttagcggactgcacctctttcagagccagcggcgcggcggcgaagaaaatcggccgcgaatggaggcgcgcgccatcatccgccgcacggctcaccactgggccgtgcgactgaccgtgacgccgaactggcgccgcagaaccgacagcagtttggaggcagggcagatctttgtcagccagttcgcctttcgcgccggcgccatcccgctgacgctggtagacgccctggagcagctggcctgttcggaccctaacacgtacatccacaaaacggagacggacgaacgaggccaatggatcatgctgtttctgcatcacgactcaccgcacccgccgaccagcgtgtttctgcacttttcggtttacacgcatcgcgccgaggtggtggcgcgacacaatccgtacccgcacctacgacgcttgccggacaacggcttccagctgttgattcccaaaagttttacgctgacgcgcatacatcccgagtacatcgtgcagatccagaatgctttcgaaaccaatcagactcacgacaccatctttttcccggaaaacatcccgggcgtctccatagaagccggcccgctacccgatcgtgtgcgaatcaccctccgcgtcacgctgaccggcgaccaggccgttcatttggaacaccgacagccgctaggccgcatccactttttccgccgtgggttttggactctcacacccggtaaaccggacaaaatcaagcgtccccaggtgcagctgcgcgccggtctctttccacggagcgacgtcgtgcgcggcgccgtctccgagtttctcccgcagtcccccggattaccacccaccgaggaagaggaggaagaagaggaagaggacgacgaagatgacctctcctccacaccgacgccgacccccctgtccgaagccatgtttgccggcttcgaggaggccagcggcgacgaggactcggacacccaagccggactgtcccgggcactgatcctgaccggacaaagacgtcgaagcggtaacaacggggctctcacgctcgtcatcccctcgtggcacgtctttgcgagccttgacgacttggtaccattaacggtgagcgtgcagcacgccgcattacgacctacctcttatctgcgcagcgacatggacggcgacgtgcgtaccgcggcagacatcagcagcacgttgcggtccgtgcccgcgccacgaccctcacccatcagcaccgcttccacttccagcaccccacgcagtcgaccccgcatctagagagagacttctttgtttttcccccgcgtgtttttcccattccctgtatttatttctaaataataaaaacacagagacgttgataataaccgcagtgtgctttattagggtatcacggtgtagaaaaaaaaagagagggaaaccctaaatatagcgtctctcagcctgagtaggaag

SEQ ID NO: 10 - RNA, 4005 nt, synthetic construct
  source 1..4005: mol_type = other RNA; note = pp71 Construct F stop pp65
aggacagaggaccctggtgttgggaaacggacactaggcgtccgcgcgatacggggttaaaacaaaaaaaatcggtggtggtgtgtgctggggtgtggtgacggtggggctttgcctctttttttttgtaataaaaaaagacactcaataatccgcggttgtctctgtgtagaacgtttttatttcgggttccgcgtttggtcgcctgcctgtgtaaggcggcggccgcaaagggcgcgccgctcagtcgcctacacccgtacgcgcaggcagcatggagtcgcgcggtcgccgttgtcccgaatagtgatccgtactgggtcccatttcggggcacgtgctgaaagccgtgtttagtcgcggcgacacgccggtgctgccgcacgagacgcgactcctgcagacgggtatccacgtacgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaggagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcagatcttcctggaggtacaagcgatacgcgagaccgtggaactgcgtcagtacgatcccgtggctgcactcttctttttcgatatcgacttgctgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagaagccctgcccgggccatgcatcgcctcgacgcccaaaaagcatcgaggttgagccacccgccgcgcacgcgcttaagacgactctataaaaacccacgtccactcagacacgcgacttttgggcgccacacctgtcgccgctgctatatttgcgacagttgccggaacccttcccgacctcccacgaagacccgttcacctttgcgcatcccctgaccctccccccatcccgccttcgcaatgtctcaggcatcgtcctcgcccggtgagggaccctcgtcggaagcggccgcgatcagcgaggccgaagccgccagcggcagctttggtcgcctgcactgccaggtgcttcggctcatcaccaacgtggaaggcggctcgctggaagccggtcgtctgcgactcctggacctgcgtaccaacatagaggtgagccgaccctcggttctctgctgttttcaggagaacaaatctccgcacgacaccgtagacctgaccgacctaaacatcaagggccgctgcgtggtgggcgaacgggaccgactgctggtggacctcaacaactttggcccacgacgcctgacgccaggctcagaaaacaacacggtctcggtgctggcctttgcgctgccgctagaccgcgtgcccgttagcggactgcacctctttcagagccagcggcgcggcggcgaagaaaatcggccgcgaatggaggcgcgcgccatcatccgccgcacggctcaccactgggccgtgcgactgaccgtgacgccgaactggcgccgcagaaccgacagcagtttggaggcagggcagatctttgtcagccagttcgcctttcgcgccggcgccatcccgctgacgctggtagacgccctggagcagctggcctgttcggaccctaacacgtacatccacaaaacggagacggacgaacgaggccaatggatcatgctgtttctgcatcacgactcaccgcacccgccgaccagcgtgtttctgcacttttcggtttacacgcatcgcgccgaggtggtggcgcgacacaatccgtacccgcacctacgacgcttgccggacaacggcttccagctgttgattcccaaaagttttacgctgacgcgcatacatcccgagtacatcgtgcagatccagaatgctttcgaaaccaatcagactcacgacaccatctttttcccggaaaacatcccgggcgtctccatagaagccggcccgctacccgatcgtgtgcgaatcaccctccgcgtcacgctgaccggcgaccaggccgttcatttggaacaccgacagccgctaggccgcatccactttttccgccgtgggttttggactctcacacccggtaaaccggacaaaatcaagcgtccccaggtgcagctgcgcgccggtctctttccacggagcgacgtcgtgcgcggcgccgtctccgagtttctcccgcagtcccccggattaccacccaccgaggaagaggaggaagaagaggaagaggacgacgaagatgacctctcctccacaccgacgccgacccccctgtccgaagccatgtttgccggcttcgaggaggccagcggcgacgaggactcggacacccaagccggactgtcccgggcactgatcctgaccggacaaagacgtcgaagcggtaacaacggggctctcacgctcgtcatcccctcgtggcacgtctttgcgagccttgacgacttggtaccattaacggtgagcgtgcagcacgccgcattacgacctacctcttatctgcgcagcgacatggacggcgacgtgcgtaccgcggcagacatcagcagcacgttgcggtccgtgcccgcgccacgaccctcacccatcagcaccgcttccacttccagcaccccacgcagtcgaccccgcatctagagagagacttctttgtttttcccccgcgtgtttttcccattccctgtatttatttctaaataataaaaacacagagacgttgataataaccgcagtgtgctttattagggtatcacggtgtagaaaaaaaaagagagggaaaccctaaatatagcgtctctcagcctgagtaggaag

SEQ ID NO: 11 - DNA, 2742 bp, synthetic construct
  source 1..2742: mol_type = other DNA; note = M conserved gag/nef/pol fusion episensus 1, DNA
accatgccaattgtgcagaatttgcagggacagatggtgcatcaggctatctccccccgcacgctcaatgcctgggtgaaggtagtggaggagaaagccttttcaccagaagttatccctatgttctctgctctgagtgagggggccaccccccaagacttgaataccatgctgaacaccgtgggtgggcaccaggcagccatgcaaatgctgaaggagactattaacgaggaggctgctgagtgggataggctgcacccagtccatgcaggaccaatcgcgccaggccaaatgagagaaccacgggggagcgacatcgcagggacaacctccacacttcaagagcagatcggatggatgaccaacaaccccccaatccccgtcggcgagatctacaagcgctggataatccttggcctgaacaagattgtgcggatgtatagccccgtttcaatcctggatattcgccagggcccaaaggagcctttccgcgattatgtggataggttttacaagacccttcgggccgaacaggccacacaggaggtgaagaactggatgacagagactctgctggttcagaatgcaaatcccgactgtaagaccatcctgaaggccctgggaccagcagcgactctggaagagatgatgacggcctgtcagggcgtgggcggaccaggccataaagcacgggtgctcgtggggtttccagtgaggcctcaggtcccactgcgccccatgacatacaagggggccctggacctgtcacacttccttaaagaaaaaggcggcttggagggcctgatctatagtaaaaagagacaagaaatcctggatctttgggtttaccacacacaggggtacttccccgattggcagaattacactcccggccccggcattaggtaccccctcacatttggctggtgcttcaaactggtgcctccccaaattaccctttggcagagaccccttgtgaccatcaaaatcggcggtcagctgaaagaggcactgctggcagatgacaccgtgcttgaggatatcaacctgcctgggaaatggaaacccaagatgataggcggaatcggtgggttcatcaaagtgagacagtacgatcaaatcctgattgagatctgcggcaagaaggcgatcgggaccgtactggtcgggccaactccagtgaatatcataggccgcaacctcctgactcagataggctgtacccttaattttccgatatctccaatcgaaaccgtgcccgttaaactcaagcctggtatggatggacccaaagtcaagcaatggccactgaccgaggagaagataaaggccctggtggaaatctgcaccgaaatggaaaaggagggaaaaatctctaaaatcggacccgagaacccttataatactcccgtctttgccattaaaaagaaagacagcacaaagtggcggaaactcgtagactttagagagttgaataaaagaactcaggacttttgggaggtgcagcttggaattccccaccccgccggtctgaaaaagaagaaatccgtgacagtgctcgatgttggcgacgcctattttagtgtgcctcttgacaaagactttcggaaatacaccgccttcaccattccaagcattaataacgaaaccccaggtattagatatcaatacaacgtgctgccccaggggtggaaaggcagcccagctatttttcagagcagcatgacaaaaattttggagcccttcagaaagcagaatccagatatcgtcatttaccagctgtatgtggggtccgatctggagattggccagcaccgaactaagatcgaagaactgcgccagcacctgctcaaatggggattcacgacacctgataaaaaacatcagaaggaaccccctttcctttggatgggctacgagcttcaccccgataaatggactgttcagccaattgtcctccctgagaaggactcttggacagtcaacgacatccaaaaactggtggggaaactcaattgggccagtcagatctaccacggccaggtggactgctcaccggggatctggcagctttgtacacatctggaggggaaagtgatcctggtcgctgttcatgttgccagcggctacattgaagcagaggttattcccgccgagactggccaggagacagcttacttcttgctgaagctggctggtcggtggcccgtgaaggtaattcacacaaatggctctaactttacctctgccgcagttaaggccgcctgctggtgggccggaattaagcaggaattcgggattccttacaatccacagtcacagggtgtagtaagcatgaataaggaattgaagaaaattataggacaggttcgcgaccaggccgagcacctgaagaccgccgttcaaatggccgtattcatccacaattttaagcgcaaaggcgggattgggggttactctgccggggagagaataatagacatcattgccaccgatatccagacaaaactgcagaaacagataacaaagatacagaactttagggtatactatcgggattccagggaccctatctggaaggggccagcgaaactgctttggaagggagaaggagccgtggtcatacaggacaattcagacatcaaagtggtaccgagaaggaaggccaagattattagagattacggaaaacagatggcgggggacgactgcgttgccgggaggcaggacgaagatcaatga

SEQ ID NO: 12 - protein, 912 aa, synthetic construct
  source 1..912: note = M conserved gag/nef/pol fusion episensus 1, protein
MPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQATQEVKNWMTETLLVQNANPDCKTILKALGPAATLEEMMTACQGVGGPGHKARVLVGFPVRPQVPLRPMTYKGALDLSHFLKEKGGLEGLIYSKKRQEILDLWVYHTQGYFPDWQNYTPGPGIRYPLTFGWCFKLVPPQITLWQRPLVTIKIGGQLKEALLADDTVLEDINLPGKWKPKMIGGIGGFIKVRQYDQILIEICGKKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTKILEPFRKQNPDIVIYQLYVGSDLEIGQHRTKIEELRQHLLKWGFTTPDKKHQKEPPFLWMGYELHPDKWTVQPIVLPEKDSWTVNDIQKLVGKLNWASQIYHGQVDCSPGIWQLCTHLEGKVILVAVHVASGYIEAEVIPAETGQETAYFLLKLAGRWPVKVIHTNGSNFTSAAVKAACWWAGIKQEFGIPYNPQSQGVVSMNKELKKIIGQVRDQAEHLKTAVQMAVFIHNFKRKGGIGGYSAGERIIDIIATDIQTKLQKQITKIQNFRVYYRDSRDPIWKGPAKLLWKGEGAVVIQDNSDIKVVPRRKAKIIRDYGKQMAGDDCVAGRQDEDQ

SEQ ID NO: 13 - protein, 1190 aa, synthetic construct
  source 1..1190: note = Ag85A ESAT 6 Rv3407 Rv2626c RpfA RpfD
MAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGATEQQWNFAGIEAAASAIQGNVTSIHSLLDEGKQSLTKLAAAWGGSGSEAYQGVQQKWDATATELNNALQNLARTISEAGQAMASTEGNVTGMFARATVGLVEAIGIRELRQHASRYLARVEAGEELGVTNKGRLVARLIPVQAAERSREALIESGVLIPARRPQNLLDVTAEPARGRKRTLSDVLNEMRDEQTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALASSGRHRKPTTSNVSVAKIAFTGAVLGGGGIAMAAQATAATDGEWDQVARCESGGNWSINTGNGYLGGLQFTQSTWAAHGGGEFAPSAQLASREQQIAVGERVLATQGRGAWPVCGRGLSNATPREVLPASAAMDAPLDAAAVNGEPAPLAPPPADPAPPVELAANDLPAPLGEPLPAAPADPAPPADLAPPAPADVAPPVELAVNDLPAPLGEPLPAAPADPAPPADLAPPAPADLAPPAPADLAPPAPADLAPPVELAVNDLPAPLGEPLPAAPAELAPPADLAPASADLAPPAPADLAPPAPAELAPPAPADLAPPAAVNEQTAPGDQPATAPGGPVGLATDLELPEPDPQPADAPPPGDVTEAPAETPQVSNIAYTKKLWQAIRAQDVCGNDALDSLAQPYVIGTPGLLTTAGAGRPRDRCARIVCTVFIETAVVATMFVALLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 14 - RNA, 2114 nt, synthetic construct; identical to SEQ ID NO: 4
  source 1..2114: mol_type = other RNA; note = pp71 Construct B full length (FT) + pseudoU/5meC
  modified_base 1..2114: mod_base = OTHER; note = where c is present, c is m5c, and where t is present, t is p

SEQ ID NO: 15 - RNA, 2045 nt, synthetic construct; identical to SEQ ID NO: 5
  source 1..2045: mol_type = other RNA; note = pp71 Construct B.2, full length (FT) + pseudoU/5meC
  modified_base 1..2045: mod_base = OTHER; note = where c is present, c is m5c, and where t is present, t is p

SEQ ID NO: 16 - RNA, 2031 nt, synthetic construct; identical to SEQ ID NO: 6
  source 1..2031: mol_type = other RNA; note = pp71 Construct B.3 full length (FT) + pseudoU/5meC
  modified_base 1..2031: mod_base = OTHER; note = where t is present, t is p, and where c is present, c is m5c

SEQ ID NO: 17 - RNA, 1928 nt, synthetic construct; identical to SEQ ID NO: 7
  source 1..1928: mol_type = other RNA; note = pp71 Construct C immediate early (IE1) + pseudoU/5meC
  modified_base 1..1928: mod_base = OTHER; note = where t is present, t is p, and where c is present, c is m5c

SEQ ID NO: 18 - RNA, 1909 nt, synthetic construct; identical to SEQ ID NO: 8
  source 1..1909: mol_type = other RNA; note = pp71 Construct D short + pseudoU/5meC
  modified_base 1..1909: mod_base = OTHER; note = where t is present, t is p, and where c is present, c is m5c

SEQ ID NO: 19 - RNA, 4005 nt, synthetic construct; identical to SEQ ID NO: 9
  source 1..4005: mol_type = other RNA; note = pp71 Construct E pp65 + pseudoU/5meC
  modified_base 1..4005: mod_base = OTHER; note = where t is present, t is p, and where c is present, c is m5c

SEQ ID NO: 20 - RNA, 4005 nt, synthetic construct; identical to SEQ ID NO: 10
  source 1..4005: mol_type = other RNA; note = pp71 Construct F stop pp65 + pseudoU/5meC
  modified_base 1..4005: mod_base = OTHER; note = where t is present, t is p, and where c is present, c is m5c

SEQ ID NO: 21 - RNA, 2114 nt, synthetic construct; identical to SEQ ID NO: 4
  source 1..2114: mol_type = other RNA; note = pp71 Construct B full length (FT) + 5moU
  modified_base 1..2114: mod_base = OTHER; note = where t is present, t is mo5u

SEQ ID NO: 22 - RNA, 2045 nt, synthetic construct; identical to SEQ ID NO: 5
  source 1..2045: mol_type = other RNA; note = pp71 Construct B.2 full length (FT) + 5moU
  modified_base 1..2045: mod_base = OTHER; note = where t is present, t is mo5u

SEQ ID NO: 23 - RNA, 2031 nt, synthetic construct; identical to SEQ ID NO: 6
  source 1..2031: mol_type = other RNA; note = pp71 Construct B.3 full length (FT) + 5moU
  modified_base 1..2031: mod_base = OTHER; note = where t is present, t is mo5u

SEQ ID NO: 24 - RNA, 1928 nt, synthetic construct; identical to SEQ ID NO: 7
  source 1..1928: mol_type = other RNA; note = pp71 Construct C immediate early (IE1) + 5moU
  modified_base 1..1928: mod_base = OTHER; note = where t is present, t is mo5u

SEQ ID NO: 25 - RNA, 1909 nt, synthetic construct; identical to SEQ ID NO: 8
  source 1..1909: mol_type = other RNA; note = pp71 Construct D short + 5moU
  modified_base 1..1909: mod_base = OTHER; note = where t is present, t is mo5u

SEQ ID NO: 26 - RNA, 4005 nt, synthetic construct; identical to SEQ ID NO: 9
  source 1..4005: mol_type = other RNA; note = pp71 Construct E pp65 + 5moU
  modified_base 1..4005: mod_base = OTHER; note = where t is present, t is mo5u

SEQ ID NO: 27 - RNA, 4005 nt, synthetic construct; identical to SEQ ID NO: 10
  source 1..4005: mol_type = other RNA; note = pp71 Construct F stop pp65 + 5moU
  modified_base 1..4005: mod_base = OTHER; note = where t is present, t is mo5u

(19) United States
(12) Patent Application Publication
(10) Pub. No.: US 2025/0136949 A1
(43) Pub. Date: May 1, 2025

(54) METHODS FOR PRODUCING CMV VECTORS

(71) Applicant: Vir Biotechnology, Inc., San Francisco, CA (US)

(72) Inventors: Sarah Ledoux, San Francisco, CA (US); Eric Bruening, San Francisco, CA (US); Janet L. Douglas, San Francisco, CA (US); Christine R. Meyer, San Francisco, CA (US)

(21) Appl. No.: 18/686,635

(22) PCT Filed: Aug. 30, 2022
(86) PCT No.: PCT/US2022/075647

§ 371 Date: Feb. 26, 2024

Related U.S. Application Data
(60) Provisional application No. 63/239,269, filed on Aug. 31, 2021.

(51) Int. Cl. C12N 7/00; C07K 14/005; C07K 14/35; C12N 15/86

(52) U.S. Cl. CPC C12N 7/00; C07K 14/005; C07K 14/35; C12N 15/86; C12N 2710/16122; C12N 2710/16143; C12N 2710/16152; C12N 2740/16022

(57) ABSTRACT

The present disclosure provides methods for producing cytomegalovirus (CMV) viral vectors. The present disclosure also provides methods for modifying host cells for use in producing CMV viral vectors.

### STATEMENT REGARDING SEQUENCE LISTING

The Sequence Listing associated with this application is provided in text format in lieu of a paper copy, and is hereby incorporated by reference into the specification. The name of the XML file containing the Sequence Listing is 930485_436WO_SequenceListing.xml. The text file is 99,639 bytes, was created on Aug. 26, 2022, and is being submitted electronically via EFS-Web.

### BACKGROUND

Vaccine vectors based on Cytomegalovirus (CMV) exploit the natural ability of this virus to elicit and maintain circulating and tissue resident effector-differentiated T cells, including the potential sites of early HIV infection. For example, rhesus CMV (RhCMV) vectors encoding simian immunodeficiency virus (SIV) antigen inserts can (1) superinfect RhCMV-immune primates and elicit high frequency effector-differentiated, SIV-specific CD4+ and CD8+ T cells in both lymphoid and organ tissues, (2) maintain these responses indefinitely, and (3) manifest early stringent control and ultimate clearance of infection with the highly pathogenic SIVmac239 strain. Current CMV manufacturing processes are limited in production and not directly scalable. Deletion of essential viral genes from vaccine vectors is a customary practice to ensure clinical safety. However, to produce a vector with an essential gene deletion, some method of gene complementation must be employed. Standard approaches involve creating stable cell lines that express the essential viral gene or its functional equivalent, however production of HCMV is complicated by the fact that it requires primary normal diploid cells for virus production. Accordingly, there remains a need for manufacturing methods capable of generating CMV vector-based vaccines that can produce vaccine in amounts necessary for clinical and commercial use. Described herein is an approach that utilizes mRNA transfection to deliver an essential viral gene to the host cell for scalable production of CMV vectors.

### BRIEF SUMMARY

In certain aspects, the present disclosure provides a method of producing a CMV viral vector, comprising: (a) introducing a mRNA molecule encoding a pp71 protein to a cell (e.g., a MRC-5 cell); (b) infecting the cell with a CMV; (c) incubating the cell; and (d) collecting the CMV viral vector.

In certain aspects, the present disclosure also provides a CMV viral vector produced by any of the aforementioned methods.

### BRIEF DESCRIPTION OF THE DRAWINGS

FIG. 1 shows that HCMV cytopathic effect (CPE) is accelerated in the presence of pp71-V5 mRNA transfection. MRC-5 cells were either (1) mock-transfected, (2) transfected with anti-DAXX siRNA, or transfected with UL82 mRNA one day prior to infection with an HCMV vector at MOI 0.01. Pictures were taken at 13 days post infection.

FIG. 2 shows that HCMV production is accelerated in the presence of UL82 mRNA transfection. MRC-5 cells were transfected with either anti-DAXX siRNA or UL82 mRNA one day prior to infection with an HCMV vector at MOI 0.01. Viral supernatants were harvested and titered on the indicated day post infection (DPI). Titering was performed using an immunofluorescent focus forming assay. ffu=focus forming units.

FIG. 3 shows pp71 expression resulting from UL82 mRNA transfection. MRC-5 cells were transfected with UL82 mRNA and harvested on the indicated day post transfection. Immunoblots for pp71 were performed on the cell lysates after SDS-PAGE. Un-transfected MRC-5 cells were included as a negative control. The blots were striped and re-probed for cellular actin for a loading control.

FIG. 4 shows BAC DNA reconstitution in the presence of pp71 expression provided by either mRNA transfection or anti-DAXX siRNA transfection. MRC-5 cells were transfected with App71-GFP TR-3 BAC and observed microscopically over the days post transfection. At 12 DPI the number of cells expressing GFP is significantly greater in the cells transfected with pp71 mRNA.

FIG. 5A-5C show cell lines transfected with UL82 mRNA (A=MRC5; B=BJ-5ta) or induced to express pp71 using doxycycline (10C), and infected with a CMV stock virus at 1:81, 1:243, 1:729, and 1:2187 viral dilutions. Error bars are one standard deviation from the mean of three replicates. FIG. 5A shows a MRC5 cell line. FIG. 5B shows a BJ-5ta cell line. FIG. 5C shows a pp71 doxycycline-inducible cell line.

FIG. 6 shows a viral growth curve comparing production using pp71 mRNA vs anti-DAXX siRNA in T150 flasks.

FIG. 7 shows a viral growth curve with pp71 mRNA transfection in production vessel, HYPERStack12s.

FIG. 8 is an immunoblot showing pp71 protein loaded in the virion. A protein assay was performed and 30 μg total protein was loaded per sample. An MRC-5 cell lysate was used as the negative control sample (cell). Virion samples were obtained by centrifuging clarified supernatant through a sorbitol cushion at 24,000 RPM for 1 hr at 4° C. Wildtype TR3 virions (WT) were the positive control and the UL82-deleted virions were complemented with the indicated pp71 mRNA concentrations (ng/cm²). The upper blot was probed with an anti-pp71 antibody and then stripped and re-probed for actin as the loading control (lower blot).

FIGS. 9A-9B show pp71 expression resulting from pp71-V5 mRNA transfection. MRC-5 cells were transfected with 50 ng/cm² of pp71-V5 mRNA using Lipofectamine 2000. FIG. 9A shows immunoblots after SDS-page for pp71 in cell lysates harvested on the indicated day post transfection. Untransfected MRC-5 cells were included as a negative control. The blots were stripped and re-probed for cellular actin as a loading control. FIG. 9B shows images from an immunofluorescence assay (IFA) of cells at 48 hours post-transfection. Cells were stained with a primary anti-V5 tag antibody followed by a Cy-5 labeled secondary antibody. Pp71-V5 protein is expressed and localized to the nuclear region, which is counterstained with the nuclear stain DAPI.

FIG. 10 shows that HCMV CPE is accelerated in the presence of pp71-V5 mRNA transfection. MRC-5 cells were either (1) mock-transfected, (2) transfected with 10 μM anti-DAXX siRNA at one day before and 10 days post infection (DPI), or (3) transfected with a pp71 mRNA (encoded by UL82) construct bearing a V5 tag (pp71-V5 mRNA) (1 and 5 DPI) with pp71-deleted HCMV at MOI 0.01. Pictures were taken at 13 days post infection.

FIG. 11 shows that HCMV production is accelerated in the presence of pp71-V5 mRNA transfection. MRC-5 cells were transfected with either anti-DAXX SIRNA (10 μM at −1 and 10 DPI) or pp71-V5 mRNA (40 ng/cm² at −1 and 6 DPI) one day prior to infection with pp71-deleted HCMV vector at MOI 0.01. Viral supernatants were harvested and titered on the indicated day post infection (DPI). Titering was performed using an immunofluorescent focus forming assay. ffu=focus forming units.

FIG. 12 is an immunoblot showing lack of pp71-V5 protein at 13 DPI in cell and virion pellets after three mRNA transfections. MRC-5 fibroblasts were transfected with pp71-V5 mRNA (40 ng/cm²) at −1, 6, and 11 DPI and infected with pp71-deleted HCMV at MOI 0.01. For each sample, 27 μg total protein was loaded. Infected cell lysate and virion lysate are shown in lanes 3 and 4, respectively. An MRC-5 cell lysate was used as the negative control sample (lane 1). A pp71-V5 transfected MRC-5 cell lysate was used as a positive control sample (lane 2). The upper blot was probed with an anti-V5 antibody, stripped, and re-probed for Glycoprotein B (gB) to show the presence of HCMV (middle blot) and then stripped and re-probed for actin (lower blot).

FIG. 13A-13C show the effects of four lipid-based mRNA transfection reagents on EGFP expression and viability in MRC-5 cells following transfection with EGFP mRNA. MRC-5 cells were transfected with three amounts of mRNA (0.5 μg, 1.0 μg, or 1.5 μg), using four lipid-based transfection reagents (Lipofectamine 2000, MessengerMax, Jet-mRNA, or Trans-IT) at four amounts (“Low”, “Mid”, “High”, and “Higher”; see Table 3 for lipid volumes) and evaluated by flow cytometry for EGFP expression one day post-transfection. FIG. 13A shows the fraction of green (transfected, EGFP-positive) cells. FIG. 13B shows mean fluorescence intensity (MFI). FIG. 13C shows the fraction of viable cells.

FIG. 14 shows loss of EGFP in MRC-5 fibroblasts transfected with EGFP-Cy5 and infected with HCMV at 9 DPI. MRC-5 fibroblasts were transfected with 50 ng/cm² EGFP-Cy5 mRNA at DPI-1 and DPI 6 and infected with WT TR3 at a MOI of 0.01 at DPI 0. Phase images show presence of CPE, while Cy5 signal indicates the presence of transfected mRNA and EGFP signal indicates the presence of protein from translated mRNA.

FIG. 15A-15B shows stabilized EGFP expression in MRC-5 fibroblasts transfected with 5moU-modified EGFP mRNAs and infected with HCMV WT TR3. FIG. 15A is a representative immunoblot. EGFP mRNA constructs contained no modifications (lanes 2 and 3), or were generated with 5-methoxyuridine (5moU, lanes 4 and 5), pseudouridine plus 5-methylcytidine (pseudoU/5meC, lanes 6 and 7), or Cy-5 labeled uridine triphosphate at a 1:3 ratio to 5moU (lanes 7 and 8). Lane 1 shows no transfection with WT TR3 infection MOI 0.01 as a positive control for pp65 expression and a negative control for EGFP expression. Lanes 2, 4, 6, 8 are transfection only while lanes 3, 5, 7, 9 were infected with WT TR3 MOI 0.01 on 0 DPI. For each sample, 40 μg total protein was loaded, unless noted (lanes 2 and 3: 25 ug). The upper blot was probed with an anti-pp65 antibody showing the presence of TR3, stripped and re-probed for GFP (lower blot) and then stripped and re-probed for actin (middle blot). FIG. 15B. shows immunofluorescence images at 6 DPI of MRC-5 fibroblasts transfected with 5moU EGFP (labeled “B”) and transfected with 5moU EGFP and infected with WT TR3 at MOI 0.01 (labeled “C”). Phase images of the same field show transfection-only MRC-5 fibroblasts (labeled “D”) or the presence of CPE in infected MRC-5 fibroblasts (labeled “D”).

FIG. 16 shows the structure of several pp71 mRNA constructs for testing protein localization. Construct A includes a synthetic 5′UTR and a mouse α-globin 3′UTR (“start-to-stop”), also referred to as “pp71-V5 mRNA” in previous figures. Construct B includes the full-length viral pp71 5′ and 3′ UTRs. Construct C contains the HCMV IE1 5′UTR and the mouse-globin 3′UTR. Construct E is a bicistronic mRNA that includes pp65. Construct F is a bicistronic mRNA with a stop codon in pp65. Construct D contains a truncated 5′UTR beginning after the TATA box and a 3′UTR that ends before the presumed poly(A) signal sequence. All constructs do not contain the V5 epitope and were made with the 5moU (5-methoxyuridine) modified nucleoside. Constructs A and B were additionally made with pseudouridine and 5-methylcytidine-modified nucleosides.

FIG. 17A-17C shows immunoblots comparing expression of different pp71 mRNA constructs (see FIG. 16) with the 5moU modification in MRC-5 cell lysates. For each blot in FIG. 17A-17C: untransfected MRC-5 fibroblasts infected with WT TR3 and harvested at 12 DPI are shown in Lane 1; uninfected but transfected samples were collected at 1, 6, and 10 or 13 DPI with the indicated mRNA; samples transfected with the indicated mRNA and infected with a pp71-deleted virus (labeled “App71”) were harvested at 9 DPI or 12 DPI as indicated. For each sample, 30 μg total protein was loaded. The blots were probed with an anti-pp71 antibody (middle blot), stripped and re-probed with an anti-gB antibody (upper blot) and stripped and re-probed for actin (lower blot). FIG. 17C was also stripped and re-probed with an anti-pp65 antibody. FIG. 17A shows transfection with full-length viral 5′ and 3′ UTRs pp71 mRNA constructs (Construct B) modified with pseudoU/5meC (lanes 2-5) or 5moU (lanes 6-9). FIG. 17B shows transfection with immediate-early (Construct C, lanes 2-5) or short (Construct D, lanes 6-9) pp71 mRNA constructs modified with 5moU. FIG. 17C shows transfection with pp65 (Construct E, lanes 2-5) or stop (Construct F, lanes 6-9) pp71 mRNA constructs modified with 5moU.

FIG. 18A-18B shows immunoblots comparing expression of pp71 protein from MRC-5 cell lysates transfected with pp71 mRNAs (100 ng/cm²) having different poly-A tail lengths. FIG. 18A shows immunoblots of cell lysates collected at 5 DPI transfected with Construct B mRNAs produced with: an enzymatic 50nt poly-A tail (labeled “(2)”), produced with an enzymatic 100nt poly-A tail (lane 3), no added poly-A tail (lane 4), or produced with an enzymatically added poly-A tail of unknown length (lane 5). Construct A (“start-to-stop pp71 mRNA construct”) produced with a 80nt poly-A tail by template is also shown (lane 6). Untransfected MRC-5 cells were used as a negative control (lane 1). For each sample, 40 μg total protein was loaded. The upper blot was probed with an anti-pp71 antibody and then stripped and re-probed for actin (lower blot). FIG. 18B shows immunoblots of cell lysates collected at 2, 4, 6, and 8 DPI following transfection with Construct B pp71 mRNAs generated with a 80nt poly-A tail by template (labeled “template pA 80 bp”) compared with Construct B pp71 mRNAs generated with a 80nt poly-A tail by template using the plasmid template poly-A production process in preparation for GMP manufacturing (labeled “template pA 80 bp scale-up”). For each sample, 40 μg total protein was loaded. The upper blot was probed with an anti-pp71 antibody and then stripped and re-probed for B-tubulin (lower blot).

FIG. 19 shows a growth curve comparing titer of virus produced using template- or enzyme-based addition of the poly-A tail to Construct B pp71 mRNAs. MRC-5 cells were transfected with Construct B pp71 mRNAs with no added poly-A tail (labeled “A”), Construct B pp71 mRNAs produced with an enzymatic 50nt poly-A tail (two replicates labeled “B-1” and “B-2”), Construct B pp71 mRNAs produced with an 80nt poly-A tail by template (two replicates, labeled “C-1” and “C-2”), or Construct B pp71 mRNAs produced with an 80nt poly-A tail by template scaled-up for GMP manufacturing (labeled “D”) on −1 DPI at 100 ng/cm² and infected with a pp71-deleted vector at a MOI of 0.01. Viral titer in FFU/mL was determined by a late antigen immunofluorescence assay (LA IFA) at multiple days post-infection.

FIG. 20 shows an immunoblot of pp71 protein expression in MRC-5 fibroblasts following transfection of increased amounts of Construct B pp71 mRNA and infection with pp71-deleted CMV. MRC-5 cells were transfected at 500 ng/cm² or 200 ng/cm² and infected with pp71-deleted virus (Tuberculosis deleted: TR3 mir124 ΔUL128-130 ΔUL146-147 ΔUL82 Ag85A-ESAT-6-Rv3407-Rv2626c-RpfA-RpfD, MOI 0.01). MRC-5 cells infected with WT TR3 at MOI 0.01 (lane 3), and uninfected MRC-5 cells transfected at 1000 ng/cm² (lane 4) were used as positive controls. Untransfected MRC-5 cells (lane 1), and untransfected MRC-5 cells infected with pp71-deleted virus were used as negative controls (lane 2). For each sample, 40 μg total protein was loaded. The blot was probed with an anti-pp71 antibody (middle blot), stripped and re-probed with an anti-gB antibody (upper blot) and stripped and re-probed for actin (lower blot).

FIG. 21 shows a growth curve comparing viral titer in MRC-5 fibroblasts transfected with varying amounts of SS (start-to-stop, Construct A) or FT (full-length construct with 5′ and 3′ UTRs, Construct B) pp71 mRNA with anti-DAXX SIRNA. MRC-5 fibroblasts were transfected with pp71 mRNA (FT or SS from 5-500 ng/cm²) or anti-DAXX siRNA (10 μM) followed by infection with a pp71-deleted virus at a MOI of 0.01. Viral titer in FFU/mL was determined by LA IFA at the indicated day post-infection.

FIG. 22 shows growth curves demonstrating production scalability of the optimized pp71 mRNA transfection method at 100 ng/cm² to the HYPERStack format. Seven runs were performed in either HYPERStack-12s (HS-12s) or HYPERStack-36s (HS-36s). Three growth curves were performed in HS-12s following the process developed in T-flasks. HS-12s were seeded at 6.67×10³ cells/cm², transfected at day 3 post-seeding with Construct B pp71 mRNA, and infected on day 4 post-seeding with a pp71 deleted virus at a MOI of 0.01. Four growth curves were performed in HS-12s and HS-36s (dotted lines) wherein cells were transfected on day 4 post-seeding or greater than 85% confluency and infected on day 5 post-seeding with a pp71 deleted virus at a MOI of 0.01. Viral titer in FFU/mL was determined by LA IFA at multiple days post-infection.

FIG. 23 shows an immunoblot of M conserved gag/nef/pol fusion episensus 1 antigen expression from uncomplemented MRC-5 fibroblasts infected with Vector 5 (TR3 Δ146-147 Δ128-130 ΔUL82 M conserved gag/nef/pol fusion episensus 1). Vector 5 virus was produced in MRC-5 fibroblasts complemented by transfection of Construct B pp71 mRNA at 100 ng/cm² and harvested at either 17 or 20 DPI. For each sample, 40 μg total protein was loaded. Purified p24 protein was used as the positive control sample (5 ng, “p24”). MRC-5 cell lysate was used as the negative control sample (“MRC-5”). The blot was probed with an anti-p24 antibody (middle blot), stripped and re-probed with an anti-gB antibody (upper blot), and stripped and re-probed for actin (lower blot).

FIG. 24A-24B shows increased plaque number at lower MOIs using virus generated with pp71 mRNA transfection (200 ng/cm²) as compared to anti-DAXX siRNA transfection. Uncomplemented (untransfected) MRC-5 fibroblasts were infected with a pp71-deleted virus either produced with anti-DAXX siRNA (top panel) or 200 ng/cm² Construct B pp71 mRNA (bottom panel) at a MOI of 0.01. Cultures were monitored for spread and number of plaques. FIG. 24A shows phase images of CPE at 13 DPI. FIG. 24B is a table showing the range of MOIs tested for each group and the corresponding plaque count at 14 DPI. Plaques were counted visually.

FIG. 25 is an immunoblot showing pp71 protein expression from ultracentrifuged and sucrose gradient purified supernatant lysates from transfection-only control samples. A BCA (Bicinchoninic Acid) protein assay was performed and 30 μg total protein was loaded per sample. Lanes 3 and 4 show the Construct B pp71 mRNA transfection control cell lysate and “virion” lysate obtained by following the T-flask production process but omitting infection. The monolayer was scraped in the supernatant at 14 DPI and subjected to three freeze/thaw (3×F/T) cycles before clarification and ultracentrifugation (lane 3) or sucrose gradient purified (lane 4). MRC-5 cell lysate was used as a negative control sample (lane 1 and 7). Positive controls are shown using pp71 mRNA transfection only (lane 2, one day post-transfectin), infection cell lysate (lane 5, DPI 12) and sucrose gradient purified virion lysate (lane 6, DPI 12) are shown in lanes 8 and 9 respectively.

### DETAILED DESCRIPTION

The instant disclosure provides methods for producing a CMV vector, by providing complementation of an essential viral gene that has been deleted in the CMV vector, through transfection of mRNA encoding the missing protein into the host cell.

### I. GLOSSARY

The following sections provide a detailed description of methods for producing CMV vectors. Prior to setting forth this disclosure in more detail, it may be helpful to an understanding thereof to provide definitions of certain terms to be used herein. Additional definitions are set forth throughout this disclosure.

In the present description, the term “about” or “approximately” means±20% of the indicated range, value, or structure, unless otherwise indicated.

The term “comprise” means the presence of the stated features, integers, steps, or components as referred to in the claims, but that it does not preclude the presence or addition of one or more other features, integers, steps, components, or groups thereof. The term “consisting essentially of” limits the scope of a claim to the specified materials or steps and those that do not materially affect the basic and novel characteristics of the claimed invention.

It should be understood that the terms “a” and “an” as used herein refer to “one or more” of the enumerated components. The use of the alternative (e.g., “or”) should be understood to mean either one, both, or any combination thereof of the alternatives, and may be used synonymously with “and/or”. As used herein, the terms “include” and “have” are used synonymously, which terms and variants thereof are intended to be construed as non-limiting.

The word “substantially” does not exclude “completely”; e.g., a composition which is “substantially free” from Y may be completely free from Y. Where necessary, the word “substantially” may be omitted from definitions provided herein.

The terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex.

Nucleic acid molecules of particular sequence can be incorporated into a vector that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as CMV vectors. Viral vectors may be constructed from wild type or attenuated virus, including replication deficient virus.

As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ, or ex vivo. An mRNA may be transcribed from a DNA sequence by an RNA polymerase enzyme, and interacts with a ribosome to synthesize genetic information encoded by DNA. Generally, mRNA are classified into two sub-classes: pre-mRNA and mature mRNA. Precursor mRNA (pre-mRNA) is mRNA that has been transcribed by RNA polymerase but has not undergone any post-transcriptional processing (e.g., 5′ capping, splicing, editing, and polyadenylation). Mature mRNA has been modified via post-transcriptional processing (e.g., spliced to remove introns and polyadenylated) and is capable of interacting with ribosomes to perform protein synthesis. mRNA can be isolated from tissues or cells by a variety of methods. For example, a total RNA extraction can be performed on cells or a cell lysate and the resulting extracted total RNA can be purified (e.g., on a column comprising oligo-dT beads) to obtain extracted mRNA.

Alternatively, mRNA can be synthesized in a cell-free environment, for example by in vitro transcription (IVT). An “in vitro transcription template” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail.

The particular nucleotide sequence composition and length of an IVT template will depend on the mRNA of interest encoded by the template.

A “5′ untranslated region (UTR)” refers to a region of an mRNA that is directly upstream (i.e., 5′) from the start codon (i.e., the first codon of an mRNA transcript translated by a ribosome) that does not encode a protein or peptide.

A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

As used herein, the term “antigen” refers to a substance, typically a protein, which is capable of inducing an immune response in a subject. The term also refers to proteins that are immunologically active (also referred to as “immunogenic”) in the sense that once administered to a subject (either directly or by administering to the subject a nucleotide sequence or vector that encodes the protein) the protein is able to evoke an immune response of the humoral and/or cellular type directed against that protein.

As used herein, the term “microRNA” refers to a major class of biomolecules involved in control of gene expression. For example, in human heart, liver, or brain, miRNAs play a role in tissue specification or cell lineage decisions. In addition, miRNAs influence a variety of processes, including early development, cell proliferation and cell death, and apoptosis and fat metabolism. The large number of miRNA genes, the diverse expression patterns, and the abundance of potential miRNA targets suggest that miRNAs may be a significant source of genetic diversity. A mature miRNA is typically an 8-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

A miRNA silences translation of one or more specific mRNA molecules by binding to a miRNA recognition element (MRE) which is defined as any sequence that directly base pairs with and interacts with the miRNA somewhere on the mRNA transcript. Often, the MRE is present in the 3′ untranslated region (UTR) of the mRNA, but it may also be present in the coding sequence or in the 5′ UTR. MREs are not necessarily perfect complements to miRNAs, usually having only a few bases of complementarity to the miRNA and often containing one or more mismatches within those bases of complementarity. The MRE may be any sequence capable of being bound by a miRNA sufficiently that the translation of a gene to which the MRE is operably linked (such as a CMV gene that is essential or augmenting for growth in vivo) is repressed by a miRNA silencing mechanism such as the RISC.

As used herein, the term “heterologous antigen” refers to any protein or fragment thereof that is not derived from CMV. Heterologous antigens may be pathogen-specific antigens, tumor virus antigens, tumor antigens, host self-antigens, or any other antigen.

Orthologs of proteins are typically characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

The term “homologous” or “homolog” refers to a molecule or activity found in or derived from a host cell, species, or strain. For example, a heterologous or exogenous molecule or gene encoding the molecule may be homologous to a native host or host cell molecule or gene that encodes the molecule, respectively, but may have an altered structure, sequence, expression level or combinations thereof.

As used herein, the identity/similarity between two or more nucleic acid sequences, or two or more amino acid sequences, is expressed in terms of the identity or similarity between the sequences. Sequence identity may be measured in terms of percentage identity; the higher the percentage, the more identical the sequences are. Sequence similarity may be measured in terms of percentage identity or similarity (which takes into account conservative amino acid substitutions); the higher the percentage, the more similar the sequences are. Polypeptides or protein domains thereof that have a significant amount of sequence identity and also function the same or similarly to one another (for example, proteins that serve the same functions in different species or mutant forms of a protein that do not change the function of the protein or the magnitude thereof) may be called “homologs.”

Methods of alignment of sequences for comparison are well known in the art. Various programs and alignment algorithms are described in: Smith & Waterman, Adv Appl Math 2, 482 (1981); Needleman & Wunsch, J Mol Biol 48, 443 (1970); Pearson & Lipman, Proc Natl Acad Sci USA 85, 2444 (1988); Higgins & Sharp, Gene 73, 237-244 (1988); Higgins & Sharp, CABIOS 5, 151-153 (1989); Corpet et al., Nuc Acids Res 16, 10881-10890 (1988); Huang et al., Computer App Biosci 8, 155-165 (1992); and Pearson et al., Meth Mol Bio 24, 307-331 (1994). In addition, Altschul et al., J Mol Biol 215, 403-410 (1990), presents a detailed consideration of sequence alignment methods and homology calculations.

The NCBI Basic Local Alignment Search Tool (BLAST) (Altschul et al., (1990) supra) is available from several sources, including the National Center for Biological Information (NCBI, National Library of Medicine, Building 38A, Room 8N805, Bethesda, MD 20894) and on the Internet, for use in connection with the sequence analysis programs blastp, blastn, blastx, tblastn and tblastx. Additional information may be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr database, swissprot database, and patented sequences database. Queries searched with the blastn program are filtered with DUST (Hancock & Armstrong, Comput Appl Biosci 10, 67-70 (1994.) Other programs use SEG. In addition, a manual alignment may be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and may possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence may be made using this degeneracy to produce multiple nucleic acid molecules that all encode substantially the same protein. Such homologous nucleic acid sequences can, for example, possess at least about 50%, 60%, 70%, 80%, 90%, 95%, 98%, or 99% sequence identity to a nucleic acid that encodes a protein.

The human cytomegalovirus UL82 gene encodes pp71, a protein that is localized in the tegument domain of the virus particle. The UL82 gene of the CMV TR strain is SEQ ID NO:1, 118811 to 120490 for GenBank Accession No. KF021605.1. As used herein, UL82 refers to SEQ ID NO:1 and orthologs or homologs thereof.

As used herein, “pp71”, refers to a protein that is localized in the tegument domain of the CMV virus particle. Pp71 may perform one or more functions, including inhibition of Daxx repression of viral gene transcription, negative regulation of STING, and evasion of cell antiviral responses (Kalejta R F, et al. Expanding the Known Functional Repertoire of the Human Cytomegalovirus pp71 Protein. Front Cell Infect Microbiol. 2020 Mar. 12; 10:95). Deletion of UL82 or disruption of UL82 by insertion of a foreign gene at the UL82 locus results in the absence of pp71 protein and consequently reduces replication in fibroblasts, endothelial cells, epithelial cells, and astrocytes (Caposio P et al., Characterization of a live-attenuated HCMV-based vaccine platform. Sci Rep. 2019 Dec. 17; 9 (1): 19236). The effects of UL82 deletion or disruption are reversible by cell kinase inhibitors. The rhesus cytomegalovirus (RhCMV) gene RhCMV 110 is homologous to human CMV UL82 (Hansen S G, et al. Complete sequence and genomic analysis of rhesus cytomegalovirus. J Virol. 2003 June; 77 (12): 6620-36).

### II. COMPLEMENTATION USING MRNA TRANSFECTION

A challenge for manufacturing HCMV vectors having desirable properties for vaccines is that the vectors are often designed to have reduced viral replication or growth. For example, some live attenuated HCMV-HIV vaccine vectors are engineered to be growth deficient by deletion of the HCMV gene UL82 (SEQ ID NO:1, GenBank Accession No.: KF021605.1 (118815 to 120494)), which encodes the tegument protein pp71 (SEQ ID NO:2, GenBank Accession No.: AGL96671.1; SEQ ID NO:3, UniProtKB-R4SH92), resulting in lower viral yield. Commercial production of live HCMV-based HIV vaccines attenuated by pp71-deletion ultimately requires a complementing cell line permissive for vector growth under good manufacturing practices (GMP).

pp71 is important for wild type HCMV infection because this tegument protein is translocated to the nucleus where it suppresses cellular Daxx function, thus allowing CMV immediate-early (IE) gene expression that triggers the replication cycle. Some manufacturing processes rely on functional complementation using transient transfection of MRC-5 cells with an siRNA targeting DAXX, which mimics one of the functions of HCMV pp71. This method allows vector growth but involves complex manipulations not readily scalable for commercial production and complements only one of the many pp71 functions. In addition, RhCMV vectors grown in pp71 complementing cells have demonstrated an increase in potency as measured by the reduction in focus-forming units (FFUs) per dose required to induce an immune response. These observations suggest that cell-derived pp71 protein can be packaged in the virion of vectors in which this gene is deleted, and the viral vaccine containing pp71 protein can be administered at lower doses due to its increased potency, thereby providing clinical benefit and reducing manufacturing needs.

In some embodiments, the recombinant RhCMV or HCMV vector comprises a deletion in a RhCMV or HCMV gene that is essential for or augments replication (e.g. UL82). CMV essential genes and augmenting have been well described in the art (see, for example, Dunn et al., Proc. Natl. Acad. Sci. USA 100 (24): 14223-14228, 2003; and Dong et al., Proc. Natl. Acad. Sci. USA 100 (21): 12396-12401, 2003). Essential CMV genes include, but are not limited to, UL32, UL34, UL37, UL44, UL46, UL48, UL48.5, UL49, UL50, UL51, UL52, UL53, UL54, UL55, UL56, UL57, UL60, UL61, UL70, UL71, UL73, UL75, UL76, UL77, UL79, UL80, UL82, UL84, UL85, UL86, UL87, UL89, UL90, UL91, UL92, UL93, UL94, UL95, UL96, UL98, UL99, UL100, UL102, UL104, UL105, UL115 and UL122. In some embodiments, the CMV essential or augmenting gene is UL82, UL94, UL32, UL99, UL115 or UL44, or a homolog thereof (i.e., the homologous gene in RhCMV). Other essential or augmenting genes are known in the art and are described herein. In particular examples, the essential gene is UL82, or a homolog thereof.

mRNA transfection can be used to enable the host cell to express the essential viral gene. Transfection of a mRNA for expressing the essential viral gene may be able to provide all of the functions of the gene that are likely to enhance the infection process, such as cell cycle stimulation, efficient virion packaging, and virus stability. In addition, protein present late in infection has the potential to be packaged in the progeny virus, which could lower the required dose of the vaccine by more efficient first round infection and establishment of persistent infection.

In some embodiments, mRNA transfection of the essential viral gene into a host cell provides functional complementation resulting in successful propagation of gene-deleted HCMV virus vector. In some embodiments, the functional complementation results in accelerated HCMV spread, increased maximal titers, earlier maximal virus titers, and/or enhanced reconstitution of virus from BAC DNA.

In some embodiments, transient transfection of mRNA is used to identify functions that permit HCMV to grow to higher titers by supplementing infection with combinatorial libraries of mRNAs from laboratory strains known to grow to high titer.

In a natural infection, infectious parental CMV particles enter cells through interaction with cellular receptors, and capsid and tegument proteins are delivered into the cytosol. The capsid enters the nucleus and delivers the CMV genome, while tegument proteins are involved in initiating viral gene expression and regulating host cell responses. The viral genome is replicated and encapsulated into capsids that have been assembled in the nucleus, and the genome-containing capsids are transported to the cytosol where they associate with tegument proteins and acquire a viral envelope in the viral assembly complex. The enveloped infectious progeny CMV particles are then released from the cell (Jean Beltran P M and Cristea I M. The life cycle and pathogenesis of human cytomegalovirus infection: lessons from proteomics. Expert Rev Proteomics. 2014 December; 11 (6): 697-711). The parental viral particles and progeny particles may be structurally and genetically identical, or may be different, for example in the case of viral recombination when the cell is co-infected by multiple parental strains. The process of natural infection is harnessed in the laboratory manufacture of CMV viral particles, where a cell line is infected with a parental CMV, the cells produce progeny CMV, and the progeny CMV are then collected. In these processes, “parental” refers to the viral particles that infect the cells, and “progeny” refers to the produced viral particles.

In some embodiments, transfected mRNA is applied to cell lines for use in determining the infectious titer of viral stocks.

MRC-5 cells are well characterized primary normal diploid fibroblasts suitable for HCMV production. In some embodiments, MRC-5 cells are used in the methods disclosed herein for generating cells that express pp71.

In some embodiments, transfection of naturally permissive MRC-5 cells with UL82 mRNA leads to higher apparent titers as compared to either transfection of BJ-5ta cells or the pp71 BJ-5ta cell line. This likely provides a more accurate titer of the material and allows better quantification of diluted material used in dose range studies. Transfection of pp71 mRNA may allow for lower titer viruses to be tested (e.g., less than 5e4 FFU/mL) with greater assay reproducibility and confidence.

In some embodiments, a method of producing a CMV viral vector is provided, comprising: (a) introducing a mRNA molecule encoding a pp71 protein to a cell; (b) infecting the cell with a CMV; (c) incubating the cell; and (d) collecting the CMV viral vector. In some embodiments, the mRNA molecule encoding a pp71 protein comprises a sequence according to SEQ ID NOs: 4-10. In some embodiments, the mRNA molecule encoding a pp71 protein is produced using full substitution with pseudouridine (pseudoU) and 5-methylcytidine (5meC), e.g. SEQ ID NOs: 14-20. In some embodiments, the mRNA molecule encoding a pp71 protein is produced using full substitution with 5-methoxyuridine (5moU), e.g. SEQ ID NOs: 21-27. In some embodiments, the mRNA molecule encoding a pp71 protein is delivered to the cell using transfection. In some embodiments, the cell is a MRC-5 cell.

The mRNA molecule can be manufactured by in vitro transcription which typically uses a double stranded DNA template in buffer with an RNA polymerase and a mix of NTPs. The polymerase can synthesize the mRNA molecule. The DNA can then be enzymatically degraded. The mRNA molecule can be purified away from polymerase, free NTPs, and degraded DNA.

Transfection of cells with unmodified mRNA molecules can lead to cell death due to activation of innate immune pathways. Modifications to the mRNA molecule can act as marks of self-RNA to reduce innate immune responses to endogenous cellular RNA and enhance stability. Such modifications include, but are not limited to, modified nucleosides, elongation of the poly-adenosine (poly(A)) tail, and modified 5′ cap structures. Furthermore, removal of dsRNA contaminants through high-performance liquid chromatography (HPLC) purification can also enhance stability and reduce immune recognition.

The term “RNA” or “mRNA” or “mRNA molecule” encompasses not only RNA molecules containing natural ribonucleotides, but also analogs and derivatives of RNA comprising one or more nucleotide/nucleoside or ribonucleotide/ribonucleoside analogs or derivatives as described herein or as known in the art. Strictly speaking, a “nucleoside” includes a nucleoside base and a deoxyribose sugar, and a “nucleotide” is a nucleoside with one, two or three phosphate moieties. Strictly speaking, a “ribonucleoside” includes a nucleoside base and a ribose sugar, and a “ribonucleotide” is a ribonucleoside with one, two or three phosphate moieties.

The RNA molecule can be modified in the nucleobase structure or in the ribose-phosphate backbone structure, e.g., as described in greater detail below. As non-limiting examples, an RNA molecule can also include at least one modified ribonucleoside including, but not limited to, a 5-methoxyuridine (5moU) modified nucleoside, a 5-methylcytidine (5meC) modified nucleoside, a N6-methyladenosine (m6A) modified nucleoside, a 5-methyluridine (m5U) modified nucleoside, a pseudouridine (pseudoU) modified nucleoside, a2-thioruridine (s2U) modified nucleoside, or any combination thereof. In another example, an RNA molecule can comprise at least two modified ribonucleosides, at least 25, at least 50, at least 100, at least 500, at least 1000, at least 2000, or more, up to the entire length of the mRNA molecule. The modifications need not be the same for each of such a plurality of modified nucleosides/ribonucleosides in an RNA molecule.

In some embodiments, the mRNA molecule encoding a pp71 protein is produced using full substitution with pseudouridine (pseudoU), 5-methylcytidine (5meC), 5-methoxyuridine (5moU), or any combination thereof. In some embodiments, the mRNA molecule encoding a pp71 protein is produced using full substitution with pseudouridine (pseudoU) and 5-methylcytidine (5meC), e.g. SEQ ID NOs: 14-20. In some embodiments, the mRNA molecule encoding a pp71 protein is produced using full substitution with 5-methoxyuridine (5moU), e.g. SEQ ID NOs: 21-27. In some embodiments, the mRNA molecule encoding a pp71 protein is produced using full substitution with pseudouridine (pseudoU). In some embodiments, the mRNA molecule encoding a pp71 protein is produced using full substitution with 5-methylcytidine (5meC).

In some embodiments, a poly-adenosine (poly(A)) tail of variable or pre-determined length can be added to the 3′ end of the mRNA molecule encoding the pp71 protein. In some embodiments, the poly(A) tail is approximately 60-100 nucleotides long. A poly(A) tail may be added in a template-dependent fashion during transcription and/or may be added enzymatically post-transcription. In certain embodiments, a poly(A) tail is added enzymatically post-transcription by a poly(A) polymerase, which variably adds a tail of approximately 60-100 nucleotides. In certain other embodiments, a poly(A) tail is synthesized from a double-stranded DNA template, e.g., a linearized plasmid template (“Run-off Transcription” (TriLink)), wherein transcription stops when RNA polymerase falls off the DNA. In further embodiments the plasmid encodes a poly(A) tail of a pre-determined length of approximately 80 nt.

The mRNA molecule can be transcribed to contain a 5′ cap structure. In some embodiments the mRNA molecule is transcribed with a 7-methylguanylate cap, creating a cap0 structure. In some embodiments the mRNA molecule is transcribed with a modified 5′-methoxyuridine (5moU) nucleoside, creating a cap1 structure.

In some embodiments, the mRNAs can be transcribed to contain the full-length viral pp71 5′ UTR and 3′ UTR (untranslated region) to enable nuclear localization.

RNA can be introduced into target cells using any of a number of different methods, for instance, commercially available methods which include, but are not limited to, lipid-mediated transfection, electroporation, multiporation, polymer encapsulation, peptide mediated transfection, or biolistic particle delivery systems.

In some embodiments, the mRNA molecule is delivered to cells using lipid-based transfection. Commercially available lipid-mediated transfection reagents include Lipofectamine 2000 (ThermoFisher), MessengerMax (ThermoFisher), Jet-mRNA (PolyPlus) and Trans-IT (Mirus Bio). In certain embodiments the mRNA is introduced to a cell with MessengerMax, which provides high transfection efficiency and low toxicity. In some embodiments, the mRNA molecule is delivered at a dose of 5 ng/cm² to 500 ng/cm². In some embodiments, the mRNA molecule is delivered at a dose of 50 ng/cm² to 100 ng/cm². In some embodiments, the mRNA molecule is delivered at a dose of 50 ng/cm². In some embodiments, the mRNA molecule is delivered at a dose of 100 ng/cm². In some embodiments, the mRNA molecule is delivered at a dose of at least 5 ng/cm², at least 50 ng/cm², at least 100 ng/cm², at least 150 ng/cm², at least 200 ng/cm², at least 250 ng/cm², at least 300 ng/cm², at least 350 ng/cm², at least 400 ng/cm², at least 450 ng/cm², or at least 500 ng/cm². In some embodiments, the mRNA molecule is delivered at a dose of 100 ng/cm². In some embodiments, the mRNA molecule is delivered at a dose of no more than 50 ng/cm², no more than 100 ng/cm², no more than 150 ng/cm², no more than 200 ng/cm², no more than 250 ng/cm², no more than 300 ng/cm², no more than 350 ng/cm², no more than 400 ng/cm², no more than 450 ng/cm², or no more than 500 ng/cm². In some embodiments, the lipid-mediated transfection reagent amount used in the transfection is 0.01 μl to 1000 μl. In some embodiments, the lipid-mediated transfection reagent amount used in the transfection is 0.1 μl to 100 μl. In some embodiments, the lipid-mediated transfection reagent amount used in the transfection is 0.1 μl to 50 μl. In some embodiments, the lipid-mediated transfection reagent amount used in the transfection is 0.1 μl to 10 μl. In some embodiments, the lipid-mediated transfection reagent amount used in the transfection is 0.5 μl. In some embodiments, the lipid-mediated transfection reagent amount used in the transfection is 0.75 μl. In some embodiments, the lipid-mediated transfection reagent amount used in the transfection is 1 μl. In some embodiments, the lipid-mediated transfection reagent amount used in the transfection is 1.25 μl. In some embodiments, the lipid-mediated transfection reagent amount used in the transfection is 1.5 μl. In some embodiments, the lipid-mediated transfection reagent amount used in the transfection is 2 μl. In some embodiments, the lipid-mediated transfection reagent amount used in the transfection is 2.5 μl. In some embodiments, the lipid-mediated transfection reagent amount used in the transfection is 3 μl. In some embodiments, the lipid-mediated transfection reagent amount used in the transfection is 3.6 μl. In some embodiments, the lipid-mediated transfection reagent amount used in the transfection is at least 0.01 μl, at least 0.1 μl, at least 0.5 μl, at least 1 μl, at least 2 μl, at least 3 μl, at least 4 μl, at least 5 μl, at least 10 μl, at least 50 μl, at least 100 μl, or at least 1000 μl. In some embodiments, the lipid-mediated transfection reagent amount used in the transfection is no more than 0.01 μl, no more than 0.1 μl, no more than 0.5 μl, no more than 1 μl, no more than 2 μl, no more than 3 μl, no more than 4 μl, no more than 5 μl, no more than 10 μl, no more than 50 μl, no more than 100 μl, or no more than 1000 μl.

### III. CMV VECTORS AND ANTIGENS

In any of the aforementioned methods and compositions, the CMV may be a HCMV or a RhCMV. In some embodiments, the CMV is a HCMV. In some embodiments, the CMV is a genetically modified TR strain of HCMV. In some embodiments, the CMV comprises a TR3 backbone.

In some embodiments, the recombinant CMV vector is or is derived from HCMV TR3. As referred to herein, “HCMV TR3” or “TR3” refers to a HCMV-TR3 vector backbone derived from the clinical isolate HCMV TR, as described in Caposio, P et al. (Characterization of a live attenuated HCMV-based vaccine platform. Scientific Reports 9, 19236 (2019)).

In some embodiments, the recombinant CMV vector (e.g., a recombinant HCMV vector comprising a TR3 backbone) comprises a nucleic acid sequence encoding a microRNA (miRNA) recognition element (MRE). In some embodiments, the HCMV vector comprises a nucleic acid sequence encoding an MRE that contains target sites for microRNAs expressed in endothelial cells. Examples of miRNAs expressed in endothelial cells are miR126, miR-126-3p, miR-130a, miR-210, miR-221/222, miR-378, miR-296, and miR-328. In some embodiments, the HCMV vector lacks UL18, UL128, UL130, UL146, and UL147 (and optionally UL82) and expresses UL40 and US28 and the MRE contains target sites for microRNAs expressed in endothelial cells.

In some embodiments, the recombinant CMV vector (e.g., a recombinant HCMV vector comprising a TR3 backbone) comprises a nucleic acid sequence encoding an MRE that contains target sites for microRNAs expressed in myeloid cells. Examples of miRNAs expressed in myeloid cells are miR-142-3p, miR-223, miR-27a, miR-652, miR-155, miR-146a, miR-132, miR-21, miR-124, and miR-125.

MREs that may be included in the recombinant CMV vector disclosed herein may be any miRNA recognition element that silences expression in the presence of a miRNA expressed by endothelial cells, or any miRNA recognition element that silences expression in the presence of a miRNA expressed by myeloid cells. Such an MRE may be the exact complement of a miRNA. Alternatively, other sequences may be used as MREs for a given miRNA. For example, MREs may be predicted from sequences using publicly available data bases. In one example, the miRNA may be searched on the website microRNA.org (www.microrna.org). In turn, a list of mRNA targets of the miRNA will be listed. For each listed target on the page, ‘alignment details’ may be accessed and putative MREs accessed. One of ordinary skill in the art may select a validated, putative, or mutated MRE sequence from the literature that would be predicted to induce silencing in the presence of a miRNA expressed in a myeloid cell such as a macrophage. One example involves the above referenced website. The person of ordinary skill in the art may then obtain an expression construct whereby a reporter gene (such as a fluorescent protein, enzyme, or other reporter gene) has expression driven by a promoter such as a constitutively active promoter or cell specific promoter. The MRE sequence may then be introduced into the expression construct. The expression construct may be transfected into an appropriate cell, and the cell transfected with the miRNA of interest. A lack of expression of the reporter gene indicates that the MRE silences gene expression in the presence of the miRNA.

In some embodiments, the CMV vector comprises a nucleic acid sequence that does not encode any MREs.

In any of the aforementioned methods and compositions, the CMV may be genetically modified. In some embodiments, the CMV comprises a deletion of a gene, or does not express an active gene, wherein the gene is UL128, UL130, UL146, UL147, UL82, or UL18, or homologs thereof. In some embodiments, the CMV comprises a deletion of a gene, or does not express an active gene, wherein the gene is UL128, UL130, UL146, UL147, UL82, or UL18, or homologs thereof. In some embodiments, the CMV does not express an active UL128 or homolog thereof, does not express an active UL130 or homolog thereof, does not express an active UL146 or homolog thereof, and does not express an active UL147 or homolog thereof. In some embodiments, the CMV does not express an active UL128 or homolog thereof, does not express an active UL130 or homolog thereof, does not express an active UL146 or homolog thereof, does not express an active UL147 or homolog thereof, and does not express an active UL82 or homolog thereof. In some embodiments, the CMV does not express an active UL128 or homolog thereof, does not express an active UL130 or homolog thereof, does not express an active UL146 or homolog thereof, does not express an active UL147 or homolog thereof, does not express an active UL82 or homolog thereof, and does not express an active UL18 or homolog thereof. In some embodiments, the CMV does not express an active UL128 or homolog thereof, does not express an active UL130 or homolog thereof, does not express an active UL146 or homolog thereof, does not express an active UL147 or homolog thereof, and does not express an active UL82 or homolog thereof, wherein the CMV additionally expresses a target sequence for mir124. In some embodiments, the CMV does not express an active UL82 or homolog thereof. In some embodiments, the CMV comprises a deletion of UL128, UL130, UL146, and UL147, or homologs thereof. In some embodiments, the CMV comprises a deletion of UL128, UL130, UL146, UL147, and UL82, or homologs thereof. In some embodiments, the CMV comprises a deletion of UL128, UL130, UL146, UL147, UL82, and UL18, or homologs thereof. In some embodiments, the CMV comprises a deletion of UL128, UL130, UL146, UL147, and UL82, or homologs thereof, wherein the CMV additionally expresses a target sequence for mir124. In some embodiments, the CMV comprises a deletion of UL82 or homolog thereof. In some embodiments, a nucleic acid encoding a heterologous antigen replaces UL128, UL130, UL146, UL147, UL82, or UL18, or homologs thereof. In some embodiments, a nucleic acid encoding a heterologous antigen replaces UL82.

In any of the aforementioned methods and compositions, the CMV may comprise a nucleic acid encoding a heterologous antigen. In some embodiments, the heterologous antigen comprises a pathogen-specific antigen or a tumor antigen. In some embodiments, the heterologous antigen comprises a pathogen-specific antigen comprising a human immunodeficiency virus (HIV) antigen, a simian immunodeficiency virus (SIV) antigen, a human cytomegalovirus (HCMV) antigen, a hepatitis B virus (HBV) antigen, a hepatitis C virus (HCV) antigen, a papilloma virus antigen (e.g., a human papilloma virus (HPV) antigen), a Plasmodium antigen, a Kaposi's sarcoma-associated herpesvirus antigen, a Varicella zoster virus (VZV) antigen, an Ebola virus, a Mycobacterium tuberculosis antigen, a Chikungunya virus antigen, a dengue virus antigen, a monkeypox virus antigen, a herpes simplex virus (HSV) 1 antigen, a herpes simplex virus (HSV) 2 antigen, an Epstein-Barr virus (EBV) antigen, a poliovirus antigen, an influenza virus antigen, or a Clostridium tetani antigen. In some embodiments, the heterologous antigen comprises a HIV antigen. In some embodiments, the heterologous antigen comprises a HIV antigen, wherein the HIV antigen is Gag, Pol, Nef, Env, Tat, Rev, Tat, Vpr, Vif, or Vpu, or an epitope or antigenic fragment thereof. In some embodiments, the heterologous antigen comprises a HIV antigen, wherein the HIV antigen comprises more than one of Gag, Pol, Nef, Env, Tat, Rev, Tat, Vpr, Vif, and Vpu, or an epitope or antigenic fragment thereof. In some embodiments, the heterologous antigen comprises a HIV antigen, wherein the HIV antigen comprises more than one of Gag, Pol, Nef, Env, Tat, Rev, Tat, Vpr, Vif, and Vpu, or an epitope or antigenic fragment thereof, comprised in a fusion molecule. In some embodiments, the heterologous antigen comprises a Mycobacterium tuberculosis antigen. In some embodiments, the heterologous antigen comprises a Mycobacterium tuberculosis antigen, wherein the Mycobacterium tuberculosis antigen is Ag85A, ESAT-6, Rv3407, Rv2626c, Rv2626c, RpfA, or RpfD or an epitope or antigenic fragment thereof. In some embodiments, the Mycobacterium tuberculosis antigen comprises more than one of Ag85A, ESAT-6, Rv3407, Rv2626c, Rv2626c, RpfA, and RpfD, or an epitope or antigenic fragment thereof. In some embodiments, the Mycobacterium tuberculosis antigen comprises more than one of Ag85A, ESAT-6, Rv3407, Rv2626c, Rv2626c, RpfA, and RpfD, or an epitope or antigenic fragment thereof, comprised in a fusion molecule.

In some embodiments, the heterologous antigen comprises a prostate cancer antigen.

In some aspects, the present disclosure provides a CMV viral vector produced by any of the aforementioned methods.

### VI. EXAMPLE EMBODIMENTS

In some embodiments, the present disclosure provides:

1. A method of producing a progeny cytomegalovirus (CMV), comprising:
(a) introducing to a cell a mRNA molecule encoding a gene that is essential for or augments CMV replication;
(b) infecting the cell with a parent CMV;
(c) incubating the cell; and
(d) collecting the progeny CMV.

2. A method of producing a progeny CMV, comprising:
(a) introducing a mRNA molecule encoding a pp71 protein to a cell;
(b) infecting the cell with a parent CMV;
(c) incubating the cell; and
(d) collecting the progeny CMV.

3. A method of producing a progeny CMV, comprising:
(a) first, introducing a mRNA molecule encoding a pp71 protein to a cell;
(b) second, infecting the cell with a parent CMV;
(c) third, incubating the cell; and
(d) fourth, collecting the progeny CMV.

4. A method of producing a CMV viral vector, comprising:
(a) introducing a mRNA molecule encoding a pp71 protein to a cell;
(b) infecting the cell with a CMV;
(c) incubating the cell; and
(d) collecting the CMV viral vector.

5. The method of embodiment 1, wherein the gene that is essential for or augments CMV replication is UL82, UL32, UL34, UL37, UL44, UL46, UL48, UL48.5, UL49, UL50, UL51, UL52, UL53, UL54, UL55, UL56, UL57, UL60, UL61, UL70, UL71, UL73, UL75, UL76, UL77, UL79, UL80, UL84, UL85, UL86, UL87, UL89, UL90, UL91, UL92, UL93, UL94, UL95, UL96, UL98, UL99, UL100, UL102, UL104, UL105, UL115, or UL122, or a homolog thereof.

6. The method of any one of embodiments 1-3 and 5, wherein the progeny CMV comprises pp71 protein.

7. The method of embodiment 4, wherein the CMV viral vector comprises pp71 protein.

8. The method of any one of embodiments 1-7, wherein the cell is a MRC-5 cell.

9 The method of any one of embodiments 1-8, wherein the mRNA molecule comprises the sequence according to SEQ ID NO:14.

10. The method of any one of embodiments 1-8, wherein the mRNA molecule comprises the sequence according to one of SEQ ID NOs: 14-20.

11. The method of any one of embodiments 1-8, wherein the mRNA molecule comprises the sequence according to one of SEQ ID NOs: 4-10.

12. The method of any one of embodiments 1-8, wherein the mRNA molecule comprises the sequence according to SEQ ID NO:4.

13. The method of any one of embodiments 1-8, 11, and 12, wherein the mRNA molecule comprises the sequence according to one of SEQ ID NOS: 4-10, wherein each uridine is substituted with pseudouridine and each cytidine is substituted with 5-methylcytidine.

14. The method of any one of embodiments 1-8, 11, and 12, wherein the mRNA molecule comprises the sequence according to one of SEQ ID NOs: 4-10, wherein each uridine is substituted with 5-methoxyuridine.

15. The method of any one of embodiments 1-8, wherein the mRNA molecule comprises the sequence according to one of SEQ ID NOs: 21-27.

16. The method of any one of embodiments 1-15, wherein the mRNA molecule further comprises a poly(A) tail.

17. The method of any one of embodiments 1-16, wherein a poly(A) tail has been added to the 3′ end of the mRNA molecule encoding the pp71 protein.

18. The method of embodiment 16 or embodiment 17, wherein the mRNA molecule was produced using a double-stranded DNA template encoding the poly(A) tail.

19. The method of embodiment 18, wherein the double-stranded DNA template is a plasmid.

20. The method of any one of embodiments 16-19, wherein the poly(A) tail is approximately 60-100 nucleotides long.

21. The method of any one of embodiments 16-19, wherein the poly(A) tail is 80 nucleotides long. 22. The method of any one of embodiments 1-10 and 16-21, wherein the mRNA molecule comprises the sequence according to one of SEQ ID NOs: 14-20, wherein each uridine is substituted with pseudouridine and each cytidine is substituted with 5-methylcytidine, and has a poly(A) tail 80 nucleotides in length.

23. The method of any one of embodiments 1-8, 11-13, and 16-21, wherein the mRNA molecule comprises the sequence according to one of SEQ ID NOs: 4-10, wherein each uridine is substituted with pseudouridine and each cytidine is substituted with 5-methylcytidine, and has a poly(A) tail 80 nucleotides in length; and wherein the poly(A) tail was produced using a plasmid template.

24. The method of any one of embodiments 1-8, 11, 14, and 16-21, wherein the mRNA molecule comprises the sequence according to SEQ ID NOs: 4-10, wherein each uridine is substituted with 5-methoxyuridine, and has a poly(A) tail 80 nucleotides in length; wherein the poly(A) tail was produced using a plasmid template.

25. The method of any one of embodiments 1-8 and 15-21, wherein the mRNA molecule comprises a sequence according to SEQ ID NOs: 21-27, wherein each uridine is substituted with 5-methoxyuridine, and has a poly(A) tail 80 nucleotides in length.

26. The method of embodiment 16 or embodiment 17, wherein the poly(A) tail is or has been added by an enzyme after transcription. 27. The method of embodiment 26, wherein the poly(A) tail is approximately 50-100 nucleotides long.

28. The method of any one of embodiments 1-27, wherein the mRNA molecule is introduced using transfection.

29. The method of embodiment 28, wherein the transfection is accomplished using a lipid transfection reagent.

30. The method of embodiment 29, wherein the lipid transfection reagent comprises MessengerMax, Lipofectamine 2000, Jet-mRNA, or Trans-IT.

31. The method of any one of embodiments 1-30, wherein the mRNA molecule is delivered at a dose of 5 ng/cm² to 500 ng/cm².

32. The method of any one of embodiments 1-31, wherein the mRNA molecule is delivered at a dose of 50 ng/cm² to 100 ng/cm².

33. The method of any one of embodiments 1-32, wherein the mRNA molecule is delivered at a dose of 50 ng/cm².

34. The method of any one of embodiments 1-33, wherein the mRNA molecule is delivered at a dose of 100 ng/cm².

35. The method of any one of embodiments 4, 7, and 8-34, wherein the CMV is a HCMV.

36. The method of any one of embodiments 1-3, 5, 6, and 8-34, wherein the parent CMV is a HCMV.

37. The method of any one of embodiments 1-3, 5, 6, 8-34, and 36, wherein the progeny CMV is a HCMV.

38. The method of embodiment 35, wherein the CMV is a genetically modified TR strain of HCMV.

39. The method of embodiment 36 or embodiment 37, wherein the parent CMV is a genetically modified TR strain of HCMV.

40. The method of any one of embodiments 36, 37, and 39, wherein the progeny CMV is a genetically modified TR strain of HCMV.

41. The method of any one of embodiments 4, 7, 8-35, and 37, wherein the CMV comprises a TR3 backbone.

42. The method of any one of embodiments 1-3, 5, 6, 8-34, 36, 37, 39, and 40, wherein the parent CMV comprises a TR3 backbone.

43. The method of any one of embodiments 1-3, 5, 6, 8-34, 36, 37, 39, 40, and 42, wherein the progeny CMV comprises a TR3 backbone. 44. The method of any one of embodiments 4, 7, 8-34, 35, 38, and 41, wherein the CMV comprises a nucleic acid encoding a heterologous antigen.

45. The method of any one of embodiments 1-3, 5, 6, 8-34, 36, 37, 39, 40, 42, and 43, wherein the parent CMV comprises a nucleic acid encoding a heterologous antigen.

46. The method of any one of embodiments 1-3, 5, 6, 8-34, 36, 37, 39, 40, 42, 43, and 45, wherein the progeny CMV comprises a nucleic acid encoding a heterologous antigen.

47. The method of any one of embodiments 44-46, where in the heterologous antigen comprises a pathogen-specific antigen or a tumor antigen.

48. The method of any one of embodiments 44-46, wherein the heterologous antigen comprises a pathogen-specific antigen comprising a human immunodeficiency virus (HIV) antigen, a simian immunodeficiency virus (SIV) antigen, a human cytomegalovirus (HCMV) antigen, a hepatitis B virus (HBV) antigen, a hepatitis C virus (HCV) antigen, a papilloma virus antigen (e.g., a human papilloma virus (HPV) antigen), a Plasmodium antigen, a Kaposi's sarcoma-associated herpesvirus antigen, a Varicella zoster virus (VZV) antigen, an Ebola virus, a Mycobacterium tuberculosis antigen, a Chikungunya virus antigen, a dengue virus antigen, a monkeypox virus antigen, a herpes simplex virus (HSV) 1 antigen, a herpes simplex virus (HSV) 2 antigen, an Epstein-Barr virus (EBV) antigen, a poliovirus antigen, an influenza virus antigen, or a Clostridium tetani antigen.

49. The method of any one of embodiments 44-46, wherein the heterologous antigen comprises a HIV antigen.

50. The method of embodiment 49, wherein the HIV antigen is Gag, Pol, Nef, Env, Tat, Rev, Tat, Vpr, Vif, or Vpu, or an epitope or antigenic fragment thereof.

51. The method of embodiment 49, wherein the HIV antigen comprises more than one of Gag, Pol, Nef, Env, Tat, Rev, Tat, Vpr, Vif, and Vpu, or an epitope or antigenic fragment thereof.

52. The method of embodiment 49, wherein the HIV antigen is a fusion protein comprising more than one of Gag, Pol, Nef, Env, Tat, Rev, Tat, Vpr, Vif, and Vpu, or an epitope or antigenic fragment thereof.

53. The method of embodiment 49, wherein the HIV antigen comprises SEQ ID NO: 11 or 12.

54. The method of any one of embodiments 44-46, wherein the heterologous antigen comprises a Mycobacterium tuberculosis antigen.

55. The method of embodiment 54, wherein the Mycobacterium tuberculosis antigen is Ag85A, ESAT-6, Rv3407, Rv2626c, Rv2626c, RpfA, or RpfD or an epitope or antigenic fragment thereof.

56. The method of embodiment 54, wherein the Mycobacterium tuberculosis antigen comprises more than one of Ag85A, ESAT-6, Rv3407, Rv2626c, Rv2626c, RpfA, and RpfD, or an epitope or antigenic fragment thereof.

57. The method of embodiment 54, wherein the Mycobacterium tuberculosis antigen comprises more than one of Ag85A, ESAT-6, Rv3407, Rv2626c, Rv2626c, RpfA, and RpfD, or an epitope or antigenic fragment thereof, comprised in a fusion molecule.

58. The method of embodiment 54, wherein the Mycobacterium tuberculosis antigen comprises SEQ ID NO:13.

59. The method of any one of embodiments 44-46, wherein the heterologous antigen comprises a prostate cancer antigen.

60. The method of any one of embodiments 4, 7, 8-34, 35, 38, 41, 44, and 47-59, wherein the CMV does not express an active UL128, UL130, UL146, UL147, UL82, or UL18, or homologs thereof.

61. The method of any one of embodiments 4, 7, 8-34, 35, 38, 41, 44, and 47-60, wherein the CMV does not express an active UL128 or homolog thereof, does not express an active UL130 or homolog thereof, does not express an active UL146 or homolog thereof, and does not express an active UL147 or homolog thereof.

62. The method of any one of embodiments 4, 7, 8-34, 35, 38, 41, 44, and 47-61, wherein the CMV does not express an active UL128 or homolog thereof, does not express an active UL130 or homolog thereof, does not express an active UL146 or homolog thereof, does not express an active UL147 or homolog thereof, and does not express an active UL82 or homolog thereof.

63. The method of any one of embodiments 4, 7, 8-34, 35, 38, 41, 44, and 47-62, wherein the CMV comprises a deletion of UL128, UL130, UL146, UL147, UL82, or UL18, or homologs thereof.

64. The method of any one of embodiments 4, 7, 8-34, 35, 38, 41, 44, and 47-63, wherein the CMV comprises a deletion of UL128 or homolog thereof, a deletion of UL130 or homolog thereof, a deletion of UL146 or homolog thereof, and a deletion of UL147 or homolog thereof.

65. The method of any one of embodiments 4, 7, 8-34, 35, 38, 41, 44, and 47-64, wherein the CMV comprises a deletion of UL128 or homolog thereof, a deletion of UL130 or homolog thereof, a deletion of UL146 or homolog thereof, a deletion of UL147 or homolog thereof, and a deletion of UL82 or homolog thereof.

66. The method of any one of embodiments 4, 7, 8-34, 35, 38, 41, 44, and 47-65, wherein the CMV does not express an active UL128 or homolog thereof, does not express an active UL130 or homolog thereof, does not express an active UL146 or homolog thereof, does not express an active UL147 or homolog thereof, does not express an active UL82 or homolog thereof, and does not express an active UL18 or homolog thereof.

67. The method of any one of embodiments 4, 7, 8-34, 35, 38, 41, 44, and 47-66, wherein the CMV further comprises a nucleic acid sequence encoding a microRNA (miRNA) recognition element (MRE), wherein the MRE contains a target site for a miRNA expressed in endothelial cells or myeloid cells.

68. The method of any one of embodiments 4, 7, 8-34, 35, 38, 41, 44, and 47-67, wherein the CMV does not express an active UL82 or homolog thereof.

69. The method of any one of embodiments 4, 7, 8-34, 35, 38, 41, 44, and 47-68, wherein the CMV comprises a deletion of UL128 or homolog thereof, a deletion of UL130 or homolog thereof, a deletion of UL146 or homolog thereof, a deletion of UL147 or homolog thereof, a deletion of UL82 or homolog thereof, and a deletion of UL18 or homolog thereof.

70. The method of any one of embodiments 4, 7, 8-34, 35, 38, 41, 44, and 47-68, wherein the CMV comprises a deletion of UL128 or homolog thereof, a deletion of UL130 or homolog thereof, a deletion of UL146 or homolog thereof, a deletion of UL147 or homolog thereof, and a deletion of UL82 or homolog thereof, wherein the CMV further comprises a nucleic acid sequence encoding a microRNA (miRNA) recognition element (MRE), wherein the MRE contains a target site for a miRNA expressed in endothelial cells or myeloid cells.

71. The method of any one of embodiments 4, 7, 8-34, 35, 38, 41, 44, and 47-70, wherein the CMV comprises a deletion of UL82 or homolog thereof.

72. The method of any one of embodiments 1-3, 5, 6, 8-34, 36, 37, 39, 40, 42, 43, and 45-59, wherein the parent CMV does not express an active UL128, UL130, UL146, UL147, UL82, or UL18, or homologs thereof.

73. The method of any one of embodiments 1-3, 5, 6, 8-34, 36, 37, 39, 40, 42, 43, 45-59, and 72, wherein the parent CMV does not express an active UL128 or homolog thereof, does not express an active UL130 or homolog thereof, does not express an active UL146 or homolog thereof, and does not express an active UL147 or homolog thereof.

74. The method of any one of embodiments 1-3, 5, 6, 8-34, 36, 37, 39, 40, 42, 43, 45-59, 72, and 73, wherein the parent CMV does not express an active UL128 or homolog thereof, does not express an active UL130 or homolog thereof, does not express an active UL146 or homolog thereof, does not express an active UL147 or homolog thereof, and does not express an active UL82 or homolog thereof.

75. The method of any one of embodiments 1-3, 5, 6, 8-34, 36, 37, 39, 40, 42, 43, 45-59, and 72-74, wherein the parent CMV comprises a deletion of UL128, UL130, UL146, UL147, UL82, or UL18, or homologs thereof.

76. The method of any one of embodiments 1-3, 5, 6, 8-34, 36, 37, 39, 40, 42, 43, 45-59, and 72-75, wherein the parent CMV comprises a deletion of UL128 or homolog thereof, a deletion of UL130 or homolog thereof, a deletion of UL146 or homolog thereof, and a deletion of UL147 or homolog thereof.

77. The method of any one of embodiments 1-3, 5, 6, 8-34, 36, 37, 39, 40, 42, 43, 45-59, and 72-76, wherein the parent CMV comprises a deletion of UL128 or homolog thereof, a deletion of UL130 or homolog thereof, a deletion of UL146 or homolog thereof, a deletion of UL147 or homolog thereof, and a deletion of UL82 or homolog thereof.

78. The method of any one of embodiments 1-3, 5, 6, 8-34, 36, 37, 39, 40, 42, 43, 45-59, and 72-77, wherein the parent CMV does not express an active UL128 or homolog thereof, does not express an active UL130 or homolog thereof, does not express an active UL146 or homolog thereof, does not express an active UL147 or homolog thereof, does not express an active UL82 or homolog thereof, and does not express an active UL18 or homolog thereof.

79. The method of any one of embodiments 1-3, 5, 6, 8-34, 36, 37, 39, 40, 42, 43, 45-59, and 72-78, wherein the parent CMV further comprises a nucleic acid sequence encoding a microRNA (miRNA) recognition element (MRE), wherein the MRE contains a target site for a miRNA expressed in endothelial cells or myeloid cells.

80. The method of any one of embodiments 1-3, 5, 6, 8-34, 36, 37, 39, 40, 42, 43, 45-59, and 72-79, wherein the parent CMV does not express an active UL82 or homolog thereof.

81. The method of any one of embodiments 1-3, 5, 6, 8-34, 36, 37, 39, 40, 42, 43, 45-59, and 72-80, wherein the parent CMV comprises a deletion of UL128 or homolog thereof, a deletion of UL130 or homolog thereof, a deletion of UL146 or homolog thereof, a deletion of UL147 or homolog thereof, a deletion of UL82 or homolog thereof, and a deletion of UL18 or homolog thereof.

82 The method of any one of embodiments 1-3, 5, 6, 8-34, 36, 37, 39, 40, 42, 43, 45-59, 72-77, and 79-81, wherein the parent CMV comprises a deletion of UL128 or homolog thereof, a deletion of UL130 or homolog thereof, a deletion of UL146 or homolog thereof, a deletion of UL147 or homolog thereof, and a deletion of UL82 or homolog thereof, wherein the parent CMV further comprises a nucleic acid sequence encoding a microRNA (miRNA) recognition element (MRE), wherein the MRE contains a target site for a miRNA expressed in endothelial cells or myeloid cells.

83. The method of any one of embodiments 1-3, 5, 6, 8-34, 36, 37, 39, 40, 42, 43, 45-59, and 72-82, wherein the parent CMV comprises a deletion of UL82 or homolog thereof.

84. The method of any one of embodiments 1-3, 5, 6, 8-34, 36, 37, 39, 40, 42, 43, 45-59, and 72-83, wherein the progeny CMV does not express an active UL128, UL130, UL146, UL147, UL82, or UL18, or homologs thereof.

85. The method of any one of embodiments 1-3, 5, 6, 8-34, 36, 37, 39, 40, 42, 43, 45-59, and 72-84, wherein the progeny CMV does not express an active UL128 or homolog thereof, does not express an active UL130 or homolog thereof, does not express an active UL146 or homolog thereof, and does not express an active UL147 or homolog thereof.

86. The method of any one of embodiments 1-3, 5, 6, 8-34, 36, 37, 39, 40, 42, 43, 45-59, and 72-85, wherein the progeny CMV does not express an active UL128 or homolog thereof, does not express an active UL130 or homolog thereof, does not express an active UL146 or homolog thereof, does not express an active UL147 or homolog thereof, and does not express an active UL82 or homolog thereof.

87. The method of any one of embodiments 1-3, 5, 6, 8-34, 36, 37, 39, 40, 42, 43, 45-59, and 72-86, wherein the progeny CMV comprises a deletion of UL128, UL130, UL146, UL147, UL82, or UL18, or homologs thereof.

88. The method of any one of embodiments 1-3, 5, 6, 8-34, 36, 37, 39, 40, 42, 43, 45-59, and 72-87, wherein the progeny CMV comprises a deletion of UL128 or homolog thereof, a deletion of UL130 or homolog thereof, a deletion of UL146 or homolog thereof, and a deletion of UL147 or homolog thereof.

89. The method of any one of embodiments 1-3, 5, 6, 8-34, 36, 37, 39, 40, 42, 43, 45-59, and 72-88, wherein the progeny CMV comprises a deletion of UL128 or homolog thereof, a deletion of UL130 or homolog thereof, a deletion of UL146 or homolog thereof, a deletion of UL147 or homolog thereof, and a deletion of UL82 or homolog thereof.

90. The method of any one of embodiments 1-3, 5, 6, 8-34, 36, 37, 39, 40, 42, 43, 45-59, and 72-89, wherein the progeny CMV does not express an active UL128 or homolog thereof, does not express an active UL130 or homolog thereof, does not express an active UL146 or homolog thereof, does not express an active UL147 or homolog thereof, does not express an active UL82 or homolog thereof, and does not express an active UL18 or homolog thereof.

91. The method of any one of embodiments 1-3, 5, 6, 8-34, 36, 37, 39, 40, 42, 43, 45-59, and 72-90, wherein the progeny CMV further comprises a nucleic acid sequence encoding a microRNA (miRNA) recognition element (MRE), wherein the MRE contains a target site for a miRNA expressed in endothelial cells or myeloid cells.

92. The method of any one of embodiments 1-3, 5, 6, 8-34, 36, 37, 39, 40, 42, 43, 45-59, and 72-91, wherein the progeny CMV does not express an active UL82 or homolog thereof.

93. The method of any one of embodiments 1-3, 5, 6, 8-34, 36, 37, 39, 40, 42, 43, 45-59, and 72-92, wherein the progeny CMV comprises a deletion of UL128 or homolog thereof, a deletion of UL130 or homolog thereof, a deletion of UL146 or homolog thereof, a deletion of UL147 or homolog thereof, a deletion of UL82 or homolog thereof, and a deletion of UL18 or homolog thereof.

94. The method of any one of embodiments 1-3, 5, 6, 8-34, 36, 37, 39, 40, 42, 43, 45-59, 72-77, 79-89, and 91-94, wherein the progeny CMV comprises a deletion of UL128 or homolog thereof, a deletion of UL130 or homolog thereof, a deletion of UL146 or homolog thereof, a deletion of UL147 or homolog thereof, and a deletion of UL82 or homolog thereof, wherein the progeny CMV further comprises a nucleic acid sequence encoding a microRNA (miRNA) recognition element (MRE), wherein the MRE contains a target site for a miRNA expressed in endothelial cells or myeloid cells.

95. The method of any one of embodiments 1-3, 5, 6, 8-34, 36, 37, 39, 40, 42, 43, 45-59, and 72-94, wherein the progeny CMV comprises a deletion of UL82 or homolog thereof.

96. The method of any one of embodiments 44-95, wherein the nucleic acid encoding the heterologous antigen replaces UL128, UL130, UL146, UL147, UL82, or UL18, or homologs thereof.

97. The method of any one of embodiments 44-96, wherein the nucleic acid encoding the heterologous antigen replaces UL82 or a homolog thereof.

98. A progeny CMV produced by the method of any one of embodiments 1-3, 5, 6, 8-34, 36, 37, 39, 40, 42, 43, 45-59, and 72-97.

99. A CMV viral vector produced by the method of any one of embodiments 4, 7, 8-34, 35, 38, 41, 44, 47-71, 96, or 97.

100. A mRNA molecule comprising the nucleotide sequence of SEQ ID NOs: 14-20.

101. A mRNA molecule comprising the nucleotide sequence of SEQ ID NOs: 4-10.

102. The mRNA molecule of embodiment 101, wherein each uridine is substituted with pseudouridine and each cytidine is substituted with 5-methylcytidine.

103. The mRNA molecule of embodiment 101, wherein each uridine is substituted with 5-methoxyuridine.

104. A mRNA molecule comprising the nucleotide sequence of SEQ ID NO: 21-27.

105. The mRNA molecule of any one of embodiments 100-104, wherein the mRNA molecule further comprises a poly(A) tail.

106. The mRNA molecule of any one of embodiments 100-104, wherein a poly(A) tail has been added to the 3′ end of the mRNA molecule.

107. The mRNA molecule of any one of embodiments 100-106, wherein the mRNA molecule was produced using a double-stranded DNA template encoding the poly(A) tail.

108. The mRNA molecule of claim 107, wherein the double-stranded DNA template is a plasmid.

109. The mRNA molecule of any one of embodiments 100-108, wherein the poly(A) tail is approximately 60-100 nucleotides long.

110. The mRNA molecule of any one of embodiments 100-109, wherein the poly(A) tail is 80 nucleotides long.

111. A mRNA molecule comprising a sequence according to SEQ ID NOs: 14-20, wherein each uridine is substituted with pseudouridine and each cytidine is substituted with 5-methylcytidine, and a poly(A) tail 80 nucleotides in length.

112. A mRNA molecule comprising a sequence according to SEQ ID NOs: 4-10, wherein each uridine is substituted with pseudouridine and each cytidine is substituted with 5-methylcytidine, and a poly(A) tail 80 nucleotides in length, wherein the mRNA molecule was produced using a plasmid template.

113. A mRNA molecule comprising a sequence according to SEQ ID NOs: 4-10, wherein each uridine is substituted with 5-methoxyuridine and the mRNA molecule a poly(A) tail 80 nucleotides in length, wherein the mRNA molecule was produced using a plasmid template.

114. A mRNA molecule comprising a sequence according to SEQ ID NOs: 21-27, wherein each uridine is substituted with 5-methoxyuridine and a poly(A) tail 80 nucleotides in length.

115. The mRNA molecule of embodiment 105, wherein the poly(A) tail has been added by an enzyme after transcription.

116. The mRNA molecule of embodiment 115, wherein the poly(A) tail is approximately 50-100 nucleotides long.

### EXAMPLES

### Example 1

Transient Transfection of UL82 mRNA in Primary Fibroblasts Complements UL82-Deleted HCMV Vectors

Deletion of essential viral genes from vaccine vectors is a customary practice to ensure clinical safety. However, to produce the vector some method of complementation must be employed. Standard approaches involve creating stable cell lines that express the essential viral gene or its functional equivalent. This is complicated in situations such as HCMV that require primary normal diploid cells for virus production. An alternative approach is to utilize mRNA transfection to deliver the essential viral gene to the host cell. In this study, UL82 mRNA was transiently transfected into primary fibroblasts (MRC-5 cells) to complement HCMV vectors deleted for UL82.

HCMV UL82 expresses the major tegument protein pp71. One of the major functions of pp71 occurs at the onset of infection, where it is involved in the degradation of the cellular gene product, Daxx. In the absence of pp71, Daxx silences viral immediate-early (IE) gene expression mediated by histone deacetylases. However, this cellular protection mechanism is effectively neutralized when pp71 is transported to the nucleus where it can mediate proteasomal degradation of Daxx releasing the block to IE gene expression. HCMV vectors deleted for pp71 show a significant growth defect that prevents virus spreading and shedding in primate models. In combination with other safety modifications, pp71 deletion protects fetal rhesus macaques in a direct injection model of primary RhCMV infection.

To be able to produce HCMV vectors deleted for UL82, siRNA targeting DAXX may be transfected into host cells, in order to functionally complement for the absence of pp71. While this method is sufficient to produce virus, it primarily inhibits de novo Daxx production, and does not provide any of the other functions of pp71 likely to enhance the infection process such as cell cycle stimulation, efficient virion packaging, and virus stability.

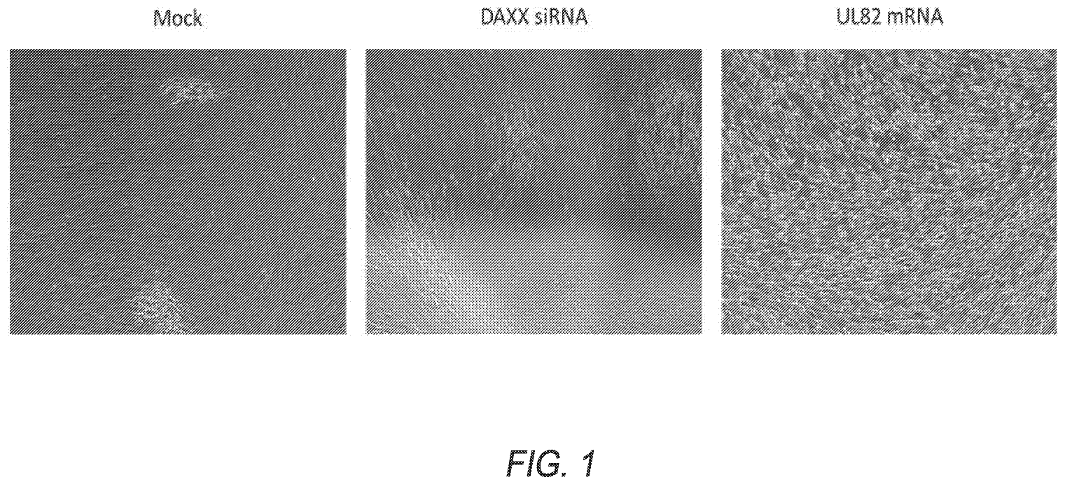

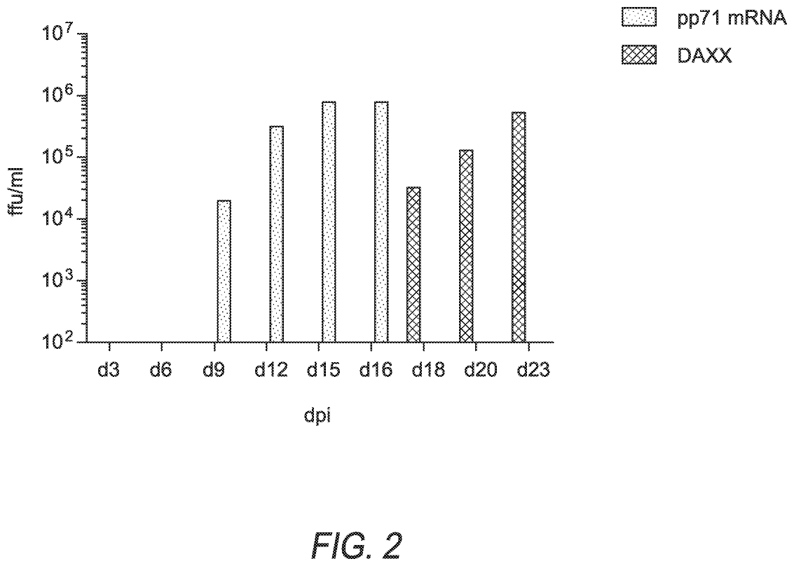

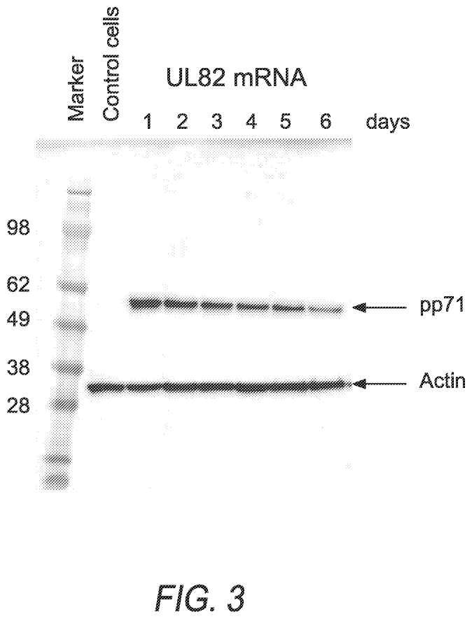

The results demonstrate that mRNA transfection of the essential viral gene UL82 can provide functional complementation resulting in successful propagation of UL82-deleted HCMV virus vector. Use of UL82 mRNA transfection accelerates HCMV spread when compared to either mock transfection or functional complementation by anti-DAXX siRNA transfection as shown in FIG. 1. HCMV vector-infected MRC-5 cultures reached 100% cytopathic effect (CPE) 6-9 days earlier than anti-DAXX siRNA-transfected cultures. In addition, maximal virus titers were achieved 6-9 days earlier in UL82 mRNA-transfected cultures compared to cultures transfected with anti-DAXX siRNA (FIG. 2). As shown in FIG. 3, UL82 mRNA transfection results in pp71 protein expression for at least 6 days, as measured by immunoblot analysis. These data indicate that UL82 mRNA transfection can efficiently complement the UL82-deleted HCMV and significantly accelerates virus production compared to a complementation method using anti-DAXX siRNA.

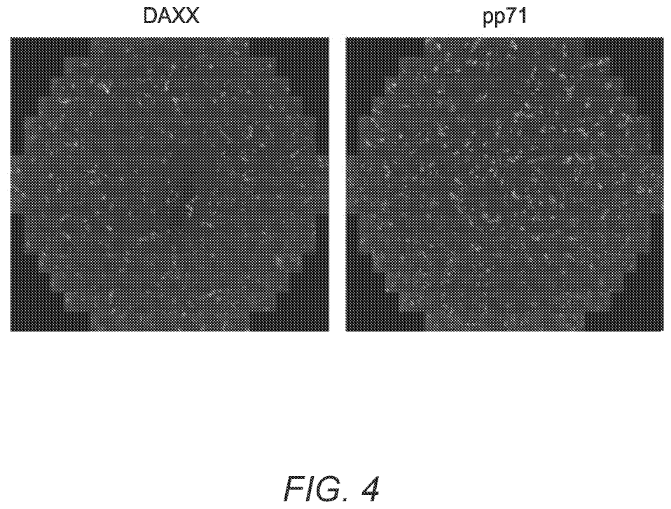

These results also suggest that UL82 mRNA transfection may enhance reconstitution of virus from BAC DNA. Experiments using a App71-GFP (UL82-deleted) virus construct indicated a more rapid progression of reconstitution from clonal BAC DNA. FIG. 4 shows a visualization of this effect in the stitched micrograph from a 6 well plate at 12 days post transfection. The number of green cells indicate the efficiency of the virus reconstitution under each condition, anti-DAXX siRNA and pp71 mRNA. The use of pp71 mRNA appears to boost the efficiency, which should result in earlier time of harvest.

The use of mRNA transfection for complementation is not limited to UL82 and could be extended to other essential HCMV genes. Transient transfection of mRNA could also be used to identify functions that permit HCMV to grow to higher titers by supplementing infection with combinatorial libraries of mRNAs from laboratory strains known to grow to high titer. In addition, pp71 protein present late in infection has the potential to be packaged in the progeny virus, which could lower the required dose of the vaccine by more efficient first-round infection and establishment of persistent infection.

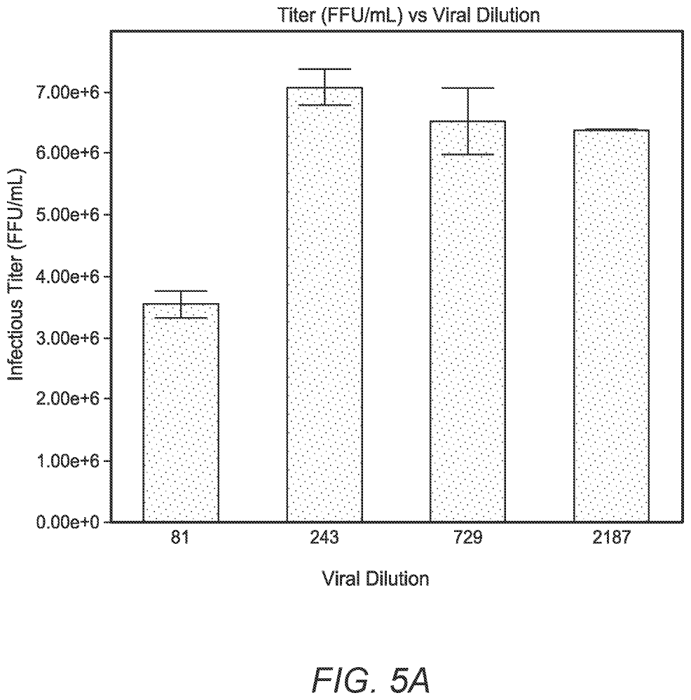

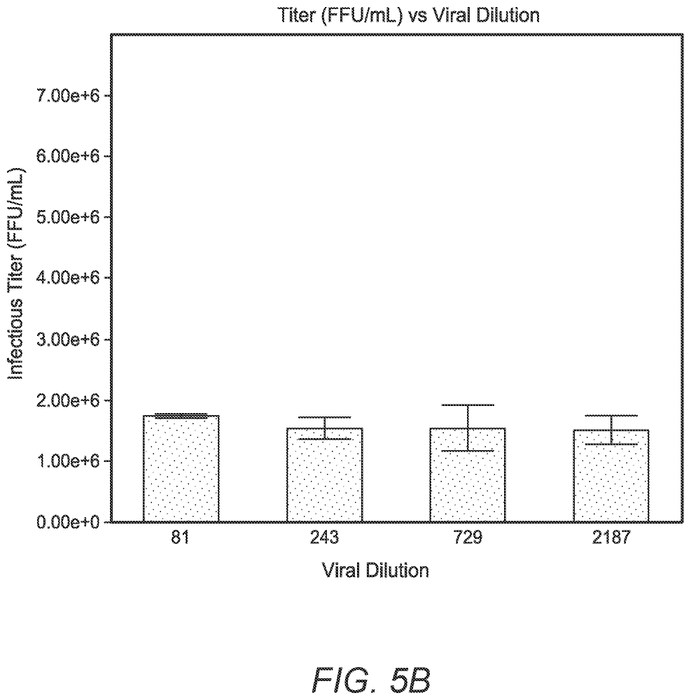

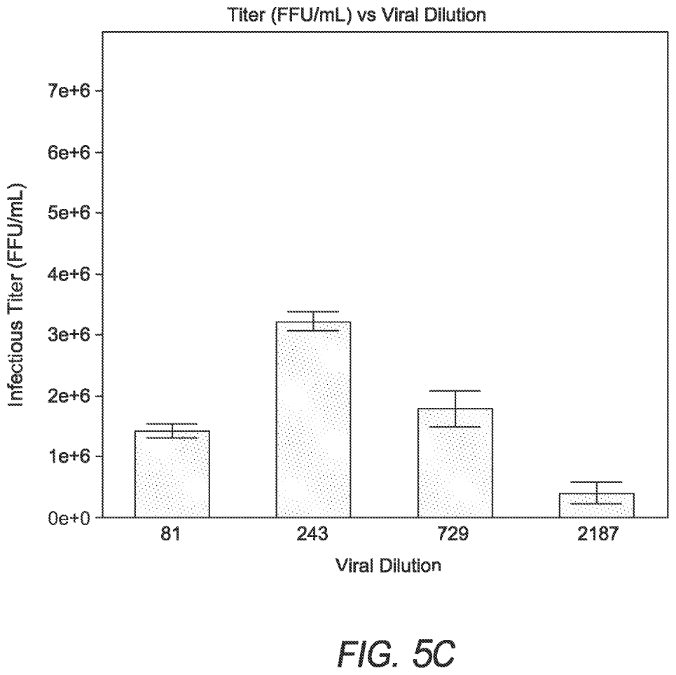

The utility of transfected UL82 mRNA can also be applied to cell lines for use in determining the infectious titer of viral stocks. Current methods for viral titering involve the use of cell lines created to produce pp71 when induced by the addition of exogenous chemicals. The creation of these cell lines is labor and time intensive and the ability to complement function by transfection of mRNA could potentially save development time. MRC-5 and BJ-5ta cells transfected with UL82 mRNA display viral titers constant across a wide viral dilution series (FIGS. 5A, 5B). This is in sharp contrast to the pp71 BJ-5ta doxycycline inducible cell line, where viral titers appear to decrease at lower inoculum, an effect which can be interpreted as insufficient levels of pp71 (FIG. 5C).

In addition, transfection of naturally permissive MRC-5 cells with UL82 mRNA leads to much higher apparent titers as compared to either transfection of BJ-5ta cells or the pp71 BJ-5ta cell line. This likely provides a more accurate titer of the material and allows better quantification of diluted material used in dose range studies. Transfection of UL82 mRNA should allow for lower titer viruses to be tested (e.g., less than 5e4 FFU/mL) with greater assay reproducibility and confidence.

### Example 2

Transient Transfection of UL82 mRNA for Complementation of UL82-Deleted HCMV Vectors

Complementation of UL82(pp71)-deleted vectors by providing the protein in trans, using pp71 mRNA transfection, was evaluated in another example. This approach may significantly reduce the dose and potentially stabilize the virus product with a full complement of tegument protein. These studies demonstrated reproducibility with additional operators, demonstrated titration of the mRNA transfection amount, and evaluated the pp71 mRNA transfection in the production process with HYPERStacks®.

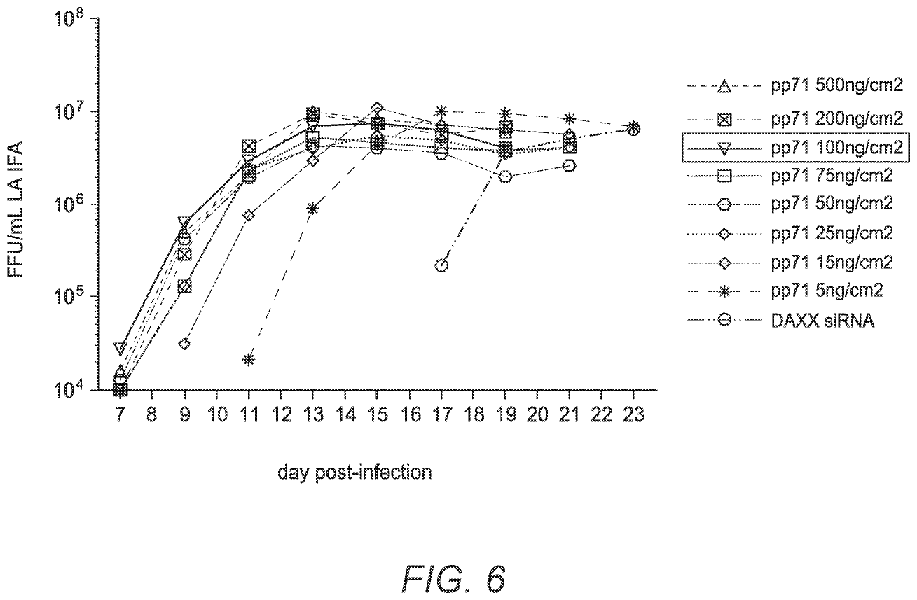

Accelerated growth kinetics were observed for pp71-deleted viruses when pp71 mRNA was transfected into MRC-5 cells prior to infection. These experiments were conducted by transfecting 100 ng/cm² of pp71 mRNA into cells seeded at 6.7×10³ cells/cm². To further improve virus growth kinetics, dose ranges of pp71 mRNA from 5 ng/cm² to 500 ng/cm² were investigated (FIG. 6). Even at 5 ng/cm², a decrease in the number of days to reach peak titer was observed compared to a previous production process using anti-DAXX SiRNA.

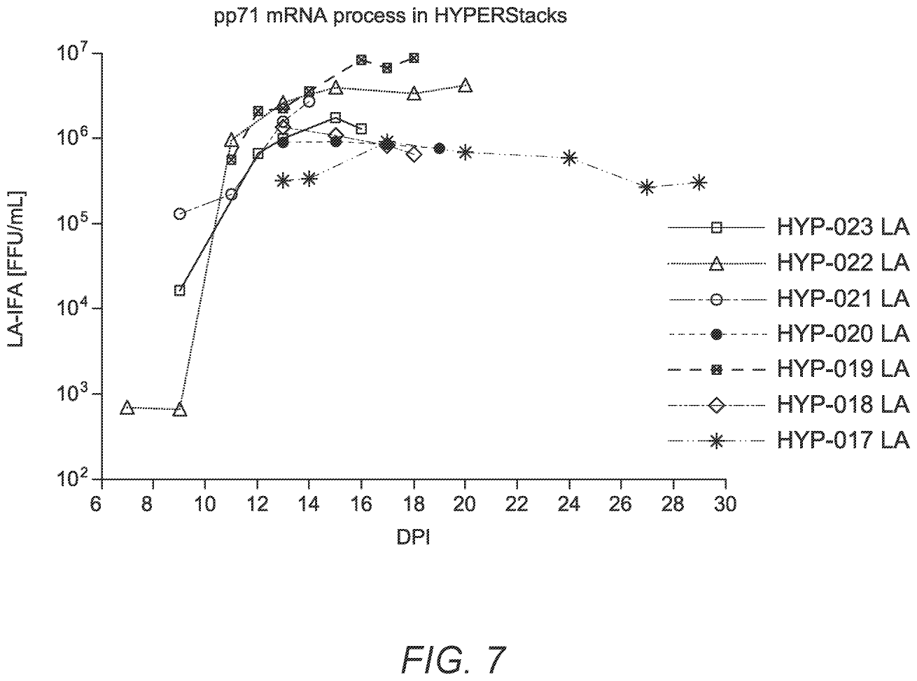

After the dose range experiments concluded, the 100 ng/cm² condition was selected to move forward into the production process using HYPERStacks®. Seven runs with pp71 mRNA were performed in either HYPERStack-12s or HYPERStack-36s to confirm process scalability (FIG. 7).

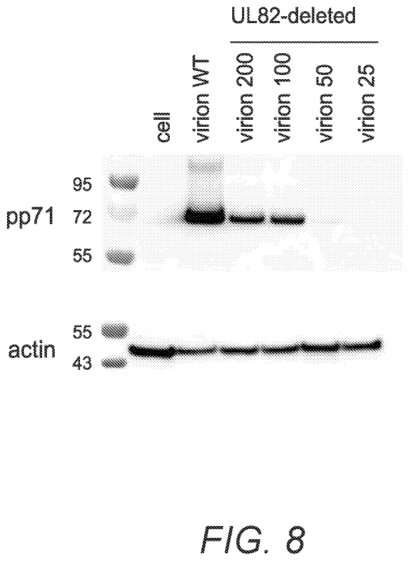

An immunoblot study was used to evaluate pp71 protein incorporation in the virion over a range of pp71 mRNA concentrations (FIG. 8). These data suggest that an mRNA concentration between 50-100 ng/cm² is necessary to detect pp71 in virions via immunoblot.

To evaluate whether the pp71 mRNA-expressed protein loaded into virions was functional, titers of pp71-deleted viruses in primary MRC-5 cells (no pp71 complementation) were compared to titers in a pBJ5TA fibroblast cell line (+pp71 complementation). A tissue culture infectious dose 50 (TCID50) titer assay was used for the uncomplemented MRC-5 cells and a late antigen immunofluorescence assay (LA IFA) was used for the pp71-complemented BJ5TA fibroblast cell line. In preliminary experiments, virus stocks produced with either anti-DAXX siRNA or pp71 mRNA were evaluated in both titer assays (Table 1). For pp71-deleted viruses produced with pp71 mRNA, the uncomplemented TCID50 titers were <1 log lower than the complemented LA IFA titers, similar to wildtype TR3 virus, suggesting that functional pp71 is incorporated in the virions. In contrast, the uncomplemented TCID50 titers for the pp71-deleted viruses produced with anti-DAXX siRNA were >2 log lower than the complemented LA IFA titers, indicating the lack of functional pp71. These results suggest that this comparative titer assay can be used to confirm pp71 protein function in pp71 mRNA-produced virus stocks and used in potency assays.

TABLE 1
Comparative titer assay to evaluate pp71 protein function.

| | LA IFA | Log titer | TCID50 | Log titer | Log |
|---|---|---|---|---|---|
| Virus | FFU/mL¹ | LA IFA | FFU/mL² | TCID50 | Difference |
| TR3 WT | 1.4E+06 | 6.1 | 4.1E+05 | 5.6 | 0.5 |
| Anti-DAXX | 1.7E+06 | 6.2 | 1.0E+04 | 4.0 | 2.2 |
| produced | | | | | |
| CMV | | | | | |
| Vector 1 | | | | | |
| pp71 | 1.2E+06 | 6.1 | 4.9E+05 | 5.7 | 0.4 |
| produced | | | | | |
| CMV | | | | | |
| Vector 1 | | | | | |
| Anti-DAXX | 6.9E+05 | 5.8 | 1.4E+03 | 3.1 | 2.7 |
| produced | | | | | |
| CMV | | | | | |
| Vector 2 | | | | | |
| pp71 | 4.0E+06 | 6.6 | 5.6E+05 | 5.7 | 0.9 |
| produced | | | | | |
| CMV | | | | | |
| Vector 2 | | | | | |

LA IFA titer is determined in pp71 complemented pBJ5TA fibroblasts.
²TCID50 titer is determined in uncomplemented primary MRC-5 fibroblasts.

### Example 3

Development of a pp71 mRNA Transfection Process for pp71-Deleted HCMV Production

### 1.0 BACKGROUND

HCMV pp71 (UL82 ORF) may be deleted from vaccines to attenuate virus replication for improved safety. Pp71 is a 71 kDa tegument phosphoprotein delivered to cells upon viral entry. Pp71 has several roles, including immediate early regulation of viral gene expression, promotion of protein translation, and immune evasion by inhibiting intrinsic cellular factors (Kalejta 2020). For efficient virus production in vitro, pp71-deleted vectors require either direct or functional pp71 complementation. Functional complementation approaches use siRNA transfection to inhibit cellular Daxx expression. Knockdown of DAXX compensates for the absence of pp71 inhibition of intrinsic cell defenses (Cantrell 2006, Preston 2006, Saffert 2006, Woodhall 2006). However, direct pp71 complementation is desirable for its potential to enhance immediate early (IE) activation compared to anti-DAXX siRNA knockdown, as well as complementing other functions of pp71.

As shown in this Example, growth of the pp71-deleted backbone vector is accelerated upon transfection of pp71 mRNA compared to the previous transfection process with anti-DAXX siRNA. Direct pp71 complementation also has the potential to load HCMV virions with pp71 protein which may lower vaccine dose by increasing the efficiency of the first round of vector replication.

Complementation of pp71 (UL82)-deleted vectors by providing the protein in trans has demonstrated the potential for an immunogenic dose reduction in the Rhesus CMV model (Marshall 2019). By adapting the anti-DAXX siRNA transfection process to pp71 mRNA complementation, the dose may be significantly reduced, providing manufacturing and clinical benefit, and potentially stabilizing the virus product with a full complement of tegument protein. Vaccine culture systems can utilize a pp71 complementing producer cell line, or anti-DAXX siRNA transfection can be replaced by the transient transfection of pp71 mRNA, whose development and implementation will be described in this report.

### 2.0 METHOD PRINCIPLE

Pp71-deleted HCMV production via anti-DAXX siRNA or pp71 mRNA are both based on transfecting cells using a lipid-based transfection reagent allowing entry of the nucleic acid into cells, followed by infection. The process flow for producing pp71 deleted HCMV with anti-DAXX siRNA is as follows:
1) 10 μM anti-DAXX siRNA transfection using Lipofectamine 2000 on −1 days post infection (DPI)
2) Infection at MOI 0.01 on 0 DPI
3) 2nd anti-DAXX siRNA transfection on 10±1 DPI
4) Media exchange to reduced serum on 11±1 DPI
5) Harvest at full CPE 20-24 DPI

The improvement to viral replication that pp71 protein enables allows for omission of the second transfection step, which is a process improvement for production because it removes a full step of the process. Another process improvement that pp71 mRNA transfection enables is culture harvest at an earlier DPI, approximately one week. The process flow for producing pp71 deleted HCMV with pp71 mRNA is as follows:
1) pp71 mRNA transfection using Lipofectamine MessengerMax on-1 DPI
2) Infection at MOI 0.01 on 0 DPI
3) Media exchange to reduced serum on 5 DPI
4) Harvest at full CPE 12-16 DPI.

### 3.0 METHOD SUMMARY

In T-flasks, MRC-5 fibroblasts are transfected on day 3 post-seeding (>70% confluent) when cells are seeded at 6.7×10³ cells per cm². In HYPERStacks, MRC-5 fibroblasts are transfected on day 4 post-seeding (>85% confluent) when cells are seeded at 6.7×10³ cells per cm².

The protocol for transfecting pp71 mRNA is as follows:
1. Determine the vessels to be transfected and calculate the total culture volume (Vf)
2. Label two tubes, one for the transfection reagent MessengerMax and one for the nucleic acid
3. To each tube add 1/16th Vf serum-free media (SFM)
4. To the transfection reagent tube add 1.9 μL MessengerMax per mL of Vf, mix by inversion and incubate for 10 minutes
5. To the nucleic acid tube add 100 ng/cm² pp71 mRNA and mix by inversion
6. Add the contents of the transfection reagent tube to the nucleic acid tube, mix by inversion and incubate for 5 minutes
7. Add transfection mix to complete growth media (CGM) in vessel(s) to be transfected and rock to distribute

The day after transfection, vessels are infected with pp71-deleted HCMV at a MOI of 0.01. On day 5 post-infection, the complete growth media (CGM; DMEM containing 9% FBS and 2 mM GlutaMax) is replaced with reduced serum media (RSM; DMEM containing 0.2% FBS and 2 mM GlutaMax).

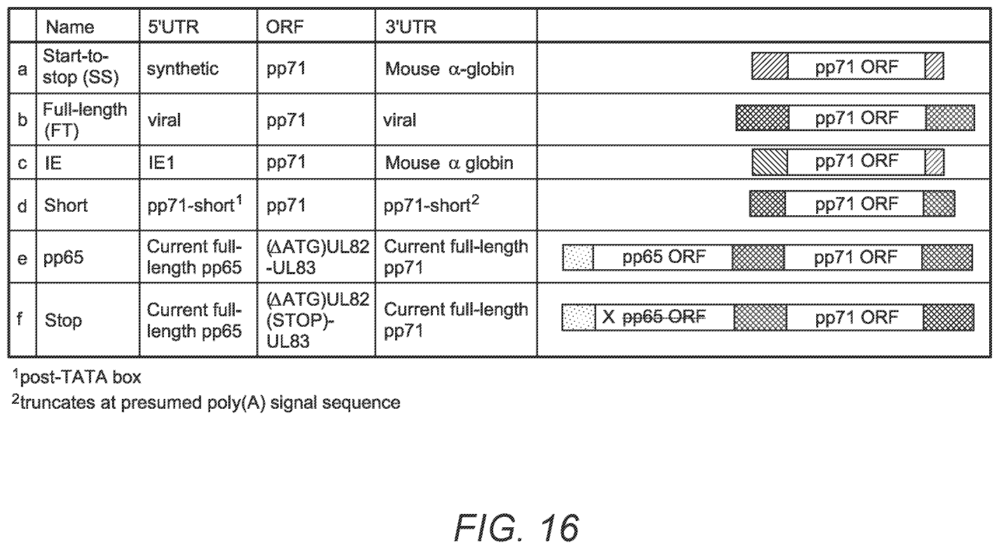

The GMP produced pp71 mRNA construct to be used in manufacturing is fully substituted with pseudouridine (pseudoU) and 5-methylcytidine (5meC), contains the natural HCMV UL82 ORF 5′ and 3′ UTRs, and is produced with a plasmid template 80 nucleotide poly-A tail (see FIG. 16, Construct B, SEQ ID NO: 4).

### 4.0 DEFINITIONS

| Term/Acronym | Definition |
|---|---|
| BDS | Bulk drug substance |
| CGM | Complete growth media (DMEM, 9% FBS, 2 mM |
| | GlutaMax) |
| CPE | Cytopathic effect |
| DAXX | Death-associated protein 6 |
| DPI | Day post-infection |
| DPT | Day post-transfection |
| EGFP | Enhanced green fluorescent protein |
| FFU | Focus forming unit |
| HCMV | Human cytomegalovirus |
| IE | Immediate-early |
| IFA | Immunofluorescence essay |
| gB | Glycoprotein B |
| LA | Late antigen |
| MOI | Multiplicity of infection |
| mRNA | Messenger RNA |
| MFI | Mean fluorescence intensity |
| ORF | Open reading frame |
| RNA | Ribonucleic acid |
| RSM | Reduced serum media (DMEM, 0.2% FBS, 2 mM |
| | GlutaMax) |
| siRNA | Small interfering RNA |
| SFM | Serum-free media (DMEM, 2 mM GlutaMax) |
| TB | Tuberculosis |
| TCID50 | Tissue culture infectious dose 50 |
| TFF | Tangential flow filtration |
| UTR | Untranslated region |
| WT | Wild type |

### 5.0 METHOD DEVELOPMENT

5.1 Generation of V5-Tagged pp71 mRNA Constructs and Detection of V5 Expression in Cell Lysates

A pp71 mRNA construct was designed from the start codon of the HCMV UL82 open reading frame to the stop codon with a V5 tag, a synthetic 5′UTR and a mouse α-globin 3′UTR (FIG. 16, Construct A). Pp71 protein expression was detected using an anti-V5 antibody.

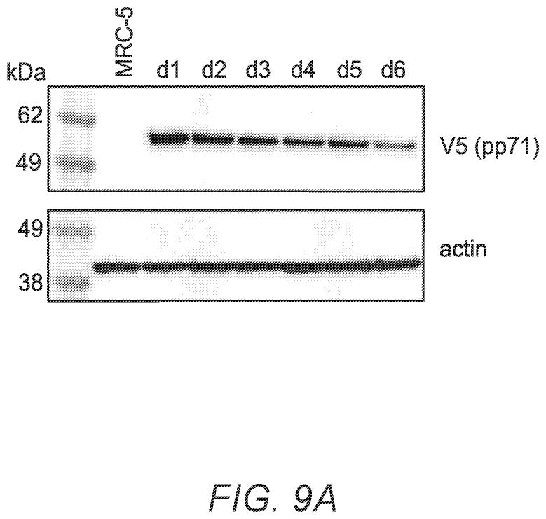

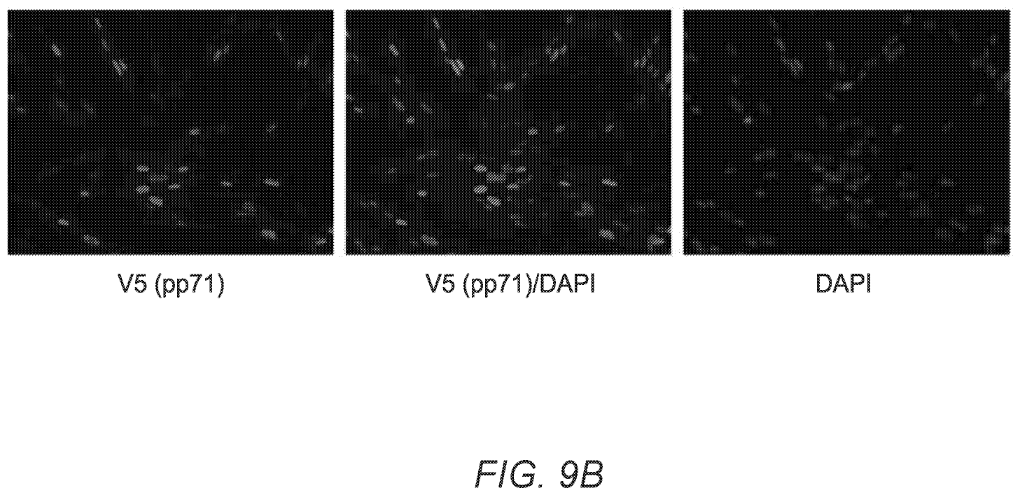

To determine if pp71 protein was detectable in MRC-5 fibroblasts after transfection of pp71-V5 mRNA, immunoblot and immunofluorescence analyses were performed. MRC-5 fibroblasts were transfected with 50 ng/cm² of pp71-V5 mRNA using the transfection reagent Lipofectamine 2000 (used for anti-DAXX siRNA transfection). For immunoblots, a cell pellet time-course was collected from day 1 to day 6 post-transfection and resuspended in the lysis buffer modified RIPA buffer plus protease inhibitors. A BCA protein assay was performed to normalize protein loading, and 50 μg total protein was loaded per sample on a NuPAGE™ 4-12% Bis-Tris gel. An MRC-5 cell lysate was used as a negative cell control sample. Immunoblot analysis of the cell lysates using a V5 antibody shows expression of pp71-V5 protein out to day 6 post-transfection (FIG. 9A). Pp71 is a 71 kDa phosphoprotein and as seen in FIG. 9A, pp71-V5 protein is running in the 50-60 kDa range. A molecular weight protein ladder study was performed to determine accuracy in the 60-80 kDa target range. The results showed that the SeeBlue™ Plus2 Pre-stained Protein Standard used in FIG. 9A did not perform as well as the Fisher BioReagents™ EZ-Run™ Prestained Rec Protein Ladder (ANT-010, NB35). Therefore, in all further immunoblots the EZ-Run ladder is used.

An immunofluorescence assay (IFA) was performed on MRC-5 fibroblasts transfected with 50 ng/cm² of pp71-V5 mRNA at 48 hours post-transfection. Cells were fixed with 4% formaldehyde in PBS and stained with a primary anti-V5 tag antibody followed by a Cy-5 labeled secondary antibody. Pp71-V5 protein is expressed and localized to the nuclear region, which is counterstained with the nuclear stain DAPI (FIG. 9B).

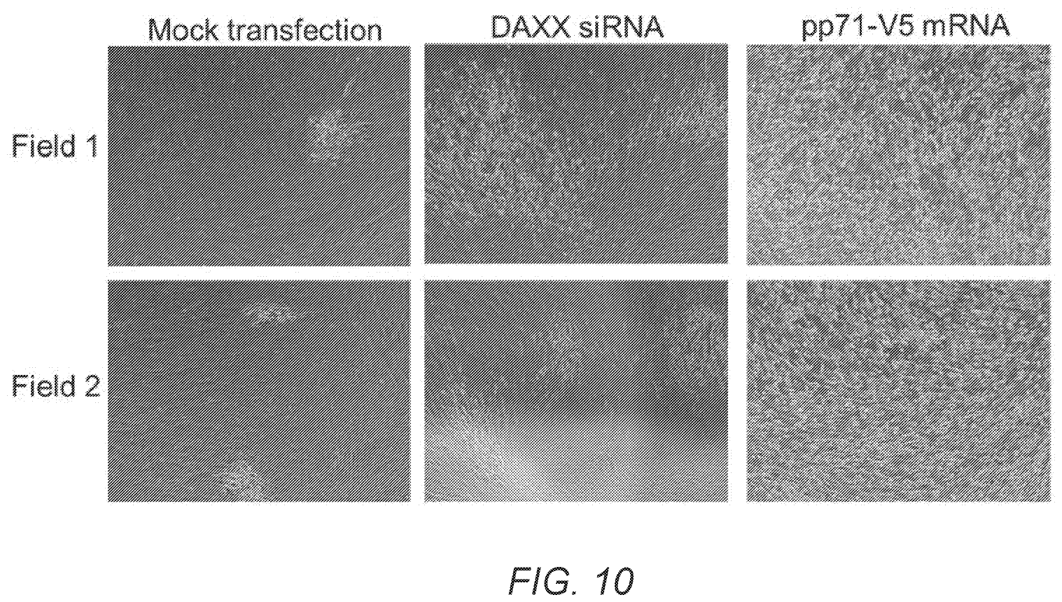

After confirming that the transfected pp71-V5 mRNA expressed pp71-V5 protein in MRC-5 fibroblasts, functional analyses of the pp71-V5 protein were performed via pp71-deleted HCMV replication assessment. Preliminary infection data indicated that viral cytopathic effect (CPE) progressed faster with transfection of pp71 mRNA compared to anti-DAXX siRNA. The pp71-deleted HCMV production process using anti-DAXX siRNA for complementation consists of two 10 μM anti-DAXX siRNA transfections, one the day before infection (DPI-1) and the second anti-DAXX siRNA transfection at DPI 10. Considering the immunoblot analysis showed pp71 protein levels decreasing at day 6 post-transfection (FIG. 9A), the second pp71 mRNA transfection was introduced earlier at DPI 5 or day post-transfection 6. CPE progression at 13 DPI was captured in phase images at 4× comparing MRC-5 fibroblasts mock transfected, transfected with 10 μM anti-DAXX siRNA at −1 and 10 DPI or transfected with 50 ng/cm² pp71-V5 mRNA at −1 and 5 DPI and infected with pp71-deleted HCMV at MOI 0.01 (FIG. 10). MRC-5 fibroblasts complemented with pp71 mRNA exhibit advanced CPE at 13 DPI compared to MRC-5 fibroblasts complemented with anti-DAXX siRNA.

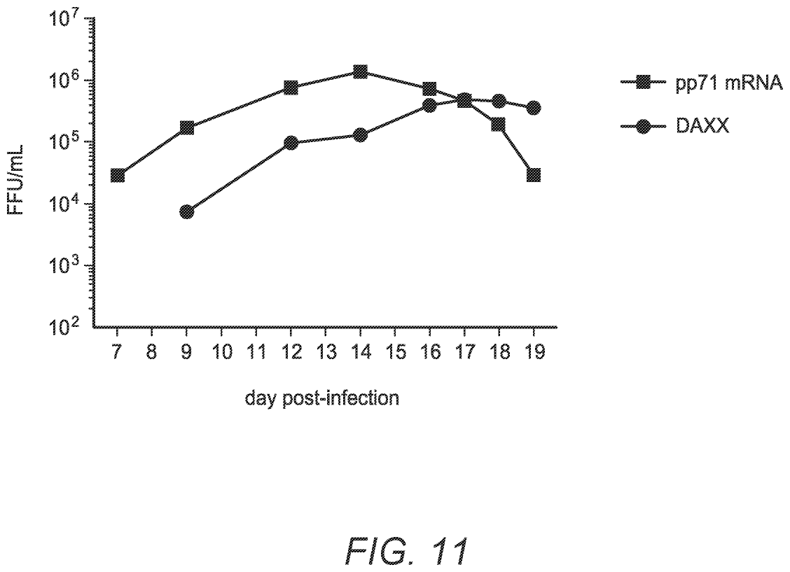

To further evaluate and quantitate the effect of pp71 mRNA complementation to viral replication, viral growth curves were performed. Briefly, MRC-5 fibroblasts were transfected with 10 μM anti-DAXX siRNA (−1 and 10 DPI) or transfected with 40 ng/cm² pp71-V5 mRNA (−1 and 6 DPI) prior to infection with pp71-deleted HCMV at MOI 0.01. Viral supernatant was harvested at multiple days post-infection and titered using the HCMV LA Immunofluorescence Titering Assay (TMD-QC-0061 v4.0). Virus was detected earlier with pp71 mRNA complementation (7 DPI vs 9 DPI), and the peak titer occurred earlier (14 DPI vs 17 DPI) when compared to virus grown using anti-DAXX siRNA transfection (FIG. 11). In addition, the peak titer was approximately 0.5 log higher with pp71 mRNA compared to anti-DAXX siRNA complementation. Both CPE evaluation (FIG. 10) as well as the viral growth curve (FIG. 11) show that transfection of pp71 mRNA improves pp71-deleted HCMV production.

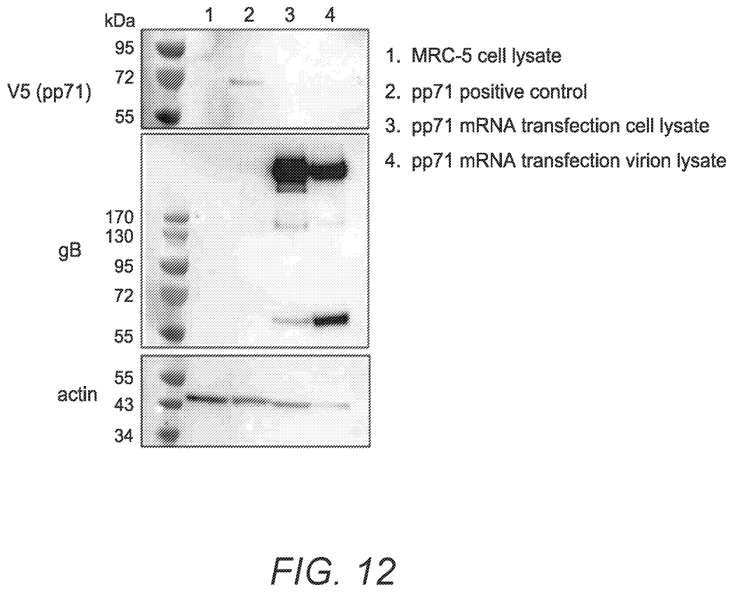

Next, whether pp71 protein from transfection was being incorporated into HCMV virions was determined. To assay for protein contained in virions, infected cell supernatant is collected at full CPE. The viral supernatant is first centrifuged at low speed (2,500×g, 15 min) to remove cellular debris, followed by high-speed centrifugation (24,000 RPM, 1 hr) to pellet virions through a 20% sorbitol cushion. The virion pellet is resuspended in lysis buffer (modified RIPA buffer plus protease inhibitors) and assayed by immunoblot. Previous data showed a lack of pp71-V5 protein expression when MRC-5 fibroblasts were transfected with 40 ng/cm² pp71-V5 mRNA at −1 and 6 DPI and infected with pp71-deleted HCMV at MOI 0.01, potentially due to the reduced expression of pp71-V5 protein by day 6 post-transfection shown in FIG. 9A. To increase the pp71 expression levels, an additional transfection with 40 ng/cm² pp71-V5 mRNA was added at 11 DPI and virions were collected at 13 DPI. Even with three mRNA transfections, no pp71-V5 protein was detected in infected cell lysate (lane 3) or in virion lysate (lane 4) by immunoblot using a V5 antibody (FIG. 12). Only the cell lysate from uninfected cells transfected with pp71 mRNA (pp71 positive control) showed pp71 expression (lane 2), suggesting that infection might inhibit pp71 expression from the exogenous mRNA. The lysate for the pp71 positive control was the same day 2 post-transfection lysate used in FIG. 9A. The presence of infection was shown using an antibody to Glycoprotein B (gB). The presence of actin in the virion lysate sample is potentially due to co-isolating proteins. Treating virion preparations with proteinase K removes cellular contamination of non-specific bound proteins, as indicated by a reduction in abundant cellular proteins including actin and tubulin (Turner 2020). A 15 ml volume of clarified viral supernatant was pelleted by ultracentrifugation and resuspended in 200 UL lysis buffer.

To investigate the lack of pp71 protein expression in cell lysates at late times during viral infection, the following actions were taken:
Tested different transfection reagents
Utilized GFP mRNA and wild-type virus infection
Tested different mRNA stabilizing nucleoside modifications
Designed alternative pp71 mRNA constructs

### 5.2 Transfection Reagent Selection

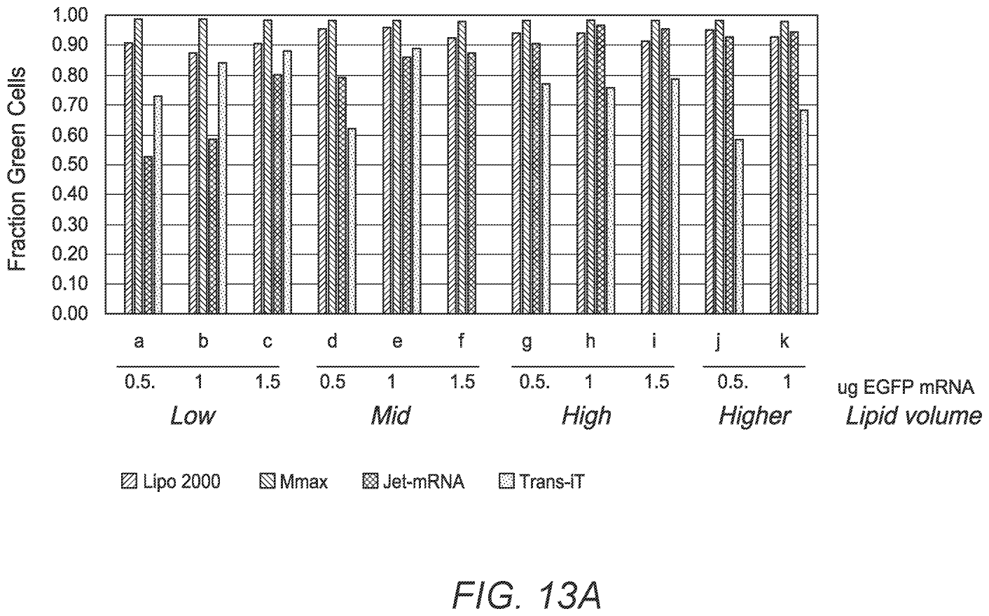

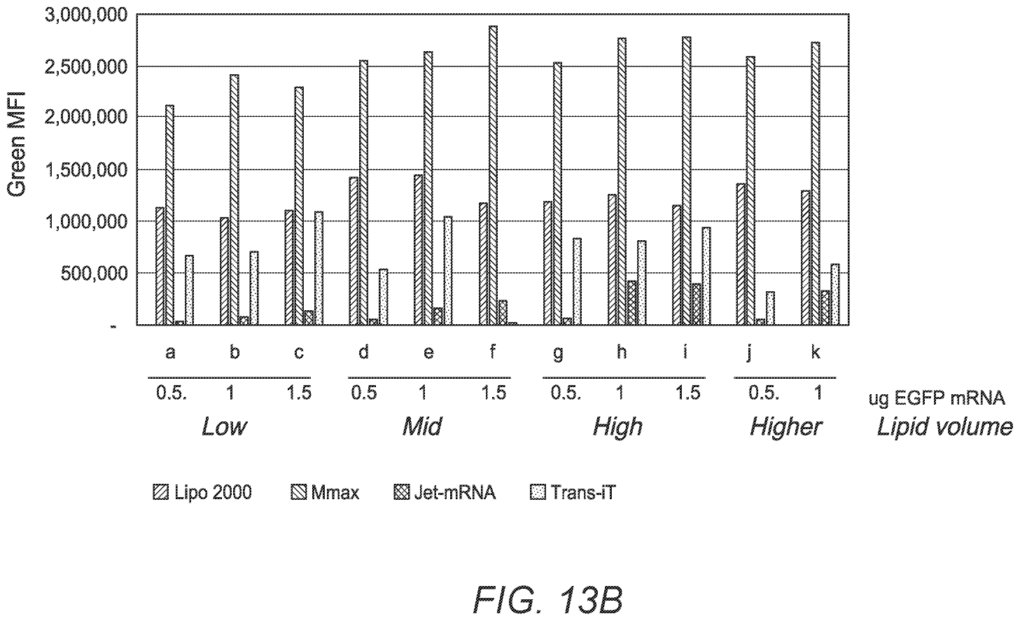

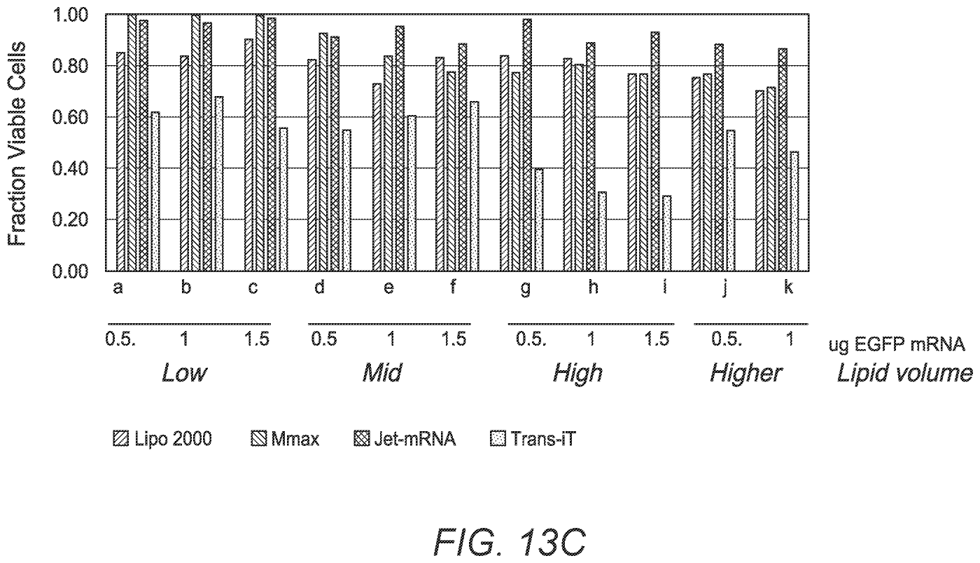

Several transfection agents were evaluated for transfection efficiency in MRC-5 fibroblasts. An EGFP mRNA similarly constructed to the pp71-V5 mRNA was used for testing. Cells were transfected with three amounts of mRNA (low, medium, high) using four transfection reagents, including Lipofectamine 2000 (ThermoFisher), MessengerMax (ThermoFisher), Jet-mRNA (PolyPlus) and Trans-IT (Mirus Bio) at four lipid levels and evaluated by flow cytometry for EGFP expression (FIG. 13A-9B; Table 3). MessengerMax resulted in the highest percentage of transfected cells (FIG. 13A), highest mean fluorescence intensity (MFI) (FIG. 13B), and highest cell viability (FIG. 13C). Lipid amounts were within the parameters of each lipid's optimal range and conditions were picked based on the ranges published in the manufacturer protocols.

TABLE 3
EGFP mRNA transfection reagent optimization in MRC5 cells.

| | | (μg) | (μl) | | | |
|---|---|---|---|---|---|---|
| | | EGFP | Lipofectamine | Messenger | Jet- | Trans- |
| | | mRNA | 2000 | Max | mRNA | IT |
| | | 0 | 0 | 0 | 0 | 0 |
| “Low” | A | 0.5 | 1 | 0.75 | 0.5 | 0.5 |
| | B | 1 | | | | |
| | C | 1.5 | | | | |
| “Mid” | D | 0.5 | 1.5 | 1 | 1 | 1 |
| | E | 1 | | | | |
| | F | 1.5 | | | | |
| “High” | G | 0.5 | 2 | 1.25 | 2 | 1.5 |
| | H | 1 | | | | |
| | I | 1.5 | | | | |
| “Higher” | J | 0.5 | 2.5 | 1.5 | 3.6 | 2 |
| | k | 1 | | | | |

All transfections were performed according to manufacturer instructions. All subsequent mRNA transfections in MRC-5 fibroblasts for virus production used the MessengerMax transfection reagent.

5.3 Additional Pp71 mRNA Constructs

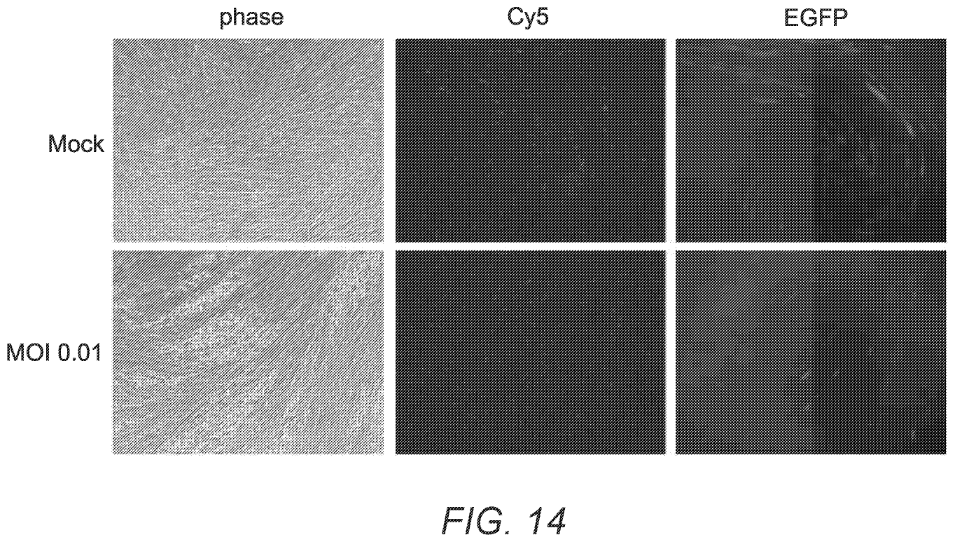

To address the lack of detectable pp71 protein at late times during HCMV infection, even after transfecting pp71 mRNA multiple times, the possibility that HCMV infection inhibits lipid-based mRNA transfection was considered. Utilizing an EGFP mRNA similarly constructed to the pp71 mRNA also containing a Cy5 tag allowed the visualization of the transfected mRNA as well as EGFP protein. MRC-5 fibroblasts were transfected with 50 ng/cm² EGFP-Cy5 mRNA at DPI-1 and DPI 6 and infected with WT TR3 at a MOI of 0.01 at DPI 0. The Cy5 signal was present during infection, but EGFP signal was lost at late times during infection (FIG. 14). Therefore, it appears that HCMV infection may inhibit some protein expression from the transfected mRNA. Multiple transfections were also performed at different times during the infection process, with no apparent boost to EGFP detection. Due to the faster CPE progression using pp71 mRNA compared to anti-DAXX siRNA, the improved transfection reagent (MessengerMax), and the lack of signal from a second transfection, a single transfection of pp71 mRNA performed prior to infection was used in further experiments.

Transfection of cells with unmodified RNAs can lead to cell death due to activation of innate immune pathways (Devoldere 2016). The following experiment addressed whether modification of the pp71 mRNA construct can stabilize the mRNA for better expression in MRC-5 fibroblasts. Stabilizers can include (Devoldere 2016):
Elongation of the poly-A tail
Modified cap structures
Modified nucleosides: 5-methylcytidine (5meC), N6-methyladenosine (m6A), 5-methyluridine (m5U), pseudouridine (pseudoU), 2-thioruridine (s2U), 5-methoxy uridine (5moU)
Removal of dsRNA contaminants through HPLC purification.

For example, it has been shown that substitution of uridine and cytidine residues with pseudouridine and 5-methylcytidine reduces innate immune recognition and that pseudouridine modified RNA is translated more efficiently (McCaffrey 2017, Kariko 2008). All pp71 mRNA constructs used in FIGS. 9-14 were stabilized with pseudoU and 5meC.

Utilizing EGFP mRNA, but with different nucleoside modifications, the expression of EGFP was compared in transfected cells with and without HCMV infection (WT TR3 at MOI 0.01). The following EGFP mRNA constructs were transfected at DPI-1 in MRC-5 fibroblasts.
1) no modification
2) 5moU
3) pseudoU/5meC
4) Cy5-UTP: 5moU

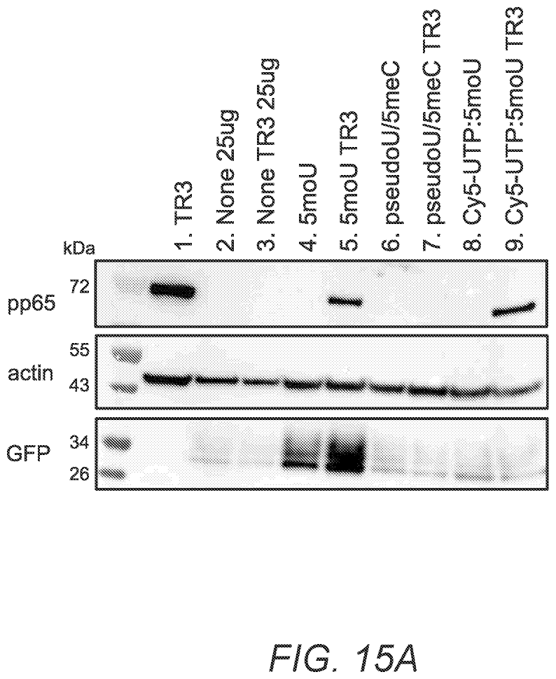

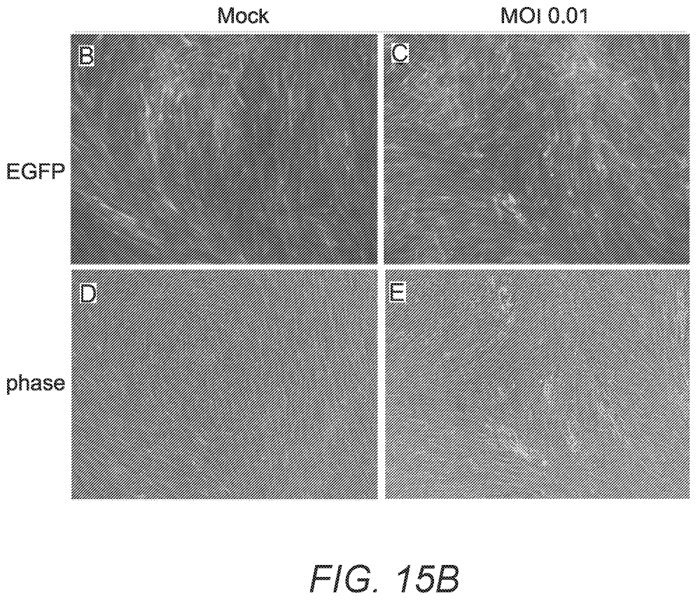

Note the Cy5-UTP: 5moU is Cy-5 labeled uridine triphosphate at a 1:3 ratio to 5moU and translation efficiency correlates inversely with Cy5-UTP substitution. EGFP mRNA was transfected at 100 ng/cm², doubling the amount previously used (FIG. 15A-15B), to assess if transfecting an increased amount of mRNA would lead to protein detection at later times during viral infection. Cell pellets were collected at DPI 7 and a representative immunoblot (FIG. 15A) showed increased EGFP expression in mRNA stabilized with 5moU during viral infection (lanes 4 and 5). Cell death was especially apparent in the monolayer transfected with mRNA containing no nucleoside modifications. The positive control for pp65 expression and the negative control for EGFP expression is TR3 infected cell lysate (lane 1). The above conditions were imaged at DPI 6; shown here is 5moU EGFP expression in transfection only (“B” in FIG. 15B) and in transfection with WT TR3 infection MOI 0.01 (“C” in FIG. 15B) of MRC-5 fibroblasts. Phase images of the same field show transfection-only MRC-5 fibroblasts (“D” in FIG. 15B) or the presence of CPE in infected MRC-5 fibroblasts (“E” in FIG. 15B). Of the nucleoside modifications tested, the 5moU modification showed increased EGFP expression and viral spread compared to the pseudoU/5meC modification in this set of conditions.

To explore the possibility that the lack of pp71 protein incorporation into the virion is due to exogenous pp71 not localizing to the correct cellular location additional pp71 constructs were designed (FIG. 16). The new constructs included an mRNA with the full-length viral pp71 5′ and 3′ UTRs (b), potentially enabling correct localization. A bicistronic mRNA that includes pp65 (UL83) (e) and an additional bicistronic mRNA with a stop codon in pp65 (f). The bicistronic mRNA is also transcribed in natural HCMV infection (Ruger 1987), potentially enabling correct localization. The addition of a stop codon in pp65 would prevent excess pp65 protein expression and the possible additional effects on viral infection. The pp71 short construct (d) contains a truncated 5′UTR beginning after the TATA box while the 3′UTR ends before the presumed poly(A) signal sequence. The final construct contains the HCMV IE1 5′UTR and the mouse α-globin 3′UTR (c), identical to the 3′UTR of the pp71 start to stop construct (a). The constructs do not contain a V5 epitope tag. All new constructs were made with the 5moU modified nucleoside. Construct (b), pp71 mRNA with the full-length viral 5′ and 3′ UTRs, was also made with the pseudoU/5meC modified nucleoside, same as the pp71 mRNA start to stop construct (a).

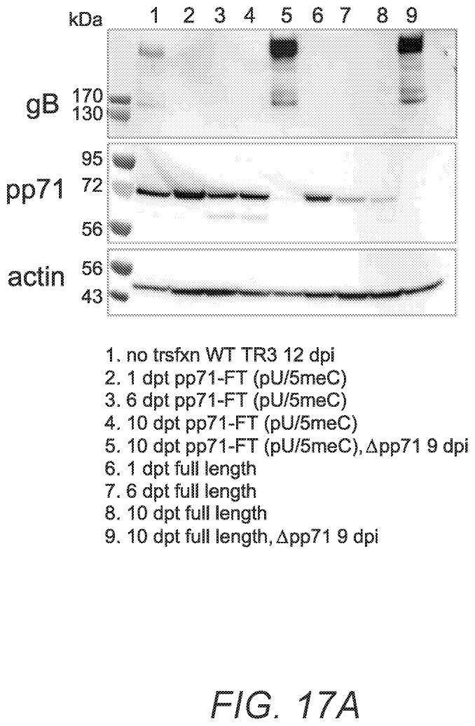

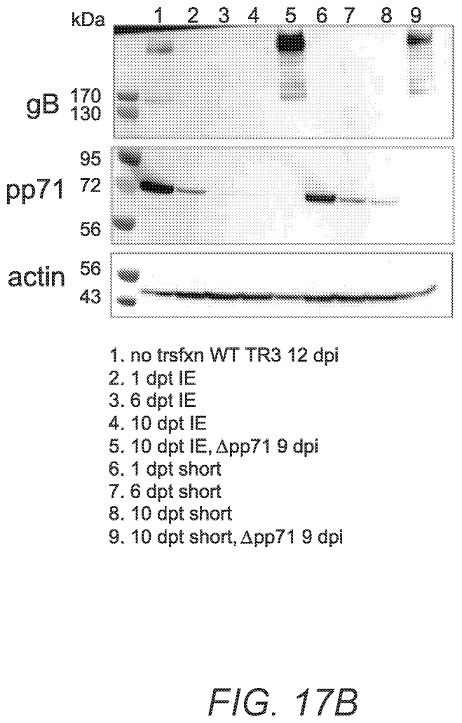

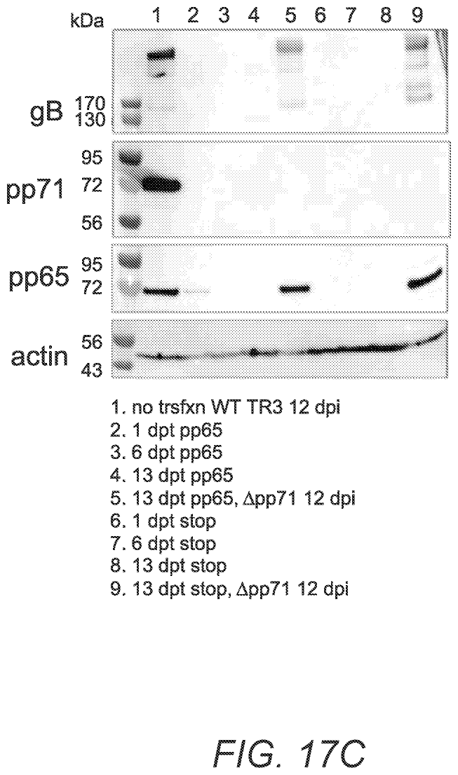

All constructs were transfected at 100 ng/cm² in MRC-5 fibroblasts with and without infection using a pp71-deleted vector at a MOI of 0.01 and cell lysates were assayed for pp71 protein expression by immunoblot (FIG. 17A-17C). Transfection only samples were harvested at 1, 6 and 10 or 13 days post-transfection (DPT) to show expression over time, while pp71-deleted infection samples were harvested at advanced CPE either 9 or 12 DPI (10 or 13 DPT respectively). WT TR3 infection at MOI 0.01 of MRC-5 fibroblasts harvested at 12 DPI serves as a positive control. Minimal to no expression of pp71 protein was detected, even in transfection only lysates. The highest expression of pp71 protein was detected in MRC-5 fibroblasts transfected with the full-length pp71 mRNA with the viral 5′ and 3′UTRs and with the pseudoU/5meC modified nucleoside (FIG. 17A, lanes 2-4). Although little pp71 protein expression was detected in the lysate with viral infection (FIG. 17A, lane 5) prior to this immunoblot, pp71 protein had not been detected. The increased expression previously detected with the 5moU EGFP mRNA combined with WT TR3 infection did not translate in the pp71 mRNA combined with TR3Δpp71 infection system. Likely using a non-viral mRNA coupled with a wild-type virus infection is not comparable to transfection with a viral mRNA coupled with a replication deficient virus because the overall infection life cycle is dissimilar. Subsequent experiments use a full-length pp71 mRNA construct with viral 5′ and 3′ UTRs (“Construct B”) wherein the viral 5′ and 3′ UTRs are modified with pseudoU/5meC.

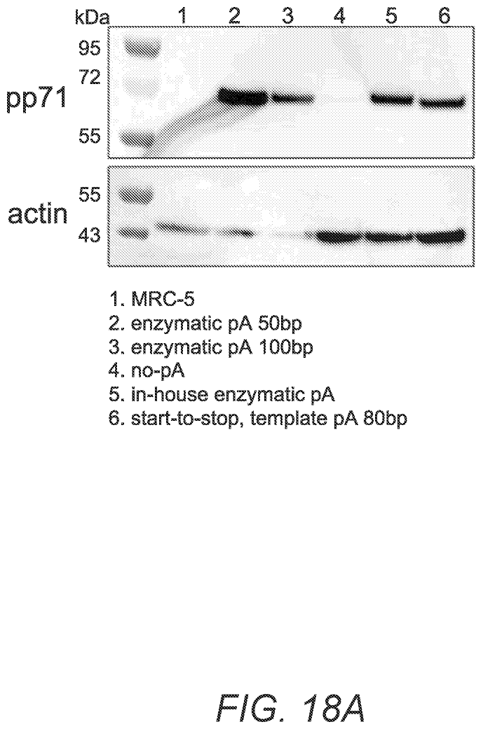

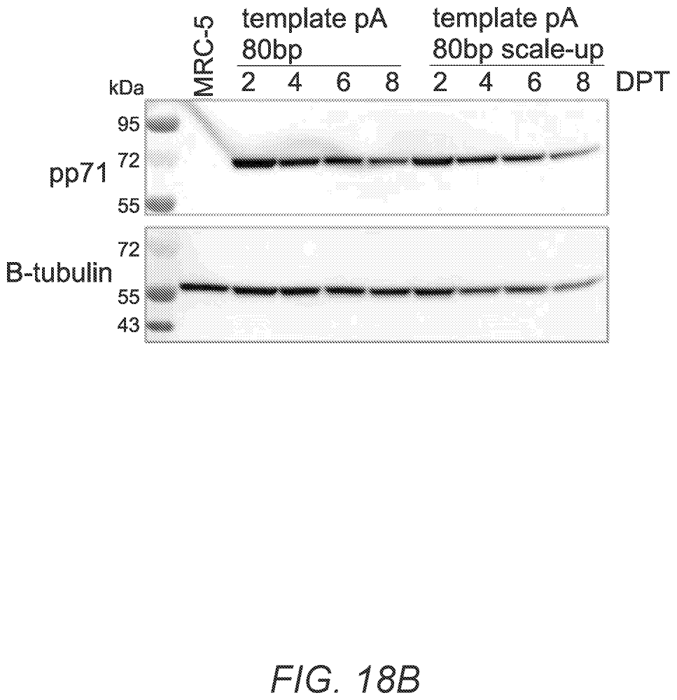

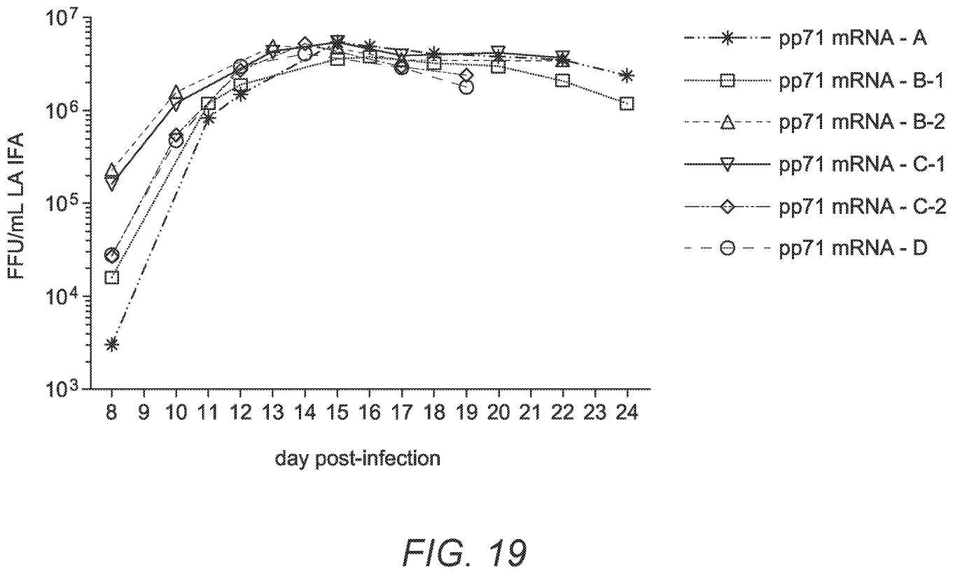

There are two production methods (TriLink Biotechnologies) to add the poly-A tail to the pp71 mRNA construct-either via (1) a plasmid template encoded tail designed to add 80 nucleotides, or (2) enzymatically, which variably adds approximately 60-100 nucleotides. A poly(A) tail was added via plasmid template to the start-to-stop pp71 mRNA construct, Construct A, with and without the V5 tag. Due to a manufacturing error the first lot of full-length pp71 mRNA with the viral 5′ and 3′ UTRs modified with pseudoU/5meC was produced with no poly-A tail. This lot of mRNA was tailed in-house enzymatically synthesizing an unknown tail length but functional protein by immunoblot (FIG. 18A) and growth curve (FIG. 19). The subsequent lot, was produced with an enzymatic 100 nucleotide tail. Due to the need for increased quantities, a next lot was produced on a larger scale with an enzymatically added poly-A tail, using the TriLink PD process. However, the TriLink PD process only produced a 50 nucleotide tail. To eliminate variable poly-A tail length from the manufacturing process the next lot of pp71 mRNA was produced with a templated 80 nucleotide poly-A tail. Due to the importance of the pp71 mRNA construct for HCMV production, immunoblots were performed comparing expression of pp71 protein from different lots of pp71 mRNA in MRC-5 cell lysates wherein the poly(A) tail was generated by different methods (FIG. 18A). MRC-5 fibroblasts were transfected with different lots of pp71 mRNA at 100 ng/cm² and cell pellets were collected at 5 days post-transfection. All lots of pp71 mRNA containing a poly-A tail by any method show pp71 protein expression up to day 5 post-transfection. Pp71 mRNA produced without a poly-A tail is shown in FIG. 18A, lane 4. FIG. 18B shows pp71 protein expression at 2-, 4-, 6-, and 8-days post-transfection testing “template pA 80 bp scale-up”, the PD scale-up run using the plasmid template poly-A production process in preparation for GMP manufacturing versus a standard-scale run “template pA 80 bp”, which also has a templated 80 nucleotide poly-A tail. MRC-5 fibroblasts were transfected with pp71 mRNA at 100 ng/cm², and cell pellets were collected. Both lots showed equivalent pp71 protein expression.

Multiple growth curves were also performed comparing pp71 mRNA lots produced with poly-A tails by template or enzymatically (FIG. 19). In separate growth curves combined in FIG. 19, MRC-5 fibroblasts were transfected at DPI −1 with the indicated lots of pp71 mRNA at 100 ng/cm² and infected with a pp71-deleted vector at MOI of 0.01. Viral titer in FFU/mL was determined by LA IFA at multiple days post-infection. The results show that all lots of pp71 mRNA can successfully complement production of pp71-deleted viruses in MRC-5 fibroblasts. Subsequent pp71 mRNAs were produced with 80 nucleotide poly(A) tails using a plasmid template.

5.4 pp71 mRNA Titration

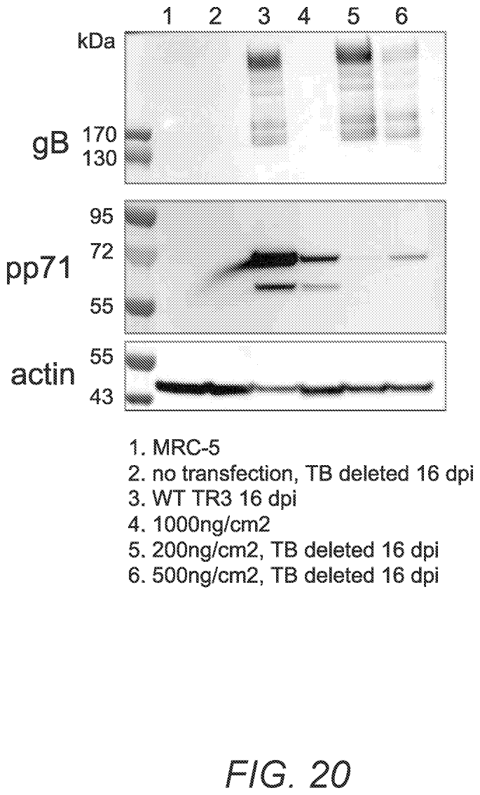

Considering the changes made to the pp71 mRNA transfection process as well as the positive pp71 protein signal visualized by immunoblot in pp71-deleted infected cell lysates (FIG. 17A-17C) the amount of pp71 mRNA transfected was investigated. In previous experiments 40-100 ng of pp71 mRNA per cm² of tissue culture area was transfected. FIG. 20 shows pp71 protein expression by immunoblot at 16 dpi in cell lysates after transfecting increased amounts (200, 500 and 1000 ng/cm²) of pp71 mRNA in MRC-5 fibroblasts combined with pp71-deleted virus (Tuberculosis deleted: TR3 mir124 ΔUL128-130 ΔUL146-147 ΔUL82 Ag85A-ESAT-6-Rv3407-Rv2626c-RpfA-RpfD) infection at MOI 0.01. An MRC-5 cell lysate was used as a negative cell control (lane 1), with untransfected MRC-5 fibroblasts infected at a MOI 0.01 with a pp71-deleted virus as the negative viral control (lane 2) and WT TR3 infection of MRC-5 fibroblasts at a MOI 0.01 as a positive control (lane 3). A pp71 mRNA transfection only cell lysate at 1000 ng/cm² shows pp71 expression out to 16 DPI (17 DPT, lane 4). Similarly, MRC-5 fibroblasts transfected with either 200 ng/cm² or 500 ng/cm² pp71 mRNA and infected at a MOI 0.01 with a pp71-deleted virus show pp71 protein expression at 16 DPI (17 DPT).

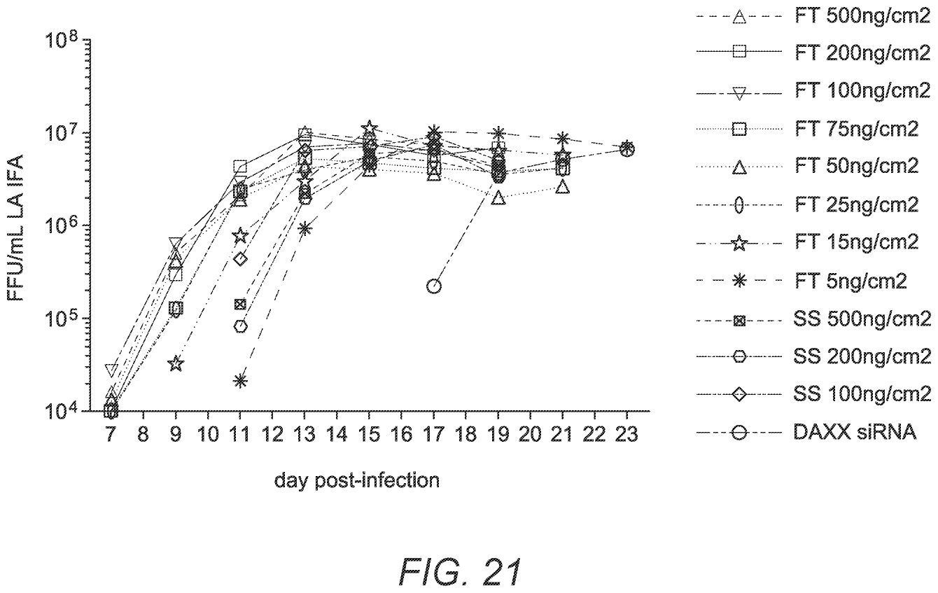

To further improve pp71-deleted virus production, growth curves were performed comparing a range of pp71 mRNA transfected from 5 ng/cm² to 500 ng/cm² as well as comparing the start-to-stop construct (SS) to the full-length construct with the viral 5′ and 3′ UTRs (FT) (FIG. 21). MRC-5 fibroblasts were transfected with pp71 mRNA (FT or SS from 5-500 ng/cm²) or anti-DAXX SIRNA (10 μM) followed by infection with a pp71-deleted virus at a MOI of 0.01 and viral titer in FFU/mL was determined by LA IFA at multiple days post-infection. The data shows that even at 5 ng/cm² of FT pp71 mRNA transfected, there is a marked decrease in the number of days to reach peak titer compared to the previous production process using anti-DAXX siRNA. At lower amounts of FT pp71 mRNA transfected, 15 ng/cm² and less, there is a delay in peak titer correlating with mRNA amount, while at FT pp71 mRNA amounts above 25 ng/cm² the growth curves are equivalent. The data also show that when transfecting the start-to-stop pp71 mRNA (SS) even at amounts up to 500 ng/cm² peak titers are delayed compared to using the full-length pp71 mRNA (FT), demonstrating the benefit of the native UTRs for virus production. Transfection with a quantity of 100 ng/cm² of pp71 mRNA was used in the HCMV pp71-deleted production process based viral growth curve kinetics (FIG. 21), pp71 protein expression by immunoblot (FIG. 17A-17C and FIG. 20), and evidence of virion-packaged pp71 protein by TCID50 (See Table 2).

TABLE 2
Comparative titer assay to evaluate pp71 protein function.

| | LA IFA | Log titer | TCID50 | Log titer | Log |
|---|---|---|---|---|---|
| Virus | FFU/mL¹ | LA IFA | FFU/mL² | TCID50 | Difference |
| TR3WT | 1.4E+06 | 6.1 | 4.1E+05 | 5.6 | 0.5 |
| TB deleted | 1.7E+06 | 6.2 | 2.5E+03 | 3.4 | 2.8 |
| DAXX | | | | | |
| TB deleted | 1.3E+06 | 6.1 | 4.8E+04 | 4.7 | 1.4 |
| pp71 200 | | | | | |
| TB deleted | 1.2E+06 | 6.1 | 4.8E+04 | 4.7 | 1.4 |
| pp71 100 | | | | | |
| TB deleted | 6.8E+05 | 5.8 | 3.5E+03 | 3.5 | 2.3 |
| pp71 50 | | | | | |
| TB deleted | 1.9E+06 | 6.3 | 4.8E+03 | 3.7 | 2.6 |
| pp71 25 | | | | | |

LA IFA titer is determined in pp71 complemented pBJ5TA fibroblasts.
²TCID50 titer is determined in uncomplemented primary MRC-5 fibroblasts.

### 5.5 Scale-Up for Manufacturing

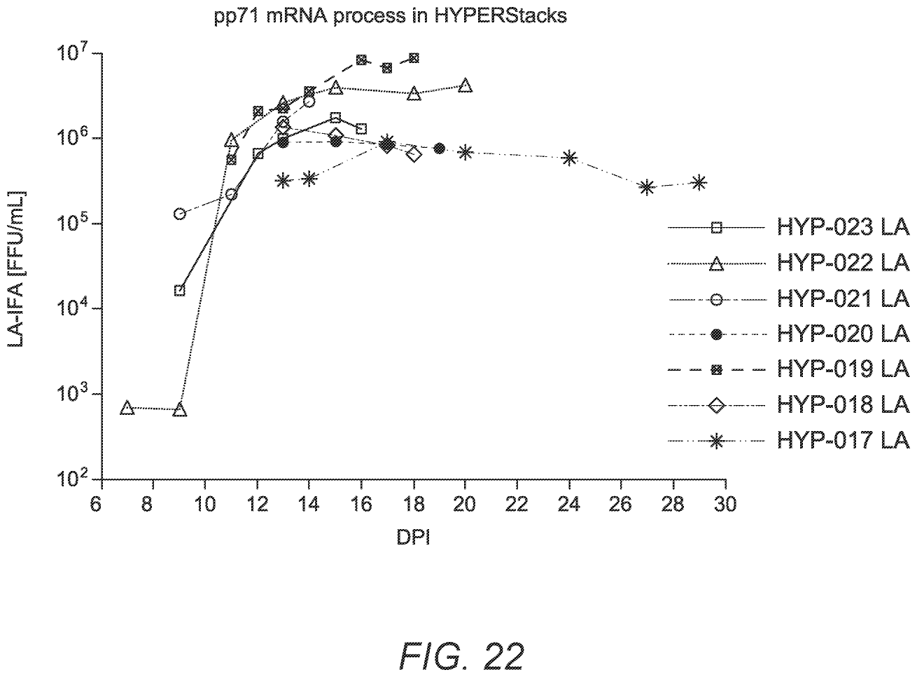

To confirm that the pp71 mRNA transfection method at 100 ng/cm² was scalable into HYPERStack format, seven runs were performed in either HYPERStack-12s or HYPERStack-36s. Three growth curves were performed in HYPERStack-12s following the process developed in T-flasks (FIG. 22). HS-12s were transfected day 3 post-seeding and infected on day 4 post-seeding with a pp71 deleted virus at a MOI of 0.01. Viral titer in FFU/mL was determined by LA IFA at multiple days post-infection. In the initial experiments following this format, the time of transfection at 3 days post-seeding revealed the HYPERStack-12s were only 70-75% confluent. Based on the observed lower sustained peak titers (less than 1×10⁶ FFU/mL), we changed the transfection procedure to 4 days post-seeding to achieve greater than 85% confluency for subsequent growth curves.

Four growth curves performed in HYPERStack-12s and HYPERStack-36s transfected on day 4 post-seeding or greater than 85% confluency and infected on day 5 post-seeding with a pp71 deleted virus at a MOI of 0.01 resulted in LA IFA titers greater than 1×10⁶ FFU/mL by 12 to 14 DPI (FIG. 22). This confirmed the scalability of the pp71 mRNA transfection method at 100 ng/cm² to HYPERStack vessels when transfection is performed 4 days post-seeding at 6.67×10³ cells/cm².

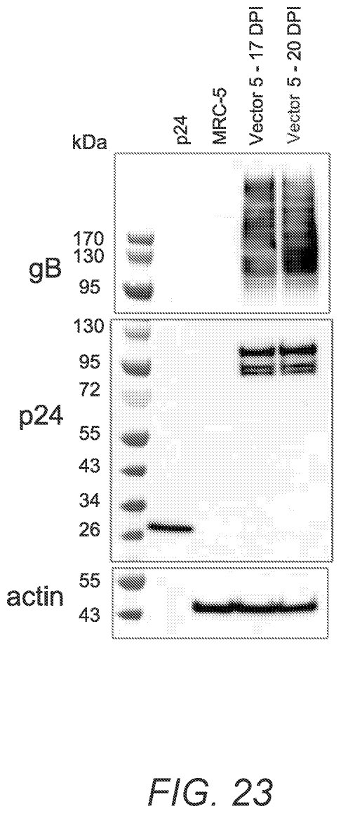

In some HCMV vectors used to express heterologous genes, such as an antigen, the gene of interest replaces the UL82 ORF. An immunoblot was performed to investigate if the process change (producing pp71-deleted vectors via pp71 mRNA transfection), affects antigen expression from the UL82 (pp71) locus (FIG. 23). Uncomplemented MRC-5 fibroblasts were harvested at 8 DPI after infection at a MOI of 0.5 with Vector 5 (TR3 Δ146-147 Δ128-130 ΔUL82 M conserved gag/nef/pol fusion episensus 1) virus produced with pp71 mRNA at 100 ng/cm² harvested at either 17 or 20 DPI. 5 ng of purified p24 protein was used as the positive control sample (p24, a component of M conserved gag/nef/pol fusion episensus 1) and MRC-5 cell lysate was used as the negative control sample (MRC-5). M conserved gag/nef/pol fusion episensus 1 antigen (SEQ ID NOs: 11-12) expression was confirmed in MRC-5 fibroblast cell lysates infected with Vector 5 produced with pp71 mRNA.

### 5.6 Exogenous Pp71 Protein Function

The next set of experiments were designed to assess whether the pp71 mRNA expressed protein loaded into virions is functional. Four virus stocks were produced by transfecting MRC-5 fibroblasts with pp71 mRNA at 25, 50, 100 and 200 ng/cm² and infecting with a pp71-deleted virus at a MOI of 0.01. The virus stock used to infect these cultures was produced with the anti-DAXX siRNA process, therefore virions used for infection did not contain any pp71 protein.

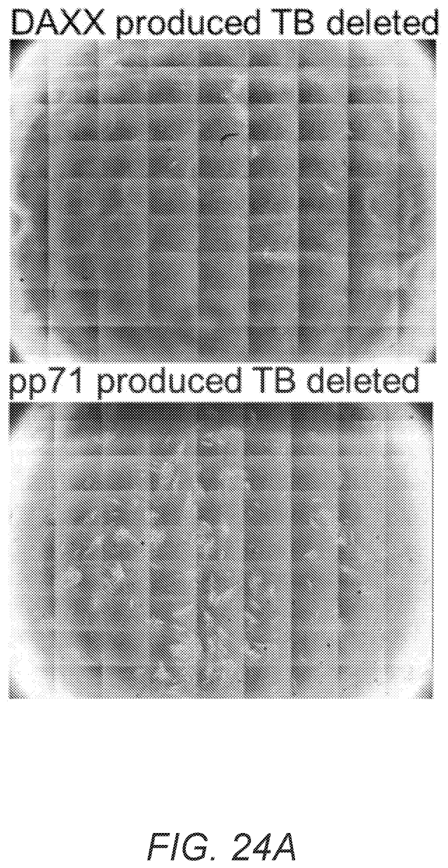

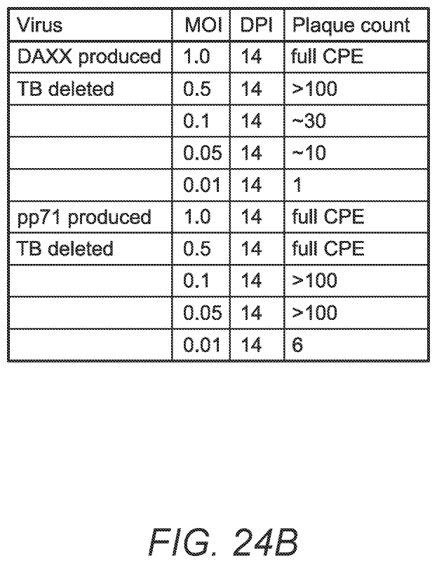

First, uncomplemented MRC-5 fibroblasts were infected over a range of MOIs with a pp71-deleted virus grown either in the presence of 10 μM anti-DAXX siRNA or pp71 mRNA at 200 ng/cm² (FIG. 24A-24B). Cultures were monitored for spread and number of plaques. Phase images show CPE at 13 DPI in uncomplemented MRC-5 fibroblasts infected with a pp71-deleted virus either produced with anti-DAXX siRNA (top panel) or pp71 mRNA (bottom panel) at a MOI of 0.01 (FIG. 24A). At 14 DPI plaques were counted visually. Virus grown in the presence of pp71 mRNA (200 ng/cm²) had an increase in plaque number at lower MOIs (FIG. 24B).

Concurrently, a tissue culture infectious dose 50 (TCID50) titer assay in uncomplemented MRC-5 fibroblasts was evaluated using the virus stocks produced with 25, 50, 100 or 200 ng/cm² pp71 mRNA compared to a virus stock produced with the anti-DAXX siRNA process as well as WT TR3 (Table 2). TCID50 titers were compared to titers from the late antigen immunofluorescence assay (TMD-QC-0061) in the pp71-complemented BJ-5TA fibroblast cell line. The log difference between the titer assays shows that as the amount of pp71 mRNA transfected is reduced the functional complementation is also reduced. The log difference for a virus stock produced with 25 ng/cm² pp71 mRNA is more similar to the log difference for a virus stock produced with anti-DAXX siRNA (>2 logs), whereas the log difference for virus stocks produced with at least 100 ng/cm² pp71 mRNA is less than 2 logs.

To further test the TCID50 assay results, 5 replicate TCID50s were performed for two viruses produced with DAXX anti-siRNA and two viruses produced with 100 ng/cm² pp71 mRNA. The two vectors were TB deleted: TR3 mir124 ΔUL128-130 ΔUL146-147 ΔUL82 Ag85A-ESAT-6-Rv3407-Rv2626c-RpfA-RpfD and Vector 2: TR3 Δ146-147 Δ128-130 ΔUL82 M conserved gag/nef/pol fusion episensus 1 ΔUL18. Average titers were compared between TCID50s in uncomplemented MRC-5 fibroblasts and the current LA IFA using the pp71-complemented BJ5TA fibroblast cell line. In pp71-deleted viruses produced with pp71 mRNA, the uncomplemented TCID50 titers were less than 1 log lower than the complemented LA IFA, similar to WT TR3 (see Table 1), suggesting that functional pp71 is incorporated in the virions. In contrast, the uncomplemented TCID50 titers for the pp71-deleted viruses produced with anti-DAXX siRNA were greater than 2 logs lower than the complemented LA IFA titers, indicating the lack of functional pp71. These results suggest that this comparative titer assay can be used to confirm pp71 protein function in pp71 mRNA produced virus stocks. The range of TCID50 titers for pp71-produced TB deleted was 3.5×10⁵ to 7.4×10⁵ FFU/mL and for pp71-produced Vector 2 was 4.1×10⁵ to 7.4×10⁵ FFU/mL. While the range of TCID50 titers for DAXX-produced TB deleted was 8.7×10³ to 1.1×10⁴ FFU/mL and for DAXX-produced Vector 2 was 1.2×10³ to 1.9×10³ FFU/mL.

### Section 5.7 Detection of Virion Pp71 Protein by Immunoblot

A series of experiments were performed to find controls and conditions for an immunoblot assay to detect exogenous pp71 protein in HCMV vector virions. Virion pp71 protein detected by immunoblot thus far could not be distinguished from cell membrane fragments or vesicles pelleted with virions by ultracentrifugation after transfection of MRC-5 fibroblasts with pp71 mRNA. Several attempts were made to create an ultracentrifuged non-infected transfection control sample to confirm pp71 protein incorporation into virions.

Negligible protein was released from pp71 mRNA transfected MRC-5 monolayers that were not been infected. Three freeze/thaw cycles were used to mimic an infected lysed MRC-5 monolayer. An alternative approach to isolate virions prior to immunoblot analysis was developed.

A two-step sucrose gradient was used to isolate virions from the T-flask production process along with transfection control samples and samples for an assay for pp71 protein by immunoblot (Dai 2014).

pp71 mRNA transfection control gradient purified lysate was obtained by following the pp71 mRNA production process in T-flasks but omitting infection. The monolayer was scraped in the media at DPI 14, collected, and subjected to three freeze/thaw cycles. The infected sample was obtained by following the pp71 mRNA production process in T-flasks, the supernatant was collected separately from the cell pellet at DPI 14. Three T150s per sample type were combined to allow for enough material to be loaded onto the gradient. Both supernatant samples were clarified by centrifugation at 5,000×g for 15 min. The clarified supernatants were ultracentrifuged at 21,000 RPM for 1 h at 15° C. and resuspended in 2 mL of PBS at pH 7.4.

The sucrose gradient protocol was as follows. In a SW41 Ti Beckman ultra-rotor, 5 mL of 50% sucrose in PBS was overlayed with 5 ml of 15% sucrose in PBS. Next, 2 mL of each sample type in PBS was overlayed. Samples were spun at 21,000 RPM for 1 hour at 15° C. and the ultracentrifuge deacceleration was set to coast. Two bands were seen in the infected sample gradient at the interface between the sucrose layers. The lower band was narrower than the upper band and the bands were too close together to collect separately. The upper band was predicted to contain more virions, while the lower band likely contained more dense bodies, but the majority of particles in both bands were likely virions with DNA-filled capsids (Dai 2014). Visually there was one band in the transfection control gradient purified sample, which was also at the interface of the sucrose layers. Approximately 1 mL was collected from each gradient and diluted with PBS to a total volume of 12 mL. A final spin to concentrate the samples was performed in the SW41 Ti at 21,000 RPM for 1 h at 15 C. The pellet was resuspended in 100 uL lysis buffer.

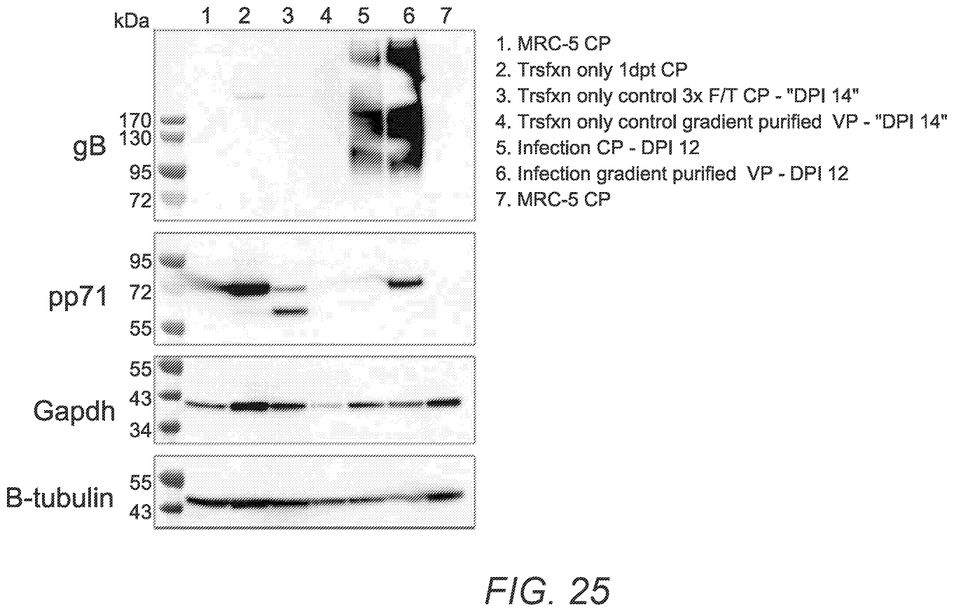

An immunoblot showed pp71 protein present in the transfection-only cell lysate control (FIG. 25, lane 3). Some protein degradation was observed, potentially due to the 3× freeze/thaw procedure. There was no pp71 protein present in the transfection only gradient purified lysate control (FIG. 25, lane 4). In previous immunoblots the ultracentrifuged control sample pp71 protein was visible. The increased clarification speed prior to ultracentrifugation potentially removed more cellular debris from the supernatant.

The gradient immunoblot was repeated with increased clarification speed at 10,000×g (data not shown). This resulted in less total protein recovered from the control transfection only gradient purified sample. While these experiments did not provide evidence that pp71 was incorporated in the virion, optimizing the separation of cellular debris from virions and/or using an alternative assay could provide further information.

### REFERENCES

Cantrell S R, and Bresnahan W A (2006). Human cytomegalovirus (HCMV) UL82 gene product (pp71) relieves hDaxx-mediated repression of HCMV replication. J Virol, 80, 6188-6191. doi: 10.1128/JVI.02676-05.
Dai X, and Zhou Z H (2014). Purification of herpesvirus virions and capsids. Bio Protoc, August 5; 4 (15).
Devoldere J, Dewitte H, De Smedt S C, Remaut K (2016). Evading innate immunity in nonviral mRNA delivery: don't shoot the messenger. Drugs Discovery Today, 21 11-25 DOI: 10.1016/j.drudis.2015.07.009.
Kalejta R F and Albright E R (2020) Expanding the Known Functional Repertoire of the Human Cytomegalovirus pp71 Protein. Front Cell Infect Microbiol, 10:95. doi: 10.3389/fcimb.2020.00095.
Karikó K, Muramatsu H, Welsh F A, Ludwig J, Kato H, Akira S, and Weissman D (2008). Incorporation of Pseudouridine Into mRNA Yields Superior Nonimmunogenic Vector with Increased Translational Capacity and Biological Stability. Mol Ther, November; 16 (11): 1833-1840.
Marshall E E, Malouli D, Hansen S G, Gilbride R M, Hughes C M, Ventura A B, Ainslie E, Selseth A N, Ford J C, Burke D, Kreklywich C N, Womack J, Legasse A W, Axthelm M K, Kahl C, Streblow D, Edlefsen P, Picker L J, Fruh K (2019). Enhancing safety of cytomegalovirus-based vaccine vectors by engaging host intrinsic immunity. Sci Transl Med, July 17; 11 (501): eaaw2603. doi: 10.1126/scitransimed.aaw260.
McCaffrey A P, Azizian K T, Shin D, Shore S, Henderson J M, Lebedev A, Jessee J, Hogrefe R I and Zon G. Poster: Maximizing Translation of mRNA Therapeutics By Sequence Engineering and Chemical Modification. 7th Cambridge Symposium on Nucleic Acids Chemistry & Biology 2017. TriLink BioTechnologies, San Diego, CA 92121, USA. MTI-GlobalStem, Gaithersburg MD 20877, USA.
Preston C M, and Nicholl M J (2006). Role of the cellular protein hDaxx in human cytomegalovirus immediate-early gene expression. J Gen Virol, 87, 1113-1121. doi: 10.1099/vir.0.81566-0.
Ruger B, Klages S, Walla B, Albrecht J, Fleckenstein B, Tomlinson P, Barrell B (1987). Primary structure and transcription of the genes coding for the two virion phosphoproteins pp65 and pp71 of human cytomegalovirus. J Virol, 61 (2): 446-453.
Saffert R T, and Kalejta R F (2006). Inactivating a cellular intrinsic immune defense mediated by Daxx is the mechanism through which the human cytomegalovirus pp71 protein stimulates viral immediate-early gene expression. J Virol, 80, 3863-3871. doi: 10.1128/JVI.80.8.3863-3871.2006.
Turner D L, Komeev D V, Purdy J G, de Marco A, Mathias R A (2020). The host exosome pathway underpins biogenesis of the human cytomegalovirus virion. ELife, 9: e58288.
Woodhall D L, Groves I J, Reeves M B, Wilkinson G, and Sinclair J H (2006). Human Daxx-mediated repression of human cytomegalovirus gene expression correlates with a repressive chromatin structure around the major immediate early promoter. J Biol Chem, 281, 37652-37660. doi: 10.1074/jbc.M604273200.

SEQUENCES

SEQ ID NO: 1 (UL82 gene-Human herpesvirus 5 strain TR (GenBank
Accession No. KF021605.1: 118811-120490))
CTAGATGCGGGGTCGACTGCGTGGGGTGCTGGAAGTGGAAGCGGTGCTG
ATGGGTGAGGGTCGTGGCGCGGGCACGGACCGCAACGTGCTGCTGATGT
CTGCCGCGGTACGCACGTCGCCGTCCATGTCGCTGCGCAGATAAGAGGT
AGGTCGTAATGCGGCGTGCTGCACGCTCACCGTTAATGGTACCAAGTCGT
CAAGGCTCGCAAAGACGTGCCACGAGGGGATGACGAGCGTGAGAGCCCC
GTTGTTACCGCTTCGACGTCTTTGTCCGGTCAGGATCAGTGCCCGGGACA
GTCCGGCTTGGGTGTCCGAGTCCTCGTCGCCGCTGGCCTCCTCGAAGCC
GGCAAACATGGCTTCGGACAGGGGGGTCGGCGTCGGTGTGGAGGAGAG
GTCATCTTCGTCGTCCTCTTCCTCTTCTTCCTCCTCTTCCTCGGTGGGTGG
TAATCCGGGGGACTGCGGGAGAAACTCGGAGACGGCGCCGCGCACGAC
GTCGCTCCGTGGAAAGAGACCGGCGCGCAGCTGCACCTGGGGACGCTTG
ATTTTGTCCGGTTTACCGGGTGTGAGAGTCCAAAACCCACGGCGGAAAAA
GTGGATGCGGCCTAGCGGCTGTCGGTGTTCCAAATGAACGGCCTGGTCG
CCGGTCAGCGTGACGCGGAGGGTGATTCGCACACGATCGGGTAGCGGG
CCGGCTTCTATGGAGACGCCCGGGATGTTTTCCGGGAAAAAGATGGTGTC
GTGAGTCTGATTGGTTTCGAAAGCATTCTGGATCTGCACGATGTACTCGG
GATGTATGCGCGTCAGCGTAAAACTTTTGGGAATCAACAGCTGGAAGCCG
TTGTCCGGCAAGCGTCGTAGGTGCGGGTACGGATTGTGTCGCGCCACCA
CCTCGGCGCGATGCGTGTAAACCGAAAAGTGCAGAAACACGCTGGTCGG
CGGGTGCGGTGAGTCGTGATGCAGAAACAGCATGATCCATTGGCCTCGTT
CGTCCGTCTCCGTTTTGTGGATGTACGTGTTAGGGTCCGAACAGGCCAGC
TGCTCCAGGGCGTCTACCAGCGTCAGCGGGATGGCGCCGGCGCGAAAG
GCGAACTGGCTGACAAAGATCTGCCCTGCCTCCAAACTGCTGTCGGTTCT
GCGGCGCCAGTTCGGCGTCACGGTCAGTCGCACGGCCCAGTGGTGAGC
CGTGCGGCGGATGATGGCGCGCGCCTCCATTCGCGGCCGATTTTCTTCG
CCGCCGCGCCGCTGGCTCTGAAAGAGGTGCAGTCCGCTAACGGGCACGC
GGTCTAGCGGCAGCGCAAAGGCCAGCACCGAGACCGTGTTGTTTTCTGA
GCCTGGCGTCAGGCGTCGTGGGCCAAAGTTGTTGAGGTCCACCAGCAGT
CGGTCCCGTTCGCCCACCACGCAGCGGCCCTTGATGTTTAGGTCGGTCA
GGTCTACGGTGTCGTGCGGAGATTTGTTCTCCTGAAAACAGCAGAGAACC
GAGGGTCGGCTCACCTCTATGTTGGTACGCAGGTCCAGGAGTCGCAGAC
GACCGGCTTCCAGCGAGCCGCCTTCCACGTTGGTGATGAGCCGAAGCAC
CTGGCAGTGCAGGCGACCAAAGCTGCCGCTGGCGGCTTCGGCCTCGCTG
ATCGCGGCCGCTTCCGACGAGGGTCCCTCACCGGGCGAGGACGATGCCT
GAGACAT
SEQ ID NO: 2 (tegument protein pp71-Human betaherpesvirus 5 (GenBank
Accession No. AGL96671.1))
MSQASSSPGEGPSSEAAAISEAEAASGSFGRLHCQVLRLITNVEGGSLEAGR
LRLLDLRTNIEVSRPSVLCCFQENKSPHDTVDLTDLNIKGRCVVGERDRLLVDL
NNFGPRRLTPGSENNTVSVLAFALPLDRVPVSGLHLFQSQRRGGEENRPRM
EARAIIRRTAHHWAVRLTVTPNWRRRTDSSLEAGQIFVSQFAFRAGAIPLTLVD
ALEQLACSDPNTYIHKTETDERGQWIMLFLHHDSPHPPTSVFLHFSVYTHRAE
VVARHNPYPHLRRLPDNGFQLLIPKSFTLTRIHPEYIVQIQNAFETNQTHDTIFF
PENIPGVSIEAGPLPDRVRITLRVTLTGDQAVHLEHRQPLGRIHFFRRGFWTLT
PGKPDKIKRPQVQLRAGLFPRSDVVRGAVSEFLPQSPGLPPTEEEEEEEEED
DEDDLSSTPTPTPLSEAMFAGFEEASGDEDSDTQAGLSRALILTGQRRRSGN
NGALTLVIPSWHV
FASLDDLVPLTVSVQHAALRPTSYLRSDMDGDVRTAADISSTLRSVPAPRPSP
ISTASTSSTPRSRPRI
SEQ ID NO: 3 (Tegument protein pp71-Human betaherpesvirus 5 (UniProtKB-
R4SH92))
MSQASSSPGEGPSSEAAAISEAEAASGSFGRLHCQVLRLITNVEGGSLEAGR
LRLLDLRTNIEVSRPSVLCCFQENKSPHDTVDLTDLNIKGRCVVGERDRLLVDL
NNFGPRRLTPGSENNTVSVLAFALPLDRVPVSGLHLFQSQRRGGEENRPRM
EARAIIRRTAHHWAVRLTVTPNWRRRTDSSLEAGQIFVSQFAFRAGAIPLTLVD
ALEQLACSDPNTYIHKTETDERGQWIMLFLHHDSPHPPTSVFLHFSVYTHRAE
VVARHNPYPHLRRLPDNGFQLLIPKSFTLTRIHPEYIVQIQNAFETNQTHDTIFF
PENIPGVSIEAGPLPDRVRITLRVTLTGDQAVHLEHRQPLGRIHFFRRGFWTLT
PGKPDKIKRPQVQLRAGLFPRSDVVRGAVSEFLPQSPGLPPTEEEEEEEEED
DEDDLSSTPTPTPLSEAMFAGFEEASGDEDSDTQAGLSRALILTGQRRRSGN
NGALTLVIPSWHVFASLDDLVPLTVSVQHAALRPTSYLRSDMDGDVRTAADIS
STLRSVPAPRPSPISTASTSSTPRSRPRI
SEQ ID NO: 4 (pp71 Construct B-Full-length (FT))
AGGGCCACCCGCCGCGCACGCGCUUAAGACGACUCUAUAAAAACCCACG
UCCACUCAGACACGCGACUUUUGGGCGCCACACCUGUCGCCGCUGCUA
UAUUUGCGACAGUUGCCGGAACCCUUCCCGACCUCCCACGAAGACCCG
UUCACCUUUGCGCAUCCCCUGACCCUCCCCCCAUCCCGCCUUCGCAAU
GUCUCAGGCAUCGUCCUCGCCCGGUGAGGGACCCUCGUCGGAAGCGGC
CGCGAUCAGCGAGGCCGAAGCCGCCAGCGGCAGCUUUGGUCGCCUGCA
CUGCCAGGUGCUUCGGCUCAUCACCAACGUGGAAGGCGGCUCGCUGGA
AGCCGGUCGUCUGCGACUCCUGGACCUGCGUACCAACAUAGAGGUGAG
CCGACCCUCGGUUCUCUGCUGUUUUCAGGAGAACAAAUCUCCGCACGA
CACCGUAGACCUGACCGACCUAAACAUCAAGGGCCGCUGCGUGGUGGG
CGAACGGGACCGACUGCUGGUGGACCUCAACAACUUUGGCCCACGACG
CCUGACGCCAGGCUCAGAAAACAACACGGUCUCGGUGCUGGCCUUUGC
GCUGCCGCUAGACCGCGUGCCCGUUAGCGGACUGCACCUCUUUCAGAG
CCAGCGGCGCGGCGGCGAAGAAAAUCGGCCGCGAAUGGAGGCGCGCGC
CAUCAUCCGCCGCACGGCUCACCACUGGGCCGUGCGACUGACCGUGAC
GCCGAACUGGCGCCGCAGAACCGACAGCAGUUUGGAGGCAGGGCAGAU
CUUUGUCAGCCAGUUCGCCUUUCGCGCCGGCGCCAUCCCGCUGACGCU
GGUAGACGCCCUGGAGCAGCUGGCCUGUUCGGACCCUAACACGUACAU
CCACAAAACGGAGACGGACGAACGAGGCCAAUGGAUCAUGCUGUUUCU
GCAUCACGACUCACCGCACCCGCCGACCAGCGUGUUUCUGCACUUUUC
GGUUUACACGCAUCGCGCCGAGGUGGUGGCGCGACACAAUCCGUACCC
GCACCUACGACGCUUGCCGGACAACGGCUUCCAGCUGUUGAUUCCCAA
AAGUUUUACGCUGACGCGCAUACAUCCCGAGUACAUCGUGCAGAUCCAG
AAUGCUUUCGAAACCAAUCAGACUCACGACACCAUCUUUUUCCCGGAAA
ACAUCCCGGGCGUCUCCAUAGAAGCCGGCCCGCUACCCGAUCGUGUGC
GAAUCACCCUCCGCGUCACGCUGACCGGCGACCAGGCCGUUCAUUUGG
AACACCGACAGCCGCUAGGCCGCAUCCACUUUUUCCGCCGUGGGUUUU
GGACUCUCACACCCGGUAAACCGGACAAAAUCAAGCGUCCCCAGGUGCA
GCUGCGCGCCGGUCUCUUUCCACGGAGCGACGUCGUGCGCGGCGCCG
UCUCCGAGUUUCUCCCGCAGUCCCCCGGAUUACCACCCACCGAGGAAG
AGGAGGAAGAAGAGGAAGAGGACGACGAAGAUGACCUCUCCUCCACACC
GACGCCGACCCCCCUGUCCGAAGCCAUGUUUGCCGGCUUCGAGGAGGC
CAGCGGCGACGAGGACUCGGACACCCAAGCCGGACUGUCCCGGGCACU
GAUCCUGACCGGACAAAGACGUCGAAGCGGUAACAACGGGGCUCUCAC
GCUCGUCAUCCCCUCGUGGCACGUCUUUGCGAGCCUUGACGACUUGGU
ACCAUUAACGGUGAGCGUGCAGCACGCCGCAUUACGACCUACCUCUUAU
CUGCGCAGCGACAUGGACGGCGACGUGCGUACCGCGGCAGACAUCAGC
AGCACGUUGCGGUCCGUGCCCGCGCCACGACCCUCACCCAUCAGCACC
GCUUCCACUUCCAGCACCCCACGCAGUCGACCCCGCAUCUAGAGAGAGA
CUUCUUUGUUUUUCCCCCGCGUGUUUUUCCCAUUCCCUGUAUUUAUUU
CUAAAUAAUAAAAACACAGAGACGUUGAUAAUAACCGCAGUGUGCUUUA
UUAGGGUAUCACGGUGUAGAAAAAAAAAAGAGAGGGAAACCCUAAAUAU
AGCGUCUCUCAAAAAAAAAAAAAAAAAAAAAAAAAAAAAAAAAAAAAAAAAA
AAAAAAAAAAAAAAAAAAAAAAAAAAAAAAAAAAAAAA
SEQ ID NO: 5 (pp71 Construct B.2-Full-length (FT))
GCCACCCGCCGCGCACGCGCUUAAGACGACUCUAUAAAAACCCACGUCC
ACUCAGACACGCGACUUUUGGGCGCCACACCUGUCGCCGCUGCUAUAU
UUGCGACAGUUGCCGGAACCCUUCCCGACCUCCCACGAAGACCCGUUC
ACCUUUGCGCAUCCCCUGACCCUCCCCCCAUCCCGCCUUCGCAAUGUC
UCAGGCAUCGUCCUCGCCCGGUGAGGGACCCUCGUCGGAAGCGGCCGC
GAUCAGCGAGGCCGAAGCCGCCAGCGGCAGCUUUGGUCGCCUGCACUG
CCAGGUGCUUCGGCUCAUCACCAACGUGGAAGGCGGCUCGCUGGAAGC
CGGUCGUCUGCGACUCCUGGACCUGCGUACCAACAUAGAGGUGAGCCG
ACCCUCGGUUCUCUGCUGUUUUCAGGAGAACAAAUCUCCGCACGACACC
GUAGACCUGACCGACCUAAACAUCAAGGGCCGCUGCGUGGUGGGCGAA
CGGGACCGACUGCUGGUGGACCUCAACAACUUUGGCCCACGACGCCUG
ACGCCAGGCUCAGAAAACAACACGGUCUCGGUGCUGGCCUUUGCGCUG
CCGCUAGACCGCGUGCCCGUUAGCGGACUGCACCUCUUUCAGAGCCAG
CGGCGCGGCGGCGAAGAAAAUCGGCCGCGAAUGGAGGCGCGCGCCAUC
AUCCGCCGCACGGCUCACCACUGGGCCGUGCGACUGACCGUGACGCCG
AACUGGCGCCGCAGAACCGACAGCAGUUUGGAGGCAGGGCAGAUCUUU
GUCAGCCAGUUCGCCUUUCGCGCCGGCGCCAUCCCGCUGACGCUGGUA
GACGCCCUGGAGCAGCUGGCCUGUUCGGACCCUAACACGUACAUCCAC
AAAACGGAGACGGACGAACGAGGCCAAUGGAUCAUGCUGUUUCUGCAU
CACGACUCACCGCACCCGCCGACCAGCGUGUUUCUGCACUUUUCGGUU
UACACGCAUCGCGCCGAGGUGGUGGCGCGACACAAUCCGUACCCGCAC
CUACGACGCUUGCCGGACAACGGCUUCCAGCUGUUGAUUCCCAAAAGU
UUUACGCUGACGCGCAUACAUCCCGAGUACAUCGUGCAGAUCCAGAAUG
CUUUCGAAACCAAUCAGACUCACGACACCAUCUUUUUCCCGGAAAACAU
CCCGGGCGUCUCCAUAGAAGCCGGCCCGCUACCCGAUCGUGUGCGAAU
CACCCUCCGCGUCACGCUGACCGGCGACCAGGCCGUUCAUUUGGAACA
CCGACAGCCGCUAGGCCGCAUCCACUUUUUCCGCCGUGGGUUUUGGAC
UCUCACACCCGGUAAACCGGACAAAAUCAAGCGUCCCCAGGUGCAGCUG
CGCGCCGGUCUCUUUCCACGGAGCGACGUCGUGCGCGGCGCCGUCUC
CGAGUUUCUCCCGCAGUCCCCCGGAUUACCACCCACCGAGGAAGAGGA
GGAAGAAGAGGAAGAGGACGACGAAGAUGACCUCUCCUCCACACCGACG
CCGACCCCCCUGUCCGAAGCCAUGUUUGCCGGCUUCGAGGAGGCCAGC
GGCGACGAGGACUCGGACACCCAAGCCGGACUGUCCCGGGCACUGAUC
CUGACCGGACAAAGACGUCGAAGCGGUAACAACGGGGCUCUCACGCUC
GUCAUCCCCUCGUGGCACGUCUUUGCGAGCCUUGACGACUUGGUACCA
UUAACGGUGAGCGUGCAGCACGCCGCAUUACGACCUACCUCUUAUCUG
CGCAGCGACAUGGACGGCGACGUGCGUACCGCGGCAGACAUCAGCAGC
ACGUUGCGGUCCGUGCCCGCGCCACGACCCUCACCCAUCAGCACCGCU
UCCACUUCCAGCACCCCACGCAGUCGACCCCGCAUCUAGAGAGAGACUU
CUUUGUUUUUCCCCCGCGUGUUUUUCCCAUUCCCUGUAUUUAUUUCUA
AAUAAUAAAAACACAGAGACGUUGAUAAUAACCGCAGUGUGCUUUAUUA
GGGUAUCACGGUGUAGAAAAAAAAAGAGAGGGAAACCCUAAAUAUAGCG
UCUCUCAGCCUGAGUAGGAAG
SEQ ID NO: 6 (pp71 Construct B.3-Full-length (FT))
GCCACCCGCCGCGCACGCGCUUAAGACGACUCUAUAAAAACCCACGUCC
ACUCAGACACGCGACUUUUGGGCGCCACACCUGUCGCCGCUGCUAUAU
UUGCGACAGUUGCCGGAACCCUUCCCGACCUCCCACGAAGACCCGUUC
ACCUUUGCGCAUCCCCUGACCCUCCCCCCAUCCCGCCUUCGCAAUGUC
UCAGGCAUCGUCCUCGCCCGGUGAGGGACCCUCGUCGGAAGCGGCCGC
GAUCAGCGAGGCCGAAGCCGCCAGCGGCAGCUUUGGUCGCCUGCACUG
CCAGGUGCUUCGGCUCAUCACCAACGUGGAAGGCGGCUCGCUGGAAGC
CGGUCGUCUGCGACUCCUGGACCUGCGUACCAACAUAGAGGUGAGCCG
ACCCUCGGUUCUCUGCUGUUUUCAGGAGAACAAAUCUCCGCACGACACC
GUAGACCUGACCGACCUAAACAUCAAGGGCCGCUGCGUGGUGGGCGAA
CGGGACCGACUGCUGGUGGACCUCAACAACUUUGGCCCACGACGCCUG
ACGCCAGGCUCAGAAAACAACACGGUCUCGGUGCUGGCCUUUGCGCUG
CCGCUAGACCGCGUGCCCGUUAGCGGACUGCACCUCUUUCAGAGCCAG
CGGCGCGGCGGCGAAGAAAAUCGGCCGCGAAUGGAGGCGCGCGCCAUC
AUCCGCCGCACGGCUCACCACUGGGCCGUGCGACUGACCGUGACGCCG
AACUGGCGCCGCAGAACCGACAGCAGUUUGGAGGCAGGGCAGAUCUUU
GUCAGCCAGUUCGCCUUUCGCGCCGGCGCCAUCCCGCUGACGCUGGUA
GACGCCCUGGAGCAGCUGGCCUGUUCGGACCCUAACACGUACAUCCAC
AAAACGGAGACGGACGAACGAGGCCAAUGGAUCAUGCUGUUUCUGCAU
CACGACUCACCGCACCCGCCGACCAGCGUGUUUCUGCACUUUUCGGUU
UACACGCAUCGCGCCGAGGUGGUGGCGCGACACAAUCCGUACCCGCAC
CUACGACGCUUGCCGGACAACGGCUUCCAGCUGUUGAUUCCCAAAAGU
UUUACGCUGACGCGCAUACAUCCCGAGUACAUCGUGCAGAUCCAGAAUG
CUUUCGAAACCAAUCAGACUCACGACACCAUCUUUUUCCCGGAAAACAU
CCCGGGCGUCUCCAUAGAAGCCGGCCCGCUACCCGAUCGUGUGCGAAU
CACCCUCCGCGUCACGCUGACCGGCGACCAGGCCGUUCAUUUGGAACA
CCGACAGCCGCUAGGCCGCAUCCACUUUUUCCGCCGUGGGUUUUGGAC
UCUCACACCCGGUAAACCGGACAAAAUCAAGCGUCCCCAGGUGCAGCUG
CGCGCCGGUCUCUUUCCACGGAGCGACGUCGUGCGCGGCGCCGUCUC
CGAGUUUCUCCCGCAGUCCCCCGGAUUACCACCCACCGAGGAAGAGGA
GGAAGAAGAGGAAGAGGACGACGAAGAUGACCUCUCCUCCACACCGACG
CCGACCCCCCUGUCCGAAGCCAUGUUUGCCGGCUUCGAGGAGGCCAGC
GGCGACGAGGACUCGGACACCCAAGCCGGACUGUCCCGGGCACUGAUC
CUGACCGGACAAAGACGUCGAAGCGGUAACAACGGGGCUCUCACGCUC
GUCAUCCCCUCGUGGCACGUCUUUGCGAGCCUUGACGACUUGGUACCA
UUAACGGUGAGCGUGCAGCACGCCGCAUUACGACCUACCUCUUAUCUG
CGCAGCGACAUGGACGGCGACGUGCGUACCGCGGCAGACAUCAGCAGC
ACGUUGCGGUCCGUGCCCGCGCCACGACCCUCACCCAUCAGCACCGCU
UCCACUUCCAGCACCCCACGCAGUCGACCCCGCAUCUAGAGAGAGACUU
CUUUGUUUUUCCCCCGCGUGUUUUUCCCAUUCCCUGUAUUUAUUUCUA
AAUAAUAAAAACACAGAGACGUUGAUAAUAACCGCAGUGUGCUUUAUUA
GGGUAUCACGGUGUAGAAAAAAAAAAGAGAGGGAAACCCUAAAUAUAGC
GUCUCUC
SEQ ID NO: 7 (pp71 Construct C-Immediate early (IE1))
AGGUCGUUUAGUGAACCGUCAGAUCGCCUGGAGACGCCAUCCACGCUG
UUUUGACCUCCAUAGAAGACACCGGGACCGAUCCAGCCUCCGCGGCCG
GGAACGGUGCAUUGGAACGCGGAUUCCCCGUGCCAAGAGUGACAUGUC
UCAGGCAUCGUCCUCGCCCGGUGAGGGACCCUCGUCGGAAGCGGCCGC
GAUCAGCGAGGCCGAAGCCGCCAGCGGCAGCUUUGGUCGCCUGCACUG
CCAGGUGCUUCGGCUCAUCACCAACGUGGAAGGCGGCUCGCUGGAAGC
CGGUCGUCUGCGACUCCUGGACCUGCGUACCAACAUAGAGGUGAGCCG
ACCCUCGGUUCUCUGCUGUUUUCAGGAGAACAAAUCUCCGCACGACACC
GUAGACCUGACCGACCUAAACAUCAAGGGCCGCUGCGUGGUGGGCGAA
CGGGACCGACUGCUGGUGGACCUCAACAACUUUGGCCCACGACGCCUG
ACGCCAGGCUCAGAAAACAACACGGUCUCGGUGCUGGCCUUUGCGCUG
CCGCUAGACCGCGUGCCCGUUAGCGGACUGCACCUCUUUCAGAGCCAG
CGGCGCGGCGGCGAAGAAAAUCGGCCGCGAAUGGAGGCGCGCGCCAUC
AUCCGCCGCACGGCUCACCACUGGGCCGUGCGACUGACCGUGACGCCG
AACUGGCGCCGCAGAACCGACAGCAGUUUGGAGGCAGGGCAGAUCUUU
GUCAGCCAGUUCGCCUUUCGCGCCGGCGCCAUCCCGCUGACGCUGGUA
GACGCCCUGGAGCAGCUGGCCUGUUCGGACCCUAACACGUACAUCCAC
AAAACGGAGACGGACGAACGAGGCCAAUGGAUCAUGCUGUUUCUGCAU
CACGACUCACCGCACCCGCCGACCAGCGUGUUUCUGCACUUUUCGGUU
UACACGCAUCGCGCCGAGGUGGUGGCGCGACACAAUCCGUACCCGCAC
CUACGACGCUUGCCGGACAACGGCUUCCAGCUGUUGAUUCCCAAAAGU
UUUACGCUGACGCGCAUACAUCCCGAGUACAUCGUGCAGAUCCAGAAUG
CUUUCGAAACCAAUCAGACUCACGACACCAUCUUUUUCCCGGAAAACAU
CCCGGGCGUCUCCAUAGAAGCCGGCCCGCUACCCGAUCGUGUGCGAAU
CACCCUCCGCGUCACGCUGACCGGCGACCAGGCCGUUCAUUUGGAACA
CCGACAGCCGCUAGGCCGCAUCCACUUUUUCCGCCGUGGGUUUUGGAC
UCUCACACCCGGUAAACCGGACAAAAUCAAGCGUCCCCAGGUGCAGCUG
CGCGCCGGUCUCUUUCCACGGAGCGACGUCGUGCGCGGCGCCGUCUC
CGAGUUUCUCCCGCAGUCCCCCGGAUUACCACCCACCGAGGAAGAGGA
GGAAGAAGAGGAAGAGGACGACGAAGAUGACCUCUCCUCCACACCGACG
CCGACCCCCCUGUCCGAAGCCAUGUUUGCCGGCUUCGAGGAGGCCAGC
GGCGACGAGGACUCGGACACCCAAGCCGGACUGUCCCGGGCACUGAUC
CUGACCGGACAAAGACGUCGAAGCGGUAACAACGGGGCUCUCACGCUC
GUCAUCCCCUCGUGGCACGUCUUUGCGAGCCUUGACGACUUGGUACCA
UUAACGGUGAGCGUGCAGCACGCCGCAUUACGACCUACCUCUUAUCUG
CGCAGCGACAUGGACGGCGACGUGCGUACCGCGGCAGACAUCAGCAGC
ACGUUGCGGUCCGUGCCCGCGCCACGACCCUCACCCAUCAGCACCGCU
UCCACUUCCAGCACCCCACGCAGUCGACCCCGCAUCUAGGCGGCCGCU
UAAUUAAGCUGCCUUCUGCGGGGCUUGCCUUCUGGCCAUGCCCUUCUU
CUCUCCCUUGCACCUGUACCUCUUGGUCUUUGAAUAAAGCCUGAGUAG
GAAG
SEQ ID NO: 8 (pp71 Construct D-short)
AGGGCGCCACACCUGUCGCCGCUGCUAUAUUUGCGACAGUUGCCGGAA
CCCUUCCCGACCUCCCACGAAGACCCGUUCACCUUUGCGCAUCCCCUG
ACCCUCCCCCCAUCCCGCCUUCGCAAUGUCUCAGGCAUCGUCCUCGCC
CGGUGAGGGACCCUCGUCGGAAGCGGCCGCGAUCAGCGAGGCCGAAGC
CGCCAGCGGCAGCUUUGGUCGCCUGCACUGCCAGGUGCUUCGGCUCAU
CACCAACGUGGAAGGCGGCUCGCUGGAAGCCGGUCGUCUGCGACUCCU
GGACCUGCGUACCAACAUAGAGGUGAGCCGACCCUCGGUUCUCUGCUG
UUUUCAGGAGAACAAAUCUCCGCACGACACCGUAGACCUGACCGACCUA
AACAUCAAGGGCCGCUGCGUGGUGGGCGAACGGGACCGACUGCUGGUG
GACCUCAACAACUUUGGCCCACGACGCCUGACGCCAGGCUCAGAAAACA
ACACGGUCUCGGUGCUGGCCUUUGCGCUGCCGCUAGACCGCGUGCCC
GUUAGCGGACUGCACCUCUUUCAGAGCCAGCGGCGCGGCGGCGAAGAA
AAUCGGCCGCGAAUGGAGGCGCGCGCCAUCAUCCGCCGCACGGCUCAC
CACUGGGCCGUGCGACUGACCGUGACGCCGAACUGGCGCCGCAGAACC
GACAGCAGUUUGGAGGCAGGGCAGAUCUUUGUCAGCCAGUUCGCCUUU
CGCGCCGGCGCCAUCCCGCUGACGCUGGUAGACGCCCUGGAGCAGCUG
GCCUGUUCGGACCCUAACACGUACAUCCACAAAACGGAGACGGACGAAC
GAGGCCAAUGGAUCAUGCUGUUUCUGCAUCACGACUCACCGCACCCGC
CGACCAGCGUGUUUCUGCACUUUUCGGUUUACACGCAUCGCGCCGAGG
UGGUGGCGCGACACAAUCCGUACCCGCACCUACGACGCUUGCCGGACA
ACGGCUUCCAGCUGUUGAUUCCCAAAAGUUUUACGCUGACGCGCAUACA
UCCCGAGUACAUCGUGCAGAUCCAGAAUGCUUUCGAAACCAAUCAGACU
CACGACACCAUCUUUUUCCCGGAAAACAUCCCGGGCGUCUCCAUAGAAG
CCGGCCCGCUACCCGAUCGUGUGCGAAUCACCCUCCGCGUCACGCUGA
CCGGCGACCAGGCCGUUCAUUUGGAACACCGACAGCCGCUAGGCCGCA
UCCACUUUUUCCGCCGUGGGUUUUGGACUCUCACACCCGGUAAACCGG
ACAAAAUCAAGCGUCCCCAGGUGCAGCUGCGCGCCGGUCUCUUUCCAC
GGAGCGACGUCGUGCGCGGCGCCGUCUCCGAGUUUCUCCCGCAGUCC
CCCGGAUUACCACCCACCGAGGAAGAGGAGGAAGAAGAGGAAGAGGAC
GACGAAGAUGACCUCUCCUCCACACCGACGCCGACCCCCCUGUCCGAA
GCCAUGUUUGCCGGCUUCGAGGAGGCCAGCGGCGACGAGGACUCGGAC
ACCCAAGCCGGACUGUCCCGGGCACUGAUCCUGACCGGACAAAGACGU
CGAAGCGGUAACAACGGGGCUCUCACGCUCGUCAUCCCCUCGUGGCAC
GUCUUUGCGAGCCUUGACGACUUGGUACCAUUAACGGUGAGCGUGCAG
CACGCCGCAUUACGACCUACCUCUUAUCUGCGCAGCGACAUGGACGGC
GACGUGCGUACCGCGGCAGACAUCAGCAGCACGUUGCGGUCCGUGCCC
GCGCCACGACCCUCACCCAUCAGCACCGCUUCCACUUCCAGCACCCCAC
GCAGUCGACCCCGCAUCUAGAGAGAGACUUCUUUGUUUUUCCCCCGCG
UGUUUUUCCCAUUCCCUGUAUUUAUUUCUAAAUAAUAAAAACACAGAGA
CGUUGAUAAUAACCGCAGCCUGAGUAGGAAG
SEQ ID NO: 9 (pp71 Construct E-pp65)
AGGACAGAGGACCCUGGUGUUGGGAAACGGACACUAGGCGUCCGCGCG
AUACGGGGUUAAAACAAAAAAAAUCGGUGGUGGUGUGUGCUGGGGUGU
GGUGACGGUGGGGCUUUGCCUCUUUUUUUUUGUAAUAAAAAAAGACAC
UCAAUAAUCCGCGGUUGUCUCUGUGUAGAACGUUUUUAUUUCGGGUUC
CGCGUUUGGUCGCCUGCCUGUGUAAGGCGGCGGCCGCAAAGGGCGCG
CCGCUCAGUCGCCUACACCCGUACGCGCAGGCAGCAUGGAGUCGCGCG
GUCGCCGUUGUCCCGAAAUGAUAUCCGUACUGGGUCCCAUUUCGGGGC
ACGUGCUGAAAGCCGUGUUUAGUCGCGGCGACACGCCGGUGCUGCCGC
ACGAGACGCGACUCCUGCAGACGGGUAUCCACGUACGCGUGAGCCAGC
CCUCGCUGAUCCUGGUGUCGCAGUACACGCCCGACUCGACGCCAUGCC
ACCGCGGCGACAAUCAGCUGCAGGUGCAGCACACGUACUUUACGGGCA
GCGAGGUGGAGAACGUGUCGGUCAACGUGCACAACCCCACGGGCCGAA
GCAUCUGCCCCAGCCAGGAGCCCAUGUCGAUCUAUGUGUACGCGCUGC
CGCUCAAGAUGCUGAACAUCCCCAGCAUCAACGUGCACCACUACCCGUC
GGCGGCCGAGCGCAAACACCGACACCUGCCCGUAGCUGACGCUGUGAU
UCACGCGUCGGGCAAGCAGAUGUGGCAGGCGCGUCUCACGGUCUCGG
GACUGGCCUGGACGCGUCAGCAGAACCAGUGGAAAGAGCCCGACGUCU
ACUACACGUCAGCGUUCGUGUUUCCCACCAAGGACGUGGCACUGCGGC
ACGUGGUGUGCGCGCACGAGCUGGUUUGCUCCAUGGAGAACACGCGCG
CAACCAAGAUGCAGGUGAUAGGUGACCAGUACGUCAAGGUGUACCUGG
AGUCCUUCUGCGAGGACGUGCCCUCCGGCAAGCUCUUUAUGCACGUCA
CGCUGGGCUCUGACGUGGAAGAGGACCUGACGAUGACCCGCAACCCGC
AACCCUUCAUGCGCCCCCACGAGCGCAACGGCUUUACGGUGUUGUGUC
CCAAAAAUAUGAUAAUCAAACCGGGCAAGAUCUCGCACAUCAUGCUGGA
UGUGGCUUUUACCUCACACGAGCAUUUUGGGCUGCUGUGUCCCAAGAG
CAUCCCGGGCCUGAGCAUCUCAGGUAACCUGUUGAUGAACGGGCAGCA
GAUCUUCCUGGAGGUACAAGCGAUACGCGAGACCGUGGAACUGCGUCA
GUACGAUCCCGUGGCUGCACUCUUCUUUUUCGAUAUCGACUUGCUGCU
GCAGCGCGGGCCUCAGUACAGCGAGCACCCCACCUUCACCAGCCAGUA
UCGCAUCCAGGGCAAGCUUGAGUACCGACACACCUGGGACCGGCACGA
CGAGGGUGCCGCCCAGGGCGACGACGACGUCUGGACCAGCGGAUCGGA
CUCCGACGAAGAACUCGUAACCACCGAGCGCAAGACGCCCCGCGUUACC
GGCGGCGGCGCCAUGGCGGGCGCCUCCACUUCCGCGGGCCGCAAACG
CAAAUCAGCAUCCUCGGCGACGGCGUGCACGGGGGGCGUUAUGACACG
CGGCCGCCUUAAGGCCGAGUCCACCGUCGCGCCCGAAGAGGACACCGA
CGAGGAUUCCGACAACGAAAUCCACAAUCCGGCCGUGUUCACCUGGCC
GCCCUGGCAGGCCGGCAUCCUGGCCCGCAACCUGGUGCCCAUGGUGG
CUACGGUUCAGGGUCAGAAUCUGAAGUACCAGGAGUUCUUCUGGGACG
CCAACGACAUCUACCGCAUCUUCGCCGAAUUGGAAGGCGUAUGGCAGC
CCGCUGCGCAACCCAAACGUCGCCGCCACCGGCAAGAAGCCCUGCCCG
GGCCAUGCAUCGCCUCGACGCCCAAAAAGCAUCGAGGUUGAGCCACCC
GCCGCGCACGCGCUUAAGACGACUCUAUAAAAACCCACGUCCACUCAGA
CACGCGACUUUUGGGCGCCACACCUGUCGCCGCUGCUAUAUUUGCGAC
AGUUGCCGGAACCCUUCCCGACCUCCCACGAAGACCCGUUCACCUUUG
CGCAUCCCCUGACCCUCCCCCCAUCCCGCCUUCGCAAUGUCUCAGGCA
UCGUCCUCGCCCGGUGAGGGACCCUCGUCGGAAGCGGCCGCGAUCAGC
GAGGCCGAAGCCGCCAGCGGCAGCUUUGGUCGCCUGCACUGCCAGGUG
CUUCGGCUCAUCACCAACGUGGAAGGCGGCUCGCUGGAAGCCGGUCGU
CUGCGACUCCUGGACCUGCGUACCAACAUAGAGGUGAGCCGACCCUCG
GUUCUCUGCUGUUUUCAGGAGAACAAAUCUCCGCACGACACCGUAGACC
UGACCGACCUAAACAUCAAGGGCCGCUGCGUGGUGGGCGAACGGGACC
GACUGCUGGUGGACCUCAACAACUUUGGCCCACGACGCCUGACGCCAG
GCUCAGAAAACAACACGGUCUCGGUGCUGGCCUUUGCGCUGCCGCUAG
ACCGCGUGCCCGUUAGCGGACUGCACCUCUUUCAGAGCCAGCGGCGCG
GCGGCGAAGAAAAUCGGCCGCGAAUGGAGGCGCGCGCCAUCAUCCGCC
GCACGGCUCACCACUGGGCCGUGCGACUGACCGUGACGCCGAACUGGC
GCCGCAGAACCGACAGCAGUUUGGAGGCAGGGCAGAUCUUUGUCAGCC
AGUUCGCCUUUCGCGCCGGCGCCAUCCCGCUGACGCUGGUAGACGCCC
UGGAGCAGCUGGCCUGUUCGGACCCUAACACGUACAUCCACAAAACGGA
GACGGACGAACGAGGCCAAUGGAUCAUGCUGUUUCUGCAUCACGACUC
ACCGCACCCGCCGACCAGCGUGUUUCUGCACUUUUCGGUUUACACGCA
UCGCGCCGAGGUGGUGGCGCGACACAAUCCGUACCCGCACCUACGACG
CUUGCCGGACAACGGCUUCCAGCUGUUGAUUCCCAAAAGUUUUACGCU
GACGCGCAUACAUCCCGAGUACAUCGUGCAGAUCCAGAAUGCUUUCGAA
ACCAAUCAGACUCACGACACCAUCUUUUUCCCGGAAAACAUCCCGGGCG
UCUCCAUAGAAGCCGGCCCGCUACCCGAUCGUGUGCGAAUCACCCUCC
GCGUCACGCUGACCGGCGACCAGGCCGUUCAUUUGGAACACCGACAGC
CGCUAGGCCGCAUCCACUUUUUCCGCCGUGGGUUUUGGACUCUCACAC
CCGGUAAACCGGACAAAAUCAAGCGUCCCCAGGUGCAGCUGCGCGCCG
GUCUCUUUCCACGGAGCGACGUCGUGCGCGGCGCCGUCUCCGAGUUUC
UCCCGCAGUCCCCCGGAUUACCACCCACCGAGGAAGAGGAGGAAGAAG
AGGAAGAGGACGACGAAGAUGACCUCUCCUCCACACCGACGCCGACCCC
CCUGUCCGAAGCCAUGUUUGCCGGCUUCGAGGAGGCCAGCGGCGACGA
GGACUCGGACACCCAAGCCGGACUGUCCCGGGCACUGAUCCUGACCGG
ACAAAGACGUCGAAGCGGUAACAACGGGGCUCUCACGCUCGUCAUCCC
CUCGUGGCACGUCUUUGCGAGCCUUGACGACUUGGUACCAUUAACGGU
GAGCGUGCAGCACGCCGCAUUACGACCUACCUCUUAUCUGCGCAGCGA
CAUGGACGGCGACGUGCGUACCGCGGCAGACAUCAGCAGCACGUUGCG
GUCCGUGCCCGCGCCACGACCCUCACCCAUCAGCACCGCUUCCACUUC
CAGCACCCCACGCAGUCGACCCCGCAUCUAGAGAGAGACUUCUUUGUU
UUUCCCCCGCGUGUUUUUCCCAUUCCCUGUAUUUAUUUCUAAAUAAUAA
AAACACAGAGACGUUGAUAAUAACCGCAGUGUGCUUUAUUAGGGUAUCA
CGGUGUAGAAAAAAAAAGAGAGGGAAACCCUAAAUAUAGCGUCUCUCAG
CCUGAGUAGGAAG
SEQ ID NO: 10 (pp71 Construct F-stop pp65)
AGGACAGAGGACCCUGGUGUUGGGAAACGGACACUAGGCGUCCGCGCG
AUACGGGGUUAAAACAAAAAAAAUCGGUGGUGGUGUGUGCUGGGGUGU
GGUGACGGUGGGGCUUUGCCUCUUUUUUUUUGUAAUAAAAAAAGACAC
UCAAUAAUCCGCGGUUGUCUCUGUGUAGAACGUUUUUAUUUCGGGUUC
CGCGUUUGGUCGCCUGCCUGUGUAAGGCGGCGGCCGCAAAGGGCGCG
CCGCUCAGUCGCCUACACCCGUACGCGCAGGCAGCAUGGAGUCGCGCG
GUCGCCGUUGUCCCGAAUAGUGAUCCGUACUGGGUCCCAUUUCGGGGC
ACGUGCUGAAAGCCGUGUUUAGUCGCGGCGACACGCCGGUGCUGCCGC
ACGAGACGCGACUCCUGCAGACGGGUAUCCACGUACGCGUGAGCCAGC
CCUCGCUGAUCCUGGUGUCGCAGUACACGCCCGACUCGACGCCAUGCC
ACCGCGGCGACAAUCAGCUGCAGGUGCAGCACACGUACUUUACGGGCA
GCGAGGUGGAGAACGUGUCGGUCAACGUGCACAACCCCACGGGCCGAA
GCAUCUGCCCCAGCCAGGAGCCCAUGUCGAUCUAUGUGUACGCGCUGC
CGCUCAAGAUGCUGAACAUCCCCAGCAUCAACGUGCACCACUACCCGUC
GGCGGCCGAGCGCAAACACCGACACCUGCCCGUAGCUGACGCUGUGAU
UCACGCGUCGGGCAAGCAGAUGUGGCAGGCGCGUCUCACGGUCUCGG
GACUGGCCUGGACGCGUCAGCAGAACCAGUGGAAAGAGCCCGACGUCU
ACUACACGUCAGCGUUCGUGUUUCCCACCAAGGACGUGGCACUGCGGC
ACGUGGUGUGCGCGCACGAGCUGGUUUGCUCCAUGGAGAACACGCGCG
CAACCAAGAUGCAGGUGAUAGGUGACCAGUACGUCAAGGUGUACCUGG
AGUCCUUCUGCGAGGACGUGCCCUCCGGCAAGCUCUUUAUGCACGUCA
CGCUGGGCUCUGACGUGGAAGAGGACCUGACGAUGACCCGCAACCCGC
AACCCUUCAUGCGCCCCCACGAGCGCAACGGCUUUACGGUGUUGUGUC
CCAAAAAUAUGAUAAUCAAACCGGGCAAGAUCUCGCACAUCAUGCUGGA
UGUGGCUUUUACCUCACACGAGCAUUUUGGGCUGCUGUGUCCCAAGAG
CAUCCCGGGCCUGAGCAUCUCAGGUAACCUGUUGAUGAACGGGCAGCA
GAUCUUCCUGGAGGUACAAGCGAUACGCGAGACCGUGGAACUGCGUCA
GUACGAUCCCGUGGCUGCACUCUUCUUUUUCGAUAUCGACUUGCUGCU
GCAGCGCGGGCCUCAGUACAGCGAGCACCCCACCUUCACCAGCCAGUA
UCGCAUCCAGGGCAAGCUUGAGUACCGACACACCUGGGACCGGCACGA
CGAGGGUGCCGCCCAGGGCGACGACGACGUCUGGACCAGCGGAUCGGA
CUCCGACGAAGAACUCGUAACCACCGAGCGCAAGACGCCCCGCGUUACC
GGCGGCGGCGCCAUGGCGGGCGCCUCCACUUCCGCGGGCCGCAAACG
CAAAUCAGCAUCCUCGGCGACGGCGUGCACGGCGGGCGUUAUGACACG
CGGCCGCCUUAAGGCCGAGUCCACCGUCGCGCCCGAAGAGGACACCGA
CGAGGAUUCCGACAACGAAAUCCACAAUCCGGCCGUGUUCACCUGGCC
GCCCUGGCAGGCCGGCAUCCUGGCCCGCAACCUGGUGCCCAUGGUGG
CUACGGUUCAGGGUCAGAAUCUGAAGUACCAGGAGUUCUUCUGGGACG
CCAACGACAUCUACCGCAUCUUCGCCGAAUUGGAAGGCGUAUGGCAGC
CCGCUGCGCAACCCAAACGUCGCCGCCACCGGCAAGAAGCCCUGCCCG
GGCCAUGCAUCGCCUCGACGCCCAAAAAGCAUCGAGGUUGAGCCACCC
GCCGCGCACGCGCUUAAGACGACUCUAUAAAAACCCACGUCCACUCAGA
CACGCGACUUUUGGGCGCCACACCUGUCGCCGCUGCUAUAUUUGCGAC
AGUUGCCGGAACCCUUCCCGACCUCCCACGAAGACCCGUUCACCUUUG
CGCAUCCCCUGACCCUCCCCCCAUCCCGCCUUCGCAAUGUCUCAGGCA
UCGUCCUCGCCCGGUGAGGGACCCUCGUCGGAAGCGGCCGCGAUCAGC
GAGGCCGAAGCCGCCAGCGGCAGCUUUGGUCGCCUGCACUGCCAGGUG
CUUCGGCUCAUCACCAACGUGGAAGGCGGCUCGCUGGAAGCCGGUCGU
CUGCGACUCCUGGACCUGCGUACCAACAUAGAGGUGAGCCGACCCUCG
GUUCUCUGCUGUUUUCAGGAGAACAAAUCUCCGCACGACACCGUAGACC
UGACCGACCUAAACAUCAAGGGCCGCUGCGUGGUGGGCGAACGGGACC
GACUGCUGGUGGACCUCAACAACUUUGGCCCACGACGCCUGACGCCAG
GCUCAGAAAACAACACGGUCUCGGUGCUGGCCUUUGCGCUGCCGCUAG
ACCGCGUGCCCGUUAGCGGACUGCACCUCUUUCAGAGCCAGCGGCGCG
GCGGCGAAGAAAAUCGGCCGCGAAUGGAGGCGCGCGCCAUCAUCCGCC
GCACGGCUCACCACUGGGCCGUGCGACUGACCGUGACGCCGAACUGGC
GCCGCAGAACCGACAGCAGUUUGGAGGCAGGGCAGAUCUUUGUCAGCC
AGUUCGCCUUUCGCGCCGGCGCCAUCCCGCUGACGCUGGUAGACGCCC
UGGAGCAGCUGGCCUGUUCGGACCCUAACACGUACAUCCACAAAACGGA
GACGGACGAACGAGGCCAAUGGAUCAUGCUGUUUCUGCAUCACGACUC
ACCGCACCCGCCGACCAGCGUGUUUCUGCACUUUUCGGUUUACACGCA
UCGCGCCGAGGUGGUGGCGCGACACAAUCCGUACCCGCACCUACGACG
CUUGCCGGACAACGGCUUCCAGCUGUUGAUUCCCAAAAGUUUUACGCU
GACGCGCAUACAUCCCGAGUACAUCGUGCAGAUCCAGAAUGCUUUCGAA
ACCAAUCAGACUCACGACACCAUCUUUUUCCCGGAAAACAUCCCGGGCG
UCUCCAUAGAAGCCGGCCCGCUACCCGAUCGUGUGCGAAUCACCCUCC
GCGUCACGCUGACCGGCGACCAGGCCGUUCAUUUGGAACACCGACAGC
CGCUAGGCCGCAUCCACUUUUUCCGCCGUGGGUUUUGGACUCUCACAC
CCGGUAAACCGGACAAAAUCAAGCGUCCCCAGGUGCAGCUGCGCGCCG
GUCUCUUUCCACGGAGCGACGUCGUGCGCGGCGCCGUCUCCGAGUUUC
UCCCGCAGUCCCCCGGAUUACCACCCACCGAGGAAGAGGAGGAAGAAG
AGGAAGAGGACGACGAAGAUGACCUCUCCUCCACACCGACGCCGACCCC
CCUGUCCGAAGCCAUGUUUGCCGGCUUCGAGGAGGCCAGCGGCGACGA
GGACUCGGACACCCAAGCCGGACUGUCCCGGGCACUGAUCCUGACCGG
ACAAAGACGUCGAAGCGGUAACAACGGGGCUCUCACGCUCGUCAUCCC
CUCGUGGCACGUCUUUGCGAGCCUUGACGACUUGGUACCAUUAACGGU
GAGCGUGCAGCACGCCGCAUUACGACCUACCUCUUAUCUGCGCAGCGA
CAUGGACGGCGACGUGCGUACCGCGGCAGACAUCAGCAGCACGUUGCG
GUCCGUGCCCGCGCCACGACCCUCACCCAUCAGCACCGCUUCCACUUC
CAGCACCCCACGCAGUCGACCCCGCAUCUAGAGAGAGACUUCUUUGUU
UUUCCCCCGCGUGUUUUUCCCAUUCCCUGUAUUUAUUUCUAAAUAAUAA
AAACACAGAGACGUUGAUAAUAACCGCAGUGUGCUUUAUUAGGGUAUCA
CGGUGUAGAAAAAAAAAGAGAGGGAAACCCUAAAUAUAGCGUCUCUCAG
CCUGAGUAGGAAG
SEQ ID NO: 11 (M conserved gag/nef/pol fusion episensus 1, DNA)
ACCATGCCAATTGTGCAGAATTTGCAGGGACAGATGGTGCATCAGGCTAT
CTCCCCCCGCACGCTCAATGCCTGGGTGAAGGTAGTGGAGGAGAAAGCC
TTTTCACCAGAAGTTATCCCTATGTTCTCTGCTCTGAGTGAGGGGGCCACC
CCCCAAGACTTGAATACCATGCTGAACACCGTGGGTGGGCACCAGGCAG
CCATGCAAATGCTGAAGGAGACTATTAACGAGGAGGCTGCTGAGTGGGAT
AGGCTGCACCCAGTCCATGCAGGACCAATCGCGCCAGGCCAAATGAGAG
AACCACGGGGGAGCGACATCGCAGGGACAACCTCCACACTTCAAGAGCA
GATCGGATGGATGACCAACAACCCCCCAATCCCCGTCGGCGAGATCTACA
AGCGCTGGATAATCCTTGGCCTGAACAAGATTGTGCGGATGTATAGCCCC
GTTTCAATCCTGGATATTCGCCAGGGCCCAAAGGAGCCTTTCCGCGATTA
TGTGGATAGGTTTTACAAGACCCTTCGGGCCGAACAGGCCACACAGGAGG
TGAAGAACTGGATGACAGAGACTCTGCTGGTTCAGAATGCAAATCCCGAC
TGTAAGACCATCCTGAAGGCCCTGGGACCAGCAGCGACTCTGGAAGAGAT
GATGACGGCCTGTCAGGGCGTGGGGGGACCAGGCCATAAAGCACGGGT
GCTCGTGGGGTTTCCAGTGAGGCCTCAGGTCCCACTGCGCCCCATGACA
TACAAGGGGGCCCTGGACCTGTCACACTTCCTTAAAGAAAAAGGCGGCTT
GGAGGGCCTGATCTATAGTAAAAAGAGACAAGAAATCCTGGATCTTTGGG
TTTACCACACACAGGGGTACTTCCCCGATTGGCAGAATTACACTCCCGGC
CCCGGCATTAGGTACCCCCTCACATTTGGCTGGTGCTTCAAACTGGTGCC
TCCCCAAATTACCCTTTGGCAGAGACCCCTTGTGACCATCAAAATCGGCG
GTCAGCTGAAAGAGGCACTGCTGGCAGATGACACCGTGCTTGAGGATATC
AACCTGCCTGGGAAATGGAAACCCAAGATGATAGGCGGAATCGGTGGGTT
CATCAAAGTGAGACAGTACGATCAAATCCTGATTGAGATCTGCGGCAAGA
AGGCGATCGGGACCGTACTGGTCGGGCCAACTCCAGTGAATATCATAGG
CCGCAACCTCCTGACTCAGATAGGCTGTACCCTTAATTTTCCGATATCTCC
AATCGAAACCGTGCCCGTTAAACTCAAGCCTGGTATGGATGGACCCAAAG
TCAAGCAATGGCCACTGACCGAGGAGAAGATAAAGGCCCTGGTGGAAATC
TGCACCGAAATGGAAAAGGAGGGAAAAATCTCTAAAATCGGACCCGAGAA
CCCTTATAATACTCCCGTCTTTGCCATTAAAAAGAAAGACAGCACAAAGTG
GCGGAAACTCGTAGACTTTAGAGAGTTGAATAAAAGAACTCAGGACTTTTG
GGAGGTGCAGCTTGGAATTCCCCACCCCGCCGGTCTGAAAAAGAAGAAAT
CCGTGACAGTGCTCGATGTTGGCGACGCCTATTTTAGTGTGCCTCTTGAC
AAAGACTTTCGGAAATACACCGCCTTCACCATTCCAAGCATTAATAACGAA
ACCCCAGGTATTAGATATCAATACAACGTGCTGCCCCAGGGGTGGAAAGG
CAGCCCAGCTATTTTTCAGAGCAGCATGACAAAAATTTTGGAGCCCTTCAG
AAAGCAGAATCCAGATATCGTCATTTACCAGCTGTATGTGGGGTCCGATCT
GGAGATTGGCCAGCACCGAACTAAGATCGAAGAACTGCGCCAGCACCTG
CTCAAATGGGGATTCACGACACCTGATAAAAAACATCAGAAGGAACCCCC
TTTCCTTTGGATGGGCTACGAGCTTCACCCCGATAAATGGACTGTTCAGCC
AATTGTCCTCCCTGAGAAGGACTCTTGGACAGTCAACGACATCCAAAAACT
GGTGGGGAAACTCAATTGGGCCAGTCAGATCTACCACGGCCAGGTGGAC
TGCTCACCGGGGATCTGGCAGCTTTGTACACATCTGGAGGGGAAAGTGAT
CCTGGTCGCTGTTCATGTTGCCAGCGGCTACATTGAAGCAGAGGTTATTC
CCGCCGAGACTGGCCAGGAGACAGCTTACTTCTTGCTGAAGCTGGCTGGT
CGGTGGCCCGTGAAGGTAATTCACACAAATGGCTCTAACTTTACCTCTGC
CGCAGTTAAGGCCGCCTGCTGGTGGGCCGGAATTAAGCAGGAATTCGGG
ATTCCTTACAATCCACAGTCACAGGGTGTAGTAAGCATGAATAAGGAATTG
AAGAAAATTATAGGACAGGTTCGCGACCAGGCCGAGCACCTGAAGACCGC
CGTTCAAATGGCCGTATTCATCCACAATTTTAAGCGCAAAGGGGGGATTG
GGGGTTACTCTGCCGGGGAGAGAATAATAGACATCATTGCCACCGATATC
CAGACAAAACTGCAGAAACAGATAACAAAGATACAGAACTTTAGGGTATAC
TATCGGGATTCCAGGGACCCTATCTGGAAGGGGCCAGCGAAACTGCTTTG
GAAGGGAGAAGGAGCCGTGGTCATACAGGACAATTCAGACATCAAAGTGG
TACCGAGAAGGAAGGCCAAGATTATTAGAGATTACGGAAAACAGATGGCG
GGGGACGACTGCGTTGCCGGGAGGCAGGACGAAGATCAATGA
SEQ ID NO: 12 (M conserved gag/nef/pol fusion episensus 1, protein)
MPIVQNLQGQMVHQAISPRTLNAWVKVVEEKAFSPEVIPMFSALSEGATPQD
LNTMLNTVGGHQAAMQMLKETINEEAAEWDRLHPVHAGPIAPGQMREPRGS
DIAGTTSTLQEQIGWMTNNPPIPVGEIYKRWIILGLNKIVRMYSPVSILDIRQGP
KEPFRDYVDRFYKTLRAEQATQEVKNWMTETLLVQNANPDCKTILKALGPAA
TLEEMMTACQGVGGPGHKARVLVGFPVRPQVPLRPMTYKGALDLSHFLKEK
GGLEGLIYSKKRQEILDLWVYHTQGYFPDWQNYTPGPGIRYPLTFGWCFKLV
PPQITLWQRPLVTIKIGGQLKEALLADDTVLEDINLPGKWKPKMIGGIGGFIKVR
QYDQILIEICGKKAIGTVLVGPTPVNIIGRNLLTQIGCTLNFPISPIETVPVKLKPG
MDGPKVKQWPLTEEKIKALVEICTEMEKEGKISKIGPENPYNTPVFAIKKKDST
KWRKLVDFRELNKRTQDFWEVQLGIPHPAGLKKKKSVTVLDVGDAYFSVPLD
KDFRKYTAFTIPSINNETPGIRYQYNVLPQGWKGSPAIFQSSMTKILEPFRKQN
PDIVIYQLYVGSDLEIGQHRTKIEELRQHLLKWGFTTPDKKHQKEPPFLWMGY
ELHPDKWTVQPIVLPEKDSWTVNDIQKLVGKLNWASQIYHGQVDCSPGIWQL
CTHLEGKVILVAVHVASGYIEAEVIPAETGQETAYFLLKLAGRWPVKVIHTNGS
NFTSAAVKAACWWAGIKQEFGIPYNPQSQGVVSMNKELKKIIGQVRDQAEHL
KTAVQMAVFIHNFKRKGGIGGYSAGERIIDIIATDIQTKLQKQITKIQNFRVYYRD
SRDPIWKGPAKLLWKGEGAVVIQDNSDIKVVPRRKAKIIRDYGKQMAGDDCV
AGRQDEDQ*
SEQ ID NO: 13 (Ag85A-ESAT-6-Rv3407-Rv2626c-RpfA-RpfD)
MAFSRPGLPVEYLQVPSPSMGRDIKVQFQSGGANSPALYLLDGLRAQDDFS
GWDINTPAFEWYDQSGLSVVMPVGGQSSFYSDWYQPACGKAGCQTYKWET
FLTSELPGWLQANRHVKPTGSAVVGLSMAASSALTLAIYHPQQFVYAGAMSG
LLDPSQAMGPTLIGLAMGDAGGYKASDMWGPKEDPAWQRNDPLLNVGKLIA
NNTRVWVYCGNGKPSDLGGNNLPAKFLEGFVRTSNIKFQDAYNAGGGHNGV
FDFPDSGTHSWEYWGAQLNAMKPDLQRALGATPNTGPAPQGATEQQWNFA
GIEAAASAIQGNVTSIHSLLDEGKQSLTKLAAAWGGSGSEAYQGVQQKWDAT
ATELNNALQNLARTISEAGQAMASTEGNVTGMFARATVGLVEAIGIRELRQHA
SRYLARVEAGEELGVTNKGRLVARLIPVQAAERSREALIESGVLIPARRPQNLL
DVTAEPARGRKRTLSDVLNEMRDEQTTARDIMNAGVTCVGEHETLTAAAQY
MREHDIGALPICGDDDRLHGMLTDRDIVIKGLAAGLDPNTATAGELARDSIYYV
DANASIQEMLNVMEEHQVRRVPVISEHRLVGIVTEADIARHLPEHAIVQFVKAI
CSPMALASSGRHRKPTTSNVSVAKIAFTGAVLGGGGIAMAAQATAATDGEWD
QVARCESGGNWSINTGNGYLGGLQFTQSTWAAHGGGEFAPSAQLASREQQI
AVGERVLATQGRGAWPVCGRGLSNATPREVLPASAAMDAPLDAAAVNGEPA
PLAPPPADPAPPVELAANDLPAPLGEPLPAAPADPAPPADLAPPAPADVAPPV
ELAVNDLPAPLGEPLPAAPADPAPPADLAPPAPADLAPPAPADLAPPAPADLA
PPVELAVNDLPAPLGEPLPAAPAELAPPADLAPASADLAPPAPADLAPPAPAE
LAPPAPADLAPPAAVNEQTAPGDQPATAPGGPVGLATDLELPEPDPQPADAP
PPGDVTEAPAETPQVSNIAYTKKLWQAIRAQDVCGNDALDSLAQPYVIGTPGL
LTTAGAGRPRDRCARIVCTVFIETAVVATMFVALLGLSTISSKADDIDWDAIAQ
CESGGNWAANTGNGLYGGLQISQATWDSNGGVGSPAAASPQQQIEVADNIM
KTQGPGAWPKCSSCSQGDAPLGSLTHILTFLAAETGGCSGSRDD
SEQ ID NO: 14 (pp71 Construct B-Full-length (FT) + pseudoU/5meC)
AGGGm5cm5cAm5cm5cm5cGm5cm5cGm5cGm5cAm5cGm5cGm5cppAAGA
m5cGAm5cpm5cpApAAAAAm5cm5cm5cAm5cGpm5cm5cAm5cpm5cAGAm5c
Am5cGm5cGAm5cppppGGGm5cGm5cm5cAm5cAm5cm5cpGpm5cGm5cm5c
Gm5cpGm5cpApApppGm5cGAm5cAGppGm5cm5cGGAAm5cm5cm5cppm5c
m5cm5cGAm5cm5cpm5cm5cm5cAm5cGAAGAm5cm5cm5cGppm5cAm5cm5c
pppGm5cGm5cApm5cm5cm5cm5cpGAm5cm5cm5cpm5cm5cm5cm5cm5cm5
cApm5cm5cm5cGm5cm5cppm5cGm5cAApGpm5cpm5cAGGm5cApm5cGpm5
cm5cpm5cGm5cm5cm5cGGpGAGGGAm5cm5cm5cpm5cGpm5cGGAAGm5c
GGm5cm5cGm5cGApm5cAGm5cGAGGm5cm5cGAAGm5cm5cGm5cm5cAG
m5cGGm5cAGm5cpppGGpm5cGm5cm5cpGm5cAm5cpGm5cm5cAGGpGm5c
ppm5cGGm5cpm5cApm5cAm5cm5cAAm5cGpGGAAGGm5cGGm5cpm5cGm
5cpGGAAGm5cm5cGGpm5cGpm5cpGm5cGAm5cpm5cm5cpGGAm5cm5cpG
m5cGpAm5cm5cAAm5cApAGAGGpGAGm5cm5cGAm5cm5cm5cpm5cGGpp
m5cpm5cpGm5cpGppppm5cAGGAGAAm5cAAApm5cpm5cm5cGm5cAm5cG
Am5cAm5cm5cGpAGAm5cm5cpGAm5cm5cGAm5cm5cpAAAm5cApm5cAAG
GGm5cm5cGm5cpGm5cGpGGpGGGm5cGAAm5cGGGAm5cm5cGAm5cpGm
5cpGGpGGAm5cm5cpm5cAAm5cAAm5cpppGGm5cm5cm5cAm5cGAm5cGm
5cm5cpGAm5cGm5cm5cAGGm5cpm5cAGAAAAm5cAAm5cAm5cGGpm5cpm
5cGGpGm5cpGGm5cm5cpppGm5cGm5cpGm5cm5cGm5cpAGAm5cm5cGm5
cGpGm5cm5cm5cGppAGm5cGGAm5cpGm5cAm5cm5cpm5cpppm5cAGAGm
5cm5cAGm5cGGm5cGm5cGGm5cGGm5cGAAGAAAApm5cGGm5cm5cGm5c
GAApGGAGGm5cGm5cGm5cGm5cm5cApm5cApm5cm5cGm5cm5cGm5cAm
5cGGm5cpm5cAm5cm5cAm5cpGGGm5cm5cGpGm5cGAm5cpGAm5cm5cGp
GAm5cGm5cm5cGAAm5cpGGm5cGm5cm5cGm5cAGAAm5cm5cGAm5cAGm
5cAGpppGGAGGm5cAGGGm5cAGApm5cpppGpm5cAGm5cm5cAGppm5cG
m5cm5cpppm5cGm5cGm5cm5cGGm5cGm5cm5cApm5cm5cm5cGm5cpGAm
5cGm5cpGGpAGAm5cGm5cm5cm5cpGGAGm5cAGm5cpGGm5cm5cpGppm
5cGGAm5cm5cm5cpAAm5cAm5cGpAm5cApm5cm5cAm5cAAAAm5cGGAGA
m5cGGAm5cGAAm5cGAGGm5cm5cAApGGApm5cApGm5cpGpppm5cpGm5
cApm5cAm5cGAm5cpm5cAm5cm5cGm5cAm5cm5cm5cGm5cm5cGAm5cm5c
AGm5cGpGpppm5cpGm5cAm5cppppm5cGGpppAm5cAm5cGm5cApm5cGm5
cGm5cm5cGAGGpGGpGGm5cGm5cGAm5cAm5cAApm5cm5cGpAm5cm5cm
5cGm5cAm5cm5cpAm5cGAm5cGm5cppGm5cm5cGGAm5cAAm5cGGm5cpp
m5cm5cAGm5cpGppGAppm5cm5cm5cAAAAGppppAm5cGm5cpGAm5cGm5c
Gm5cApAm5cApm5cm5cm5cGAGpAm5cApm5cGpGm5cAGApm5cm5cAGAA
pGm5cpppm5cGAAAm5cm5cAApm5cAGAm5cpm5cAm5cGAm5cAm5cm5cAp
m5cpppppm5cm5cm5cGGAAAAm5cApm5cm5cm5cGGGm5cGpm5cpm5cm5c
ApAGAAGm5cm5cGGm5cm5cm5cGm5cpAm5cm5cm5cGApm5cGpGpGm5c
GAApm5cAm5cm5cm5cpm5cm5cGm5cGpm5cAm5cGm5cpGAm5cm5cGGm5
CGAm5cm5cAGGm5cm5cGppm5cApppGGAAm5cAm5cm5cGAm5cAGm5cm5
cGm5cpAGGm5cm5cGm5cApm5cm5cAm5cpppppm5cm5cGm5cm5cGpGGGp
pppGGAm5cpm5cpm5cAm5cAm5cm5cm5cGGpAAAm5cm5cGGAm5cAAAAp
m5cAAGm5cGpm5cm5cm5cm5cAGGpGm5cAGm5cpGm5cGm5cGm5cm5cG
Gpm5cpm5cpppm5cm5cAm5cGGAGm5cGAm5cGpm5cGpGm5cGm5cGGm5c
Gm5cm5cGpm5cpm5cm5cGAGpppm5cpm5cm5cm5cGm5cAGpm5cm5cm5cm
5cm5cGGAppAm5cm5cAm5cm5cm5cAm5cm5cGAGGAAGAGGAGGAAGAAG
AGGAAGAGGAm5cGAm5cGAAGApGAm5cm5cpm5cpm5cm5cpm5cm5cAm5
cAm5cm5cGAm5cGm5cm5cGAm5cm5cm5cm5cm5cm5cpGpm5cm5cGAAGm
5cm5cApGpppGm5cm5cGGm5cppm5cGAGGAGGm5cm5cAGm5cGGm5cGA
m5cGAGGAm5cpm5cGGAm5cAm5cm5cm5cAAGm5cm5cGGAm5cpGpm5cm
5cm5cGGGm5cAm5cpGApm5cm5cpGAm5cm5cGGAm5cAAAGAm5cGpm5cG
AAGm5cGGpAAm5cAAm5cGGGGm5cpm5cpm5cAm5cGm5cpm5cGpm5cAp
m5cm5cm5cm5cpm5cGpGGm5cAm5cGpm5cpppGm5cGAGm5cm5cppGAm5
cGAm5cppGGpAm5cm5cAppAAm5cGGpGAGm5cGpGm5cAGm5cAm5cGm5c
m5cGm5cAppAm5cGAm5cm5cpAm5cm5cpm5cppApm5cpGm5cGm5cAGm5c
GAm5cApGGAm5cGGm5cGAm5cGpGm5cGpAm5cm5cGm5cGGm5cAGAm5
cApm5cAGm5cAGm5cAm5cGppGm5cGGpm5cm5cGpGm5cm5cm5cGm5cG
m5cm5cAm5cGAm5cm5cm5cpm5cAm5cm5cm5cApm5cAGm5cAm5cm5cGm5
cppm5cm5cAm5cppm5cm5cAGm5cAm5cm5cm5cm5cAm5cGm5cAGpm5cGA
m5cm5cm5cm5cGm5cApm5cpAGAGAGAGAm5cppm5cpppGpppppm5cm5cm
5cm5cm5cGm5cGpGpppppm5cm5cm5cAppm5cm5cm5cpGpApppApppm5cpA
AApAApAAAAAm5cAm5cAGAGAm5cGppGApAApAAm5cm5cGm5cAGpGpG
m5cpppAppAGGGpApm5cAm5cGGpGpAGAAAAAAAAAAGAGAGGGAAAm5c
m5cm5cpAAApApAGm5cGpm5cpm5cpm5cAAAAAAAAAAAAAAAAAAAAAAA
AAAAA
SEQ ID NO: 15 (pp71 Construct B.2-Full-length (FT) + pseudoU/5meC)
Gm5cm5cAm5cm5cm5cGm5cm5cGm5cGm5cAm5cGm5cGm5cppAAGAm5cG
Am5cpm5cpApAAAAAm5cm5cm5cAm5cGpm5cm5cAm5cpm5cAGAm5cAm5c
Gm5cGAm5cppppGGGm5cGm5cm5cAm5cAm5cm5cpGpm5cGm5cm5cGm5c
pGm5cpApApppGm5cGAm5cAGppGm5cm5cGGAAm5cm5cm5cppm5cm5cm5
cGAm5cm5cpm5cm5cm5cAm5cGAAGAm5cm5cm5cGppm5cAm5cm5cpppGm
5cGm5cApm5cm5cm5cm5cpGAm5cm5cm5cpm5cm5cm5cm5cm5cm5cApm5c
m5cm5cGm5cm5cppm5cGm5cAApGpm5cpm5cAGGm5cApm5cGpm5cm5cpm
5cGm5cm5cm5cGGpGAGGGAm5cm5cm5cpm5cGpm5cGGAAGm5cGGm5cm
5cGm5cGApm5cAGm5cGAGGm5cm5cGAAGm5cm5cGm5cm5cAGm5cGGm5
cAGm5cpppGGpm5cGm5cm5cpGm5cAm5cpGm5cm5cAGGpGm5cppm5cGG
m5cpm5cApm5cAm5cm5cAAm5cGpGGAAGGm5cGGm5cpm5cGm5cpGGAA
Gm5cm5cGGpm5cGpm5cpGm5cGAm5cpm5cm5cpGGAm5cm5cpGm5cGpAm
5cm5cAAm5cApAGAGGpGAGm5cm5cGAm5cm5cm5cpm5cGGppm5cpm5cp
Gm5cpGppppm5cAGGAGAAm5cAAApm5cpm5cm5cGm5cAm5cGAm5cAm5c
m5cGpAGAm5cm5cpGAm5cm5cGAm5cm5cpAAAm5cApm5cAAGGGm5cm5c
Gm5cpGm5cGpGGpGGGm5cGAAm5cGGGAm5cm5cGAm5cpGm5cpGGpGG
Am5cm5cpm5cAAm5cAAm5cpppGGm5cm5cm5cAm5cGAm5cGm5cm5cpGA
m5cGm5cm5cAGGm5cpm5cAGAAAAm5cAAm5cAm5cGGpm5cpm5cGGpGm
5cpGGm5cm5cpppGm5cGm5cpGm5cm5cGm5cpAGAm5cm5cGm5cGpGm5c
m5cm5cGppAGm5cGGAm5cpGm5cAm5cm5cpm5cpppm5cAGAGm5cm5cAG
m5cGGm5cGm5cGGm5cGGm5cGAAGAAAApm5cGGm5cm5cGm5cGAApGG
AGGm5cGm5cGm5cGm5cm5cApm5cApm5cm5cGm5cm5cGm5cAm5cGGm5
cpm5cAm5cm5cAm5cpGGGm5cm5cGpGm5cGAm5cpGAm5cm5cGpGAm5cG
m5cm5cGAAm5cpGGm5cGm5cm5cGm5cAGAAm5cm5cGAm5cAGm5cAGpp
pGGAGGm5cAGGGm5cAGApm5cpppGpm5cAGm5cm5cAGppm5cGm5cm5c
pppm5cGm5cGm5cm5cGGm5cGm5cm5cApm5cm5cm5cGm5cpGAm5cGm5c
pGGpAGAm5cGm5cm5cm5cpGGAGm5cAGm5cpGGm5cm5cpGppm5cGGAm
5cm5cm5cpAAm5cAm5cGpAm5cApm5cm5cAm5cAAAAm5cGGAGAm5cGGA
m5cGAAm5cGAGGm5cm5cAApGGApm5cApGm5cpGpppm5cpGm5cApm5cA
m5cGAm5cpm5cAm5cm5cGm5cAm5cm5cm5cGm5cm5cGAm5cm5cAGm5cG
pGpppm5cpGm5cAm5cppppm5cGGpppAm5cAm5cGm5cApm5cGm5cGm5cm
5cGAGGpGGpGGm5cGm5cGAm5cAm5cAApm5cm5cGpAm5cm5cm5cGm5c
Am5cm5cpAm5cGAm5cGm5cppGm5cm5cGGAm5cAAm5cGGm5cppm5cm5c
AGm5cpGppGAppm5cm5cm5cAAAAGppppAm5cGm5cpGAm5cGm5cGm5cA
pAm5cApm5cm5cm5cGAGpAm5cApm5cGpGm5cAGApm5cm5cAGAApGm5c
pppm5cGAAAm5cm5cAApm5cAGAm5cpm5cAm5cGAm5cAm5cm5cApm5cpp
pppm5cm5cm5cGGAAAAm5cApm5cm5cm5cGGGm5cGpm5cpm5cm5cApAG
AAGm5cm5cGGm5cm5cm5cGm5cpAm5cm5cm5cGApm5cGpGpGm5cGAAp
m5cAm5cm5cm5cpm5cm5cGm5cGpm5cAm5cGm5cpGAm5cm5cGGm5cGAm
5cm5cAGGm5cm5cGppm5cApppGGAAm5cAm5cm5cGAm5cAGm5cm5cGm5
cpAGGm5cm5cGm5cApm5cm5cAm5cpppppm5cm5cGm5cm5cGpGGGppppG
GAm5cpm5cpm5cAm5cAm5cm5cm5cGGpAAAm5cm5cGGAm5cAAAApm5cA
AGm5cGpm5cm5cm5cm5cAGGpGm5cAGm5cpGm5cGm5cGm5cm5cGGpm5
cpm5cpppm5cm5cAm5cGGAGm5cGAm5cGpm5cGpGm5cGm5cGGm5cGm5c
m5cGpm5cpm5cm5cGAGpppm5cpm5cm5cm5cGm5cAGpm5cm5cm5cm5cm5
cGGAppAm5cm5cAm5cm5cm5cAm5cm5cGAGGAAGAGGAGGAAGAAGAGG
AAGAGGAm5cGAm5cGAAGApGAm5cm5cpm5cpm5cm5cpm5cm5cAm5cAm5
cm5cGAm5cGm5cm5cGAm5cm5cm5cm5cm5cm5cpGpm5cm5cGAAGm5cm5
cApGpppGm5cm5cGGm5cppm5cGAGGAGGm5cm5cAGm5cGGm5cGAm5cG
AGGAm5cpm5cGGAm5cAm5cm5cm5cAAGm5cm5cGGAm5cpGpm5cm5cm5c
GGGm5cAm5cpGApm5cm5cpGAm5cm5cGGAm5cAAAGAm5cGpm5cGAAGm
5cGGpAAm5cAAm5cGGGGm5cpm5cpm5cAm5cGm5cpm5cGpm5cApm5cm5
cm5cm5cpm5cGpGGm5cAm5cGpm5cpppGm5cGAGm5cm5cppGAm5cGAm5
cppGGpAm5cm5cAppAAm5cGGpGAGm5cGpGm5cAGm5cAm5cGm5cm5cG
m5cAppAm5cGAm5cm5cpAm5cm5cpm5cppApm5cpGm5cGm5cAGm5cGAm5
cApGGAm5cGGm5cGAm5cGpGm5cGpAm5cm5cGm5cGGm5cAGAm5cApm5
cAGm5cAGm5cAm5cGppGm5cGGpm5cm5cGpGm5cm5cm5cGm5cGm5cm5c
Am5cGAm5cm5cm5cpm5cAm5cm5cm5cApm5cAGm5cAm5cm5cGm5cppm5c
m5cAm5cppm5cm5cAGm5cAm5cm5cm5cm5cAm5cGm5cAGpm5cGAm5cm5c
m5cm5cGm5cApm5cpAGAGAGAGAm5cppm5cpppGpppppm5cm5cm5cm5cm
5cGm5cGpGpppppm5cm5cm5cAppm5cm5cm5cpGpApppApppm5cpAAApAAp
AAAAAm5cAm5cAGAGAm5cGppGApAApAAm5cm5cGm5cAGpGpGm5cpppA
ppAGGGpApm5cAm5cGGpGpAGAAAAAAAAAGAGAGGGAAAm5cm5cm5cp
AAApApAGm5cGpm5cpm5cpm5cAGm5cm5cpGAGpAGGAAG
SEQ ID NO: 16 (pp71 Construct B.3-Full-length (FT) + pseudoU/5meC)
Gm5cm5cAm5cm5cm5cGm5cm5cGm5cGm5cAm5cGm5cGm5cppAAGAm5cG
Am5cpm5cpApAAAAAm5cm5cm5cAm5cGpm5cm5cAm5cpm5cAGAm5cAm5c
Gm5cGAm5cppppGGGm5cGm5cm5cAm5cAm5cm5cpGpm5cGm5cm5cGm5c
pGm5cpApApppGm5cGAm5cAGppGm5cm5cGGAAm5cm5cm5cppm5cm5cm5
cGAm5cm5cpm5cm5cm5cAm5cGAAGAm5cm5cm5cGppm5cAm5cm5cpppGm
5cGm5cApm5cm5cm5cm5cpGAm5cm5cm5cpm5cm5cm5cm5cm5cm5cApm5c
m5cm5cGm5cm5cppm5cGm5cAApGpm5cpm5cAGGm5cApm5cGpm5cm5cpm
5cGm5cm5cm5cGGpGAGGGAm5cm5cm5cpm5cGpm5cGGAAGm5cGGm5cm
5cGm5cGApm5cAGm5cGAGGm5cm5cGAAGm5cm5cGm5cm5cAGm5cGGm5
cAGm5cpppGGpm5cGm5cm5cpGm5cAm5cpGm5cm5cAGGpGm5cppm5cGG
m5cpm5cApm5cAm5cm5cAAm5cGpGGAAGGm5cGGm5cpm5cGm5cpGGAA
Gm5cm5cGGpm5cGpm5cpGm5cGAm5cpm5cm5cpGGAm5cm5cpGm5cGpAm
5cm5cAAm5cApAGAGGpGAGm5cm5cGAm5cm5cm5cpm5cGGppm5cpm5cp
Gm5cpGppppm5cAGGAGAAm5cAAApm5cpm5cm5cGm5cAm5cGAm5cAm5c
m5cGpAGAm5cm5cpGAm5cm5cGAm5cm5cpAAAm5cApm5cAAGGGm5cm5c
Gm5cpGm5cGpGGpGGGm5cGAAm5cGGGAm5cm5cGAm5cpGm5cpGGpGG
Am5cm5cpm5cAAm5cAAm5cpppGGm5cm5cm5cAm5cGAm5cGm5cm5cpGA
m5cGm5cm5cAGGm5cpm5cAGAAAAm5cAAm5cAm5cGGpm5cpm5cGGpGm
5cpGGm5cm5cpppGm5cGm5cpGm5cm5cGm5cpAGAm5cm5cGm5cGpGm5c
m5cm5cGppAGm5cGGAm5cpGm5cAm5cm5cpm5cpppm5cAGAGm5cm5cAG
m5cGGm5cGm5cGGm5cGGm5cGAAGAAAApm5cGGm5cm5cGm5cGAApGG
AGGm5cGm5cGm5cGm5cm5cApm5cApm5cm5cGm5cm5cGm5cAm5cGGm5
cpm5cAm5cm5cAm5cpGGGm5cm5cGpGm5cGAm5cpGAm5cm5cGpGAm5cG
m5cm5cGAAm5cpGGm5cGm5cm5cGm5cAGAAm5cm5cGAm5cAGm5cAGpp
pGGAGGm5cAGGGm5cAGApm5cpppGpm5cAGm5cm5cAGppm5cGm5cm5c
pppm5cGm5cGm5cm5cGGm5cGm5cm5cApm5cm5cm5cGm5cpGAm5cGm5c
pGGpAGAm5cGm5cm5cm5cpGGAGm5cAGm5cpGGm5cm5cpGppm5cGGAm
5cm5cm5cpAAm5cAm5cGpAm5cApm5cm5cAm5cAAAAm5cGGAGAm5cGGA
m5cGAAm5cGAGGm5cm5cAApGGApm5cApGm5cpGpppm5cpGm5cApm5cA
m5cGAm5cpm5cAm5cm5cGm5cAm5cm5cm5cGm5cm5cGAm5cm5cAGm5cG
pGpppm5cpGm5cAm5cppppm5cGGpppAm5cAm5cGm5cApm5cGm5cGm5cm
5cGAGGpGGpGGm5cGm5cGAm5cAm5cAApm5cm5cGpAm5cm5cm5cGm5c
Am5cm5cpAm5cGAm5cGm5cppGm5cm5cGGAm5cAAm5cGGm5cppm5cm5c
AGm5cpGppGAppm5cm5cm5cAAAAGppppAm5cGm5cpGAm5cGm5cGm5cA
pAm5cApm5cm5cm5cGAGpAm5cApm5cGpGm5cAGApm5cm5cAGAApGm5c
pppm5cGAAAm5cm5cAApm5cAGAm5cpm5cAm5cGAm5cAm5cm5cApm5cpp
pppm5cm5cm5cGGAAAAm5cApm5cm5cm5cGGGm5cGpm5cpm5cm5cApAG
AAGm5cm5cGGm5cm5cm5cGm5cpAm5cm5cm5cGApm5cGpGpGm5cGAAp
m5cAm5cm5cm5cpm5cm5cGm5cGpm5cAm5cGm5cpGAm5cm5cGGm5cGAm
5cm5cAGGm5cm5cGppm5cApppGGAAm5cAm5cm5cGAm5cAGm5cm5cGm5
cpAGGm5cm5cGm5cApm5cm5cAm5cpppppm5cm5cGm5cm5cGpGGGppppG
GAm5cpm5cpm5cAm5cAm5cm5cm5cGGpAAAm5cm5cGGAm5cAAAApm5cA
AGm5cGpm5cm5cm5cm5cAGGpGm5cAGm5cpGm5cGm5cGm5cm5cGGpm5
cpm5cpppm5cm5cAm5cGGAGm5cGAm5cGpm5cGpGm5cGm5cGGm5cGm5c
m5cGpm5cpm5cm5cGAGpppm5cpm5cm5cm5cGm5cAGpm5cm5cm5cm5cm5
cGGAppAm5cm5cAm5cm5cm5cAm5cm5cGAGGAAGAGGAGGAAGAAGAGG
AAGAGGAm5cGAm5cGAAGApGAm5cm5cpm5cpm5cm5cpm5cm5cAm5cAm5
cm5cGAm5cGm5cm5cGAm5cm5cm5cm5cm5cm5cpGpm5cm5cGAAGm5cm5
cApGpppGm5cm5cGGm5cppm5cGAGGAGGm5cm5cAGm5cGGm5cGAm5cG
AGGAm5cpm5cGGAm5cAm5cm5cm5cAAGm5cm5cGGAm5cpGpm5cm5cm5c
GGGm5cAm5cpGApm5cm5cpGAm5cm5cGGAm5cAAAGAm5cGpm5cGAAGm
5cGGpAAm5cAAm5cGGGGm5cpm5cpm5cAm5cGm5cpm5cGpm5cApm5cm5
cm5cm5cpm5cGpGGm5cAm5cGpm5cpppGm5cGAGm5cm5cppGAm5cGAm5
cppGGpAm5cm5cAppAAm5cGGpGAGm5cGpGm5cAGm5cAm5cGm5cm5cG
m5cAppAm5cGAm5cm5cpAm5cm5cpm5cppApm5cpGm5cGm5cAGm5cGAm5
cApGGAm5cGGm5cGAm5cGpGm5cGpAm5cm5cGm5cGGm5cAGAm5cApm5
cAGm5cAGm5cAm5cGppGm5cGGpm5cm5cGpGm5cm5cm5cGm5cGm5cm5c
Am5cGAm5cm5cm5cpm5cAm5cm5cm5cApm5cAGm5cAm5cm5cGm5cppm5c
m5cAm5cppm5cm5cAGm5cAm5cm5cm5cm5cAm5cGm5cAGpm5cGAm5cm5c
m5cm5cGm5cApm5cpAGAGAGAGAm5cppm5cpppGpppppm5cm5cm5cm5cm
5cGm5cGpGpppppm5cm5cm5cAppm5cm5cm5cpGpApppApppm5cpAAApAAp
AAAAAm5cAm5cAGAGAm5cGppGApAApAAm5cm5cGm5cAGpGpGm5cpppA
ppAGGGpApm5cAm5cGGpGpAGAAAAAAAAAAGAGAGGGAAAm5cm5cm5c
pAAApApAGm5cGpm5cpm5cpm5c
SEQ ID NO: 17 (pp71 Construct C-Immediate early (IE1) + pseudoU/5meC)
AGGpm5cGpppAGpGAAm5cm5cGpm5cAGApm5cGm5cm5cpGGAGAm5cGm
5cm5cApm5cm5cAm5cGm5cpGppppGAm5cm5cpm5cm5cApAGAAGAm5cAm
5cm5cGGGAm5cm5cGApm5cm5cAGm5cm5cpm5cm5cGm5cGGm5cm5cGG
GAAm5cGGpGm5cAppGGAAm5cGm5cGGAppm5cm5cm5cm5cGpGm5cm5c
AAGAGpGAm5cApGpm5cpm5cAGGm5cApm5cGpm5cm5cpm5cGm5cm5cm5
cGGpGAGGGAm5cm5cm5cpm5cGpm5cGGAAGm5cGGm5cm5cGm5cGApm
5cAGm5cGAGGm5cm5cGAAGm5cm5cGm5cm5cAGm5cGGm5cAGm5cpppG
Gpm5cGm5cm5cpGm5cAm5cpGm5cm5cAGGpGm5cppm5cGGm5cpm5cApm
5cAm5cm5cAAm5cGpGGAAGGm5cGGm5cpm5cGm5cpGGAAGm5cm5cGGp
m5cGpm5cpGm5cGAm5cpm5cm5cpGGAm5cm5cpGm5cGpAm5cm5cAAm5c
ApAGAGGpGAGm5cm5cGAm5cm5cm5cpm5cGGppm5cpm5cpGm5cpGpppp
m5cAGGAGAAm5cAAApm5cpm5cm5cGm5cAm5cGAm5cAm5cm5cGpAGAm
5cm5cpGAm5cm5cGAm5cm5cpAAAm5cApm5cAAGGGm5cm5cGm5cpGm5c
GpGGpGGGm5cGAAm5cGGGAm5cm5cGAm5cpGm5cpGGpGGAm5cm5cpm
5cAAm5cAAm5cpppGGm5cm5cm5cAm5cGAm5cGm5cm5cpGAm5cGm5cm5c
AGGm5cpm5cAGAAAAm5cAAm5cAm5cGGpm5cpm5cGGpGm5cpGGm5cm5
cpppGm5cGm5cpGm5cm5cGm5cpAGAm5cm5cGm5cGpGm5cm5cm5cGppA
Gm5cGGAm5cpGm5cAm5cm5cpm5cpppm5cAGAGm5cm5cAGm5cGGm5cG
m5cGGm5cGGm5cGAAGAAAApm5cGGm5cm5cGm5cGAApGGAGGm5cGm5
cGm5cGm5cm5cApm5cApm5cm5cGm5cm5cGm5cAm5cGGm5cpm5cAm5cm
5cAm5cpGGGm5cm5cGpGm5cGAm5cpGAm5cm5cGpGAm5cGm5cm5cGAA
m5cpGGm5cGm5cm5cGm5cAGAAm5cm5cGAm5cAGm5cAGpppGGAGGm5c
AGGGm5cAGApm5cpppGpm5cAGm5cm5cAGppm5cGm5cm5cpppm5cGm5c
Gm5cm5cGGm5cGm5cm5cApm5cm5cm5cGm5cpGAm5cGm5cpGGpAGAm5
cGm5cm5cm5cpGGAGm5cAGm5cpGGm5cm5cpGppm5cGGAm5cm5cm5cpA
Am5cAm5cGpAm5cApm5cm5cAm5cAAAAm5cGGAGAm5cGGAm5cGAAm5c
GAGGm5cm5cAApGGApm5cApGm5cpGpppm5cpGm5cApm5cAm5cGAm5cp
m5cAm5cm5cGm5cAm5cm5cm5cGm5cm5cGAm5cm5cAGm5cGpGpppm5cp
Gm5cAm5cppppm5cGGpppAm5cAm5cGm5cApm5cGm5cGm5cm5cGAGGpG
GpGGm5cGm5cGAm5cAm5cAApm5cm5cGpAm5cm5cm5cGm5cAm5cm5cpA
m5cGAm5cGm5cppGm5cm5cGGAm5cAAm5cGGm5cppm5cm5cAGm5cpGpp
GAppm5cm5cm5cAAAAGppppAm5cGm5cpGAm5cGm5cGm5cApAm5cApm5c
m5cm5cGAGpAm5cApm5cGpGm5cAGApm5cm5cAGAApGm5cpppm5cGAAA
m5cm5cAApm5cAGAm5cpm5cAm5cGAm5cAm5cm5cApm5cpppppm5cm5cm
5cGGAAAAm5cApm5cm5cm5cGGGm5cGpm5cpm5cm5cApAGAAGm5cm5cG
Gm5cm5cm5cGm5cpAm5cm5cm5cGApm5cGpGpGm5cGAApm5cAm5cm5cm
5cpm5cm5cGm5cGpm5cAm5cGm5cpGAm5cm5cGGm5cGAm5cm5cAGGm5c
m5cGppm5cApppGGAAm5cAm5cm5cGAm5cAGm5cm5cGm5cpAGGm5cm5c
Gm5cApm5cm5cAm5cpppppm5cm5cGm5cm5cGpGGGppppGGAm5cpm5cpm
5cAm5cAm5cm5cm5cGGpAAAm5cm5cGGAm5cAAAApm5cAAGm5cGpm5cm
5cm5cm5cAGGpGm5cAGm5cpGm5cGm5cGm5cm5cGGpm5cpm5cpppm5cm
5cAm5cGGAGm5cGAm5cGpm5cGpGm5cGm5cGGm5cGm5cm5cGpm5cpm5
cm5cGAGpppm5cpm5cm5cm5cGm5cAGpm5cm5cm5cm5cm5cGGAppAm5cm
5cAm5cm5cm5cAm5cm5cGAGGAAGAGGAGGAAGAAGAGGAAGAGGAm5c
GAm5cGAAGApGAm5cm5cpm5cpm5cm5cpm5cm5cAm5cAm5cm5cGAm5cG
m5cm5cGAm5cm5cm5cm5cm5cm5cpGpm5cm5cGAAGm5cm5cApGpppGm5c
m5cGGm5cppm5cGAGGAGGm5cm5cAGm5cGGm5cGAm5cGAGGAm5cpm5
cGGAm5cAm5cm5cm5cAAGm5cm5cGGAm5cpGpm5cm5cm5cGGGm5cAm5c
pGApm5cm5cpGAm5cm5cGGAm5cAAAGAm5cGpm5cGAAGm5cGGpAAm5c
AAm5cGGGGm5cpm5cpm5cAm5cGm5cpm5cGpm5cApm5cm5cm5cm5cpm5c
GpGGm5cAm5cGpm5cpppGm5cGAGm5cm5cppGAm5cGAm5cppGGpAm5cm
5cAppAAm5cGGpGAGm5cGpGm5cAGm5cAm5cGm5cm5cGm5cAppAm5cGA
m5cm5cpAm5cm5cpm5cppApm5cpGm5cGm5cAGm5cGAm5cApGGAm5cGG
m5cGAm5cGpGm5cGpAm5cm5cGm5cGGm5cAGAm5cApm5cAGm5cAGm5c
Am5cGppGm5cGGpm5cm5cGpGm5cm5cm5cGm5cGm5cm5cAm5cGAm5cm5
cm5cpm5cAm5cm5cm5cApm5cAGm5cAm5cm5cGm5cppm5cm5cAm5cppm5c
m5cAGm5cAm5cm5cm5cm5cAm5cGm5cAGpm5cGAm5cm5cm5cm5cGm5cA
pm5cpAGGm5cGGm5cm5cGm5cppAAppAAGm5cpGm5cm5cppm5cpGm5cG
GGGm5cppGm5cm5cppm5cpGGm5cm5cApGm5cm5cm5cppm5cppm5cpm5c
pm5cm5cm5cppGm5cAm5cm5cpGpAm5cm5cpm5cppGGpm5cpppGAApAAA
Gm5cm5cpGAGpAGGAAG
SEQ ID NO: 18 (pp71 Construct D-short + pseudoU/5meC)
AGGGm5cGm5cm5cAm5cAm5cm5cpGpm5cGm5cm5cGm5cpGm5cpApAppp
Gm5cGAm5cAGppGm5cm5cGGAAm5cm5cm5cppm5cm5cm5cGAm5cm5cpm
5cm5cm5cAm5cGAAGAm5cm5cm5cGppm5cAm5cm5cpppGm5cGm5cApm5c
m5cm5cm5cpGAm5cm5cm5cpm5cm5cm5cm5cm5cm5cApm5cm5cm5cGm5c
m5cppm5cGm5cAApGpm5cpm5cAGGm5cApm5cGpm5cm5cpm5cGm5cm5cm
5cGGpGAGGGAm5cm5cm5cpm5cGpm5cGGAAGm5cGGm5cm5cGm5cGAp
m5cAGm5cGAGGm5cm5cGAAGm5cm5cGm5cm5cAGm5cGGm5cAGm5cppp
GGpm5cGm5cm5cpGm5cAm5cpGm5cm5cAGGpGm5cppm5cGGm5cpm5cAp
m5cAm5cm5cAAm5cGpGGAAGGm5cGGm5cpm5cGm5cpGGAAGm5cm5cG
Gpm5cGpm5cpGm5cGAm5cpm5cm5cpGGAm5cm5cpGm5cGpAm5cm5cAAm
5cApAGAGGpGAGm5cm5cGAm5cm5cm5cpm5cGGppm5cpm5cpGm5cpGppp
pm5cAGGAGAAm5cAAApm5cpm5cm5cGm5cAm5cGAm5cAm5cm5cGpAGA
m5cm5cpGAm5cm5cGAm5cm5cpAAAm5cApm5cAAGGGm5cm5cGm5cpGm5
cGpGGpGGGm5cGAAm5cGGGAm5cm5cGAm5cpGm5cpGGpGGAm5cm5cp
m5cAAm5cAAm5cpppGGm5cm5cm5cAm5cGAm5cGm5cm5cpGAm5cGm5cm
5cAGGm5cpm5cAGAAAAm5cAAm5cAm5cGGpm5cpm5cGGpGm5cpGGm5c
m5cpppGm5cGm5cpGm5cm5cGm5cpAGAm5cm5cGm5cGpGm5cm5cm5cGp
pAGm5cGGAm5cpGm5cAm5cm5cpm5cpppm5cAGAGm5cm5cAGm5cGGm5c
Gm5cGGm5cGGm5cGAAGAAAApm5cGGm5cm5cGm5cGAApGGAGGm5cG
m5cGm5cGm5cm5cApm5cApm5cm5cGm5cm5cGm5cAm5cGGm5cpm5cAm5
cm5cAm5cpGGGm5cm5cGpGm5cGAm5cpGAm5cm5cGpGAm5cGm5cm5cG
AAm5cpGGm5cGm5cm5cGm5cAGAAm5cm5cGAm5cAGm5cAGpppGGAGG
m5cAGGGm5cAGApm5cpppGpm5cAGm5cm5cAGppm5cGm5cm5cpppm5cG
m5cGm5cm5cGGm5cGm5cm5cApm5cm5cm5cGm5cpGAm5cGm5cpGGpAG
Am5cGm5cm5cm5cpGGAGm5cAGm5cpGGm5cm5cpGppm5cGGAm5cm5cm
5cpAAm5cAm5cGpAm5cApm5cm5cAm5cAAAAm5cGGAGAm5cGGAm5cGAA
m5cGAGGm5cm5cAApGGApm5cApGm5cpGpppm5cpGm5cApm5cAm5cGAm
5cpm5cAm5cm5cGm5cAm5cm5cm5cGm5cm5cGAm5cm5cAGm5cGpGpppm5
cpGm5cAm5cppppm5cGGpppAm5cAm5cGm5cApm5cGm5cGm5cm5cGAGGp
GGpGGm5cGm5cGAm5cAm5cAApm5cm5cGpAm5cm5cm5cGm5cAm5cm5cp
Am5cGAm5cGm5cppGm5cm5cGGAm5cAAm5cGGm5cppm5cm5cAGm5cpGp
pGAppm5cm5cm5cAAAAGppppAm5cGm5cpGAm5cGm5cGm5cApAm5cApm5
cm5cm5cGAGpAm5cApm5cGpGm5cAGApm5cm5cAGAApGm5cpppm5cGAA
Am5cm5cAApm5cAGAm5cpm5cAm5cGAm5cAm5cm5cApm5cpppppm5cm5c
m5cGGAAAAm5cApm5cm5cm5cGGGm5cGpm5cpm5cm5cApAGAAGm5cm5c
GGm5cm5cm5cGm5cpAm5cm5cm5cGApm5cGpGpGm5cGAApm5cAm5cm5c
m5cpm5cm5cGm5cGpm5cAm5cGm5cpGAm5cm5cGGm5cGAm5cm5cAGGm
5cm5cGppm5cApppGGAAm5cAm5cm5cGAm5cAGm5cm5cGm5cpAGGm5cm
5cGm5cApm5cm5cAm5cpppppm5cm5cGm5cm5cGpGGGppppGGAm5cpm5c
pm5cAm5cAm5cm5cm5cGGpAAAm5cm5cGGAm5cAAAApm5cAAGm5cGpm5
cm5cm5cm5cAGGpGm5cAGm5cpGm5cGm5cGm5cm5cGGpm5cpm5cpppm5
cm5cAm5cGGAGm5cGAm5cGpm5cGpGm5cGm5cGGm5cGm5cm5cGpm5cp
m5cm5cGAGpppm5cpm5cm5cm5cGm5cAGpm5cm5cm5cm5cm5cGGAppAm5
cm5cAm5cm5cm5cAm5cm5cGAGGAAGAGGAGGAAGAAGAGGAAGAGGAm
5cGAm5cGAAGApGAm5cm5cpm5cpm5cm5cpm5cm5cAm5cAm5cm5cGAm5c
Gm5cm5cGAm5cm5cm5cm5cm5cm5cpGpm5cm5cGAAGm5cm5cApGpppGm
5cm5cGGm5cppm5cGAGGAGGm5cm5cAGm5cGGm5cGAm5cGAGGAm5cp
m5cGGAm5cAm5cm5cm5cAAGm5cm5cGGAm5cpGpm5cm5cm5cGGGm5cA
m5cpGApm5cm5cpGAm5cm5cGGAm5cAAAGAm5cGpm5cGAAGm5cGGpAA
m5cAAm5cGGGGm5cpm5cpm5cAm5cGm5cpm5cGpm5cApm5cm5cm5cm5cp
m5cGpGGm5cAm5cGpm5cpppGm5cGAGm5cm5cppGAm5cGAm5cppGGpAm
5cm5cAppAAm5cGGpGAGm5cGpGm5cAGm5cAm5cGm5cm5cGm5cAppAm5
cGAm5cm5cpAm5cm5cpm5cppApm5cpGm5cGm5cAGm5cGAm5cApGGAm5c
GGm5cGAm5cGpGm5cGpAm5cm5cGm5cGGm5cAGAm5cApm5cAGm5cAG
m5cAm5cGppGm5cGGpm5cm5cGpGm5cm5cm5cGm5cGm5cm5cAm5cGAm5
cm5cm5cpm5cAm5cm5cm5cApm5cAGm5cAm5cm5cGm5cppm5cm5cAm5cpp
m5cm5cAGm5cAm5cm5cm5cm5cAm5cGm5cAGpm5cGAm5cm5cm5cm5cGm
5cApm5cpAGAGAGAGAm5cppm5cpppGpppppm5cm5cm5cm5cm5cGm5cGp
Gpppppm5cm5cm5cAppm5cm5cm5cpGpApppApppm5cpAAApAApAAAAAm5
cAm5cAGAGAm5cGppGApAApAAm5cm5cGm5cAGm5cm5cpGAGpAGGAAG
SEQ ID NO: 19 (pp71 Construct E-pp65 + pseudoU/5meC)
AGGAm5cAGAGGAm5cm5cm5cpGGpGppGGGAAAm5cGGAm5cAm5cpAGG
m5cGpm5cm5cGm5cGm5cGApAm5cGGGGppAAAAm5cAAAAAAAApm5cGG
pGGpGGpGpGpGm5cpGGGGpGpGGpGAm5cGGpGGGGm5cpppGm5cm5cp
m5cpppppppppGpAApAAAAAAAGAm5cAm5cpm5cAApAApm5cm5cGm5cGG
ppGpm5cpm5cpGpGpAGAAm5cGpppppApppm5cGGGppm5cm5cGm5cGppp
GGpm5cGm5cm5cpGm5cm5cpGpGpAAGGm5cGGm5cGGm5cm5cGm5cAAA
GGGm5cGm5cGm5cm5cGm5cpm5cAGpm5cGm5cm5cpAm5cAm5cm5cm5cG
pAm5cGm5cGm5cAGGm5cAGm5cApGGAGpm5cGm5cGm5cGGpm5cGm5c
m5cGppGpm5cm5cm5cGAAApGApApm5cm5cGpAm5cpGGGpm5cm5cm5cA
pppm5cGGGGm5cAm5cGpGm5cpGAAAGm5cm5cGpGpppAGpm5cGm5cGG
m5cGAm5cAm5cGm5cm5cGGpGm5cpGm5cm5cGm5cAm5cGAGAm5cGm5c
GAm5cpm5cm5cpGm5cAGAm5cGGGpApm5cm5cAm5cGpAm5cGm5cGpGA
Gm5cm5cAGm5cm5cm5cpm5cGm5cpGApm5cm5cpGGpGpm5cGm5cAGpAm
5cAm5cGm5cm5cm5cGAm5cpm5cGAm5cGm5cm5cApGm5cm5cAm5cm5cG
m5cGGm5cGAm5cAApm5cAGm5cpGm5cAGGpGm5cAGm5cAm5cAm5cGpA
m5cpppAm5cGGGm5cAGm5cGAGGpGGAGAAm5cGpGpm5cGGpm5cAAm5c
GpGm5cAm5cAAm5cm5cm5cm5cAm5cGGGm5cm5cGAAGm5cApm5cpGm5c
m5cm5cm5cAGm5cm5cAGGAGm5cm5cm5cApGpm5cGApm5cpApGpGpAm5
cGm5cGm5cpGm5cm5cGm5cpm5cAAGApGm5cpGAAm5cApm5cm5cm5cm5
CAGm5cApm5cAAm5cGpGm5cAm5cm5cAm5cpAm5cm5cm5cGpm5cGGm5c
GGm5cm5cGAGm5cGm5cAAAm5cAm5cm5cGAm5cAm5cm5cpGm5cm5cm5c
GpAGm5cpGAm5cGm5cpGpGAppm5cAm5cGm5cGpm5cGGGm5cAAGm5cA
GApGpGGm5cAGGm5cGm5cGpm5cpm5cAm5cGGpm5cpm5cGGGAm5cpGG
m5cm5cpGGAm5cGm5cGpm5cAGm5cAGAAm5cm5cAGpGGAAAGAGm5cm5
cm5cGAm5cGpm5cpAm5cpAm5cAm5cGpm5cAGm5cGppm5cGpGpppm5cm5
cm5cAm5cm5cAAGGAm5cGpGGm5cAm5cpGm5cGGm5cAm5cGpGGpGpGm
5cGm5cGm5cAm5cGAGm5cpGGpppGm5cpm5cm5cApGGAGAAm5cAm5cG
m5cGm5cGm5cAAm5cm5cAAGApGm5cAGGpGApAGGpGAm5cm5cAGpAm5
cGpm5cAAGGpGpAm5cm5cpGGAGpm5cm5cppm5cpGm5cGAGGAm5cGpG
m5cm5cm5cpm5cm5cGGm5cAAGm5cpm5cpppApGm5cAm5cGpm5cAm5cG
m5cpGGGm5cpm5cpGAm5cGpGGAAGAGGAm5cm5cpGAm5cGApGAm5cm5
cm5cGm5cAAm5cm5cm5cGm5cAAm5cm5cm5cppm5cApGm5cGm5cm5cm5c
m5cm5cAm5cGAGm5cGm5cAAm5cGGm5cpppAm5cGGpGppGpGpm5cm5cm
5cAAAAApApGApAApm5cAAAm5cm5cGGGm5cAAGApm5cpm5cGm5cAm5c
Apm5cApGm5cpGGApGpGGm5cppppAm5cm5cpm5cAm5cAm5cGAGm5cApp
ppGGGm5cpGm5cpGpGpm5cm5cm5cAAGAGm5cApm5cm5cm5cGGGm5cm
5cpGAGm5cApm5cpm5cAGGpAAm5cm5cpGppGApGAAm5cGGGm5cAGm5c
AGApm5cppm5cm5cpGGAGGpAm5cAAGm5cGApAm5cGm5cGAGAm5cm5c
GpGGAAm5cpGm5cGpm5cAGpAm5cGApm5cm5cm5cGpGGm5cpGm5cAm5
cpm5cppm5cpppppm5cGApApm5cGAm5cppGm5cpGm5cpGm5cAGm5cGm5c
GGGm5cm5cpm5cAGpAm5cAGm5cGAGm5cAm5cm5cm5cm5cAm5cm5cppm
5cAm5cm5cAGm5cm5cAGpApm5cGm5cApm5cm5cAGGGm5cAAGm5cppGA
GpAm5cm5cGAm5cAm5cAm5cm5cpGGGAm5cm5cGGm5cAm5cGAm5cGAG
GGpGm5cm5cGm5cm5cm5cAGGGm5cGAm5cGAm5cGAm5cGpm5cpGGAm
5cm5cAGm5cGGApm5cGGAm5cpm5cm5cGAm5cGAAGAAm5cpm5cGpAAm
5cm5cAm5cm5cGAGm5cGm5cAAGAm5cGm5cm5cm5cm5cGm5cGppAm5cm
5cGGm5cGGm5cGGm5cGm5cm5cApGGm5cGGGm5cGm5cm5cpm5cm5cAm
5cppm5cm5cGm5cGGGm5cm5cGm5cAAAm5cGm5cAAApm5cAGm5cApm5c
m5cpm5cGGm5cGAm5cGGm5cGpGm5cAm5cGGm5cGGGm5cGppApGAm5c
Am5cGm5cGGm5cm5cGm5cm5cppAAGGm5cm5cGAGpm5cm5cAm5cm5cGp
m5cGm5cGm5cm5cm5cGAAGAGGAm5cAm5cm5cGAm5cGAGGAppm5cm5c
GAm5cAAm5cGAAApm5cm5cAm5cAApm5cm5cGGm5cm5cGpGppm5cAm5c
m5cpGGm5cm5cGm5cm5cm5cpGGm5cAGGm5cm5cGGm5cApm5cm5cpGG
m5cm5cm5cGm5cAAm5cm5cpGGpGm5cm5cm5cApGGpGGm5cpAm5cGGpp
m5cAGGGpm5cAGAApm5cpGAAGpAm5cm5cAGGAGppm5cppm5cpGGGAm
5cGm5cm5cAAm5cGAm5cApm5cpAm5cm5cGm5cApm5cppm5cGm5cm5cGA
AppGGAAGGm5cGpApGGm5cAGm5cm5cm5cGm5cpGm5cGm5cAAm5cm5c
m5cAAAm5cGpm5cGm5cm5cGm5cm5cAm5cm5cGGm5cAAGAAGm5cm5cm
5cpGm5cm5cm5cGGGm5cm5cApGm5cApm5cGm5cm5cpm5cGAm5cGm5cm
5cm5cAAAAAGm5cApm5cGAGGppGAGm5cm5cAm5cm5cm5cGm5cm5cGm5
cGm5cAm5cGm5cGm5cppAAGAm5cGAm5cpm5cpApAAAAAm5cm5cm5cAm
5cGpm5cm5cAm5cpm5cAGAm5cAm5cGm5cGAm5cppppGGGm5cGm5cm5c
Am5cAm5cm5cpGpm5cGm5cm5cGm5cpGm5cpApApppGm5cGAm5cAGppG
m5cm5cGGAAm5cm5cm5cppm5cm5cm5cGAm5cm5cpm5cm5cm5cAm5cGAA
GAm5cm5cm5cGppm5cAm5cm5cpppGm5cGm5cApm5cm5cm5cm5cpGAm5c
m5cm5cpm5cm5cm5cm5cm5cm5cApm5cm5cm5cGm5cm5cppm5cGm5cAAp
Gpm5cpm5cAGGm5cApm5cGpm5cm5cpm5cGm5cm5cm5cGGpGAGGGAm5
cm5cm5cpm5cGpm5cGGAAGm5cGGm5cm5cGm5cGApm5cAGm5cGAGGm5
cm5cGAAGm5cm5cGm5cm5cAGm5cGGm5cAGm5cpppGGpm5cGm5cm5cpG
m5cAm5cpGm5cm5cAGGpGm5cppm5cGGm5cpm5cApm5cAm5cm5cAAm5c
GpGGAAGGm5cGGm5cpm5cGm5cpGGAAGm5cm5cGGpm5cGpm5cpGm5c
GAm5cpm5cm5cpGGAm5cm5cpGm5cGpAm5cm5cAAm5cApAGAGGpGAGm
5cm5cGAm5cm5cm5cpm5cGGppm5cpm5cpGm5cpGppppm5cAGGAGAAm5c
AAApm5cpm5cm5cGm5cAm5cGAm5cAm5cm5cGpAGAm5cm5cpGAm5cm5c
GAm5cm5cpAAAm5cApm5cAAGGGm5cm5cGm5cpGm5cGpGGpGGGm5cGA
Am5cGGGAm5cm5cGAm5cpGm5cpGGpGGAm5cm5cpm5cAAm5cAAm5cppp
GGm5cm5cm5cAm5cGAm5cGm5cm5cpGAm5cGm5cm5cAGGm5cpm5cAGA
AAAm5cAAm5cAm5cGGpm5cpm5cGGpGm5cpGGm5cm5cpppGm5cGm5cpG
m5cm5cGm5cpAGAm5cm5cGm5cGpGm5cm5cm5cGppAGm5cGGAm5cpGm
5cAm5cm5cpm5cpppm5cAGAGm5cm5cAGm5cGGm5cGm5cGGm5cGGm5cG
AAGAAAApm5cGGm5cm5cGm5cGAApGGAGGm5cGm5cGm5cGm5cm5cAp
m5cApm5cm5cGm5cm5cGm5cAm5cGGm5cpm5cAm5cm5cAm5cpGGGm5cm
5cGpGm5cGAm5cpGAm5cm5cGpGAm5cGm5cm5cGAAm5cpGGm5cGm5cm
5cGm5cAGAAm5cm5cGAm5cAGm5cAGpppGGAGGm5cAGGGm5cAGApm5c
pppGpm5cAGm5cm5cAGppm5cGm5cm5cpppm5cGm5cGm5cm5cGGm5cGm
5cm5cApm5cm5cm5cGm5cpGAm5cGm5cpGGpAGAm5cGm5cm5cm5cpGGA
Gm5cAGm5cpGGm5cm5cpGppm5cGGAm5cm5cm5cpAAm5cAm5cGpAm5cA
pm5cm5cAm5cAAAAm5cGGAGAm5cGGAm5cGAAm5cGAGGm5cm5cAApG
GApm5cApGm5cpGpppm5cpGm5cApm5cAm5cGAm5cpm5cAm5cm5cGm5cA
m5cm5cm5cGm5cm5cGAm5cm5cAGm5cGpGpppm5cpGm5cAm5cppppm5cG
GpppAm5cAm5cGm5cApm5cGm5cGm5cm5cGAGGpGGpGGm5cGm5cGAm5
cAm5cAApm5cm5cGpAm5cm5cm5cGm5cAm5cm5cpAm5cGAm5cGm5cppG
m5cm5cGGAm5cAAm5cGGm5cppm5cm5cAGm5cpGppGAppm5cm5cm5cAA
AAGppppAm5cGm5cpGAm5cGm5cGm5cApAm5cApm5cm5cm5cGAGpAm5c
Apm5cGpGm5cAGApm5cm5cAGAApGm5cpppm5cGAAAm5cm5cAApm5cAG
Am5cpm5cAm5cGAm5cAm5cm5cApm5cpppppm5cm5cm5cGGAAAAm5cApm
5cm5cm5cGGGm5cGpm5cpm5cm5cApAGAAGm5cm5cGGm5cm5cm5cGm5c
pAm5cm5cm5cGApm5cGpGpGm5cGAApm5cAm5cm5cm5cpm5cm5cGm5cG
pm5cAm5cGm5cpGAm5cm5cGGm5cGAm5cm5cAGGm5cm5cGppm5cApppG
GAAm5cAm5cm5cGAm5cAGm5cm5cGm5cpAGGm5cm5cGm5cApm5cm5cA
m5cpppppm5cm5cGm5cm5cGpGGGppppGGAm5cpm5cpm5cAm5cAm5cm5c
m5cGGpAAAm5cm5cGGAm5cAAAApm5cAAGm5cGpm5cm5cm5cm5cAGGp
Gm5cAGm5cpGm5cGm5cGm5cm5cGGpm5cpm5cpppm5cm5cAm5cGGAGm5
cGAm5cGpm5cGpGm5cGm5cGGm5cGm5cm5cGpm5cpm5cm5cGAGpppm5c
pm5cm5cm5cGm5cAGpm5cm5cm5cm5cm5cGGAppAm5cm5cAm5cm5cm5cA
m5cm5cGAGGAAGAGGAGGAAGAAGAGGAAGAGGAm5cGAm5cGAAGApG
Am5cm5cpm5cpm5cm5cpm5cm5cAm5cAm5cm5cGAm5cGm5cm5cGAm5cm5
cm5cm5cm5cm5cpGpm5cm5cGAAGm5cm5cApGpppGm5cm5cGGm5cppm5c
GAGGAGGm5cm5cAGm5cGGm5cGAm5cGAGGAm5cpm5cGGAm5cAm5cm5
cm5cAAGm5cm5cGGAm5cpGpm5cm5cm5cGGGm5cAm5cpGApm5cm5cpGA
m5cm5cGGAm5cAAAGAm5cGpm5cGAAGm5cGGpAAm5cAAm5cGGGGm5c
pm5cpm5cAm5cGm5cpm5cGpm5cApm5cm5cm5cm5cpm5cGpGGm5cAm5cG
pm5cpppGm5cGAGm5cm5cppGAm5cGAm5cppGGpAm5cm5cAppAAm5cGG
pGAGm5cGpGm5cAGm5cAm5cGm5cm5cGm5cAppAm5cGAm5cm5cpAm5cm
5cpm5cppApm5cpGm5cGm5cAGm5cGAm5cApGGAm5cGGm5cGAm5cGpG
m5cGpAm5cm5cGm5cGGm5cAGAm5cApm5cAGm5cAGm5cAm5cGppGm5c
GGpm5cm5cGpGm5cm5cm5cGm5cGm5cm5cAm5cGAm5cm5cm5cpm5cAm5
cm5cm5cApm5cAGm5cAm5cm5cGm5cppm5cm5cAm5cppm5cm5cAGm5cAm
5cm5cm5cm5cAm5cGm5cAGpm5cGAm5cm5cm5cm5cGm5cApm5cpAGAGA
GAGAm5cppm5cpppGpppppm5cm5cm5cm5cm5cGm5cGpGpppppm5cm5cm5
cAppm5cm5cm5cpGpApppApppm5cpAAApAApAAAAAm5cAm5cAGAGAm5c
GppGApAApAAm5cm5cGm5cAGpGpGm5cpppAppAGGGpApm5cAm5cGGpG
pAGAAAAAAAAAGAGAGGGAAAm5cm5cm5cpAAApApAGm5cGpm5cpm5cp
m5cAGm5cm5cpGAGpAGGAAG
SEQ ID NO: 20 (pp71 Construct F-stop pp65 + pseudoU/5meC)
AGGAm5cAGAGGAm5cm5cm5cpGGpGppGGGAAAm5cGGAm5cAm5cpAGG
m5cGpm5cm5cGm5cGm5cGApAm5cGGGGppAAAAm5cAAAAAAAApm5cGG
pGGpGGpGpGpGm5cpGGGGpGpGGpGAm5cGGpGGGGm5cpppGm5cm5cp
m5cpppppppppGpAApAAAAAAAGAm5cAm5cpm5cAApAApm5cm5cGm5cGG
ppGpm5cpm5cpGpGpAGAAm5cGpppppApppm5cGGGppm5cm5cGm5cGppp
GGpm5cGm5cm5cpGm5cm5cpGpGpAAGGm5cGGm5cGGm5cm5cGm5cAAA
GGGm5cGm5cGm5cm5cGm5cpm5cAGpm5cGm5cm5cpAm5cAm5cm5cm5cG
pAm5cGm5cGm5cAGGm5cAGm5cApGGAGpm5cGm5cGm5cGGpm5cGm5c
m5cGppGpm5cm5cm5cGAApAGpGApm5cm5cGpAm5cpGGGpm5cm5cm5cA
pppm5cGGGGm5cAm5cGpGm5cpGAAAGm5cm5cGpGpppAGpm5cGm5cGG
m5cGAm5cAm5cGm5cm5cGGpGm5cpGm5cm5cGm5cAm5cGAGAm5cGm5c
GAm5cpm5cm5cpGm5cAGAm5cGGGpApm5cm5cAm5cGpAm5cGm5cGpGA
Gm5cm5cAGm5cm5cm5cpm5cGm5cpGApm5cm5cpGGpGpm5cGm5cAGpAm
5cAm5cGm5cm5cm5cGAm5cpm5cGAm5cGm5cm5cApGm5cm5cAm5cm5cG
m5cGGm5cGAm5cAApm5cAGm5cpGm5cAGGpGm5cAGm5cAm5cAm5cGpA
m5cpppAm5cGGGm5cAGm5cGAGGpGGAGAAm5cGpGpm5cGGpm5cAAm5c
GpGm5cAm5cAAm5cm5cm5cm5cAm5cGGGm5cm5cGAAGm5cApm5cpGm5c
m5cm5cm5cAGm5cm5cAGGAGm5cm5cm5cApGpm5cGApm5cpApGpGpAm5
cGm5cGm5cpGm5cm5cGm5cpm5cAAGApGm5cpGAAm5cApm5cm5cm5cm5
cAGm5cApm5cAAm5cGpGm5cAm5cm5cAm5cpAm5cm5cm5cGpm5cGGm5c
GGm5cm5cGAGm5cGm5cAAAm5cAm5cm5cGAm5cAm5cm5cpGm5cm5cm5c
GpAGm5cpGAm5cGm5cpGpGAppm5cAm5cGm5cGpm5cGGGm5cAAGm5cA
GApGpGGm5cAGGm5cGm5cGpm5cpm5cAm5cGGpm5cpm5cGGGAm5cpGG
m5cm5cpGGAm5cGm5cGpm5cAGm5cAGAAm5cm5cAGpGGAAAGAGm5cm5
cm5cGAm5cGpm5cpAm5cpAm5cAm5cGpm5cAGm5cGppm5cGpGpppm5cm5
cm5cAm5cm5cAAGGAm5cGpGGm5cAm5cpGm5cGGm5cAm5cGpGGpGpGm
5cGm5cGm5cAm5cGAGm5cpGGpppGm5cpm5cm5cApGGAGAAm5cAm5cG
m5cGm5cGm5cAAm5cm5cAAGApGm5cAGGpGApAGGpGAm5cm5cAGpAm5
cGpm5cAAGGpGpAm5cm5cpGGAGpm5cm5cppm5cpGm5cGAGGAm5cGpG
m5cm5cm5cpm5cm5cGGm5cAAGm5cpm5cpppApGm5cAm5cGpm5cAm5cG
m5cpGGGm5cpm5cpGAm5cGpGGAAGAGGAm5cm5cpGAm5cGApGAm5cm5
cm5cGm5cAAm5cm5cm5cGm5cAAm5cm5cm5cppm5cApGm5cGm5cm5cm5c
m5cm5cAm5cGAGm5cGm5cAAm5cGGm5cpppAm5cGGpGppGpGpm5cm5cm
5cAAAAApApGApAApm5cAAAm5cm5cGGGm5cAAGApm5cpm5cGm5cAm5c
Apm5cApGm5cpGGApGpGGm5cppppAm5cm5cpm5cAm5cAm5cGAGm5cApp
ppGGGm5cpGm5cpGpGpm5cm5cm5cAAGAGm5cApm5cm5cm5cGGGm5cm
5cpGAGm5cApm5cpm5cAGGpAAm5cm5cpGppGApGAAm5cGGGm5cAGm5c
AGApm5cppm5cm5cpGGAGGpAm5cAAGm5cGApAm5cGm5cGAGAm5cm5c
GpGGAAm5cpGm5cGpm5cAGpAm5cGApm5cm5cm5cGpGGm5cpGm5cAm5
cpm5cppm5cpppppm5cGApApm5cGAm5cppGm5cpGm5cpGm5cAGm5cGm5c
GGGm5cm5cpm5cAGpAm5cAGm5cGAGm5cAm5cm5cm5cm5cAm5cm5cppm
5cAm5cm5cAGm5cm5cAGpApm5cGm5cApm5cm5cAGGGm5cAAGm5cppGA
GpAm5cm5cGAm5cAm5cAm5cm5cpGGGAm5cm5cGGm5cAm5cGAm5cGAG
GGpGm5cm5cGm5cm5cm5cAGGGm5cGAm5cGAm5cGAm5cGpm5cpGGAm
5cm5cAGm5cGGApm5cGGAm5cpm5cm5cGAm5cGAAGAAm5cpm5cGpAAm
5cm5cAm5cm5cGAGm5cGm5cAAGAm5cGm5cm5cm5cm5cGm5cGppAm5cm
5cGGm5cGGm5cGGm5cGm5cm5cApGGm5cGGGm5cGm5cm5cpm5cm5cAm
5cppm5cm5cGm5cGGGm5cm5cGm5cAAAm5cGm5cAAApm5cAGm5cApm5c
m5cpm5cGGm5cGAm5cGGm5cGpGm5cAm5cGGm5cGGGm5cGppApGAm5c
Am5cGm5cGGm5cm5cGm5cm5cppAAGGm5cm5cGAGpm5cm5cAm5cm5cGp
m5cGm5cGm5cm5cm5cGAAGAGGAm5cAm5cm5cGAm5cGAGGAppm5cm5c
GAm5cAAm5cGAAApm5cm5cAm5cAApm5cm5cGGm5cm5cGpGppm5cAm5c
m5cpGGm5cm5cGm5cm5cm5cpGGm5cAGGm5cm5cGGm5cApm5cm5cpGG
m5cm5cm5cGm5cAAm5cm5cpGGpGm5cm5cm5cApGGpGGm5cpAm5cGGpp
m5cAGGGpm5cAGAApm5cpGAAGpAm5cm5cAGGAGppm5cppm5cpGGGAm
5cGm5cm5cAAm5cGAm5cApm5cpAm5cm5cGm5cApm5cppm5cGm5cm5cGA
AppGGAAGGm5cGpApGGm5cAGm5cm5cm5cGm5cpGm5cGm5cAAm5cm5c
m5cAAAm5cGpm5cGm5cm5cGm5cm5cAm5cm5cGGm5cAAGAAGm5cm5cm
5cpGm5cm5cm5cGGGm5cm5cApGm5cApm5cGm5cm5cpm5cGAm5cGm5cm
5cm5cAAAAAGm5cApm5cGAGGppGAGm5cm5cAm5cm5cm5cGm5cm5cGm5
cGm5cAm5cGm5cGm5cppAAGAm5cGAm5cpm5cpApAAAAAm5cm5cm5cAm
5cGpm5cm5cAm5cpm5cAGAm5cAm5cGm5cGAm5cppppGGGm5cGm5cm5c
Am5cAm5cm5cpGpm5cGm5cm5cGm5cpGm5cpApApppGm5cGAm5cAGppG
m5cm5cGGAAm5cm5cm5cppm5cm5cm5cGAm5cm5cpm5cm5cm5cAm5cGAA
GAm5cm5cm5cGppm5cAm5cm5cpppGm5cGm5cApm5cm5cm5cm5cpGAm5c
m5cm5cpm5cm5cm5cm5cm5cm5cApm5cm5cm5cGm5cm5cppm5cGm5cAAp
Gpm5cpm5cAGGm5cApm5cGpm5cm5cpm5cGm5cm5cm5cGGpGAGGGAm5
cm5cm5cpm5cGpm5cGGAAGm5cGGm5cm5cGm5cGApm5cAGm5cGAGGm5
cm5cGAAGm5cm5cGm5cm5cAGm5cGGm5cAGm5cpppGGpm5cGm5cm5cpG
m5cAm5cpGm5cm5cAGGpGm5cppm5cGGm5cpm5cApm5cAm5cm5cAAm5c
GpGGAAGGm5cGGm5cpm5cGm5cpGGAAGm5cm5cGGpm5cGpm5cpGm5c
GAm5cpm5cm5cpGGAm5cm5cpGm5cGpAm5cm5cAAm5cApAGAGGpGAGm
5cm5cGAm5cm5cm5cpm5cGGppm5cpm5cpGm5cpGppppm5cAGGAGAAm5c
AAApm5cpm5cm5cGm5cAm5cGAm5cAm5cm5cGpAGAm5cm5cpGAm5cm5c
GAm5cm5cpAAAm5cApm5cAAGGGm5cm5cGm5cpGm5cGpGGpGGGm5cGA
Am5cGGGAm5cm5cGAm5cpGm5cpGGpGGAm5cm5cpm5cAAm5cAAm5cppp
GGm5cm5cm5cAm5cGAm5cGm5cm5cpGAm5cGm5cm5cAGGm5cpm5cAGA
AAAm5cAAm5cAm5cGGpm5cpm5cGGpGm5cpGGm5cm5cpppGm5cGm5cpG
m5cm5cGm5cpAGAm5cm5cGm5cGpGm5cm5cm5cGppAGm5cGGAm5cpGm
5cAm5cm5cpm5cpppm5cAGAGm5cm5cAGm5cGGm5cGm5cGGm5cGGm5cG
AAGAAAApm5cGGm5cm5cGm5cGAApGGAGGm5cGm5cGm5cGm5cm5cAp
m5cApm5cm5cGm5cm5cGm5cAm5cGGm5cpm5cAm5cm5cAm5cpGGGm5cm
5cGpGm5cGAm5cpGAm5cm5cGpGAm5cGm5cm5cGAAm5cpGGm5cGm5cm
5cGm5cAGAAm5cm5cGAm5cAGm5cAGpppGGAGGm5cAGGGm5cAGApm5c
pppGpm5cAGm5cm5cAGppm5cGm5cm5cpppm5cGm5cGm5cm5cGGm5cGm
5cm5cApm5cm5cm5cGm5cpGAm5cGm5cpGGpAGAm5cGm5cm5cm5cpGGA
Gm5cAGm5cpGGm5cm5cpGppm5cGGAm5cm5cm5cpAAm5cAm5cGpAm5cA
pm5cm5cAm5cAAAAm5cGGAGAm5cGGAm5cGAAm5cGAGGm5cm5cAApG
GApm5cApGm5cpGpppm5cpGm5cApm5cAm5cGAm5cpm5cAm5cm5cGm5cA
m5cm5cm5cGm5cm5cGAm5cm5cAGm5cGpGpppm5cpGm5cAm5cppppm5cG
GpppAm5cAm5cGm5cApm5cGm5cGm5cm5cGAGGpGGpGGm5cGm5cGAm5
cAm5cAApm5cm5cGpAm5cm5cm5cGm5cAm5cm5cpAm5cGAm5cGm5cppG
m5cm5cGGAm5cAAm5cGGm5cppm5cm5cAGm5cpGppGAppm5cm5cm5cAA
AAGppppAm5cGm5cpGAm5cGm5cGm5cApAm5cApm5cm5cm5cGAGpAm5c
Apm5cGpGm5cAGApm5cm5cAGAApGm5cpppm5cGAAAm5cm5cAApm5cAG
Am5cpm5cAm5cGAm5cAm5cm5cApm5cpppppm5cm5cm5cGGAAAAm5cApm
5cm5cm5cGGGm5cGpm5cpm5cm5cApAGAAGm5cm5cGGm5cm5cm5cGm5c
pAm5cm5cm5cGApm5cGpGpGm5cGAApm5cAm5cm5cm5cpm5cm5cGm5cG
pm5cAm5cGm5cpGAm5cm5cGGm5cGAm5cm5cAGGm5cm5cGppm5cApppG
GAAm5cAm5cm5cGAm5cAGm5cm5cGm5cpAGGm5cm5cGm5cApm5cm5cA
m5cpppppm5cm5cGm5cm5cGpGGGppppGGAm5cpm5cpm5cAm5cAm5cm5c
m5cGGpAAAm5cm5cGGAm5cAAAApm5cAAGm5cGpm5cm5cm5cm5cAGGp
Gm5cAGm5cpGm5cGm5cGm5cm5cGGpm5cpm5cpppm5cm5cAm5cGGAGm5
cGAm5cGpm5cGpGm5cGm5cGGm5cGm5cm5cGpm5cpm5cm5cGAGpppm5c
pm5cm5cm5cGm5cAGpm5cm5cm5cm5cm5cGGAppAm5cm5cAm5cm5cm5cA
m5cm5cGAGGAAGAGGAGGAAGAAGAGGAAGAGGAm5cGAm5cGAAGApG
Am5cm5cpm5cpm5cm5cpm5cm5cAm5cAm5cm5cGAm5cGm5cm5cGAm5cm5
cm5cm5cm5cm5cpGpm5cm5cGAAGm5cm5cApGpppGm5cm5cGGm5cppm5c
GAGGAGGm5cm5cAGm5cGGm5cGAm5cGAGGAm5cpm5cGGAm5cAm5cm5
cm5cAAGm5cm5cGGAm5cpGpm5cm5cm5cGGGm5cAm5cpGApm5cm5cpGA
m5cm5cGGAm5cAAAGAm5cGpm5cGAAGm5cGGpAAm5cAAm5cGGGGm5c
pm5cpm5cAm5cGm5cpm5cGpm5cApm5cm5cm5cm5cpm5cGpGGm5cAm5cG
pm5cpppGm5cGAGm5cm5cppGAm5cGAm5cppGGpAm5cm5cAppAAm5cGG
pGAGm5cGpGm5cAGm5cAm5cGm5cm5cGm5cAppAm5cGAm5cm5cpAm5cm
5cpm5cppApm5cpGm5cGm5cAGm5cGAm5cApGGAm5cGGm5cGAm5cGpG
m5cGpAm5cm5cGm5cGGm5cAGAm5cApm5cAGm5cAGm5cAm5cGppGm5c
GGpm5cm5cGpGm5cm5cm5cGm5cGm5cm5cAm5cGAm5cm5cm5cpm5cAm5
cm5cm5cApm5cAGm5cAm5cm5cGm5cppm5cm5cAm5cppm5cm5cAGm5cAm
5cm5cm5cm5cAm5cGm5cAGpm5cGAm5cm5cm5cm5cGm5cApm5cpAGAGA
GAGAm5cppm5cpppGpppppm5cm5cm5cm5cm5cGm5cGpGpppppm5cm5cm5
cAppm5cm5cm5cpGpApppApppm5cpAAApAApAAAAAm5cAm5cAGAGAm5c
GppGApAApAAm5cm5cGm5cAGpGpGm5cpppAppAGGGpApm5cAm5cGGpG
pAGAAAAAAAAAGAGAGGGAAAm5cm5cm5cpAAApApAGm5cGpm5cpm5cp
m5cAGm5cm5cpGAGpAGGAAG
SEQ ID NO: 21 (pp71 Construct B-Full-length (FT) + 5moU)
AGGGCCACCCGCCGCGCACGCGCmo5umo5uAAGACGACmo5uCmo5uAm
o5uAAAAACCCACGmo5uCCACmo5uCAGACACGCGACmo5umo5umo5umo5
uGGGCGCCACACCmo5uGmo5uCGCCGCmo5uGCmo5uAmo5uAmo5umo5u
mo5uGCGACAGmo5umo5uGCCGGAACCCmo5umo5uCCCGACCmo5uCCCA
CGAAGACCCGmo5umo5uCACCmo5umo5umo5uGCGCAmo5uCCCCmo5uG
ACCCmo5uCCCCCCAmo5uCCCGCCmo5umo5uCGCAAmo5uGmo5uCmo5u
CAGGCAmo5uCGmo5uCCmo5uCGCCCGGmo5uGAGGGACCCmo5uCGmo5
uCGGAAGCGGCCGCGAmo5uCAGCGAGGCCGAAGCCGCCAGCGGCAGC
mo5umo5umo5uGGmo5uCGCCmo5uGCACmo5uGCCAGGmo5uGCmo5umo5
uCGGCmo5uCAmo5uCACCAACGmo5uGGAAGGCGGCmo5uCGCmo5uGGA
AGCCGGmo5uCGmo5uCmo5uGCGACmo5uCCmo5uGGACCmo5uGCGmo5u
ACCAACAmo5uAGAGGmo5uGAGCCGACCCmo5uCGGmo5umo5uCmo5uC
mo5uGCmo5uGmo5umo5umo5umo5uCAGGAGAACAAAmo5uCmo5uCCGCA
CGACACCGmo5uAGACCmo5uGACCGACCmo5uAAACAmo5uCAAGGGCCG
Cmo5uGCGmo5uGGmo5uGGGCGAACGGGACCGACmo5uGCmo5uGGmo5u
GGACCmo5uCAACAACmo5umo5umo5uGGCCCACGACGCCmo5uGACGCC
AGGCmo5uCAGAAAACAACACGGmo5uCmo5uCGGmo5uGCmo5uGGCCmo
5umo5umo5uGCGCmo5uGCCGCmo5uAGACCGCGmo5uGCCCGmo5umo5u
AGCGGACmo5uGCACCmo5uCmo5umo5umo5uCAGAGCCAGCGGCGCGGC
GGCGAAGAAAAmo5uCGGCCGCGAAmo5uGGAGGCGCGCGCCAmo5uCAm
05uCCGCCGCACGGCmo5uCACCACmo5uGGGCCGmo5uGCGACmo5uGAC
CGmo5uGACGCCGAACmo5uGGCGCCGCAGAACCGACAGCAGmo5umo5u
mo5uGGAGGCAGGGCAGAmo5uCmo5umo5umo5uGmo5uCAGCCAGmo5um
o5uCGCCmo5umo5umo5uCGCGCCGGCGCCAmo5uCCCGCmo5uGACGCm
o5uGGmo5uAGACGCCCmo5uGGAGCAGCmo5uGGCCmo5uGmo5umo5uCG
GACCCmo5uAACACGmo5uACAmo5uCCACAAAACGGAGACGGACGAACGA
GGCCAAmo5uGGAmo5uCAmo5uGCmo5uGmo5umo5umo5uCmo5uGCAmo5
uCACGACmo5uCACCGCACCCGCCGACCAGCGmo5uGmo5umo5umo5uCm
o5uGCACmo5umo5umo5umo5uCGGmo5umo5umo5uACACGCAmo5uCGCG
CCGAGGmo5uGGmo5uGGCGCGACACAAmo5uCCGmo5uACCCGCACCmo5
uACGACGCmo5umo5uGCCGGACAACGGCmo5umo5uCCAGCmo5uGmo5u
mo5uGAmo5umo5uCCCAAAAGmo5umo5umo5umo5uACGCmo5uGACGCGC
Amo5uACAmo5uCCCGAGmo5uACAmo5uCGmo5uGCAGAmo5uCCAGAAmo
5uGCmo5umo5umo5uCGAAACCAAmo5uCAGACmo5uCACGACACCAmo5uC
mo5umo5umo5umo5umo5uCCCGGAAAACAmo5uCCCGGGCGmo5uCmo5uC
CAmo5uAGAAGCCGGCCCGCmo5uACCCGAmo5uCGmo5uGmo5uGCGAAm
o5uCACCCmo5uCCGCGmo5uCACGCmo5uGACCGGCGACCAGGCCGmo5u
mo5uCAmo5umo5umo5uGGAACACCGACAGCCGCmo5uAGGCCGCAmo5uC
CACmo5umo5umo5umo5umo5uCCGCCGmo5uGGGmo5umo5umo5umo5uG
GACmo5uCmo5uCACACCCGGmo5uAAACCGGACAAAAmo5uCAAGCGmo5u
CCCCAGGmo5uGCAGCmo5uGCGCGCCGGmo5uCmo5uCmo5umo5umo5uC
CACGGAGCGACGmo5uCGmo5uGCGCGGCGCCGmo5uCmo5uCCGAGmo5
umo5umo5uCmo5uCCCGCAGmo5uCCCCCGGAmo5umo5uACCACCCACCG
AGGAAGAGGAGGAAGAAGAGGAAGAGGACGACGAAGAmo5uGACCmo5uC
mo5uCCmo5uCCACACCGACGCCGACCCCCCmo5uGmo5uCCGAAGCCAmo
5uGmo5umo5umo5uGCCGGCmo5umo5uCGAGGAGGCCAGCGGCGACGAG
GACmo5uCGGACACCCAAGCCGGACmo5uGmo5uCCCGGGCACmo5uGAm
o5uCCmo5uGACCGGACAAAGACGmo5uCGAAGCGGmo5uAACAACGGGGC
mo5uCmo5uCACGCmo5uCGmo5uCAmo5uCCCCmo5uCGmo5uGGCACGmo
5uCmo5umo5umo5uGCGAGCCmo5umo5uGACGACmo5umo5uGGmo5uACC
Amo5umo5uAACGGmo5uGAGCGmo5uGCAGCACGCCGCAmo5umo5uACGA
CCmo5uACCmo5uCmo5umo5uAmo5uCmo5uGCGCAGCGACAmo5uGGACG
GCGACGmo5uGCGmo5uACCGCGGCAGACAmo5uCAGCAGCACGmo5umo5
uGCGGmo5uCCGmo5uGCCCGCGCCACGACCCmo5uCACCCAmo5uCAGCA
CCGCmo5umo5uCCACmo5umo5uCCAGCACCCCACGCAGmo5uCGACCCC
GCAmo5uCmo5uAGAGAGAGACmo5umo5uCmo5umo5umo5uGmo5umo5um
05umo5umo5uCCCCCGCGmo5uGmo5umo5umo5umo5umo5uCCCAmo5umo
5uCCCmo5uGmo5uAmo5umo5umo5uAmo5umo5umo5uCmo5uAAAmo5uAAm
o5uAAAAACACAGAGACGmo5umo5uGAmo5uAAmo5uAACCGCAGmo5uGm
o5uGCmo5umo5umo5uAmo5umo5uAGGGmo5uAmo5uCACGGmo5uGmo5uA
GAAAAAAAAAAGAGAGGGAAACCCmo5uAAAmo5uAmo5uAGCGmo5uCmo5
uCmo5uCAAAAAAAAAAAAAAAAAAAAAAAAAAAAAAAAAAAAAAAAAAAAAA
AAAAAAAAAAAAAAAAAAAAAAAAAAAAAAAAAA
SEQ ID NO: 22 (pp71 Construct B.2-Full-length (FT) + 5moU)
GCCACCCGCCGCGCACGCGCmo5umo5uAAGACGACmo5uCmo5uAmo5uA
AAAACCCACGmo5uCCACmo5uCAGACACGCGACmo5umo5umo5umo5uGG
GCGCCACACCmo5uGmo5uCGCCGCmo5uGCmo5uAmo5uAmo5umo5umo5u
GCGACAGmo5umo5uGCCGGAACCCmo5umo5uCCCGACCmo5uCCCACGA
AGACCCGmo5umo5uCACCmo5umo5umo5uGCGCAmo5uCCCCmo5uGACC
Cmo5uCCCCCCAmo5uCCCGCCmo5umo5uCGCAAmo5uGmo5uCmo5uCAG
GCAmo5uCGmo5uCCmo5uCGCCCGGmo5uGAGGGACCCmo5uCGmo5uCG
GAAGCGGCCGCGAmo5uCAGCGAGGCCGAAGCCGCCAGCGGCAGCmo5u
mo5umo5uGGmo5uCGCCmo5uGCACmo5uGCCAGGmo5uGCmo5umo5uCG
GCmo5uCAmo5uCACCAACGmo5uGGAAGGCGGCmo5uCGCmo5uGGAAGC
CGGmo5uCGmo5uCmo5uGCGACmo5uCCmo5uGGACCmo5uGCGmo5uACC
AACAmo5uAGAGGmo5uGAGCCGACCCmo5uCGGmo5umo5uCmo5uCmo5u
GCmo5uGmo5umo5umo5umo5uCAGGAGAACAAAmo5uCmo5uCCGCACGA
CACCGmo5uAGACCmo5uGACCGACCmo5uAAACAmo5uCAAGGGCCGCmo
5uGCGmo5uGGmo5uGGGCGAACGGGACCGACmo5uGCmo5uGGmo5uGGA
CCmo5uCAACAACmo5umo5umo5uGGCCCACGACGCCmo5uGACGCCAGG
Cmo5uCAGAAAACAACACGGmo5uCmo5uCGGmo5uGCmo5uGGCCmo5umo
5umo5uGCGCmo5uGCCGCmo5uAGACCGCGmo5uGCCCGmo5umo5uAGC
GGACmo5uGCACCmo5uCmo5umo5umo5uCAGAGCCAGCGGCGCGGCGG
CGAAGAAAAmo5uCGGCCGCGAAmo5uGGAGGCGCGCGCCAmo5uCAmo5
uCCGCCGCACGGCmo5uCACCACmo5uGGGCCGmo5uGCGACmo5uGACC
Gmo5uGACGCCGAACmo5uGGCGCCGCAGAACCGACAGCAGmo5umo5um
05uGGAGGCAGGGCAGAmo5uCmo5umo5umo5uGmo5uCAGCCAGmo5umo
5uCGCCmo5umo5umo5uCGCGCCGGCGCCAmo5uCCCGCmo5uGACGCmo
5uGGmo5uAGACGCCCmo5uGGAGCAGCmo5uGGCCmo5uGmo5umo5uCG
GACCCmo5uAACACGmo5uACAmo5uCCACAAAACGGAGACGGACGAACGA
GGCCAAmo5uGGAmo5uCAmo5uGCmo5uGmo5umo5umo5uCmo5uGCAmo5
uCACGACmo5uCACCGCACCCGCCGACCAGCGmo5uGmo5umo5umo5uCm
05uGCACmo5umo5umo5umo5uCGGmo5umo5umo5uACACGCAmo5uCGCG
CCGAGGmo5uGGmo5uGGCGCGACACAAmo5uCCGmo5uACCCGCACCmo5
uACGACGCmo5umo5uGCCGGACAACGGCmo5umo5uCCAGCmo5uGmo5u
mo5uGAmo5umo5uCCCAAAAGmo5umo5umo5umo5uACGCmo5uGACGCGC
Amo5uACAmo5uCCCGAGmo5uACAmo5uCGmo5uGCAGAmo5uCCAGAAmo
5uGCmo5umo5umo5uCGAAACCAAmo5uCAGACmo5uCACGACACCAmo5uC
mo5umo5umo5umo5umo5uCCCGGAAAACAmo5uCCCGGGCGmo5uCmo5uC
CAmo5uAGAAGCCGGCCCGCmo5uACCCGAmo5uCGmo5uGmo5uGCGAAm
o5uCACCCmo5uCCGCGmo5uCACGCmo5uGACCGGCGACCAGGCCGmo5u
mo5uCAmo5umo5umo5uGGAACACCGACAGCCGCmo5uAGGCCGCAmo5uC
CACmo5umo5umo5umo5umo5uCCGCCGmo5uGGGmo5umo5umo5umo5uG
GACmo5uCmo5uCACACCCGGmo5uAAACCGGACAAAAmo5uCAAGCGmo5u
CCCCAGGmo5uGCAGCmo5uGCGCGCCGGmo5uCmo5uCmo5umo5umo5uC
CACGGAGCGACGmo5uCGmo5uGCGCGGCGCCGmo5uCmo5uCCGAGmo5
umo5umo5uCmo5uCCCGCAGmo5uCCCCCGGAmo5umo5uACCACCCACCG
AGGAAGAGGAGGAAGAAGAGGAAGAGGACGACGAAGAmo5uGACCmo5uC
mo5uCCmo5uCCACACCGACGCCGACCCCCCmo5uGmo5uCCGAAGCCAmo
5uGmo5umo5umo5uGCCGGCmo5umo5uCGAGGAGGCCAGCGGCGACGAG
GACmo5uCGGACACCCAAGCCGGACmo5uGmo5uCCCGGGCACmo5uGAm
05uCCmo5uGACCGGACAAAGACGmo5uCGAAGCGGmo5uAACAACGGGGC
mo5uCmo5uCACGCmo5uCGmo5uCAmo5uCCCCmo5uCGmo5uGGCACGmo
5uCmo5umo5umo5uGCGAGCCmo5umo5uGACGACmo5umo5uGGmo5uACC
Amo5umo5uAACGGmo5uGAGCGmo5uGCAGCACGCCGCAmo5umo5uACGA
CCmo5uACCmo5uCmo5umo5uAmo5uCmo5uGCGCAGCGACAmo5uGGACG
GCGACGmo5uGCGmo5uACCGCGGCAGACAmo5uCAGCAGCACGmo5umo5
uGCGGmo5uCCGmo5uGCCCGCGCCACGACCCmo5uCACCCAmo5uCAGCA
CCGCmo5umo5uCCACmo5umo5uCCAGCACCCCACGCAGmo5uCGACCCC
GCAmo5uCmo5uAGAGAGAGACmo5umo5uCmo5umo5umo5uGmo5umo5um
o5umo5umo5uCCCCCGCGmo5uGmo5umo5umo5umo5umo5uCCCAmo5umo
SuCCCmo5uGmo5uAmo5umo5umo5uAmo5umo5umo5uCmo5uAAAmo5uAAm
05uAAAAACACAGAGACGmo5umo5uGAmo5uAAmo5uAACCGCAGmo5uGm
o5uGCmo5umo5umo5uAmo5umo5uAGGGmo5uAmo5uCACGGmo5uGmo5uA
GAAAAAAAAAGAGAGGGAAACCCmo5uAAAmo5uAmo5uAGCGmo5uCmo5u
Cmo5uCAGCCmo5uGAGmo5uAGGAAG
SEQ ID NO: 23 (pp71 Construct B.3-Full-length (FT) + 5moU)
GCCACCCGCCGCGCACGCGCmo5umo5uAAGACGACmo5uCmo5uAmo5uA
AAAACCCACGmo5uCCACmo5uCAGACACGCGACmo5umo5umo5umo5uGG
GCGCCACACCmo5uGmo5uCGCCGCmo5uGCmo5uAmo5uAmo5umo5umo5u
GCGACAGmo5umo5uGCCGGAACCCmo5umo5uCCCGACCmo5uCCCACGA
AGACCCGmo5umo5uCACCmo5umo5umo5uGCGCAmo5uCCCCmo5uGACC
Cmo5uCCCCCCAmo5uCCCGCCmo5umo5uCGCAAmo5uGmo5uCmo5uCAG
GCAmo5uCGmo5uCCmo5uCGCCCGGmo5uGAGGGACCCmo5uCGmo5uCG
GAAGCGGCCGCGAmo5uCAGCGAGGCCGAAGCCGCCAGCGGCAGCmo5u
mo5umo5uGGmo5uCGCCmo5uGCACmo5uGCCAGGmo5uGCmo5umo5uCG
GCmo5uCAmo5uCACCAACGmo5uGGAAGGCGGCmo5uCGCmo5uGGAAGC
CGGmo5uCGmo5uCmo5uGCGACmo5uCCmo5uGGACCmo5uGCGmo5uACC
AACAmo5uAGAGGmo5uGAGCCGACCCmo5uCGGmo5umo5uCmo5uCmo5u
GCmo5uGmo5umo5umo5umo5uCAGGAGAACAAAmo5uCmo5uCCGCACGA
CACCGmo5uAGACCmo5uGACCGACCmo5uAAACAmo5uCAAGGGCCGCmo
5uGCGmo5uGGmo5uGGGCGAACGGGACCGACmo5uGCmo5uGGmo5uGGA
CCmo5uCAACAACmo5umo5umo5uGGCCCACGACGCCmo5uGACGCCAGG
Cmo5uCAGAAAACAACACGGmo5uCmo5uCGGmo5uGCmo5uGGCCmo5umo
5umo5uGCGCmo5uGCCGCmo5uAGACCGCGmo5uGCCCGmo5umo5uAGC
GGACmo5uGCACCmo5uCmo5umo5umo5uCAGAGCCAGCGGCGCGGCGG
CGAAGAAAAmo5uCGGCCGCGAAmo5uGGAGGCGCGCGCCAmo5uCAmo5
uCCGCCGCACGGCmo5uCACCACmo5uGGGCCGmo5uGCGACmo5uGACC
Gmo5uGACGCCGAACmo5uGGCGCCGCAGAACCGACAGCAGmo5umo5um
o5uGGAGGCAGGGCAGAmo5uCmo5umo5umo5uGmo5uCAGCCAGmo5umo
5uCGCCmo5umo5umo5uCGCGCCGGCGCCAmo5uCCCGCmo5uGACGCmo
5uGGmo5uAGACGCCCmo5uGGAGCAGCmo5uGGCCmo5uGmo5umo5uCG
GACCCmo5uAACACGmo5uACAmo5uCCACAAAACGGAGACGGACGAACGA
GGCCAAmo5uGGAmo5uCAmo5uGCmo5uGmo5umo5umo5uCmo5uGCAmo5
uCACGACmo5uCACCGCACCCGCCGACCAGCGmo5uGmo5umo5umo5uCm
o5uGCACmo5umo5umo5umo5uCGGmo5umo5umo5uACACGCAmo5uCGCG
CCGAGGmo5uGGmo5uGGCGCGACACAAmo5uCCGmo5uACCCGCACCmo5
uACGACGCmo5umo5uGCCGGACAACGGCmo5umo5uCCAGCmo5uGmo5u
mo5uGAmo5umo5uCCCAAAAGmo5umo5umo5umo5uACGCmo5uGACGCGC
Amo5uACAmo5uCCCGAGmo5uACAmo5uCGmo5uGCAGAmo5uCCAGAAmo
5uGCmo5umo5umo5uCGAAACCAAmo5uCAGACmo5uCACGACACCAmo5uC
mo5umo5umo5umo5umo5uCCCGGAAAACAmo5uCCCGGGCGmo5uCmo5uC
CAmo5uAGAAGCCGGCCCGCmo5uACCCGAmo5uCGmo5uGmo5uGCGAAm
05uCACCCmo5uCCGCGmo5uCACGCmo5uGACCGGCGACCAGGCCGmo5u
mo5uCAmo5umo5umo5uGGAACACCGACAGCCGCmo5uAGGCCGCAmo5uC
CACmo5umo5umo5umo5umo5uCCGCCGmo5uGGGmo5umo5umo5umo5uG
GACmo5uCmo5uCACACCCGGmo5uAAACCGGACAAAAmo5uCAAGCGmo5u
CCCCAGGmo5uGCAGCmo5uGCGCGCCGGmo5uCmo5uCmo5umo5umo5uC
CACGGAGCGACGmo5uCGmo5uGCGCGGCGCCGmo5uCmo5uCCGAGmo5
umo5umo5uCmo5uCCCGCAGmo5uCCCCCGGAmo5umo5uACCACCCACCG
AGGAAGAGGAGGAAGAAGAGGAAGAGGACGACGAAGAmo5uGACCmo5uC
mo5uCCmo5uCCACACCGACGCCGACCCCCCmo5uGmo5uCCGAAGCCAmo
5uGmo5umo5umo5uGCCGGCmo5umo5uCGAGGAGGCCAGCGGCGACGAG
GACmo5uCGGACACCCAAGCCGGACmo5uGmo5uCCCGGGCACmo5uGAm
o5uCCmo5uGACCGGACAAAGACGmo5uCGAAGCGGmo5uAACAACGGGGC
mo5uCmo5uCACGCmo5uCGmo5uCAmo5uCCCCmo5uCGmo5uGGCACGmo
5uCmo5umo5umo5uGCGAGCCmo5umo5uGACGACmo5umo5uGGmo5uACC
Amo5umo5uAACGGmo5uGAGCGmo5uGCAGCACGCCGCAmo5umo5uACGA
CCmo5uACCmo5uCmo5umo5uAmo5uCmo5uGCGCAGCGACAmo5uGGACG
GCGACGmo5uGCGmo5uACCGCGGCAGACAmo5uCAGCAGCACGmo5umo5
uGCGGmo5uCCGmo5uGCCCGCGCCACGACCCmo5uCACCCAmo5uCAGCA
CCGCmo5umo5uCCACmo5umo5uCCAGCACCCCACGCAGmo5uCGACCCC
GCAmo5uCmo5uAGAGAGAGACmo5umo5uCmo5umo5umo5uGmo5umo5um
05umo5umo5uCCCCCGCGmo5uGmo5umo5umo5umo5umo5uCCCAmo5umo
5uCCCmo5uGmo5uAmo5umo5umo5uAmo5umo5umo5uCmo5uAAAmo5uAAm
o5uAAAAACACAGAGACGmo5umo5uGAmo5uAAmo5uAACCGCAGmo5uGm
o5uGCmo5umo5umo5uAmo5umo5uAGGGmo5uAmo5uCACGGmo5uGmo5uA
GAAAAAAAAAAGAGAGGGAAACCCmo5uAAAmo5uAmo5uAGCGmo5uCmo5
uCmo5uC
SEQ ID NO: 24 (pp71 Construct C-Immediate early (IE1) + 5moU)
AGGmo5uCGmo5umo5umo5uAGmo5uGAACCGmo5uCAGAmo5uCGCCmo5u
GGAGACGCCAmo5uCCACGCmo5uGmo5umo5umo5umo5uGACCmo5uCCA
mo5uAGAAGACACCGGGACCGAmo5uCCAGCCmo5uCCGCGGCCGGGAAC
GGmo5uGCAmo5umo5uGGAACGCGGAmo5umo5uCCCCGmo5uGCCAAGA
Gmo5uGACAmo5uGmo5uCmo5uCAGGCAmo5uCGmo5uCCmo5uCGCCCGG
mo5uGAGGGACCCmo5uCGmo5uCGGAAGCGGCCGCGAmo5uCAGCGAGG
CCGAAGCCGCCAGCGGCAGCmo5umo5umo5uGGmo5uCGCCmo5uGCAC
mo5uGCCAGGmo5uGCmo5umo5uCGGCmo5uCAmo5uCACCAACGmo5uGG
AAGGCGGCmo5uCGCmo5uGGAAGCCGGmo5uCGmo5uCmo5uGCGACmo5
uCCmo5uGGACCmo5uGCGmo5uACCAACAmo5uAGAGGmo5uGAGCCGAC
CCmo5uCGGmo5umo5uCmo5uCmo5uGCmo5uGmo5umo5umo5umo5uCAG
GAGAACAAAmo5uCmo5uCCGCACGACACCGmo5uAGACCmo5uGACCGAC
Cmo5uAAACAmo5uCAAGGGCCGCmo5uGCGmo5uGGmo5uGGGCGAACGG
GACCGACmo5uGCmo5uGGmo5uGGACCmo5uCAACAACmo5umo5umo5uG
GCCCACGACGCCmo5uGACGCCAGGCmo5uCAGAAAACAACACGGmo5uC
mo5uCGGmo5uGCmo5uGGCCmo5umo5umo5uGCGCmo5uGCCGCmo5uAG
ACCGCGmo5uGCCCGmo5umo5uAGCGGACmo5uGCACCmo5uCmo5umo5u
mo5uCAGAGCCAGCGGCGCGGCGGCGAAGAAAAmo5uCGGCCGCGAAmo
5uGGAGGCGCGCGCCAmo5uCAmo5uCCGCCGCACGGCmo5uCACCACmo
5uGGGCCGmo5uGCGACmo5uGACCGmo5uGACGCCGAACmo5uGGCGCC
GCAGAACCGACAGCAGmo5umo5umo5uGGAGGCAGGGCAGAmo5uCmo5u
mo5umo5uGmo5uCAGCCAGmo5umo5uCGCCmo5umo5umo5uCGCGCCGG
CGCCAmo5uCCCGCmo5uGACGCmo5uGGmo5uAGACGCCCmo5uGGAGCA
GCmo5uGGCCmo5uGmo5umo5uCGGACCCmo5uAACACGmo5uACAmo5uC
CACAAAACGGAGACGGACGAACGAGGCCAAmo5uGGAmo5uCAmo5uGCm
05uGmo5umo5umo5uCmo5uGCAmo5uCACGACmo5uCACCGCACCCGCCG
ACCAGCGmo5uGmo5umo5umo5uCmo5uGCACmo5umo5umo5umo5uCGGm
05umo5umo5uACACGCAmo5uCGCGCCGAGGmo5uGGmo5uGGCGCGACA
CAAmo5uCCGmo5uACCCGCACCmo5uACGACGCmo5umo5uGCCGGACAA
CGGCmo5umo5uCCAGCmo5uGmo5umo5uGAmo5umo5uCCCAAAAGmo5um
o5umo5umo5uACGCmo5uGACGCGCAmo5uACAmo5uCCCGAGmo5uACAmo
5uCGmo5uGCAGAmo5uCCAGAAmo5uGCmo5umo5umo5uCGAAACCAAmo5
uCAGACmo5uCACGACACCAmo5uCmo5umo5umo5umo5umo5uCCCGGAAA
ACAmo5uCCCGGGCGmo5uCmo5uCCAmo5uAGAAGCCGGCCCGCmo5uAC
CCGAmo5uCGmo5uGmo5uGCGAAmo5uCACCCmo5uCCGCGmo5uCACGC
mo5uGACCGGCGACCAGGCCGmo5umo5uCAmo5umo5umo5uGGAACACC
GACAGCCGCmo5uAGGCCGCAmo5uCCACmo5umo5umo5umo5umo5uCCG
CCGmo5uGGGmo5umo5umo5umo5uGGACmo5uCmo5uCACACCCGGmo5u
AAACCGGACAAAAmo5uCAAGCGmo5uCCCCAGGmo5uGCAGCmo5uGCGC
GCCGGmo5uCmo5uCmo5umo5umo5uCCACGGAGCGACGmo5uCGmo5uGC
GCGGCGCCGmo5uCmo5uCCGAGmo5umo5umo5uCmo5uCCCGCAGmo5uC
CCCCGGAmo5umo5uACCACCCACCGAGGAAGAGGAGGAAGAAGAGGAAG
AGGACGACGAAGAmo5uGACCmo5uCmo5uCCmo5uCCACACCGACGCCGA
CCCCCCmo5uGmo5uCCGAAGCCAmo5uGmo5umo5umo5uGCCGGCmo5um
05uCGAGGAGGCCAGCGGCGACGAGGACmo5uCGGACACCCAAGCCGGA
Cmo5uGmo5uCCCGGGCACmo5uGAmo5uCCmo5uGACCGGACAAAGACGm
o5uCGAAGCGGmo5uAACAACGGGGCmo5uCmo5uCACGCmo5uCGmo5uCA
mo5uCCCCmo5uCGmo5uGGCACGmo5uCmo5umo5umo5uGCGAGCCmo5u
mo5uGACGACmo5umo5uGGmo5uACCAmo5umo5uAACGGmo5uGAGCGmo
5uGCAGCACGCCGCAmo5umo5uACGACCmo5uACCmo5uCmo5umo5uAmo5
uCmo5uGCGCAGCGACAmo5uGGACGGCGACGmo5uGCGmo5uACCGCGG
CAGACAmo5uCAGCAGCACGmo5umo5uGCGGmo5uCCGmo5uGCCCGCGC
CACGACCCmo5uCACCCAmo5uCAGCACCGCmo5umo5uCCACmo5umo5uC
CAGCACCCCACGCAGmo5uCGACCCCGCAmo5uCmo5uAGGCGGCCGCmo
5umo5uAAmo5umo5uAAGCmo5uGCCmo5umo5uCmo5uGCGGGGCmo5umo
5uGCCmo5umo5uCmo5uGGCCAmo5uGCCCmo5umo5uCmo5umo5uCmo5u
Cmo5uCCCmo5umo5uGCACCmo5uGmo5uACCmo5uCmo5umo5uGGmo5uC
mo5umo5umo5uGAAmo5uAAAGCCmo5uGAGmo5uAGGAAG
SEQ ID NO: 25 (pp71 Construct D-short + 5moU)
AGGGCGCCACACCmo5uGmo5uCGCCGCmo5uGCmo5uAmo5uAmo5umo5u
mo5uGCGACAGmo5umo5uGCCGGAACCCmo5umo5uCCCGACCmo5uCCCA
CGAAGACCCGmo5umo5uCACCmo5umo5umo5uGCGCAmo5uCCCCmo5uG
ACCCmo5uCCCCCCAmo5uCCCGCCmo5umo5uCGCAAmo5uGmo5uCmo5u
CAGGCAmo5uCGmo5uCCmo5uCGCCCGGmo5uGAGGGACCCmo5uCGmo5
uCGGAAGCGGCCGCGAmo5uCAGCGAGGCCGAAGCCGCCAGCGGCAGC
mo5umo5umo5uGGmo5uCGCCmo5uGCACmo5uGCCAGGmo5uGCmo5umo5
uCGGCmo5uCAmo5uCACCAACGmo5uGGAAGGCGGCmo5uCGCmo5uGGA
AGCCGGmo5uCGmo5uCmo5uGCGACmo5uCCmo5uGGACCmo5uGCGmo5u
ACCAACAmo5uAGAGGmo5uGAGCCGACCCmo5uCGGmo5umo5uCmo5uC
mo5uGCmo5uGmo5umo5umo5umo5uCAGGAGAACAAAmo5uCmo5uCCGCA
CGACACCGmo5uAGACCmo5uGACCGACCmo5uAAACAmo5uCAAGGGCCG
Cmo5uGCGmo5uGGmo5uGGGCGAACGGGACCGACmo5uGCmo5uGGmo5u
GGACCmo5uCAACAACmo5umo5umo5uGGCCCACGACGCCmo5uGACGCC
AGGCmo5uCAGAAAACAACACGGmo5uCmo5uCGGmo5uGCmo5uGGCCmo
5umo5umo5uGCGCmo5uGCCGCmo5uAGACCGCGmo5uGCCCGmo5umo5u
AGCGGACmo5uGCACCmo5uCmo5umo5umo5uCAGAGCCAGCGGCGCGGC
GGCGAAGAAAAmo5uCGGCCGCGAAmo5uGGAGGCGCGCGCCAmo5uCAm
o5uCCGCCGCACGGCmo5uCACCACmo5uGGGCCGmo5uGCGACmo5uGAC
CGmo5uGACGCCGAACmo5uGGCGCCGCAGAACCGACAGCAGmo5umo5u
mo5uGGAGGCAGGGCAGAmo5uCmo5umo5umo5uGmo5uCAGCCAGmo5um
05uCGCCmo5umo5umo5uCGCGCCGGCGCCAmo5uCCCGCmo5uGACGCm
o5uGGmo5uAGACGCCCmo5uGGAGCAGCmo5uGGCCmo5uGmo5umo5uCG
GACCCmo5uAACACGmo5uACAmo5uCCACAAAACGGAGACGGACGAACGA
GGCCAAmo5uGGAmo5uCAmo5uGCmo5uGmo5umo5umo5uCmo5uGCAmo5
uCACGACmo5uCACCGCACCCGCCGACCAGCGmo5uGmo5umo5umo5uCm
o5uGCACmo5umo5umo5umo5uCGGmo5umo5umo5uACACGCAmo5uCGCG
CCGAGGmo5uGGmo5uGGCGCGACACAAmo5uCCGmo5uACCCGCACCmo5
uACGACGCmo5umo5uGCCGGACAACGGCmo5umo5uCCAGCmo5uGmo5u
mo5uGAmo5umo5uCCCAAAAGmo5umo5umo5umo5uACGCmo5uGACGCGC
Amo5uACAmo5uCCCGAGmo5uACAmo5uCGmo5uGCAGAmo5uCCAGAAmo
5uGCmo5umo5umo5uCGAAACCAAmo5uCAGACmo5uCACGACACCAmo5uC
mo5umo5umo5umo5umo5uCCCGGAAAACAmo5uCCCGGGCGmo5uCmo5uC
CAmo5uAGAAGCCGGCCCGCmo5uACCCGAmo5uCGmo5uGmo5uGCGAAm
05uCACCCmo5uCCGCGmo5uCACGCmo5uGACCGGCGACCAGGCCGmo5u
mo5uCAmo5umo5umo5uGGAACACCGACAGCCGCmo5uAGGCCGCAmo5uC
CACmo5umo5umo5umo5umo5uCCGCCGmo5uGGGmo5umo5umo5umo5uG
GACmo5uCmo5uCACACCCGGmo5uAAACCGGACAAAAmo5uCAAGCGmo5u
CCCCAGGmo5uGCAGCmo5uGCGCGCCGGmo5uCmo5uCmo5umo5umo5uC
CACGGAGCGACGmo5uCGmo5uGCGCGGCGCCGmo5uCmo5uCCGAGmo5
umo5umo5uCmo5uCCCGCAGmo5uCCCCCGGAmo5umo5uACCACCCACCG
AGGAAGAGGAGGAAGAAGAGGAAGAGGACGACGAAGAmo5uGACCmo5uC
mo5uCCmo5uCCACACCGACGCCGACCCCCCmo5uGmo5uCCGAAGCCAmo
5uGmo5umo5umo5uGCCGGCmo5umo5uCGAGGAGGCCAGCGGCGACGAG
GACmo5uCGGACACCCAAGCCGGACmo5uGmo5uCCCGGGCACmo5uGAm
05uCCmo5uGACCGGACAAAGACGmo5uCGAAGCGGmo5uAACAACGGGGC
mo5uCmo5uCACGCmo5uCGmo5uCAmo5uCCCCmo5uCGmo5uGGCACGmo
5uCmo5umo5umo5uGCGAGCCmo5umo5uGACGACmo5umo5uGGmo5uACC
Amo5umo5uAACGGmo5uGAGCGmo5uGCAGCACGCCGCAmo5umo5uACGA
CCmo5uACCmo5uCmo5umo5uAmo5uCmo5uGCGCAGCGACAmo5uGGACG
GCGACGmo5uGCGmo5uACCGCGGCAGACAmo5uCAGCAGCACGmo5umo5
uGCGGmo5uCCGmo5uGCCCGCGCCACGACCCmo5uCACCCAmo5uCAGCA
CCGCmo5umo5uCCACmo5umo5uCCAGCACCCCACGCAGmo5uCGACCCC
GCAmo5uCmo5uAGAGAGAGACmo5umo5uCmo5umo5umo5uGmo5umo5um
o5umo5umo5uCCCCCGCGmo5uGmo5umo5umo5umo5umo5uCCCAmo5umo
5uCCCmo5uGmo5uAmo5umo5umo5uAmo5umo5umo5uCmo5uAAAmo5uAAm
o5uAAAAACACAGAGACGmo5umo5uGAmo5uAAmo5uAACCGCAGCCmo5u
GAGmo5uAGGAAG
SEQ ID NO: 26 (pp71 Construct E-pp65 + 5moU)
AGGACAGAGGACCCmo5uGGmo5uGmo5umo5uGGGAAACGGACACmo5uA
GGCGmo5uCCGCGCGAmo5uACGGGGmo5umo5uAAAACAAAAAAAAmo5u
CGGmo5uGGmo5uGGmo5uGmo5uGmo5uGCmo5uGGGGmo5uGmo5uGGmo
5uGACGGmo5uGGGGCmo5umo5umo5uGCCmo5uCmo5umo5umo5umo5um
o5umo5umo5umo5umo5uGmo5uAAmo5uAAAAAAAGACACmo5uCAAmo5uA
Amo5uCCGCGGmo5umo5uGmo5uCmo5uCmo5uGmo5uGmo5uAGAACGmo5
umo5umo5umo5umo5uAmo5umo5umo5uCGGGmo5umo5uCCGCGmo5umo5
umo5uGGmo5uCGCCmo5uGCCmo5uGmo5uGmo5uAAGGCGGCGGCCGCA
AAGGGCGCGCCGCmo5uCAGmo5uCGCCmo5uACACCCGmo5uACGCGCA
GGCAGCAmo5uGGAGmo5uCGCGCGGmo5uCGCCGmo5umo5uGmo5uCCC
GAAAmo5uGAmo5uAmo5uCCGmo5uACmo5uGGGmo5uCCCAmo5umo5umo
5uCGGGGCACGmo5uGCmo5uGAAAGCCGmo5uGmo5umo5umo5uAGmo5u
CGCGGCGACACGCCGGmo5uGCmo5uGCCGCACGAGACGCGACmo5uCC
mo5uGCAGACGGGmo5uAmo5uCCACGmo5uACGCGmo5uGAGCCAGCCCm
05uCGCmo5uGAmo5uCCmo5uGGmo5uGmo5uCGCAGmo5uACACGCCCGA
Cmo5uCGACGCCAmo5uGCCACCGCGGCGACAAmo5uCAGCmo5uGCAGG
mo5uGCAGCACACGmo5uACmo5umo5umo5uACGGGCAGCGAGGmo5uGG
AGAACGmo5uGmo5uCGGmo5uCAACGmo5uGCACAACCCCACGGGCCGAA
GCAmo5uCmo5uGCCCCAGCCAGGAGCCCAmo5uGmo5uCGAmo5uCmo5uA
mo5uGmo5uGmo5uACGCGCmo5uGCCGCmo5uCAAGAmo5uGCmo5uGAAC
Amo5uCCCCAGCAmo5uCAACGmo5uGCACCACmo5uACCCGmo5uCGGCG
GCCGAGCGCAAACACCGACACCmo5uGCCCGmo5uAGCmo5uGACGCmo5
uGmo5uGAmo5umo5uCACGCGmo5uCGGGCAAGCAGAmo5uGmo5uGGCA
GGCGCGmo5uCmo5uCACGGmo5uCmo5uCGGGACmo5uGGCCmo5uGGAC
GCGmo5uCAGCAGAACCAGmo5uGGAAAGAGCCCGACGmo5uCmo5uACmo
5uACACGmo5uCAGCGmo5umo5uCGmo5uGmo5umo5umo5uCCCACCAAGG
ACGmo5uGGCACmo5uGCGGCACGmo5uGGmo5uGmo5uGCGCGCACGAG
Cmo5uGGmo5umo5umo5uGCmo5uCCAmo5uGGAGAACACGCGCGCAACCA
AGAmo5uGCAGGmo5uGAmo5uAGGmo5uGACCAGmo5uACGmo5uCAAGG
mo5uGmo5uACCmo5uGGAGmo5uCCmo5umo5uCmo5uGCGAGGACGmo5u
GCCCmo5uCCGGCAAGCmo5uCmo5umo5umo5uAmo5uGCACGmo5uCACG
Cmo5uGGGCmo5uCmo5uGACGmo5uGGAAGAGGACCmo5uGACGAmo5uG
ACCCGCAACCCGCAACCCmo5umo5uCAmo5uGCGCCCCCACGAGCGCAA
CGGCmo5umo5umo5uACGGmo5uGmo5umo5uGmo5uGmo5uCCCAAAAAmo
5uAmo5uGAmo5uAAmo5uCAAACCGGGCAAGAmo5uCmo5uCGCACAmo5uC
Amo5uGCmo5uGGAmo5uGmo5uGGCmo5umo5umo5umo5uACCmo5uCACA
CGAGCAmo5umo5umo5umo5uGGGCmo5uGCmo5uGmo5uGmo5uCCCAAG
AGCAmo5uCCCGGGCCmo5uGAGCAmo5uCmo5uCAGGmo5uAACCmo5uG
mo5umo5uGAmo5uGAACGGGCAGCAGAmo5uCmo5umo5uCCmo5uGGAGG
mo5uACAAGCGAmo5uACGCGAGACCGmo5uGGAACmo5uGCGmo5uCAGm
o5uACGAmo5uCCCGmo5uGGCmo5uGCACmo5uCmo5umo5uCmo5umo5um
o5umo5umo5uCGAmo5uAmo5uCGACmo5umo5uGCmo5uGCmo5uGCAGCG
CGGGCCmo5uCAGmo5uACAGCGAGCACCCCACCmo5umo5uCACCAGCCA
Gmo5uAmo5uCGCAmo5uCCAGGGCAAGCmo5umo5uGAGmo5uACCGACAC
ACCmo5uGGGACCGGCACGACGAGGGmo5uGCCGCCCAGGGCGACGACG
ACGmo5uCmo5uGGACCAGCGGAmo5uCGGACmo5uCCGACGAAGAACmo5
uCGmo5uAACCACCGAGCGCAAGACGCCCCGCGmo5umo5uACCGGCGGC
GGCGCCAmo5uGGCGGGCGCCmo5uCCACmo5umo5uCCGCGGGCCGCAA
ACGCAAAmo5uCAGCAmo5uCCmo5uCGGCGACGGCGmo5uGCACGGCGG
GCGmo5umo5uAmo5uGACACGCGGCCGCCmo5umo5uAAGGCCGAGmo5u
CCACCGmo5uCGCGCCCGAAGAGGACACCGACGAGGAmo5umo5uCCGAC
AACGAAAmo5uCCACAAmo5uCCGGCCGmo5uGmo5umo5uCACCmo5uGGC
CGCCCmo5uGGCAGGCCGGCAmo5uCCmo5uGGCCCGCAACCmo5uGGmo
5uGCCCAmo5uGGmo5uGGCmo5uACGGmo5umo5uCAGGGmo5uCAGAAmo
5uCmo5uGAAGmo5uACCAGGAGmo5umo5uCmo5umo5uCmo5uGGGACGC
CAACGACAmo5uCmo5uACCGCAmo5uCmo5umo5uCGCCGAAmo5umo5uG
GAAGGCGmo5uAmo5uGGCAGCCCGCmo5uGCGCAACCCAAACGmo5uCG
CCGCCACCGGCAAGAAGCCCmo5uGCCCGGGCCAmo5uGCAmo5uCGCCm
05uCGACGCCCAAAAAGCAmo5uCGAGGmo5umo5uGAGCCACCCGCCGCG
CACGCGCmo5umo5uAAGACGACmo5uCmo5uAmo5uAAAAACCCACGmo5u
CCACmo5uCAGACACGCGACmo5umo5umo5umo5uGGGCGCCACACCmo5u
Gmo5uCGCCGCmo5uGCmo5uAmo5uAmo5umo5umo5uGCGACAGmo5umo5
uGCCGGAACCCmo5umo5uCCCGACCmo5uCCCACGAAGACCCGmo5umo5
uCACCmo5umo5umo5uGCGCAmo5uCCCCmo5uGACCCmo5uCCCCCCAmo
5uCCCGCCmo5umo5uCGCAAmo5uGmo5uCmo5uCAGGCAmo5uCGmo5uC
Cmo5uCGCCCGGmo5uGAGGGACCCmo5uCGmo5uCGGAAGCGGCCGCGA
mo5uCAGCGAGGCCGAAGCCGCCAGCGGCAGCmo5umo5umo5uGGmo5uC
GCCmo5uGCACmo5uGCCAGGmo5uGCmo5umo5uCGGCmo5uCAmo5uCAC
CAACGmo5uGGAAGGCGGCmo5uCGCmo5uGGAAGCCGGmo5uCGmo5uC
mo5uGCGACmo5uCCmo5uGGACCmo5uGCGmo5uACCAACAmo5uAGAGG
mo5uGAGCCGACCCmo5uCGGmo5umo5uCmo5uCmo5uGCmo5uGmo5umo5
umo5umo5uCAGGAGAACAAAmo5uCmo5uCCGCACGACACCGmo5uAGACC
mo5uGACCGACCmo5uAAACAmo5uCAAGGGCCGCmo5uGCGmo5uGGmo5
uGGGCGAACGGGACCGACmo5uGCmo5uGGmo5uGGACCmo5uCAACAAC
mo5umo5umo5uGGCCCACGACGCCmo5uGACGCCAGGCmo5uCAGAAAAC
AACACGGmo5uCmo5uCGGmo5uGCmo5uGGCCmo5umo5umo5uGCGCmo5
uGCCGCmo5uAGACCGCGmo5uGCCCGmo5umo5uAGCGGACmo5uGCACC
mo5uCmo5umo5umo5uCAGAGCCAGCGGCGCGGCGGCGAAGAAAAmo5uC
GGCCGCGAAmo5uGGAGGCGCGCGCCAmo5uCAmo5uCCGCCGCACGGC
mo5uCACCACmo5uGGGCCGmo5uGCGACmo5uGACCGmo5uGACGCCGAA
Cmo5uGGCGCCGCAGAACCGACAGCAGmo5umo5umo5uGGAGGCAGGGC
AGAmo5uCmo5umo5umo5uGmo5uCAGCCAGmo5umo5uCGCCmo5umo5um
05uCGCGCCGGCGCCAmo5uCCCGCmo5uGACGCmo5uGGmo5uAGACGCC
Cmo5uGGAGCAGCmo5uGGCCmo5uGmo5umo5uCGGACCCmo5uAACACG
mo5uACAmo5uCCACAAAACGGAGACGGACGAACGAGGCCAAmo5uGGAm
o5uCAmo5uGCmo5uGmo5umo5umo5uCmo5uGCAmo5uCACGACmo5uCAC
CGCACCCGCCGACCAGCGmo5uGmo5umo5umo5uCmo5uGCACmo5umo5u
mo5umo5uCGGmo5umo5umo5uACACGCAmo5uCGCGCCGAGGmo5uGGmo
5uGGCGCGACACAAmo5uCCGmo5uACCCGCACCmo5uACGACGCmo5umo
5uGCCGGACAACGGCmo5umo5uCCAGCmo5uGmo5umo5uGAmo5umo5uC
CCAAAAGmo5umo5umo5umo5uACGCmo5uGACGCGCAmo5uACAmo5uCC
CGAGmo5uACAmo5uCGmo5uGCAGAmo5uCCAGAAmo5uGCmo5umo5umo5
uCGAAACCAAmo5uCAGACmo5uCACGACACCAmo5uCmo5umo5umo5umo5
umo5uCCCGGAAAACAmo5uCCCGGGCGmo5uCmo5uCCAmo5uAGAAGCC
GGCCCGCmo5uACCCGAmo5uCGmo5uGmo5uGCGAAmo5uCACCCmo5uC
CGCGmo5uCACGCmo5uGACCGGCGACCAGGCCGmo5umo5uCAmo5umo5
umo5uGGAACACCGACAGCCGCmo5uAGGCCGCAmo5uCCACmo5umo5um
05umo5umo5uCCGCCGmo5uGGGmo5umo5umo5umo5uGGACmo5uCmo5u
CACACCCGGmo5uAAACCGGACAAAAmo5uCAAGCGmo5uCCCCAGGmo5u
GCAGCmo5uGCGCGCCGGmo5uCmo5uCmo5umo5umo5uCCACGGAGCGA
CGmo5uCGmo5uGCGCGGCGCCGmo5uCmo5uCCGAGmo5umo5umo5uCmo
5uCCCGCAGmo5uCCCCCGGAmo5umo5uACCACCCACCGAGGAAGAGGA
GGAAGAAGAGGAAGAGGACGACGAAGAmo5uGACCmo5uCmo5uCCmo5uC
CACACCGACGCCGACCCCCCmo5uGmo5uCCGAAGCCAmo5uGmo5umo5u
mo5uGCCGGCmo5umo5uCGAGGAGGCCAGCGGCGACGAGGACmo5uCGG
ACACCCAAGCCGGACmo5uGmo5uCCCGGGCACmo5uGAmo5uCCmo5uGA
CCGGACAAAGACGmo5uCGAAGCGGmo5uAACAACGGGGCmo5uCmo5uCA
CGCmo5uCGmo5uCAmo5uCCCCmo5uCGmo5uGGCACGmo5uCmo5umo5u
mo5uGCGAGCCmo5umo5uGACGACmo5umo5uGGmo5uACCAmo5umo5uAA
CGGmo5uGAGCGmo5uGCAGCACGCCGCAmo5umo5uACGACCmo5uACCm
o5uCmo5umo5uAmo5uCmo5uGCGCAGCGACAmo5uGGACGGCGACGmo5u
GCGmo5uACCGCGGCAGACAmo5uCAGCAGCACGmo5umo5uGCGGmo5uC
CGmo5uGCCCGCGCCACGACCCmo5uCACCCAmo5uCAGCACCGCmo5umo
5uCCACmo5umo5uCCAGCACCCCACGCAGmo5uCGACCCCGCAmo5uCmo
5uAGAGAGAGACmo5umo5uCmo5umo5umo5uGmo5umo5umo5umo5umo5u
CCCCCGCGmo5uGmo5umo5umo5umo5umo5uCCCAmo5umo5uCCCmo5uG
mo5uAmo5umo5umo5uAmo5umo5umo5uCmo5uAAAmo5uAAmo5uAAAAACA
CAGAGACGmo5umo5uGAmo5uAAmo5uAACCGCAGmo5uGmo5uGCmo5um
05umo5uAmo5umo5uAGGGmo5uAmo5uCACGGmo5uGmo5uAGAAAAAAAA
AGAGAGGGAAACCCmo5uAAAmo5uAmo5uAGCGmo5uCmo5uCmo5uCAGC
Cmo5uGAGmo5uAGGAAG
SEQ ID NO: 27 (pp71 Construct F-stop pp65 + 5moU)
AGGACAGAGGACCCmo5uGGmo5uGmo5umo5uGGGAAACGGACACmo5uA
GGCGmo5uCCGCGCGAmo5uACGGGGmo5umo5uAAAAAAAAAAAAmo5u
CGGmo5uGGmo5uGGmo5uGmo5uGmo5uGCmo5uGGGGmo5uGmo5uGGmo
5uGACGGmo5uGGGGCmo5umo5umo5uGCCmo5uCmo5umo5umo5umo5um
o5umo5umo5umo5umo5uGmo5uAAmo5uAAAAAAAGACACmo5uCAAmoSuA
Amo5uCCGCGGmo5umo5uGmo5uCmo5uCmo5uGmo5uGmo5uAGAACGmo5
umo5umo5umo5umo5uAmo5umo5umo5uCGGGmo5umo5uCCGCGmo5umo5
umo5uGGmo5uCGCCmo5uGCCmo5uGmo5uGmo5uAAGGCGGCGGCCGCA
AAGGGCGCGCCGCmo5uCAGmo5uCGCCmo5uACACCCGmo5uACGCGCA
GGCAGCAmo5uGGAGmo5uCGCGCGGmo5uCGCCGmo5umo5uGmo5uCCC
GAAmo5uAGmo5uGAmo5uCCGmo5uACmo5uGGGmo5uCCCAmo5umo5umo
5uCGGGGCACGmo5uGCmo5uGAAAGCCGmo5uGmo5umo5umo5uAGmo5u
CGCGGCGACACGCCGGmo5uGCmo5uGCCGCACGAGACGCGACmo5uCC
mo5uGCAGACGGGmo5uAmo5uCCACGmo5uACGCGmo5uGAGCCAGCCCm
o5uCGCmo5uGAmo5uCCmo5uGGmo5uGmo5uCGCAGmo5uACACGCCCGA
Cmo5uCGACGCCAmo5uGCCACCGCGGCGACAAmo5uCAGCmo5uGCAGG
mo5uGCAGCACACGmo5uACmo5umo5umo5uACGGGCAGCGAGGmo5uGG
AGAACGmo5uGmo5uCGGmo5uCAACGmo5uGCACAACCCCACGGGCCGAA
GCAmo5uCmo5uGCCCCAGCCAGGAGCCCAmo5uGmo5uCGAmo5uCmo5uA
mo5uGmo5uGmo5uACGCGCmo5uGCCGCmo5uCAAGAmo5uGCmo5uGAAC
Amo5uCCCCAGCAmo5uCAACGmo5uGCACCACmo5uACCCGmo5uCGGCG
GCCGAGCGCAAACACCGACACCmo5uGCCCGmo5uAGCmo5uGACGCmo5
uGmo5uGAmo5umo5uCACGCGmo5uCGGGCAAGCAGAmo5uGmo5uGGCA
GGCGCGmo5uCmo5uCACGGmo5uCmo5uCGGGACmo5uGGCCmo5uGGAC
GCGmo5uCAGCAGAACCAGmo5uGGAAAGAGCCCGACGmo5uCmo5uACmo
5uACACGmo5uCAGCGmo5umo5uCGmo5uGmo5umo5umo5uCCCACCAAGG
ACGmo5uGGCACmo5uGCGGCACGmo5uGGmo5uGmo5uGCGCGCACGAG
Cmo5uGGmo5umo5umo5uGCmo5uCCAmo5uGGAGAACACGCGCGCAACCA
AGAmo5uGCAGGmo5uGAmo5uAGGmo5uGACCAGmo5uACGmo5uCAAGG
mo5uGmo5uACCmo5uGGAGmo5uCCmo5umo5uCmo5uGCGAGGACGmo5u
GCCCmo5uCCGGCAAGCmo5uCmo5umo5umo5uAmo5uGCACGmo5uCACG
Cmo5uGGGCmo5uCmo5uGACGmo5uGGAAGAGGACCmo5uGACGAmo5uG
ACCCGCAACCCGCAACCCmo5umo5uCAmo5uGCGCCCCCACGAGCGCAA
CGGCmo5umo5umo5uACGGmo5uGmo5umo5uGmo5uGmo5uCCCAAAAAmo
5uAmo5uGAmo5uAAmo5uCAAACCGGGCAAGAmo5uCmo5uCGCACAmo5uC
Amo5uGCmo5uGGAmo5uGmo5uGGCmo5umo5umo5umo5uACCmo5uCACA
CGAGCAmo5umo5umo5umo5uGGGCmo5uGCmo5uGmo5uGmo5uCCCAAG
AGCAmo5uCCCGGGCCmo5uGAGCAmo5uCmo5uCAGGmo5uAACCmo5uG
mo5umo5uGAmo5uGAACGGGCAGCAGAmo5uCmo5umo5uCCmo5uGGAGG
mo5uACAAGCGAmo5uACGCGAGACCGmo5uGGAACmo5uGCGmo5uCAGm
o5uACGAmo5uCCCGmo5uGGCmo5uGCACmo5uCmo5umo5uCmo5umo5um
o5umo5umo5uCGAmo5uAmo5uCGACmo5umo5uGCmo5uGCmo5uGCAGCG
CGGGCCmo5uCAGmo5uACAGCGAGCACCCCACCmo5umo5uCACCAGCCA
Gmo5uAmo5uCGCAmo5uCCAGGGCAAGCmo5umo5uGAGmo5uACCGACAC
ACCmo5uGGGACCGGCACGACGAGGGmo5uGCCGCCCAGGGCGACGACG
ACGmo5uCmo5uGGACCAGCGGAmo5uCGGACmo5uCCGACGAAGAACmo5
uCGmo5uAACCACCGAGCGCAAGACGCCCCGCGmo5umo5uACCGGCGGC
GGCGCCAmo5uGGCGGGCGCCmo5uCCACmo5umo5uCCGCGGGCCGCAA
ACGCAAAmo5uCAGCAmo5uCCmo5uCGGCGACGGCGmo5uGCACGGCGG
GCGmo5umo5uAmo5uGACACGCGGCCGCCmo5umo5uAAGGCCGAGmo5u
CCACCGmo5uCGCGCCCGAAGAGGACACCGACGAGGAmo5umo5uCCGAC
AACGAAAmo5uCCACAAmo5uCCGGCCGmo5uGmo5umo5uCACCmo5uGGC
CGCCCmo5uGGCAGGCCGGCAmo5uCCmo5uGGCCCGCAACCmo5uGGmo
5uGCCCAmo5uGGmo5uGGCmo5uACGGmo5umo5uCAGGGmo5uCAGAAmo
5uCmo5uGAAGmo5uACCAGGAGmo5umo5uCmo5umo5uCmo5uGGGACGC
CAACGACAmo5uCmo5uACCGCAmo5uCmo5umo5uCGCCGAAmo5umo5uG
GAAGGCGmo5uAmo5uGGCAGCCCGCmo5uGCGCAACCCAAACGmo5uCG
CCGCCACCGGCAAGAAGCCCmo5uGCCCGGGCCAmo5uGCAmo5uCGCCm
o5uCGACGCCCAAAAAGCAmo5uCGAGGmo5umo5uGAGCCACCCGCCGCG
CACGCGCmo5umo5uAAGACGACmo5uCmo5uAmo5uAAAAACCCACGmo5u
CCACmo5uCAGACACGCGACmo5umo5umo5umo5uGGGCGCCACACCmo5u
Gmo5uCGCCGCmo5uGCmo5uAmo5uAmo5umo5umo5uGCGACAGmo5umo5
uGCCGGAACCCmo5umo5uCCCGACCmo5uCCCACGAAGACCCGmo5umo5
uCACCmo5umo5umo5uGCGCAmo5uCCCCmo5uGACCCmo5uCCCCCCAmo
5uCCCGCCmo5umo5uCGCAAmo5uGmo5uCmo5uCAGGCAmo5uCGmo5uC
Cmo5uCGCCCGGmo5uGAGGGACCCmo5uCGmo5uCGGAAGCGGCCGCGA
mo5uCAGCGAGGCCGAAGCCGCCAGCGGCAGCmo5umo5umo5uGGmo5uC
GCCmo5uGCACmo5uGCCAGGmo5uGCmo5umo5uCGGCmo5uCAmo5uCAC
CAACGmo5uGGAAGGCGGCmo5uCGCmo5uGGAAGCCGGmo5uCGmo5uC
mo5uGCGACmo5uCCmo5uGGACCmo5uGCGmo5uACCAACAmo5uAGAGG
mo5uGAGCCGACCCmo5uCGGmo5umo5uCmo5uCmo5uGCmo5uGmo5umo5
umo5umo5uCAGGAGAACAAAmo5uCmo5uCCGCACGACACCGmo5uAGACC
mo5uGACCGACCmo5uAAACAmo5uCAAGGGCCGCmo5uGCGmo5uGGmo5
uGGGCGAACGGGACCGACmo5uGCmo5uGGmo5uGGACCmo5uCAACAAC
mo5umo5umo5uGGCCCACGACGCCmo5uGACGCCAGGCmo5uCAGAAAAC
AACACGGmo5uCmo5uCGGmo5uGCmo5uGGCCmo5umo5umo5uGCGCmo5
uGCCGCmo5uAGACCGCGmo5uGCCCGmo5umo5uAGCGGACmo5uGCACC
mo5uCmo5umo5umo5uCAGAGCCAGCGGCGCGGCGGCGAAGAAAAmo5uC
GGCCGCGAAmo5uGGAGGCGCGCGCCAmo5uCAmo5uCCGCCGCACGGC
mo5uCACCACmo5uGGGCCGmo5uGCGACmo5uGACCGmo5uGACGCCGAA
Cmo5uGGCGCCGCAGAACCGACAGCAGmo5umo5umo5uGGAGGCAGGGC
AGAmo5uCmo5umo5umo5uGmo5uCAGCCAGmo5umo5uCGCCmo5umo5um
o5uCGCGCCGGCGCCAmo5uCCCGCmo5uGACGCmo5uGGmo5uAGACGCC
Cmo5uGGAGCAGCmo5uGGCCmo5uGmo5umo5uCGGACCCmo5uAACACG
mo5uACAmo5uCCACAAAACGGAGACGGACGAACGAGGCCAAmo5uGGAm
o5uCAmo5uGCmo5uGmo5umo5umo5uCmo5uGCAmo5uCACGACmo5uCAC
CGCACCCGCCGACCAGCGmo5uGmo5umo5umo5uCmo5uGCACmo5umo5u
mo5umo5uCGGmo5umo5umo5uACACGCAmo5uCGCGCCGAGGmo5uGGmo
5uGGCGCGACACAAmo5uCCGmo5uACCCGCACCmo5uACGACGCmo5umo
5uGCCGGACAACGGCmo5umo5uCCAGCmo5uGmo5umo5uGAmo5umo5uC
CCAAAAGmo5umo5umo5umo5uACGCmo5uGACGCGCAmo5uACAmo5uCC
CGAGmo5uACAmo5uCGmo5uGCAGAmo5uCCAGAAmo5uGCmo5umo5umo5
uCGAAACCAAmo5uCAGACmo5uCACGACACCAmo5uCmo5umo5umo5umo5
umo5uCCCGGAAAACAmo5uCCCGGGCGmo5uCmo5uCCAmo5uAGAAGCC
GGCCCGCmo5uACCCGAmo5uCGmo5uGmo5uGCGAAmo5uCACCCmo5uC
CGCGmo5uCACGCmo5uGACCGGCGACCAGGCCGmo5umo5uCAmo5umo5
umo5uGGAACACCGACAGCCGCmo5uAGGCCGCAmo5uCCACmo5umo5um
o5umo5umo5uCCGCCGmo5uGGGmo5umo5umo5umo5uGGACmo5uCmo5u
CACACCCGGmo5uAAACCGGACAAAAmo5uCAAGCGmo5uCCCCAGGmo5u
GCAGCmo5uGCGCGCCGGmo5uCmo5uCmo5umo5umo5uCCACGGAGCGA
CGmo5uCGmo5uGCGCGGCGCCGmo5uCmo5uCCGAGmo5umo5umo5uCmo
5uCCCGCAGmo5uCCCCCGGAmo5umo5uACCACCCACCGAGGAAGAGGA
GGAAGAAGAGGAAGAGGACGACGAAGAmo5uGACCmo5uCmo5uCCmo5uC
CACACCGACGCCGACCCCCCmo5uGmo5uCCGAAGCCAmo5uGmo5umo5u
mo5uGCCGGCmo5umo5uCGAGGAGGCCAGCGGCGACGAGGACmo5uCGG
ACACCCAAGCCGGACmo5uGmo5uCCCGGGCACmo5uGAmo5uCCmo5uGA
CCGGACAAAGACGmo5uCGAAGCGGmo5uAACAACGGGGCmo5uCmo5uCA
CGCmo5uCGmo5uCAmo5uCCCCmo5uCGmo5uGGCACGmo5uCmo5umo5u
mo5uGCGAGCCmo5umo5uGACGACmo5umo5uGGmo5uACCAmo5umo5uAA
CGGmo5uGAGCGmo5uGCAGCACGCCGCAmo5umo5uACGACCmo5uACCm
o5uCmo5umo5uAmo5uCmo5uGCGCAGCGACAmo5uGGACGGCGACGmo5u
GCGmo5uACCGCGGCAGACAmo5uCAGCAGCACGmo5umo5uGCGGmo5uC
CGmo5uGCCCGCGCCACGACCCmo5uCACCCAmo5uCAGCACCGCmo5umo
5uCCACmo5umo5uCCAGCACCCCACGCAGmo5uCGACCCCGCAmo5uCmo
SuAGAGAGAGACmo5umo5uCmo5umo5umo5uGmo5umo5umo5umo5umo5u
CCCCCGCGmo5uGmo5umo5umo5umo5umo5uCCCAmo5umo5uCCCmo5uG
mo5uAmo5umo5umo5uAmo5umo5umo5uCmo5uAAAmo5uAAmo5uAAAAACA
CAGAGACGmo5umo5uGAmo5uAAmo5uAACCGCAGmo5uGmo5uGCmo5um
o5umo5uAmo5umo5uAGGGmo5uAmo5uCACGGmo5uGmo5uAGAAAAAAAA
AGAGAGGGAAACCCmo5uAAAmo5uAmo5uAGCGmo5uCmo5uCmo5uCAGC
Cmo5uGAGmo5uAGGAAG

While specific embodiments have been illustrated and described, it will be readily appreciated that the various embodiments described above can be combined to provide further embodiments, and the various embodiments described above can be combined to provide further embodiments.

All of the U.S. patents, U.S. patent application publications, U.S. patent applications, foreign patents, foreign patent applications, and non-patent publications referred to in this specification and/or listed in the Application Data Sheet, including U.S. Provisional Patent Applications No. 63/239,269 filed Aug. 31, 2021 is incorporated herein by reference, in its entirety, unless explicitly stated otherwise. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of producing a progeny cytomegalovirus (CMV), comprising:
(a) introducing to a cell a mRNA molecule encoding a gene that is essential for or augments CMV replication;
(b) infecting the cell with a parent CMV;
(c) incubating the cell; and
(d) collecting the progeny CMV.

2. (canceled)

3. (canceled)

4. The method of claim 1, wherein the gene that is essential for or augments CMV replication is UL82, UL32, UL34, UL37, UL44, UL46, UL48, UL48.5, UL49, UL50, UL51, UL52, UL53, UL54, UL55, UL56, UL57, UL60, UL61, UL70, UL71, UL73, UL75, UL76, UL77, UL79, UL80, UL84, UL85, UL86, UL87, UL89, UL90, UL91, UL92, UL93, UL94, UL95, UL96, UL98, UL99, UL100, UL102, UL10⁴, UL10⁵, UL115, or UL122, or a homolog thereof.

5. The method of claim 1, wherein the progeny CMV comprises pp71 protein.

6. The method of claim 1, wherein the cell is an MRC-5 cell.

7. (canceled)

8. The method of claim 1, wherein the mRNA molecule comprises the sequence according to any one of SEQ ID NOs: 14-20, 4-10, or 21-27.

9.-13. (canceled)

14. The method of claim 1, wherein the mRNA molecule further comprises a poly(A) tail.

15. (canceled)

16. (canceled)

17. The method of claim 14, wherein the mRNA molecule was produced using a double-stranded DNA template encoding the poly(A) tail, wherein the double-stranded DNA template is a plasmid.

18.-20. (canceled)

21. The method of claim 1, wherein the mRNA molecule comprises the sequence according to one of SEQ ID NOs: 4-10, wherein each uridine is substituted with pseudouridine and each cytidine is substituted with 5-methylcytidine, and has a poly(A) tail 80 nucleotides in length; and wherein the poly(A) tail was produced using a plasmid template.

22.-25. (canceled)

26. The method of claim 1, wherein the mRNA molecule is introduced using transfection.

27. The method of claim 26, wherein the transfection is accomplished using a lipid transfection reagent.

28.-32. (canceled)

33. The method of claim 1, wherein the parent or progeny CMV is a HCMV.

34. The method of claim 33, wherein the parent or progeny CMV is a genetically modified TR strain of HCMV and optionally, comprises a TR3 backbone.

35. (canceled)

36. The method of claim 1, wherein the parent or progeny CMV comprises a nucleic acid encoding a heterologous antigen.

37. The method of claim 36, where in the heterologous antigen comprises a pathogen-specific antigen or a tumor antigen.

38. The method of claim 36, wherein the heterologous antigen comprises a pathogen-specific antigen comprising a human immunodeficiency virus (HIV) antigen, a simian immunodeficiency virus (SIV) antigen, a human cytomegalovirus (HCMV) antigen, a hepatitis B virus (HBV) antigen, a hepatitis C virus (HCV) antigen, a papilloma virus antigen (e.g., a human papilloma virus (HPV) antigen), a Plasmodium antigen, a Kaposi's sarcoma-associated herpesvirus antigen, a Varicella zoster virus (VZV) antigen, an Ebola virus, a Mycobacterium tuberculosis antigen, a Chikungunya virus antigen, a dengue virus antigen, a monkeypox virus antigen, a herpes simplex virus (HSV) 1 antigen, a herpes simplex virus (HSV) 2 antigen, an Epstein-Barr virus (EBV) antigen, a poliovirus antigen, an influenza virus antigen, or a Clostridium tetani antigen.

39.-42. (canceled)

43. The method of claim 38, wherein the pathogen-specific antigen is an HIV antigen and comprises SEQ ID NO: 11 or 12.

44.-47. (canceled)

48. The method of claim 38, wherein the pathogen-specific antigen is a Mycobacterium tuberculosis antigen and comprises SEQ ID NO:13.

49. The method of claim 36, wherein the heterologous antigen comprises a prostate cancer antigen.

50. The method of claim 1, wherein the parent or progeny CMV does not express an active UL128, UL130, UL146, UL147, UL82, or UL18, or homologs thereof.

51. (canceled)

52. The method of claim 50, wherein the parent or progeny CMV does not express an active UL128 or homolog thereof, does not express an active UL130 or homolog thereof, does not express an active UL146 or homolog thereof, does not express an active UL147 or homolog thereof, and does not express an active UL82 or homolog thereof.

53.-56. (canceled)

57. The method of claim 50, wherein the parent or progeny CMV further comprises a nucleic acid sequence encoding a microRNA (miRNA) recognition element (MRE), wherein the MRE contains a target site for a miRNA expressed in endothelial cells or myeloid cells.

58. The method of claim 50, wherein the parent or progeny CMV does not express an active UL82 or homolog thereof.

59. (canceled)

60. The method of claim 50, wherein the parent or progeny CMV comprises a deletion of UL128 or homolog thereof, a deletion of UL130 or homolog thereof, a deletion of UL146 or homolog thereof, a deletion of UL147 or homolog thereof, and a deletion of UL82 or homolog thereof, wherein the parent or progeny CMV further comprises a nucleic acid sequence encoding a microRNA (miRNA) recognition element (MRE), wherein the MRE contains a target site for a miRNA expressed in endothelial cells or myeloid cells.

61. (canceled)

62. The method of claim 36, wherein the nucleic acid encoding the heterologous antigen replaces UL128, UL130, UL146, UL147, UL82, or UL18, or homologs thereof.

63. (canceled)

64. A progeny CMV produced by the method of claim 1.

65. A mRNA molecule comprising the nucleotide sequence of SEQ ID NO: 14-20.

66. A mRNA molecule comprising the nucleotide sequence of SEQ ID NO: 4-10.

67.-81. (canceled)
